(19) United States
(12) Patent Application Publication
(10) Pub. No.: US 2022/0265842 A1
(43) Pub. Date: Aug. 25, 2022

(54) ANTI-CD40 ANTIBODY DRUG CONJUGATES

(71) Applicant: AbbVie Inc., North Chicago, IL (US)

(72) Inventors: Adrian D. Hobson, Shrewsbury, MA (US); Jason Z. Oh, Shrewsbury, MA (US); Michael J. McPherson, Ashby, MA (US); Wendy Waegell, Brookfield, MA (US); Shaughn H. Bryant, Worcester, MA (US); Axel Hernandez, Jr., Charlton, MA (US); Claire L. Ihle, Denver, CO (US); Christopher C. Marvin, Grayslake, IL (US); Olivia A. Perng, Dublin, CA (US); Ling C. Santora, Shrewsbury, MA (US); Lu Wang, Northborough, MA (US); Lu Wang, Westborough, MA (US)

(73) Assignee: AbbVie Inc., North Chicago, IL (US)

(21) Appl. No.: 16/768,616

(22) PCT Filed: Nov. 29, 2018
(86) PCT No.: PCT/IB2018/059480

§ 371 Date: May 29, 2020

Related U.S. Application Data
(60) Provisional application No. 62/593,807, filed on Dec. 1, 2017.
(60) Provisional application No. 62/595,045, filed on Dec. 5, 2017.

(51) Int. Cl. A61K 47/68; A61P 37/00; C07K 16/28

(52) U.S. Cl. CPC A61K 47/6803; A61K 47/6849; A61K 2039/505; C07K 16/2878; A61P 37/00

(57) ABSTRACT

Provided herein are anti-CD40 antibody drug conjugates comprising a radical of Formula (I), wherein R¹, R², and R³ are as defined herein. Further provided are anti-CD40 antibody drug conjugates of Formula (II), wherein Z, R, AA1, AA2, AA3, m, p, q, n, w, R¹, R², and R³ are as defined herein. Further provided are pharmaceutical compositions and kits thereof, and methods of using same.

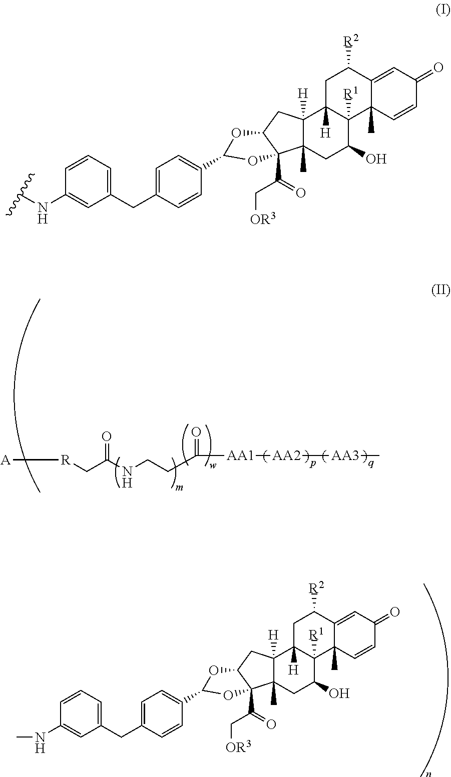

### RELATED APPLICATIONS

The present application is submitted under 35 U.S.C. § 371 as a U.S. National Stage filing of International Application No. PCT/IB2018/059480, filed on Nov. 29, 2018, which claims priority to U.S. Provisional Application No. 62/593,807, filed Dec. 1, 2017, and U.S. Provisional Application No. 62/595,045, filed Dec. 5, 2017. The entire contents of the aforementioned applications are hereby incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 29, 2018, is named A103017_1480 WO_SL.txt and is 13,520 bytes in size.

### BACKGROUND

CD40 is a 48 kDa type I transmembrane protein (van Kooten, J Leukoc Biol. 2000 January; 67(1):2-17) that is expressed on a wide range of hematopoietic (lymphocytes, monocytes, dendritic) and non-hematopoietic (epithelium, endothelium, fibroblasts) cell types. CD40 is a tumor necrosis factor (TNF) receptor family member that plays an important role in B cell development, lymphocyte activation, and antigen presenting cell (APC) function.

CD40/CD40L signaling pathway has been implicated in the pathogenesis of many autoimmune diseases including systemic lupus erythematosus (SLE), inflammatory bowel disease (IBD), multiple sclerosis, rheumatoid arthritis, and Sjogren's syndrome (Law and Grewal, Adv Exp Med Biol. 2009; 647:8-36). CD40 expression is elevated on macrophages, endothelium, epithelium, and B cells in tissues damaged by chronic autoimmunity including kidney, intestine, and joints (Borcherding, Am J Pathol. 2010 April; 176(4):1816-27; Sawada-Hase, Am J Gastroenterol. 2000 June; 95(6):1516-23). Soluble CD40L is elevated in subjects suffering from SLE, IBD, and Sjogren's syndrome consistent with inflammatory burden in these subjects.

Some of the earliest evidence the CD40/CD40L pathway in chronic intestinal inflammation came from preclinical models where anti-CD40L mAbs protected rodents from experimental colitis (de Jong, Gastroenterology. 2000 September; 119(3):715-23; Liu, J Immunol. 2000 Jun. 1; 164(11):6005-14; Stuber, J Exp Med 1996 Feb. 1, 183(2):693-8). Reduction in disease activity scores were associated with reduced pro-inflammatory cytokine production in the gut and protection from chronic body weight loss. Similar results were observed in animals that were genetically deficient for CD40 or CD40L (de Jong, Gastroenterology. 2000 September; 119(3):715-23). Treatment of mice with anti-CD40L mAbs after disease onset is still effective in reducing disease activity suggesting that this pathway is critical for maintenance of chronic inflammatory disease. In addition, CD40 agonist antibodies are sufficient to drive intestinal inflammation in mice that lack lymphocytes (Uhlig, Immunity. 2006 August; 25(2):309-18). More recent data using CD40 siRNA also point to an important role for CD40 signaling in colitis (Arranz, J Control Release. 2013 Feb. 10; 165(3):163-72). In Crohn's disease, lamina propria monocytes and epithelium express high levels of CD40 and CD40+ monocytes are enriched in peripheral blood. Furthermore, polymorphisms in the CD40 locus have been linked to increased susceptibility to IBD. In Crohn's subjects treated with anti-TNF antibodies, transcriptional profiling indicates that CD40 mRNA levels decrease in subjects with an adequate drug treatment response. However, in subjects with a poor response to TNF inhibitors, CD40 mRNA levels are unchanged suggesting that CD40-dependent, TNF-independent, pathways may promote inflammation in these subjects. Studies suggest that inhibition of CD40 mediated signaling is important in the pathogenesis of IBD as well as other autoimmune diseases.

There continues to remain a need, however, for new CD40 antagonists useful in the treatment of various inflammatory and autoimmune conditions.

### SUMMARY

In one aspect, the present disclosure provides an antibody drug conjugate comprising: (a) an anti-CD40 antibody comprising complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12; and (b) a radical of a glucocorticoid receptor agonist of Formula (I):

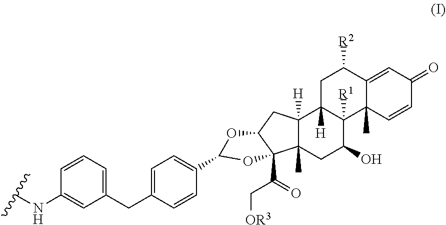

wherein:

R¹ is hydrogen or fluoro;

R² is hydrogen or fluoro; and

R³ is hydrogen or —P(═O)(OH)₂;

further wherein the antibody is conjugated to the glucocorticoid receptor agonist via a linker represented by the following formula:

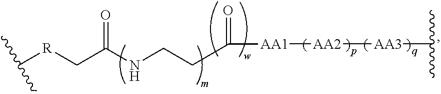

wherein R is a bond,

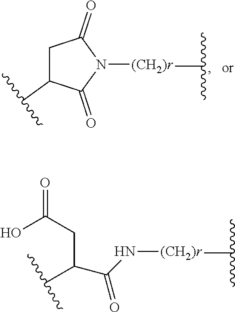

and r is 0 or 1; AA1, AA2, and AA3 are independently selected from the group consisting of Alanine (Ala), Glycine (Gly), Isoleucine (Ile), Leucine (Leu), Proline (Pro), Valine (Val), Phenylalanine (Phe), Tryptophan (Trp), Tyrosine (Tyr), Aspartic acid (Asp), Glutamic acid (Glu), Arginine (Arg), Histidine (His), Lysine (Lys), Serine (Ser), Threonine (Thr), Cysteine (Cys), Methionine (Met), Asparagine (Asn), and Glutamine (Gln);
m is 0 or 1;
w is 0 or 1;
p is 0 or 1; and
q is 0 or 1.

In another embodiment, the present disclosure provides an antibody drug conjugate according to Formula (II):

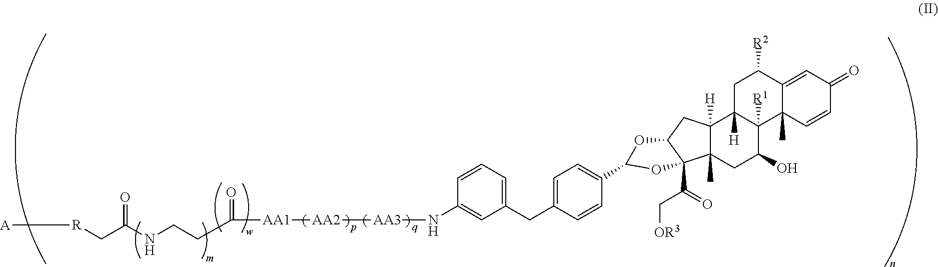

wherein A is the anti-CD40 antibody and n is 1, 2, 3, 4, 5, 6, 7, 8, 9, or 10.

In certain embodiments, n is 2, 4, 6, or 8. In certain embodiments, n is 2. In certain embodiments, n is 4.

In one embodiment, the present disclosure provides an antibody drug conjugate according to any preceding embodiment, wherein R¹ is hydrogen and R² is hydrogen. In one embodiment, the present disclosure provides an antibody drug conjugate according to any preceding embodiment, wherein R¹ is fluoro and R² is hydrogen. In one embodiment, the present disclosure provides an antibody drug conjugate according to any preceding embodiment, wherein R¹ is fluoro and R² is fluoro. In one embodiment, the present disclosure provides an antibody drug conjugate, wherein R³ is —P(═O)(OH)₂. In another embodiment, the present disclosure provides an antibody drug conjugate where R³ is hydrogen.

In one embodiment, the present disclosure provides an antibody drug conjugate according to any preceding embodiment, wherein -AA1-(AA2)ₚ-(AA3)_{q}- is selected from the group consisting of: -Gly-Glu-; -Ala-Ala-; -Glu-Ala-Ala-; -Gly-Lys-; -Glu-; -Glu-Ser-Lys-; and -Gly-Ser-Lys-. In certain embodiments, AA1-(AA2)ₚ-(AA3)_{q} is selected from the group consisting of -Gly-Glu-; -Gly-Lys-; -Glu-Ser-Lys-; and -Gly-Ser-Lys-. In certain embodiments, AA1-(AA2)ₚ-(AA3)_{q} is -Gly-Glu- or -Gly-Lys-. In certain embodiments, AA1-(AA2)ₚ-(AA3)_{q} is -Glu-Ser-Lys- or -Gly-Ser-Lys-.

In one embodiment, the present disclosure provides an antibody drug conjugate according to any preceding embodiment, wherein m is 0; q is 0; and
R is

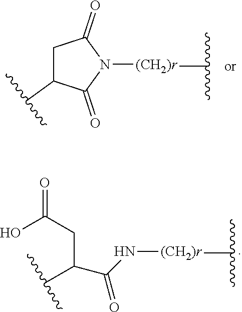

In one embodiment, the present disclosure provides an antibody drug conjugate according to any preceding embodiment, wherein m is 0 or 1; p is 1; and R is a bond.

In one embodiment, the present disclosure provides an antibody drug conjugate according to any preceding embodiment, wherein m is 1; w is 1; and q is 0.

In one embodiment, the present disclosure provides an antibody drug conjugate according to any preceding embodiment, wherein m is 0.

In certain embodiments, the present disclosure provides an antibody drug conjugate according to any preceding embodiment, wherein R is a bond, p is 1, m is 0, w is 0, and q is 0. In certain embodiments, the present disclosure provides an antibody drug conjugate according to any preceding embodiment, wherein R is a bond, p is 1, m is 0, w is 0, and q is 1.

In one embodiment, the present disclosure provides an antibody drug conjugate according to any preceding embodiment, selected from the group consisting of compounds listed in Table 5, wherein n is 1, 2, 3, 4, 5, 6, 7, 8, 9, or 10. In certain embodiments, n is 2, 4, 6, or 8. In certain embodiments, n is 2. In certain embodiments, n is 4.

In one embodiment, the present disclosure provides an antibody drug conjugate according to any preceding embodiment, selected from the group consisting of Example 4-conjugated, Example 28-conjugated, and Example 47-conjugated. In certain embodiments, the present disclosure provides an antibody drug conjugate according to any preceding embodiment, wherein the antibody drug conjugate is Example 47-conjugated, and wherein n is 2. In certain embodiments, the present disclosure provides an antibody drug conjugate according to any preceding embodiment, wherein the antibody drug conjugate is Example 47-conjugated, and wherein n is 4. In certain embodiments, the present disclosure provides an antibody drug conjugate according to any preceding embodiment, wherein the antibody drug conjugate is Example 28-conjugated, and wherein n is 2. In certain embodiments, the present disclosure provides an antibody drug conjugate according to any preceding embodiment, wherein the antibody drug conjugate is Example 28-conjugated, and wherein n is 4.

In one embodiment, the present disclosure provides an antibody drug conjugate according to any preceding embodiment, selected from the group consisting of compounds listed in Table 6A or 6B, wherein n is 1, 2, 3, 4, 5, 6, 7, 8, 9, or 10. In certain embodiments, n is 2, 4, 6, or 8. In certain embodiments, n is 2. In certain embodiments, n is 4.

In one embodiment, the present disclosure provides an antibody drug conjugate according to any preceding embodiment, selected from the group consisting of Example 6-conjugated, Example 6-hydrolyzed, Example 7-conjugated, Example 7-hydrolyzed, Example 12-conjugated, Example 12-hydrolyzed, Example 13-conjugated, and Example 13-hydrolyzed.

In one embodiment, the present disclosure provides an antibody drug conjugate according to any preceding embodiment, selected from the group consisting of Example 12-hydrolyzed, Example 13-hydrolyzed.

In certain embodiments, the antibody of the antibody drug conjugate comprises a heavy chain variable region set forth as SEQ ID NO: 5 and a light chain variable region set forth as SEQ ID NO: 6.

In certain embodiments, the antibody of the antibody drug conjugate comprises a heavy chain set forth as SEQ ID NO: 3. In certain embodiments, the antibody of the antibody drug conjugate comprises a light chain set forth as SEQ ID NO: 4. In certain embodiments, the antibody of the antibody drug conjugate comprises a heavy chain set forth as SEQ ID NO: 3 and a light chain set forth as SEQ ID NO: 4.

In one embodiment, the present disclosure provides a pharmaceutical composition comprising the antibody drug conjugate according to any preceding embodiment and a pharmaceutically acceptable carrier.

In one embodiment, the present disclosure provides a method of treating a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS), in a subject in need thereof, comprising administering an effective amount of the antibody drug conjugate according to any preceding embodiment or the pharmaceutical composition according to any preceding embodiment to the subject.

In one embodiment, the present disclosure provides a kit, comprising: (a) a container comprising the antibody drug conjugate according to any preceding embodiment or the pharmaceutical composition according to any preceding embodiment; and (b) a label or package insert on or associated with the one or more containers, wherein the label or package insert indicates that the antibody drug conjugate or pharmaceutical composition is used for treating a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS).

In any of the preceding embodiments, the IBD is ulcerative colitis (UC) or Crohn's disease.

In one embodiment, the present disclosure provides a method of delivering a glucocorticoid receptor agonist to a CD40-expressing cell, comprising the step of contacting the cell with the antibody drug conjugate according to any preceding embodiment.

In one embodiment, the present disclosure provides a method of determining anti-inflammatory activity of an antibody drug conjugate comprising: (a) contacting a CD40-expressing cell with the antibody drug conjugate according to any preceding embodiment; and (b) determining reduced release of pro-inflammatory cytokines from the cell as compared to a control cell.

### BRIEF DESCRIPTION OF THE DRAWINGS

FIG. 1A depicts the deconvoluted mass spectroscopy data as provided in ADC Example 2, of Example 4-conjugated (human) (n=4). The 25140.73 peak corresponds to the light chain (SEQ ID NO: 4) with one drug linker molecule conjugated. The 50917.59 peak corresponds to the heavy chain (SEQ ID NO: 3) with one drug linker molecule conjugated.

FIG. 1B depicts anionic exchange chromatographic (AEC) data as provided in ADC Example 2 for Example 28-conjugated (human) (n=2), with retention time of about 7.5 minutes.

FIG. 1C depicts the deconvoluted mass spectroscopy data as provided in ADC Example 2 of Example 4-conjugated (human) (n=2). The 25176.72 peak corresponds to the light chain (SEQ ID NO: 2) with one drug linker molecule conjugated. The 50954.63 peak corresponds to the heavy chain (SEQ ID NO: 1) with one drug linker molecule conjugated

FIG. 1D depicts anionic exchange chromatographic (AEC) data as provided in ADC Example 2 for Example 28-conjugated (human) (n=4), with retention time of about 13 minutes.

FIG. 1E depicts the deconvoluted mass spectroscopy data as provided in ADC Example 2 of Example 4-conjugated (human) (n=4). The 25176.88 peak corresponds to the light chain (SEQ ID NO: 2) with one drug linker molecule conjugated. The 50954.80 peak corresponds to the heavy chain (SEQ ID NO: 1) with one drug linker molecule conjugated.

FIG. 2 depicts the in vitro activity of anti-human CD40 ADCs in LPS and CD40L-stimulated human MoDC assay as described in Example C. The data in FIG. 2 demonstrates that the maximum capacity to inhibit immune cell activation by either of the two ADC compounds tested exceeds inhibition provided by the parental antagonist antibody.

FIG. 3 depicts in vitro activity of anti-mouse CD40 ADC in LPS and CD40L-stimulated murine BMDC assay as described in Example D. The results shown in FIG. 3 demonstrate that the maximum capacity to inhibit immune cell activation by Example 6-hydrolyzed (mouse) exceeds inhibition provided by the parental antagonist antibody.

FIG. 4 depicts in vivo activity of Example 6-hydrolyzed (mouse) (n=4) in LPS-induced acute inflammation, as described in Example E. The results shown in FIG. 4 demonstrate that the CD40 ADC exhibits greater efficacy in suppressing DC activation in vivo than the parental antagonist antibody or isotype ADC.

FIG. 5A depicts in vivo activity of an anti-mouse CD40 ADC (Example 12-hydrolyzed (mouse)) in DTH response, and FIG. 5B depicts in vivo activity of an anti-mouse CD40 ADC (Example 28-conjugated (mouse)) in DTH response, as described in Example F. Data in FIGS. 5A and 5B demonstrates the enhanced efficacy of CD40 ADC to more potently inhibit T-cell mediated inflammation in vivo than parental antagonist antibody or non-targeted ADC alone.

FIG. 6 depicts in vivo activity of anti-mouse CD40 ADCs in mouse collagen induced arthritis (CIA), as described in Example H. Data in FIG. 6 demonstrate that a single dose of anti-mouse CD40 steroid ADC can exhibit an extended duration of action through amelioration of paw swelling for 6 weeks compared to the Controls 1 and 2.

### DEFINITIONS

The terms “human CD40” and “human CD40 wild type” (abbreviated herein as hCD40, hCD40 wt), as used herein, refers to a type I transmembrane protein. In one embodiment, the term human CD40 is intended to include recombinant human CD40 (rhCD40), which can be prepared by standard recombinant expression methods. Table 1 provides the amino acid sequence of human CD40 (i.e., SEQ ID NO. 1), and the extracellular domain thereof (i.e., SEQ ID NO: 2).

TABLE 1
Sequence of human CD40

| Protein | Sequence |
|---|---|
| Human CD40 | MVRLPLQCVLWGCLLTAVHPEPPT |
| | ACREKQYLINSQCCSLCQPGQKLV |
| | SDCTEFTETECLPCGESEFLDTWN |
| | RETHCHQHKYCDPNLGLRVQQKGT |
| | SETDTICTCEEGWHCTSEACESCV |
| | LHRSCSPGFGVKQIATGVSDTICE |
| | PCPVGFFSNVSSAFEKCHPWTSCE |
| | TKDLVVQQAGTNKTDVVCGPQDRL |
| | RALVVIPIIFGILFAILLVLVFIK |
| | KVAKKPTNKAPHPKQEPQEINFPD |
| | DLPGSNTAAPVQETLHGCQPVTQE |
| | DGKESRISVQERQ |
| | (SEQ ID NO: 1) |
| Human CD40 | EPPTACREKQYLINSQCCSLCQPG |
| Extracellular | QKLVSDCTEFTETECLPCGESEFL |
| Domain | DTWNRETHCHQHKYCDPNLGLRVQ |
| | QKGTSETDTICTCEEGWHCTSEAC |
| | ESCV |
| | (SEQ ID NO: 2) |

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or simply “antibody portion”), as used herein, refers to one or more fragments of an antibody that retain the ability to specifically bind to an antigen (e.g., TNFα). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

A “variable region” of an antibody refers to the variable region of the antibody light chain or the variable region of the antibody heavy chain, either alone or in combination. The variable regions of the heavy and light chain each have four framework regions (FR) and three complementarity determining regions (CDRs) also known as hypervariable regions. The CDRs contribute to the formation of the antigen-binding site of antibodies. There are at least two techniques for determining CDRs: (1) an approach based on cross-species sequence variability (e.g., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). In addition, combinations of these two approaches are sometimes used in the art to determine CDRs.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence according to the present disclosure, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies of the disclosure do not abrogate the binding of the antibody containing the amino acid sequence, to the antigen(s), e.g., the CD40 to which the antibody binds. Methods of identifying nucleotide and amino acid conservative substitutions which do not eliminate antigen binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

“Binding affinity” generally refers to the strength of the sum total of noncovalent interactions between a single binding site of a molecule (e.g., an antibody or antigen-binding portion thereof) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound longer. A variety of methods of measuring binding affinity are known in the art.

The term “antagonist” as used herein, refers to an antibody or or antigen-binding portion thereof that blocks or reduces the biological or immunological activity of human CD40 (hCD40). An antagonist antibody, or antigen-binding portion thereof, of hCD40 may, for example, inhibit CD86 upregulation of primary human B cells that are cultured with (or exposed to) CD40L (such as culturing the B cells with CD40L-expressing human T cells). In one embodiment, an antagonist anti-CD40 antibody, or antigen-binding portion thereof, that is substantially free of agonist activity is defined as having a level of activity that is equivalent to or within one standard deviation from a negative control in an agonist assay, such as the agonist monocyte assay described in Example 7 of PCT Publication No. WO 2016/196314. Agonist and antagonist activity can also be assessed using methods known in the art, e.g., using a CD40 expressing reporter cell line expressing human CD40 linked to NFkB mediated alkaline phosphatase (AP) or a B cell assay.

A “radical of a glucocorticosteroid” is derived from the removal of a hydrogen atom from an amino group of a parent glucocorticosteroid. The removal of the hydrogen atom facilitates the attachment of the parent glucocorticosteroid to a linker.

The term “drug loading” and “drug antibody ratio” (DAR) are used interchangeably herein, and refer to the number of glucocorticosteroid radicals connected, via a linker, to an antibody. The “drug loading” or “drug antibody ratio” (DAR) of an antibody drug conjugate comprising a radical of Formula (I), or an antibody drug conjugate of Formula (II), for example, and representing an individual ADC, refers to the number of glucocorticosteroid molecules linked to the individual antibody (e.g., drug loading of 1, 2, 3, 4, 5, 6, 7, 8, 9, or 10, or variable n is 1, 2, 3, 4, 5, 6, 7, 8, 9, or 10, respectively) (“compound DAR”). Further, the drug antibody ratio (DAR) of a population of antibody drug conjugates (e.g., provided in a composition or fraction collected) refers to an average number of glucocorticosteroid molecules linked to an antibody in the given population, e.g., drug loading or n as an integer or fraction of 1 to 10±0.5, ±0.4, ±0.3, ±0.2, or ±0.1 (“population DAR”).

The term “subject” refers to any animal (e.g., a mammal), including, but not limited to humans, non-human primates, rodents, and the like, which is to be the recipient of a particular treatment.

An “effective amount” of an antibody drug conjugate as disclosed herein is an amount sufficient to carry out a specifically stated purpose. An “effective amount” can be determined in relation to the stated purpose.

The term “therapeutically effective amount” refers to an amount of an antibody drug conjugate effective to “treat” a disease or disorder in a subject or mammal. A “prophylactically effective amount” refers to an amount effective to achieve the desired prophylactic result.

Terms such as “treating” or “treatment” or “to treat” or “alleviating” or “to alleviate” refer to therapeutic measures that cure, slow down, lessen one or more symptoms of, and/or slow or halt progression of a diagnosed pathologic condition or disorder (“therapeutic treatment”). Thus, those in need of therapeutic treatment include those already diagnosed with or suspected of having the disorder. Prophylactic or preventative measures refer to measures that prevent the development of a targeted pathological condition or disorder (“prophylactic treatment”). Thus, those in need of prophylactic treatment include those prone to have the disorder and those in whom the disorder is to be prevented.

### DETAILED DESCRIPTION

The present disclosure provides antibody drug conjugates (ADCs) comprising a glucocorticoid receptor agonist linked to an anti-CD40 antibody.

In inflammatory bowel diseases, such as Crohn's Disease and ulcerative colitis, loss of gut barrier integrity allows commensal bacteria to invade the intestinal mucosa. The corresponding host immune response results in exacerbated inflammation. Microbial activation of immune cells can occur independently of CD40 signaling and thus would remain unaffected by treatment with an anti-CD40 antagonist, such as Ab-102, limiting its efficacy. However, an glucocorticoid receptor agonist linked to the anti-CD40 antibody would not only block CD40-mediated activation but, upon internalization and release of its glucocorticoid receptor agonist payload, also inhibit inflammatory signaling of microbe-derived molecules via Toll-like Receptors (TLRs). This dual mechanism would exhibit maximal inhibitory response towards activated inflammatory cells resulting in enhanced efficacy compared to an anti-CD40 antagonist alone

The data of Example C, and as provided in FIG. 2 and Table 18, confirm this hypothesis. Semi-adherent monocyte-derived dendritic cells (derived from primary human peripheral blood mononuclear cells) were pre-stimulated with lipopolysaccharide (LPS) to induce up-regulation of cell-surface CD40 expression. After washing and pre-treatment with anti-CD40 antibody alone (Control 1) versus a selection of anti-human CD40 ADCs, cells were activated with LPS and/or CD40L and the secretion of pro-inflammatory cytokine IL-6 was quantified. The data demonstrate that while the anti-CD40 antibody alone (Control 1) partially suppresses the inflammatory signaling, the anti-human CD40 ADCs tested fully suppress the additional inflammatory, CD40-independent, signaling (i.e., to level prior to LPS pre-stimulation (dotted line)).

I. Anti-CD40 Antibody

The terms “anti-CD40 antibody” and “anti-CD40 antigen-binding portion” refer to a full-length antibody and an antigen-binding portion, respectively, which is an antagonist of human CD40. The full-length amino acid sequence for human CD40 is provided in Table 1, SEQ ID NO: 1. The extracellular domain of human CD40 contains amino acids is provided in Table 1, SEQ ID NO: 2.

In one embodiment, the antibody, or antigen binding portion thereof, is an antagonist antibody, or antigen binding portion thereof, which causes a decrease in CD40 activity or function as compared to CD40 activity or function in the absence of the antibody, or antigen binding portion thereof. In particular embodiments, the antibody, or antigen binding portion thereof, is substantially free of agonist activity, i.e., the antibody, or antigen binding portion thereof, does not cause an increase in the magnitude of CD40 activity or function as compared to CD40 activity or function in the absence of the antibody, or antigen binding portion thereof. In certain embodiments, the anti-CD40 antibody is a polyclonal antibody, monoclonal antibody, chimeric antibody, humanized antibody, human antibody, or an antigen binding portion thereof.

In certain embodiments, the anti-CD40 antibody is lucatumumab (Novartis; as described in U.S. Pat. No. 8,277,810); antibodies 5D12, 3A8 and 3C6, or humanized versions thereof (Novartis; as described in U.S. Pat. No. 5,874,082); antibody 15B8 (Novartis; as described in U.S. Pat. No. 7,445,780); antibody 4D11 (Kyowa Hakko Kirin; as described in U.S. Pat. No. 7,193,064); temeliximab (Bristol Myers Squibb; as described in U.S. Pat. No. 6,051,228); antibody PG102 (PanGenetics; as described in U.S. Pat. No. 8,669,352); antibody 2C10 (Primatope; US Patent Application Pub. No. 20140093497); anti-CD40 antibodies described in U.S. Pat. Nos. 8,591,900 and 8,778,345 (Boehringer Ingelheim); anti-CD40 antibodies described in U.S. Pat. No. 5,801,227 (Amgen); or APX005 (Boehringer Ingelheim; as described in US Patent Appln. Pub. No. 20120301488).

In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12.

In certain embodiments, the anti-CD40 antibody comprises a heavy chain variable region set forth as SEQ ID NO: 5. In certain embodiments, the anti-CD40 antibody comprises a light chain variable region set forth as SEQ ID NO: 6. In certain embodiments, the anti-CD40 antibody comprises a heavy chain variable region set forth as SEQ ID NO: 5 and a light chain variable region set forth as SEQ ID NO: 6.

In certain embodiments, the anti-CD40 antibody comprises a heavy chain set forth as SEQ ID NO: 3. In certain embodiments, the anti-CD40 antibody comprises a light chain set forth as SEQ ID NO: 4. In certain embodiments, the anti-CD40 antibody is the full-length antibody, Ab102, described in U.S. Publication No. 2016/0347850, and which comprises a heavy chain set forth as SEQ ID NO: 3 and a light chain set forth as SEQ ID NO: 4 (CDR regions bolded; constant regions underlined).

TABLE 3
anti-CD40 antibody sequence (Ab102)

| Antibody Region | Amino Acid Sequence |
|---|---|
| Human | EVQLVESGGGLVKPGGSLRL |
| Ab102-HC | SCAASGFTFSDYGMNWVRQA |
| (Heavy chain) | PGRGLEWIAYISSGRGNIYY |
| | ADTVKGRFTISRDNAKNSLY |
| | LQMNSLRAEDTAVYYCARSW |
| | GYFDVWGQGTTVTVSSAAST |
| | KGPSVFPLAPSSKSTSGGTA |
| | ALGCLVKDYFPEPVTVSWNS |
| | GALTSGVHTFPAVLQSSGLY |
| | SLSSVVTVPSSSLGTQTYIC |
| | NVNHKPSNTKVDKKVEPKSC |
| | DKTHTCPPCPAPEAAGGPSV |
| | FLFPPKPKDQLMISRTPEVT |
| | CVVVDVSHEDPEVKFNWYVD |
| | GVEVHNAKTKPREEQYNSTY |
| | RVVSVLTVLHQDWLNGKEYK |
| | CKVSNKALPAPIEKTISKAK |
| | GQPREPQVYTLPPSREEMTK |
| | NQVSLTCLVKGFYPSDIAVE |
| | WESNGQPENNYKTTPPVLDS |
| | DGSFFLYSKLTVDKSRWQQG |
| | NVFSCSVLHEALHNHYTQKS |
| | LSLSPGK |
| | (SEQ ID NO: 3) |
| Human | DIVMTQSPDSLAVSLGERAT |
| Ab102-LC | INCKSSQSLLNRGNQKNYLT |
| (Light chain) | WFQQKPGQPPKLLIYWASTR |
| | ESGVPDRFSGSGSGTDFTLT |
| | ISSLQAEDVAVYYCQNDYTY |
| | PLTFGQGTKLEIKRTVAAPS |
| | VFIFPPSDEQLKSGTASVVC |
| | LLNNFYPREAKVQWKVDNAL |
| | QSGNSQESVTEQDSKDSTYS |
| | LSSTLTLSKADYEKHKVYAC |
| | EVTHQGLSSPVTKSFNRGEC |
| | (SEQ ID NO: 4) |
| Heavy chain | EVQLVESGGGLVRPGGSLRL |
| variable region | SCAASGFTFSDYGMNWVRQA |
| | PGRGLEWIAYISSGRGNIYY |
| | ADTVKGRFTISRDNARNSLY |
| | LQMNSLRAEDTAVYYCARSW |
| | GYFDVWGQGTTVTVSS |
| | (SEQ ID NO: 5) |
| Light chain | DIVMTQSPDSLAVSLGERAT |
| variable region | INCKSSQSLLNRGNQKNYLT |
| | WFQQKPGQPPKLLIYWASTR |
| | ESGVPDRFSGSGSGTDFTLT |
| | ISSLQAEDVAVYYCQNDYTY |
| | PLTFGQGTKLEIK |
| | (SEQ ID NO: 6) |
| VH-CDR1 | GFTFSDYGMN |
| | (SEQ ID NO: 7) |
| VH-CDR2 | YISSGRGNIYYADTVKG |
| | (SEQ ID NO: 8) |
| VH-CDR3 | SWGYFDV |
| | (SSEQ ID NO: 9) |
| VL-CDR1 | KSSQSLLNRGNQKNYLT |
| | (SEQ ID NO: 10) |
| VL-CDR2 | WASTRES |
| | (SEQ ID NO: 11) |
| VL-CDR3 | QNDYTYPLT |
| | (SEQ ID NO: 12) |

It will be appreciated that the anti-CD40 antibody can be provided by the partial deletion or substitution of a few or even a single amino acid. For example, the mutation of a single amino acid in selected areas of the CH2 domain can be enough to substantially reduce Fc binding. Similarly, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, the constant regions of the disclosed antibodies can be modified through the mutation or substitution of one or more amino acids that enhances the profile of the resulting construct. In this respect it can be possible to disrupt the activity provided by a conserved binding site (e.g., Fc binding) while substantially maintaining the configuration and immunogenic profile of the antibodies. Certain embodiments can comprise the addition of one or more amino acids to the constant region to enhance desirable characteristics such as decreasing or increasing effector function or provide for more glucocorticoid receptor agonist attachment. In such embodiments it can be desirable to insert or replicate specific sequences derived from selected constant region domains.

The present disclosure further embraces variants and equivalents which are substantially homologous to anti-CD40 antibody set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

The anti-CD40 antibody can be recombinant polypeptides, natural polypeptides, or synthetic polypeptides of an antibody. It will be recognized in the art that some amino acid sequences of the disclosure can be varied without significant effect of the structure or function of the protein. Thus, the disclosure further includes variations of the polypeptides which show substantial activity or which include regions of an antibody. Such mutants include deletions, insertions, inversions, repeats, and type substitutions.

The anti-CD40 antibodies described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., Proc. Nat'l. Acad. Sci. USA 81:5662-5066 (1984) and U.S. Pat. No. 4,588,585.

In some embodiments a DNA sequence encoding an anti-CD40 antibody would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest.

In certain embodiments, recombinant expression vectors are used to amplify and express DNA encoding antibodies anti-CD40 antibodies. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of anti-CD40 antibodies include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985). Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823.

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods for purifying antibodies include, for example, those described in U.S. Patent Publication Nos. 2008/0312425, 2008/0177048, and 2009/0187005.

II. Anti-CD40 Antibody Linked to a Glucocorticoid Receptor Agonist

Antibody drug conjugates (ADCs) comprising a glucocorticoid receptor agonist linked to an anti-CD40 antibody are provided herein. In some embodiments, the ADC binds to Fc gamma receptor. In some embodiments, the ADC is active in a Jurkat cell reporter assay. In some embodiments, the ADC is active in a CD40L reporter assay. In some embodiments, the ADC shows reduced immunogenicity (reduced anti-drug immune response (ADA)) as compared to the anti-CD40 antibody alone.

In one embodiment, provided is an antibody drug conjugate comprising: (a) an anti-CD40 antibody; and (b) a radical of a glucocorticoid receptor agonist of Formula (I):

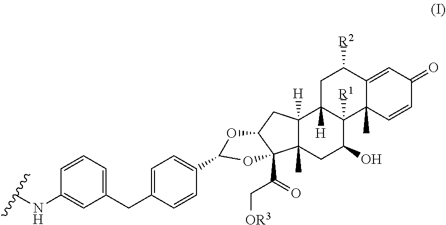

wherein:
R¹ is hydrogen or fluoro;
R² is hydrogen or fluoro; and
R³ is hydrogen or —P(═O)(OH)₂; and

further wherein the antibody is conjugated to the glucocorticoid receptor agonist by a linker of formula:

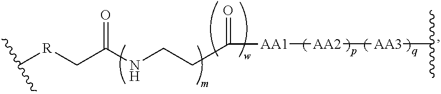

R is a bond,

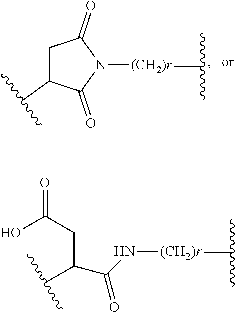

wherein r is 0 or 1;
AA1, AA2, and AA3 are independently selected from the group consisting of Alanine (Ala), Glycine (Gly), Isoleucine (Ile), Leucine (Leu), Proline (Pro), Valine (Val), Phenylalanine (Phe), Tryptophan (Trp), Tyrosine (Tyr), Aspartic acid (Asp), Glutamic acid (Glu), Arginine (Arg), Histidine (His), Lysine (Lys), Serine (Ser), Threonine (Thr), Cysteine (Cys), Methionine (Met), Asparagine (Asn), and Glutamine (Gln);
m is 0 or 1;
w is 0 or 1;
p is 0 or 1; and
q is 0 or 1.

In another embodiment, provided is an antibody drug conjugate of Formula (II):

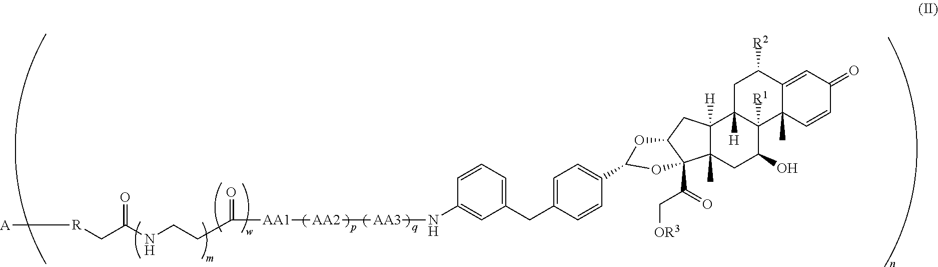

wherein:

A is an anti-CD40 antibody; and

n is 1, 2, 3, 4, 5, 6, 7, 8, 9, or 10.

In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4.

The antibody may be linked to variable R by any moiety on the antibody bearing a nucleophilic group, e.g., such as an OH group (to provide an —O— group, when linked), an —SH group (to provide an —S— group, when linked), or an —NH₂ group (to provide an —NH— group, when linked. In certain embodiments, the point of attachment of the antibody to variable R is via an SH group of a cysteine residue of the antibody (to provide an —S— group, when linked).

In certain embodiments, R¹ is hydrogen and R² is hydrogen.

In certain embodiments, R¹ is fluoro and R² is hydrogen.

In certain embodiments, R¹ is fluoro and R² is fluoro.

In certain embodiments, R³ is hydrogen.

In certain embodiments, R³ is —P(═O)(OH)₂.

In certain preferred embodiments, at least one of R¹ and R² is fluoro and R³ is —P(═O)(OH)₂.

In certain embodiments, -AA1-(AA2)ₚ-(AA3)_{q}- is selected from the group consisting of: -Gly-Glu-; -Ala-Ala-; -Glu-Ala-Ala-; -Gly-Lys-; -Glu-; -Glu-Ser-Lys-; and -Gly-Ser-Lys-. It should be understood this listing of amino acids should be read left to right, wherein the furthest left hand amino acid corresponds to AA1, and the farthest right hand amino acid corresponds to AA2 (when q is 0), or AA3 (when q is 1).

In certain preferred embodiments, the linker moiety comprises 1, 2, or 3 hydrophilic amino acids -AA1-(AA2)ₚ-(AA3)_{q}-, e.g., wherein the side chain of AA1, AA2, and/or AA3 comprises a hydrogen bonding group such as ═O and/or a hydrogen donating group such as an —OH, —NH₂, or —SH. Increasing the hydrophilicity of the linker may lead to long term stability and storage of the ADC. For example, in certain preferred embodiments, -AA1-(AA2)ₚ-(AA3)_{q}- is selected from the group consisting of -Gly-Glu-; -Gly-Lys-; -Glu-Ser-Lys-; and -Gly-Ser-Lys-.

In certain embodiments, wherein m is 0, then w is 0. In certain embodiments, wherein m is 1, then w is 1.

In certain embodiments, m is 0; q is 0; and R is

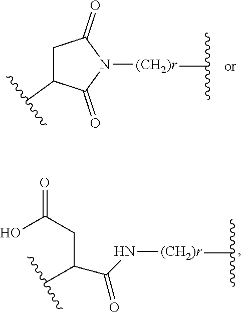

wherein r is 0 or 1. In certain embodiments, w is 0. In certain embodiments, r is 0. In certain embodiments, r is 1.

In certain preferred embodiments, R is a bond. ADCs comprising an R group of formula

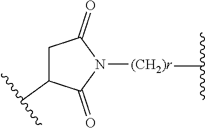

may be unstable in vivo. ADCs comprising a ring-opened R group of formula

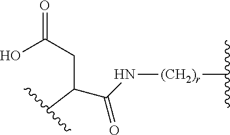

may ring-close to a group of formula

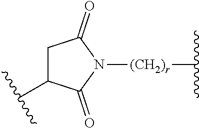

over prolonged storage in liquid excipients. Furthermore, preparing the ring-opened ADC from the ring-closed ADC may require basic pH conditions for extended period of time, which may lead to longer production times and higher manufacturing costs, as well as undesirable decomposition of the ADC due to the high pH.

In certain embodiments, m is 0 or 1; p is 1; and R is a bond. In certain embodiments, w is 0. In certain embodiments q is 0. In certain embodiments, q is 1.

In certain embodiments, m is 1; and q is 0. In certain embodiments, w is 1. In certain embodiments, m is 1; and w is 1. In certain embodiments, m is 1; w is 1; and q is 0.

In certain embodiments, m is 0.

In certain preferred embodiments, p is 1. In certain preferred embodiments, p is 1 and m is 0. In certain preferred embodiments, p is 1, m is 0, and w is 0. In certain preferred embodiments, p is 1, m is 0, w is 0, q is 0, and R is a bond. In certain alternative preferred embodiments, p is 1, m is 0, w is 0, q is 1, and R is a bond.

In certain embodiments, the antibody drug conjugate comprising a radical of Formula (I), the drug loading is 1, 2, 3, 4, 5, 6, 7, 8, 9, or 10. In certain embodiments, the drug loading is 2, 3, 4, 5, 6, 7, or 8. In another embodiment, the drug loading is 1, 2, 3, 4, or 5. In another embodiment, the drug loading is 2, 3, 4, or 5. In another embodiment, the drug loading is 2, 4, 6, or 8. In another embodiment, the drug loading is 1. In another embodiment, the drug loading is 2. In another embodiment, the drug loading is 3. In another embodiment, the drug loading is 4. In another embodiment, the drug loading is 5. In another embodiment, the drug loading is 6. In another embodiment, the drug loading is 7. In another embodiment, the drug loading is 8. In a preferred embodiment, the drug loading is 2 or 4.

In certain embodiments of Formula (II), n is 2, 3, 4, 5, 6, 7, or 8. In certain embodiments of Formula (II), n is 1, 2, 3, 4, or 5. In certain embodiments of Formula (II), n is 2, 3, 4, or 5. In certain embodiments of Formula (II), n is 2, 4, 6, or 8. In certain embodiments of Formula (II), n is 1. In certain embodiments of Formula (II), n is 2. In certain embodiments of Formula (II), n is 3. In certain embodiments of Formula (II), n is 4. In certain embodiments of Formula (II), n is 5. In certain embodiments of Formula (II), n is 6. In certain embodiments of Formula (II), n is 7. In certain embodiments of Formula (II), n is 8. In a preferred embodiment of Formula (II), n is 2 or 4.

Various combinations of the above described embodiments are further contemplated herein.

For example, in certain embodiments, wherein R¹ is hydrogen, R² is hydrogen, and R³ is hydrogen, provided is an antibody drug conjugate comprising a radical of Formula (I-a), or an antibody drug conjugate of Formula (II-a):

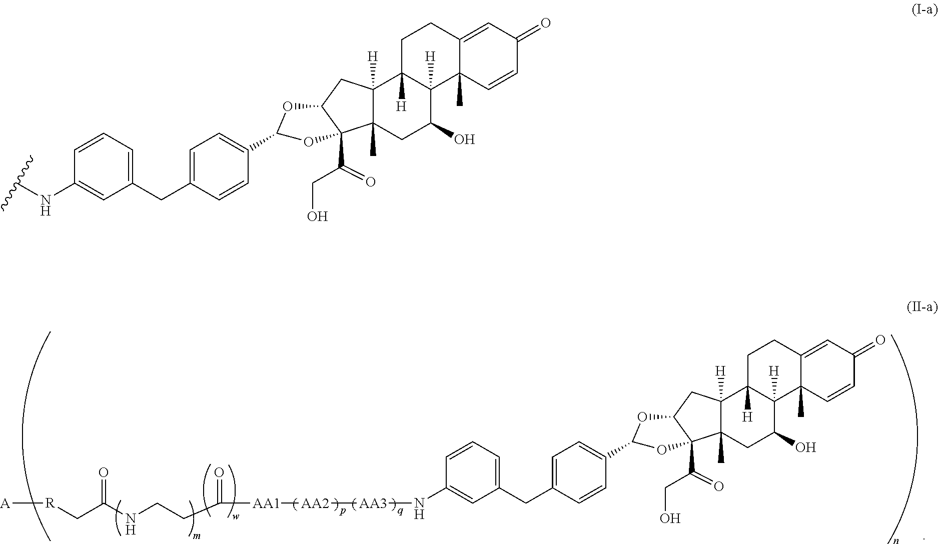

In certain embodiments, R is a bond. In certain embodiments, R is a bond, m is 1, p is 1, and q is 0. In certain embodiments, R is a bond, m is 1, p is 1, q is 0, and -AA1-(AA2)ₚ-(AA3)_{q}- is selected from the group consisting of: -Gly-Glu-; -Ala-Ala-; and -Gly-Lys-. In certain embodiments, R is a bond, m is 0, p is 1, and q is 0 or 1. In certain embodiments, R is a bond, m is 0, p is 1, q is 0 or 1, and -AA1-(AA2)ₚ-(AA3)_{q}- is selected from the group consisting of: -Gly-Glu-; -Ala-Ala-; -Glu-Ala-Ala-; -Gly-Lys-; -Glu-Ser-Lys-; and -Gly-Ser-Lys-. However, in certain embodiments, -Ala-Ala- and -Glu-Ala-Ala- are excluded. In certain embodiments, R is a bond, m is 0, p is 1, q is 0, and -AA1-(AA2)ₚ-(AA3)_{q}- is -Gly-Glu- or -Gly-Lys-. In certain embodiments, R is a bond, m is 0, p is 1, q is 1, and -AA1-(AA2)ₚ-(AA3)_{q}- is -Glu-Ser-Lys-; and -Gly-Ser-Lys-. In certain embodiments, wherein m is 0, then w is 0. In certain embodiments, wherein m is 1, then w is 1. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

In certain embodiments, wherein R¹ is hydrogen, R² is hydrogen, and R³ is —P(═O)(OH)₂, provided is an antibody drug conjugate comprising a radical of Formula (I-b), or an antibody drug conjugate of Formula (II-b):

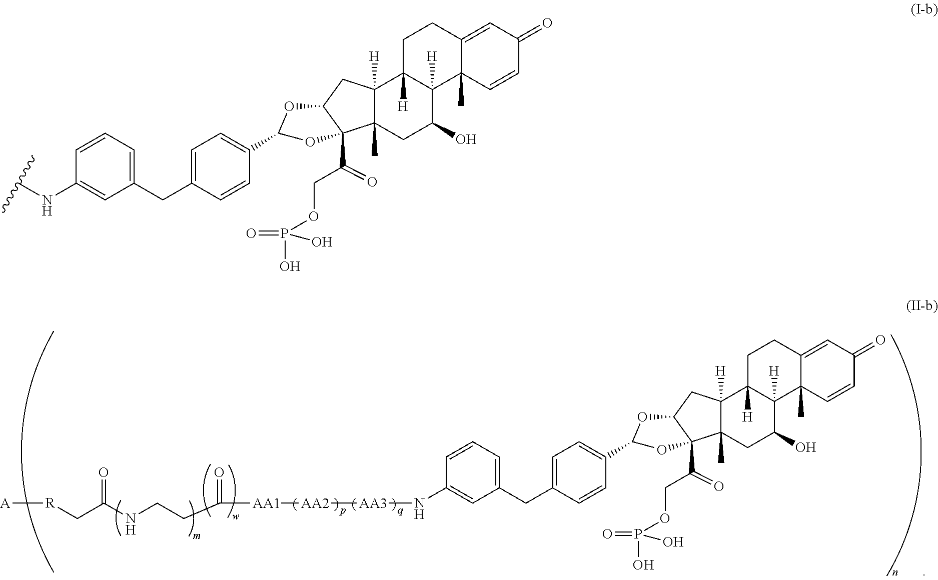

In certain embodiments, R is a bond. In certain embodiments, R is a bond, m is 1, p is 1, and q is 0. In certain embodiments, R is a bond, m is 1, p is 1, q is 0, and -AA1-(AA2)ₚ-(AA3)_{q}- is selected from the group consisting of: -Gly-Glu-; -Ala-Ala-; and -Gly-Lys-. In certain embodiments, R is a bond, m is 0, p is 1, and q is 0 or 1. In certain embodiments, R is a bond, m is 0, p is 1, q is 0 or 1, and -AA1-(AA2)ₚ-(AA3)_{q}- is selected from the group consisting of: -Gly-Glu-; -Ala-Ala-; -Glu-Ala-Ala-; -Gly-Lys-; -Glu-Ser-Lys-; and -Gly-Ser-Lys-. However, in certain embodiments, -Ala-Ala- and -Glu-Ala-Ala- are excluded. In certain embodiments, R is a bond, m is 0, p is 1, q is 0, and -AA1-(AA2)ₚ-(AA3)_{q}- is -Gly-Glu- or -Gly-Lys-. In certain embodiments, R is a bond, m is 0, p is 1, q is 1, and -AA1-(AA2)ₚ-(AA3)_{q}- is -Glu-Ser-Lys-; and -Gly-Ser-Lys-. In certain embodiments, wherein m is 0, then w is 0. In certain embodiments, wherein m is 1, then w is 1. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

In certain embodiments, wherein R¹ is fluoro, R² is fluoro, and R³ is hydrogen, provided is an antibody drug conjugate comprising a radical of Formula (I-c), or an antibody drug conjugate of Formula (II-c):

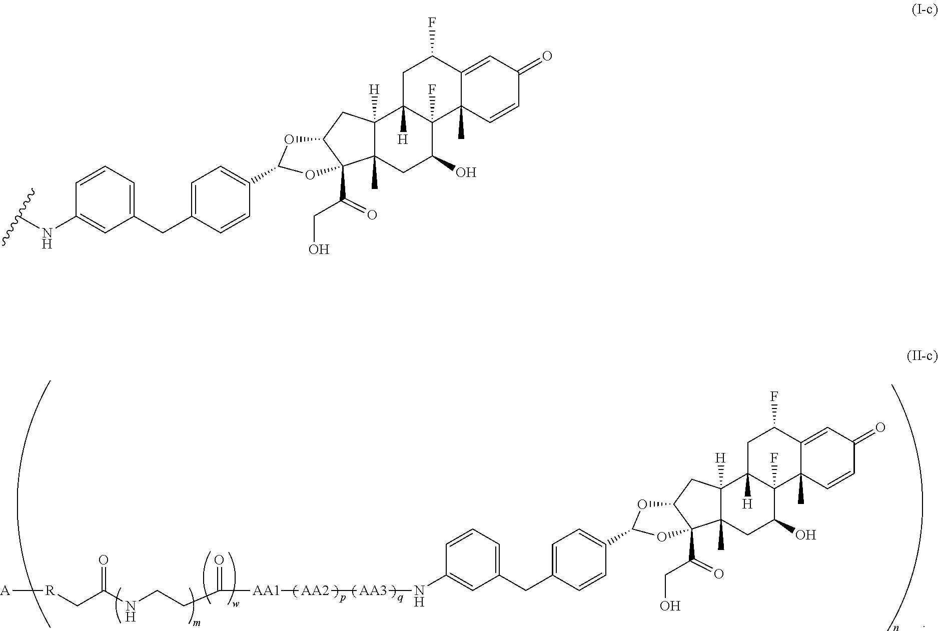

In certain embodiments, R is a bond. In certain embodiments, R is a bond, m is 1, p is 1, and q is 0. In certain embodiments, R is a bond, m is 1, p is 1, q is 0, and -AA1-(AA2)ₚ-(AA3)_{q}- is selected from the group consisting of: -Gly-Glu-; -Ala-Ala-; and -Gly-Lys-. In certain embodiments, R is a bond, m is 0, p is 1, and q is 0 or 1. In certain embodiments, R is a bond, m is 0, p is 1, q is 0 or 1, and -AA1-(AA2)ₚ-(AA3)_{q}- is selected from the group consisting of: -Gly-Glu-; -Ala-Ala-; -Glu-Ala-Ala-; -Gly-Lys-; -Glu-Ser-Lys-; and -Gly-Ser-Lys-. However, in certain embodiments, -Ala-Ala- and -Glu-Ala-Ala- are excluded. In certain embodiments, R is a bond, m is 0, p is 1, q is 0, and -AA1-(AA2)ₚ-(AA3)_{q}- is -Gly-Glu- or -Gly-Lys-. In certain embodiments, R is a bond, m is 0, p is 1, q is 1, and -AA1-(AA2)ₚ-(AA3)_{q}- is -Glu-Ser-Lys-; and -Gly-Ser-Lys-. In certain embodiments, wherein m is 0, then w is 0. In certain embodiments, wherein m is 1, then w is 1. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

In certain embodiments, wherein R¹ is fluoro, R² is fluoro, and R³ is —P(═O)(OH)₂, provided is an antibody drug conjugate comprising a radical of Formula (I-d), or an antibody drug conjugate of Formula (II-d):

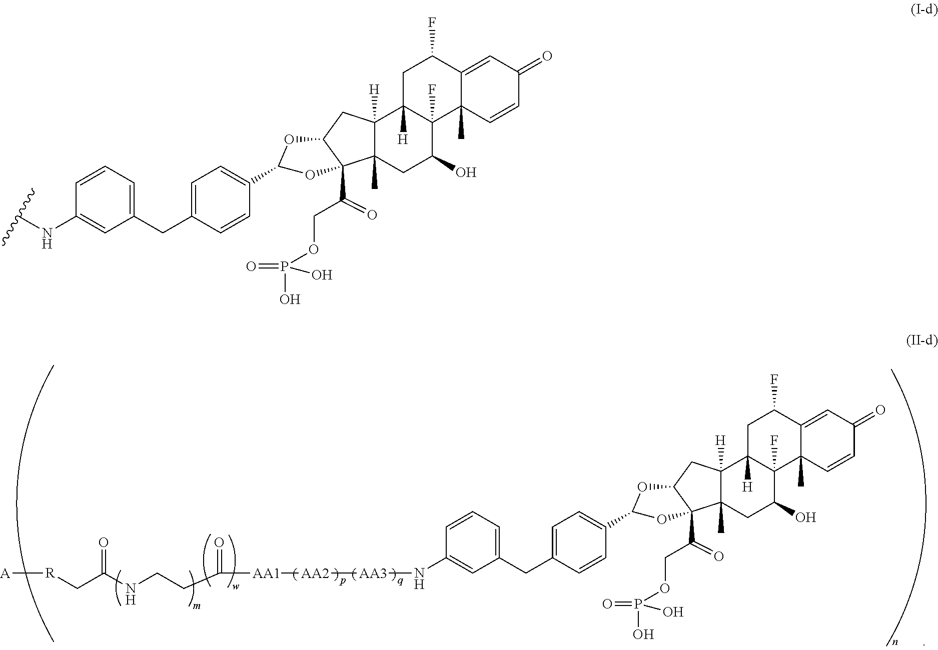

In certain embodiments, R is a bond. In certain embodiments, R is a bond, m is 1, p is 1, and q is 0. In certain embodiments, R is a bond, m is 1, p is 1, q is 0, and -AA1-(AA2)ₚ-(AA3)_{q}- is selected from the group consisting of: -Gly-Glu-; -Ala-Ala-; and -Gly-Lys-. In certain embodiments, R is a bond, m is 0, p is 1, and q is 0 or 1. In certain embodiments, R is a bond, m is 0, p is 1, q is 0 or 1, and -AA1-(AA2)ₚ-(AA3)_{q}- is selected from the group consisting of: -Gly-Glu-; -Ala-Ala-; -Glu-Ala-Ala-; -Gly-Lys-; -Glu-Ser-Lys-; and -Gly-Ser-Lys-. However, in certain embodiments, -Ala-Ala- and -Glu-Ala-Ala- are excluded. In certain embodiments, R is a bond, m is 0, p is 1, q is 0, and -AA1-(AA2)ₚ-(AA3)_{q}- is -Gly-Glu- or -Gly-Lys-. In certain embodiments, R is a bond, m is 0, p is 1, q is 1, and -AA1-(AA2)ₚ-(AA3)_{q}- is -Glu-Ser-Lys-; and -Gly-Ser-Lys-. In certain embodiments, wherein m is 0, then w is 0. In certain embodiments, wherein m is 1, then w is 1. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

In certain embodiments, wherein R¹ is fluoro, R² is hydrogen, and R³ is hydrogen, provided is an antibody drug conjugate comprising a radical of Formula (I-e), or an antibody drug conjugate of Formula (II-e):

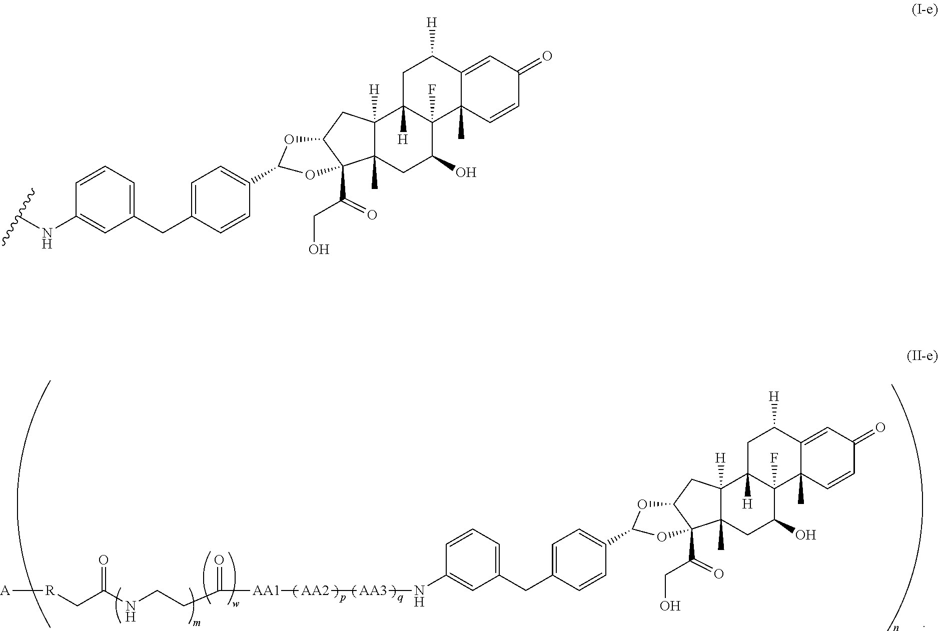

In certain embodiments, R is a bond. In certain embodiments, R is a bond, m is 1, p is 1, and q is 0. In certain embodiments, R is a bond, m is 1, p is 1, q is 0, and -AA1-(AA2)ₚ-(AA3)_{q}- is selected from the group consisting of: -Gly-Glu-; -Ala-Ala-; and -Gly-Lys-. In certain embodiments, R is a bond, m is 0, p is 1, and q is 0 or 1. In certain embodiments, R is a bond, m is 0, p is 1, q is 0 or 1, and -AA1-(AA2)ₚ-(AA3)_{q}- is selected from the group consisting of: -Gly-Glu-; -Ala-Ala-; -Glu-Ala-Ala-; -Gly-Lys-; -Glu-Ser-Lys-; and -Gly-Ser-Lys-. However, in certain embodiments, -Ala-Ala- and -Glu-Ala-Ala- are excluded. In certain embodiments, R is a bond, m is 0, p is 1, q is 0, and -AA1-(AA2)ₚ-(AA3)_{q}- is -Gly-Glu- or -Gly-Lys-. In certain embodiments, R is a bond, m is 0, p is 1, q is 1, and -AA1-(AA2)ₚ-(AA3)_{q}- is -Glu-Ser-Lys-; and -Gly-Ser-Lys-. In certain embodiments, wherein m is 0, then w is 0. In certain embodiments, wherein m is 1, then w is 1. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

In certain embodiments, wherein R¹ is fluoro, R² is hydrogen, and R³ is —P(═O)(OH)₂, provided is an antibody drug conjugate comprising a radical of Formula (I-f), or an antibody drug conjugate of Formula (II-f):

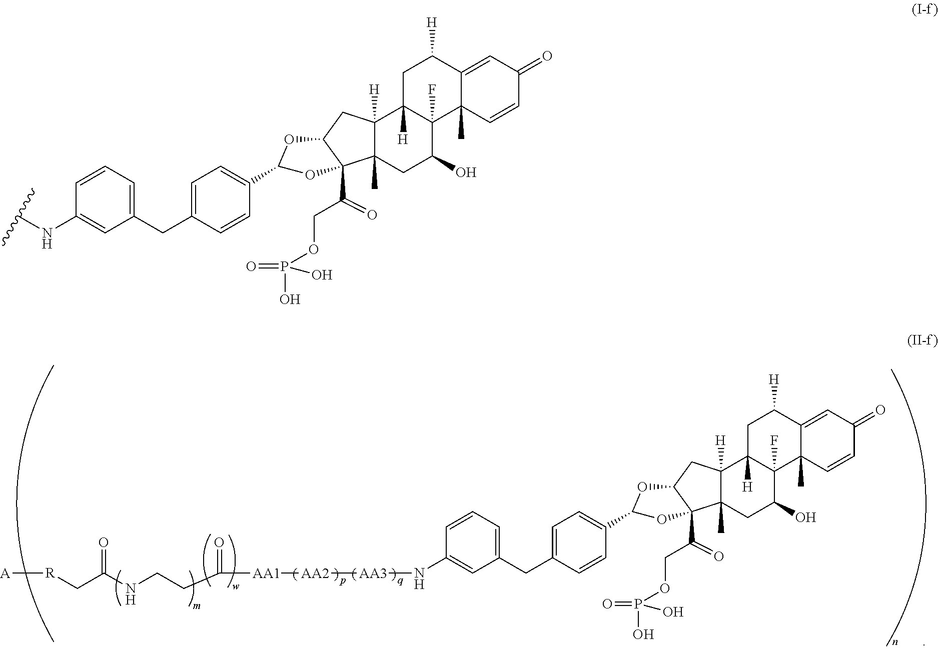

In certain embodiments, R is a bond. In certain embodiments, R is a bond, m is 1, p is 1, and q is 0. In certain embodiments, R is a bond, m is 1, p is 1, q is 0, and -AA1-(AA2)ₚ-(AA3)_{q}- is selected from the group consisting of: -Gly-Glu-; -Ala-Ala-; and -Gly-Lys-. In certain embodiments, R is a bond, m is 0, p is 1, and q is 0 or 1. In certain embodiments, R is a bond, m is 0, p is 1, q is 0 or 1, and -AA1-(AA2)ₚ-(AA3)_{q}- is selected from the group consisting of: -Gly-Glu-; -Ala-Ala-; -Glu-Ala-Ala-; -Gly-Lys-; -Glu-Ser-Lys-; and -Gly-Ser-Lys-. However, in certain embodiments, -Ala-Ala- and -Glu-Ala-Ala- are excluded. In certain embodiments, R is a bond, m is 0, p is 1, q is 0, and -AA1-(AA2)ₚ-(AA3)_{q}- is -Gly-Glu- or -Gly-Lys-. In certain embodiments, R is a bond, m is 0, p is 1, q is 1, and -AA1-(AA2)ₚ-(AA3)_{q}- is -Glu-Ser-Lys-; and -Gly-Ser-Lys-. In certain embodiments, wherein m is 0, then w is 0. In certain embodiments, wherein m is 1, then w is 1. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

Exemplary antibody drug conjugates comprising a radical of Formula (I), and antibody drug conjugates of Formula (II), include antibody drug conjugates listed in Tables 5, 6A, and 6B, wherein n is 1, 2, 3, 4, 5, 6, 7, 8, 9, or 10, and A is anti-CD40 antibody.

TABLE 5
Conjugated AD

| | |
|---|---|
| 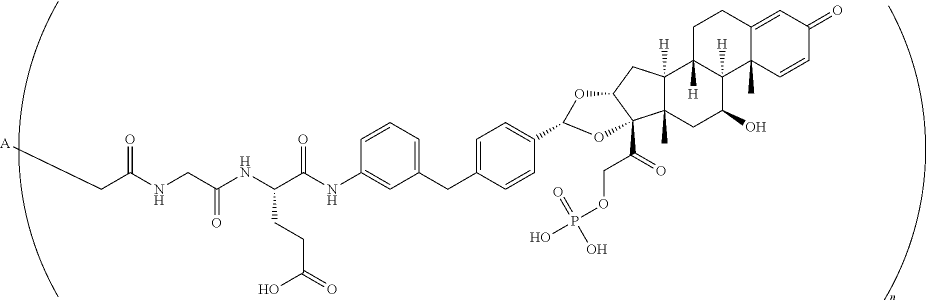 | Example 4 - conjugated |
| 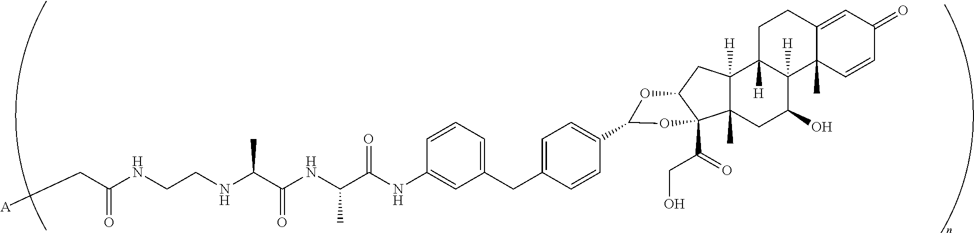 | Example 14A - conjugated |
| 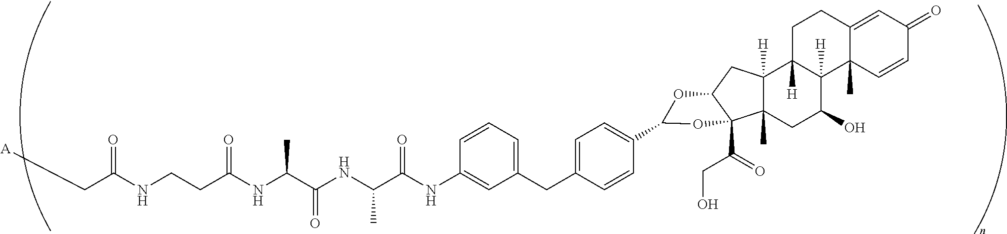 | Example 14B - conjugated |
| 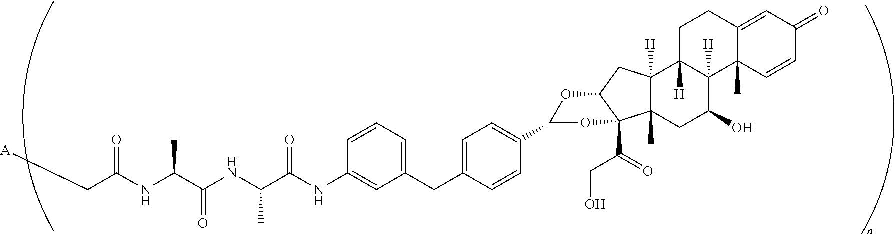 | Example 15 - conjugated |
| 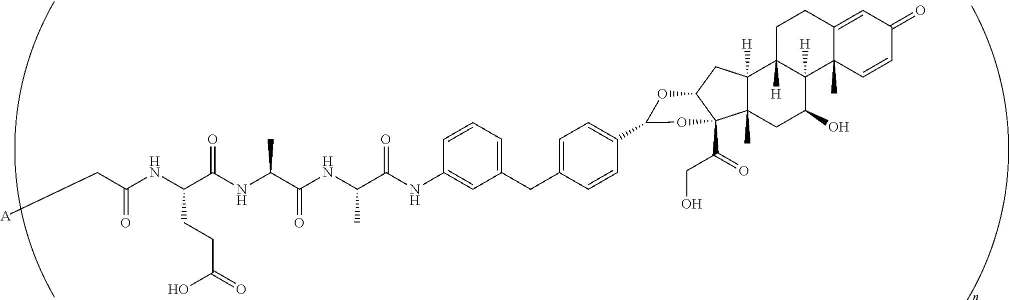 | Example 16 - conjugated |
| 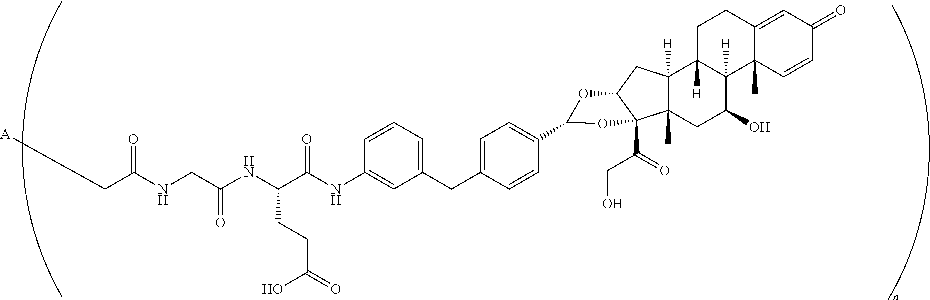 | Example 17 - conjugated |
| 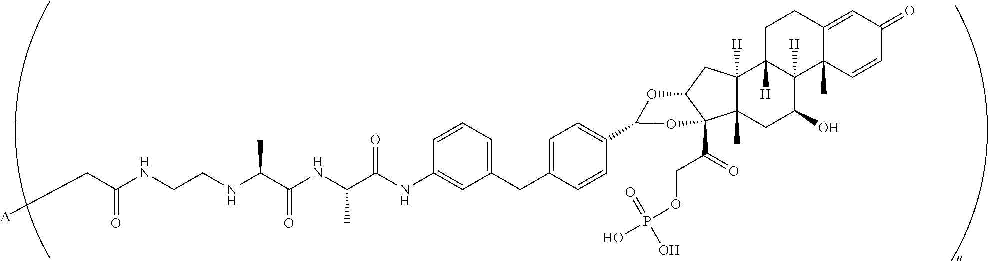 | Example 18A - conjugated |
| 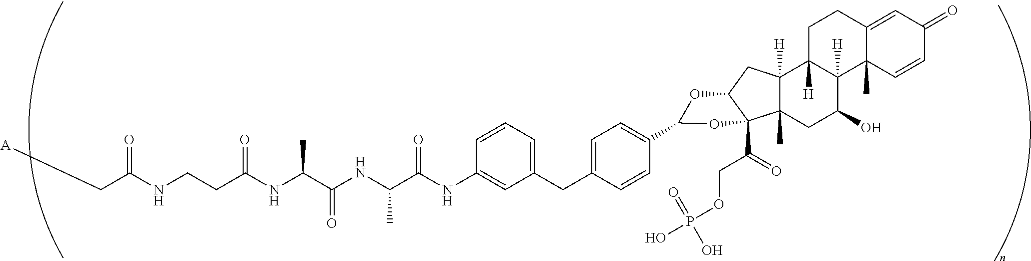 | Example 18B - conjugated |
| 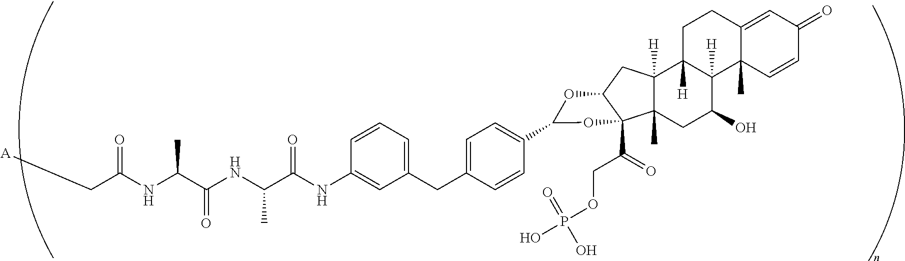 | Example 19 - conjugated |
| 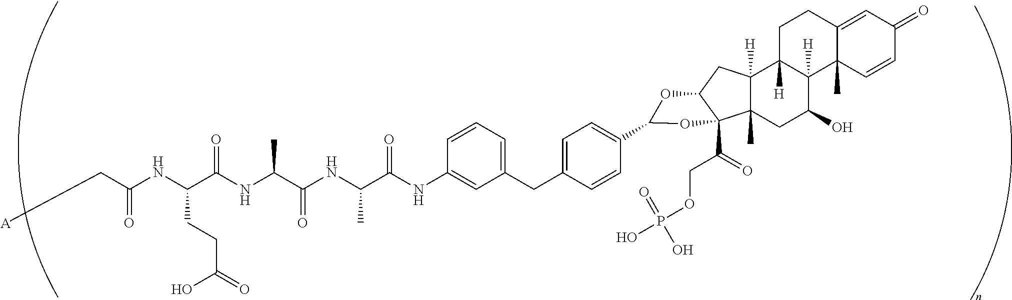 | Example 20 - conjugated |
| 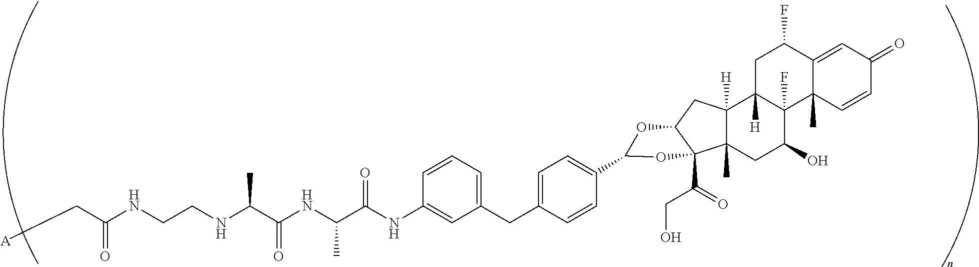 | Example 21A - conjugated |
| 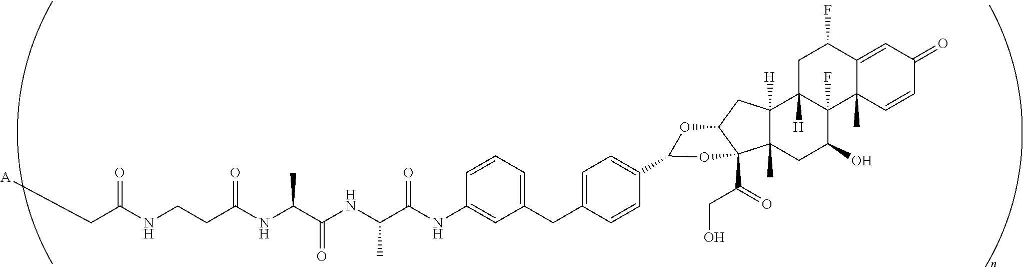 | Example 21B - conjugated |
| 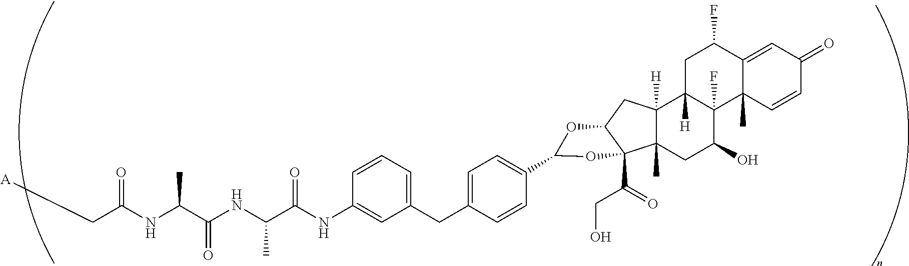 | Example 22 - conjugated |
| 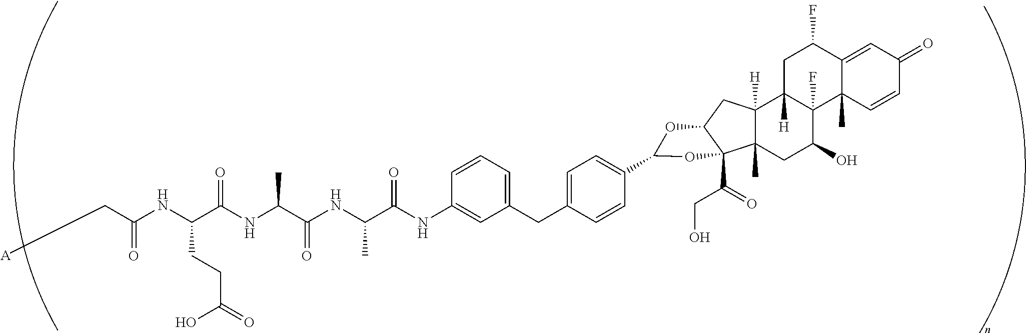 | Example 23 - conjugated |
| 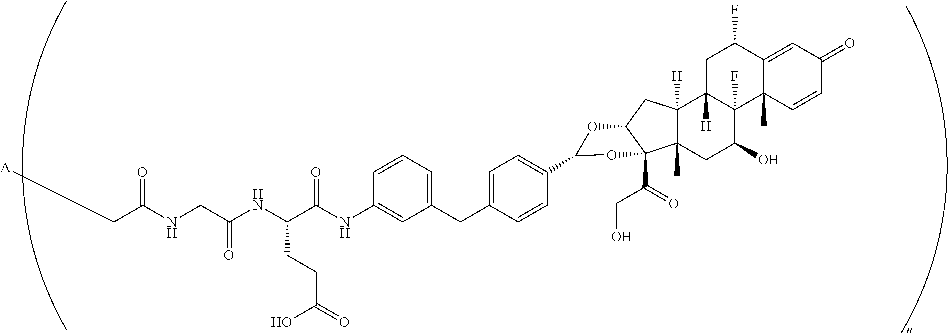 | Example 24 - conjugated |
| 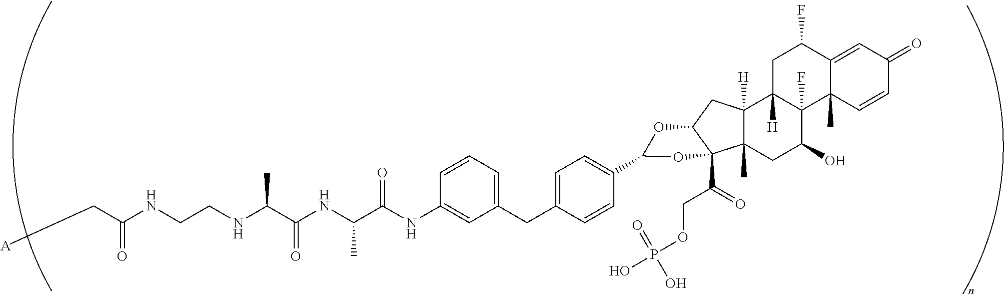 | Example 25A - conjugated |
| 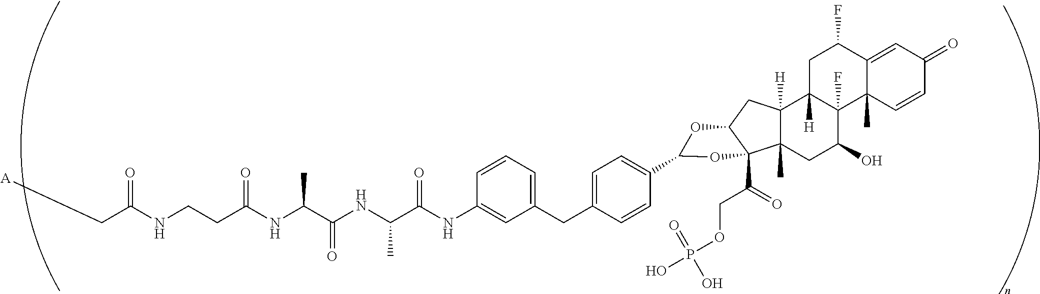 | Example 25B - conjugated |
| 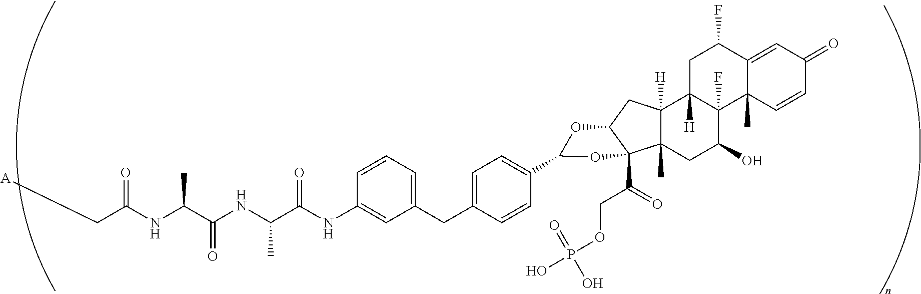 | Example 26 - conjugated |
| 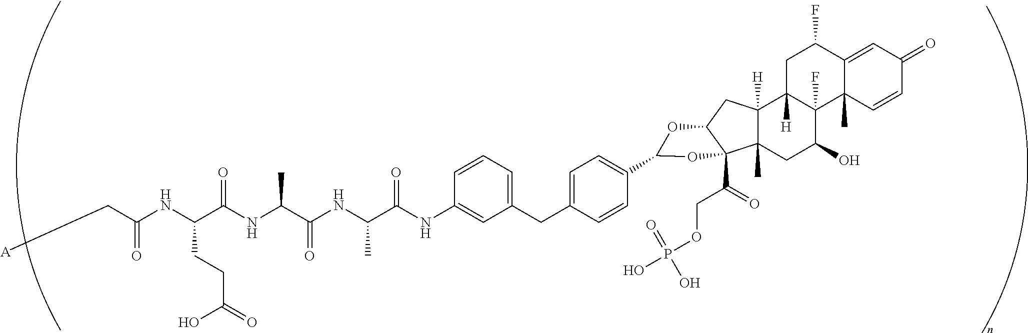 | Example 27 - conjugated |
| 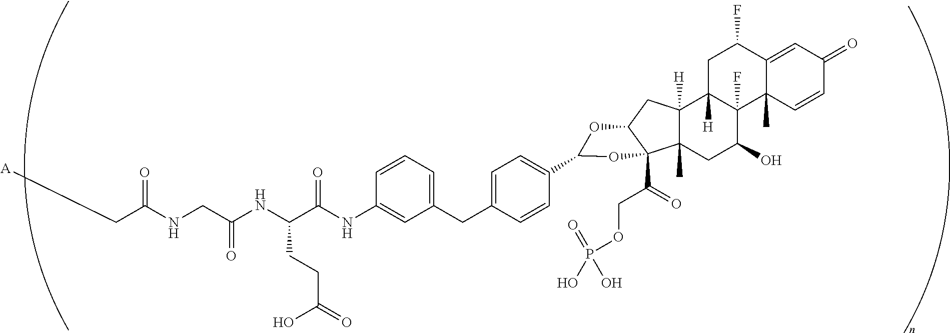 | Example 28 - conjugated |
| 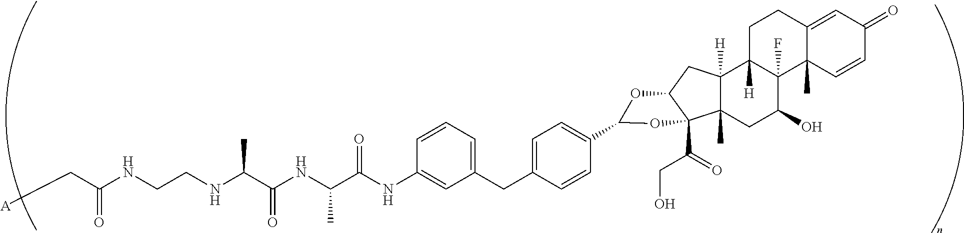 | Example 29A - conjugated |
| 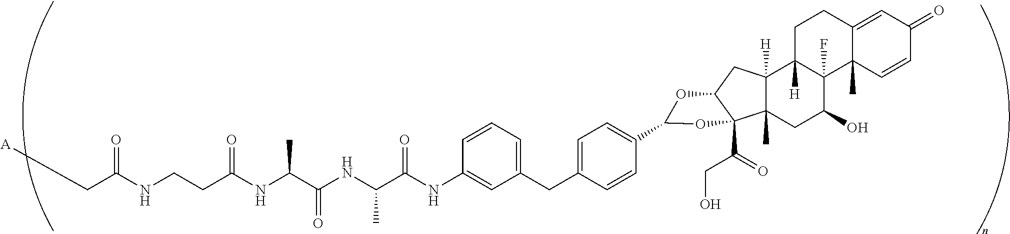 | Example 29B - conjugated |
| 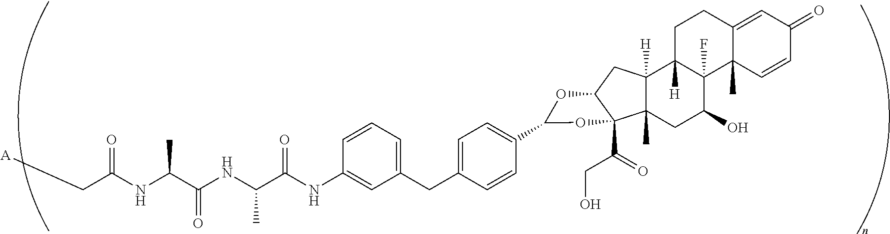 | Example 30 - conjugated |
| 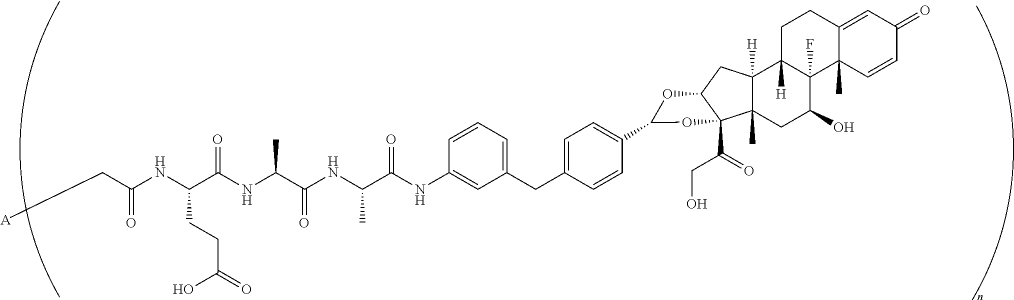 | Example 31 - conjugated |
| 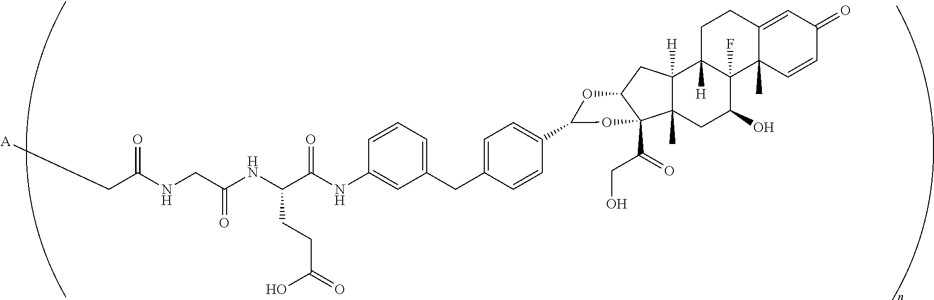 | Example 32 - conjugated |
| 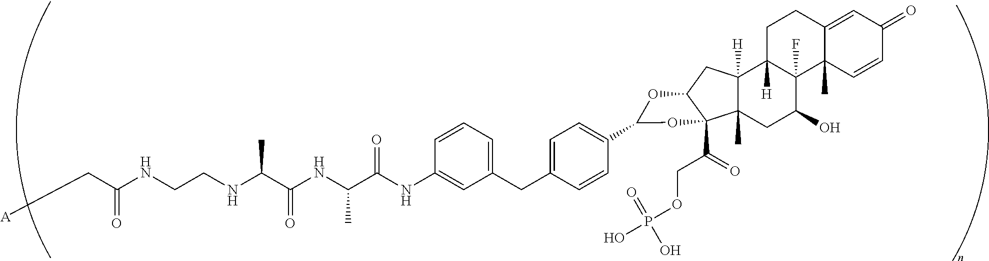 | Example 33A - conjugated |
| 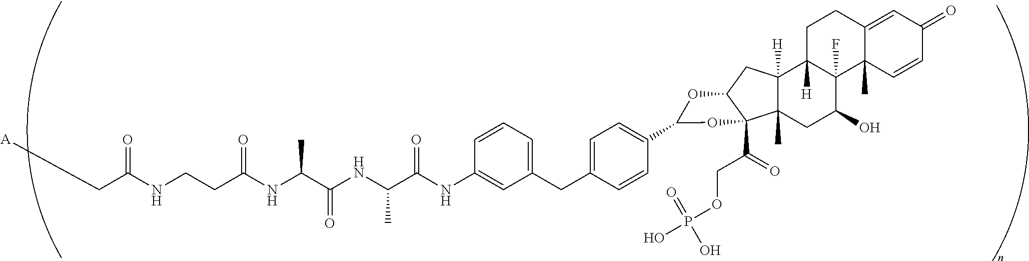 | Example 33B - conjugated |
| 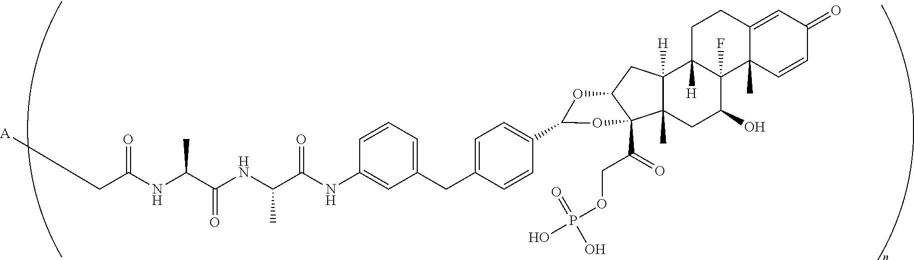 | Example 34 - conjugated |
| 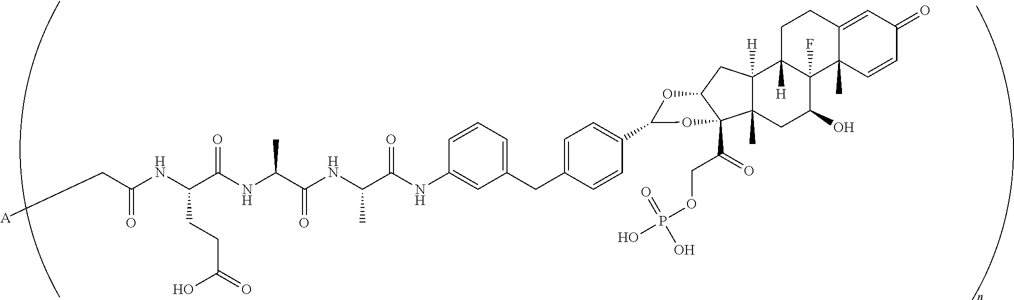 | Example 35 - conjugated |
| 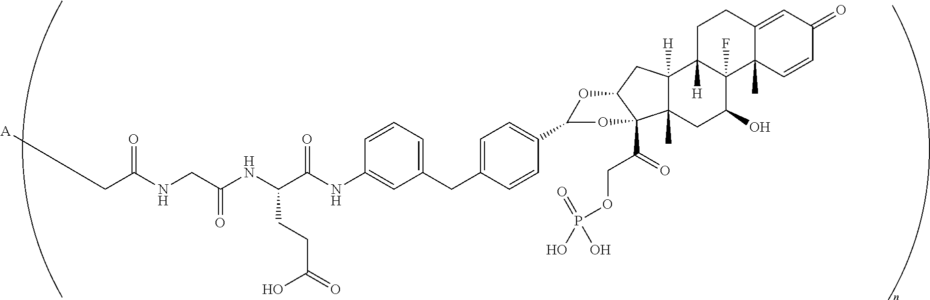 | Example 36 - conjugated |
| 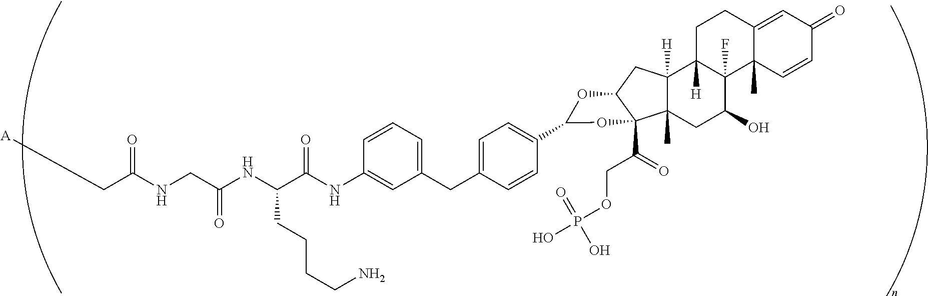 | Example 37 - conjugated |
| 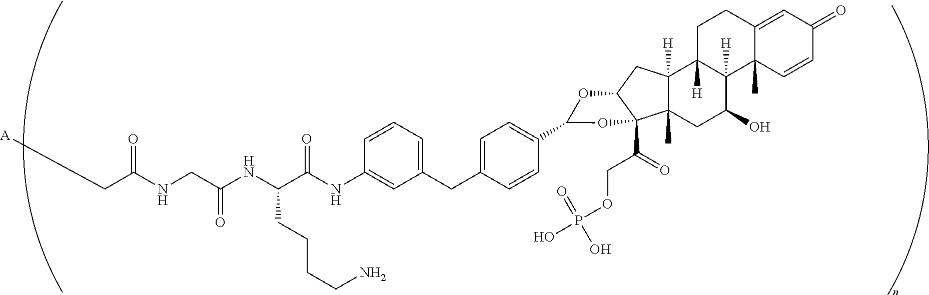 | Example 38 - conjugated |
| 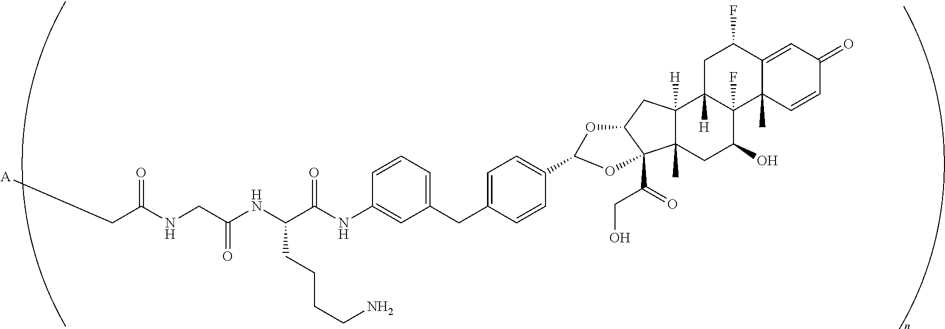 | Example 39 - conjugated |
| 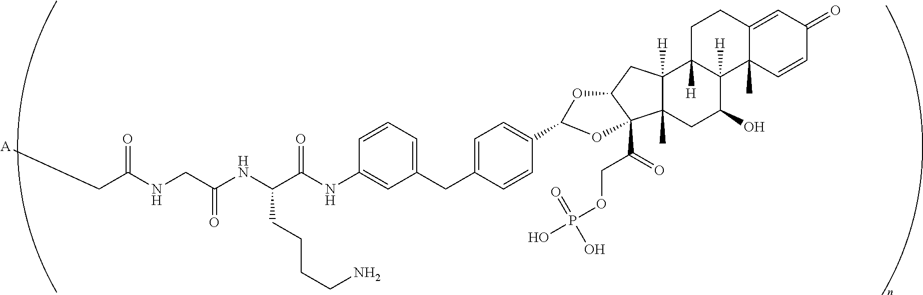 | Example 40 - conjugated |
| 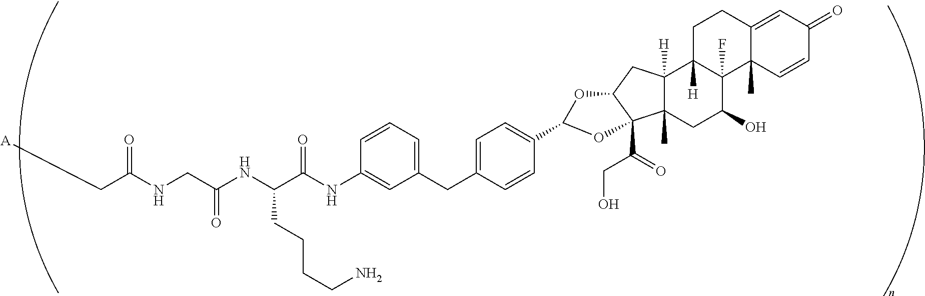 | Example 41 - conjugated |
| 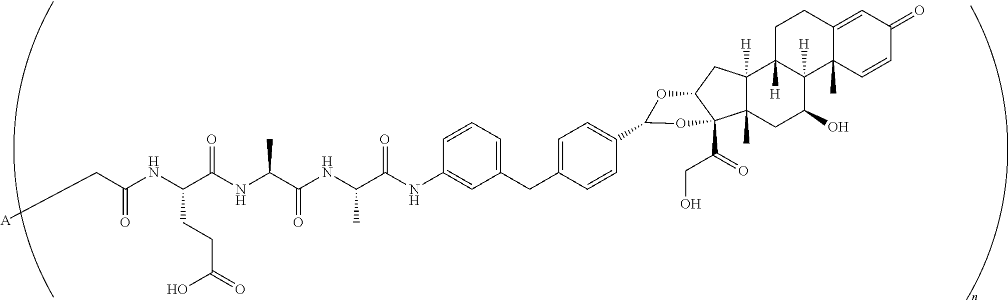 | Example 42 - conjugated |
| 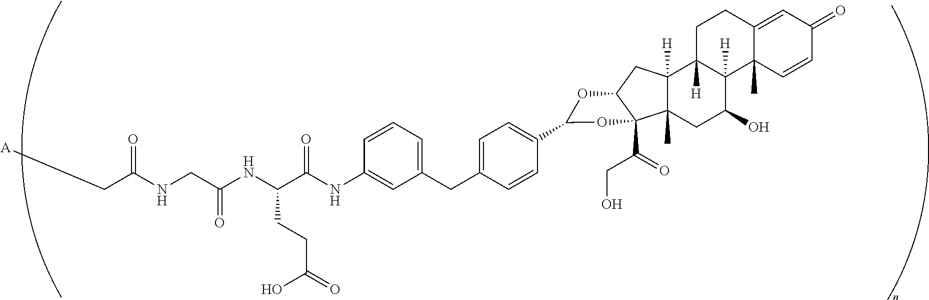 | Example 43 - conjugated |
| 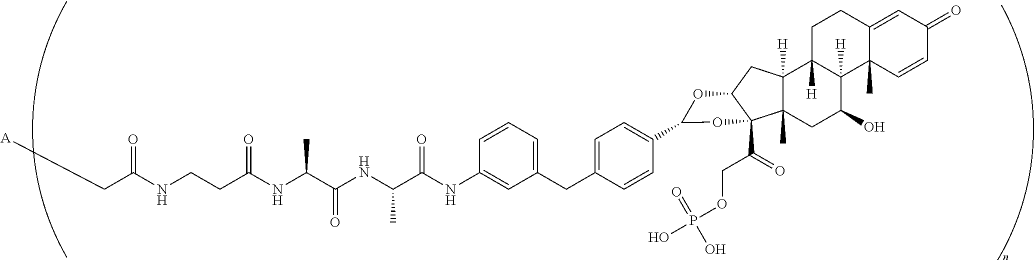 | Example 44 - conjugated |
| 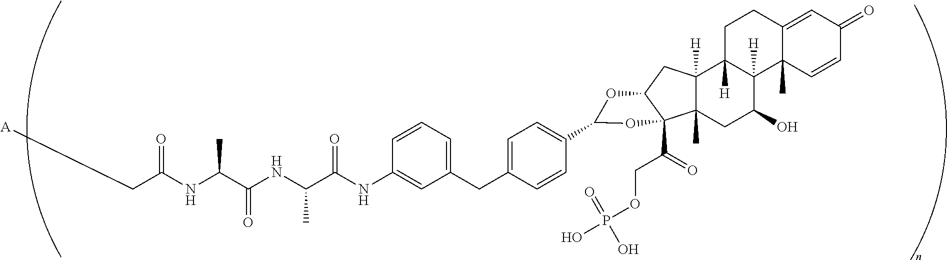 | Example 45 - conjugated |
| 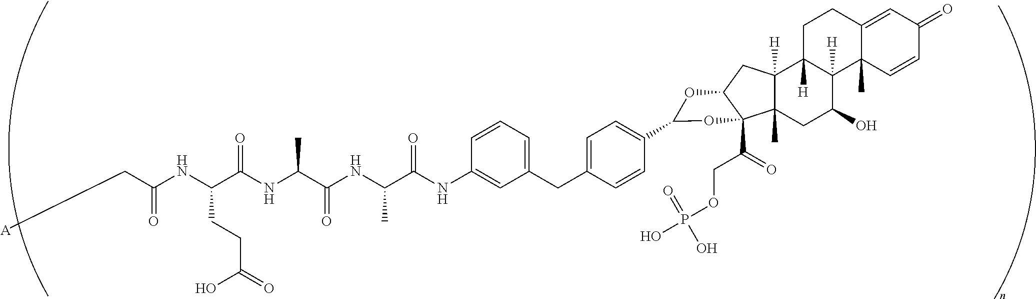 | Example 46 - conjugated |
| 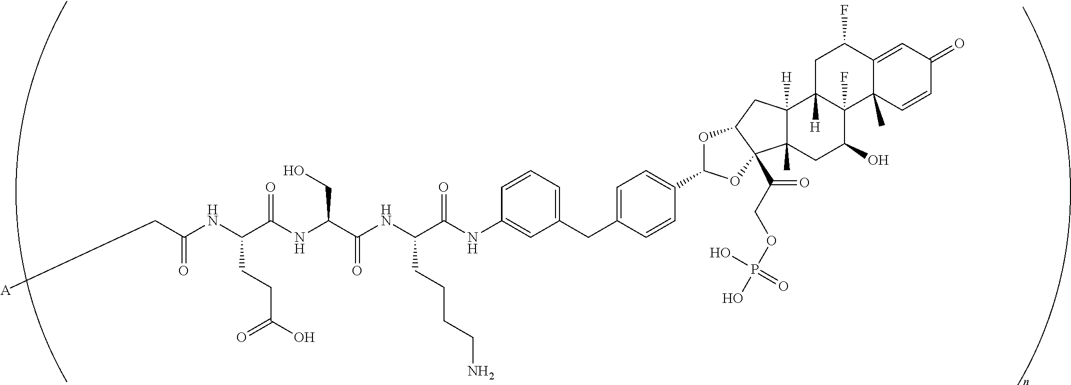 | Example 47 - conjugated |
| 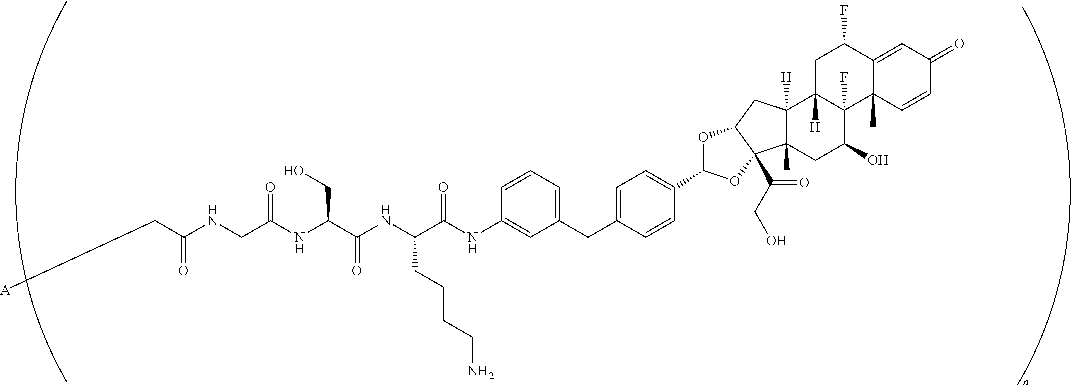 | Example 48 - conjugated |

TABLE 6A
Conjugated ADC

| | |
|---|---|
| 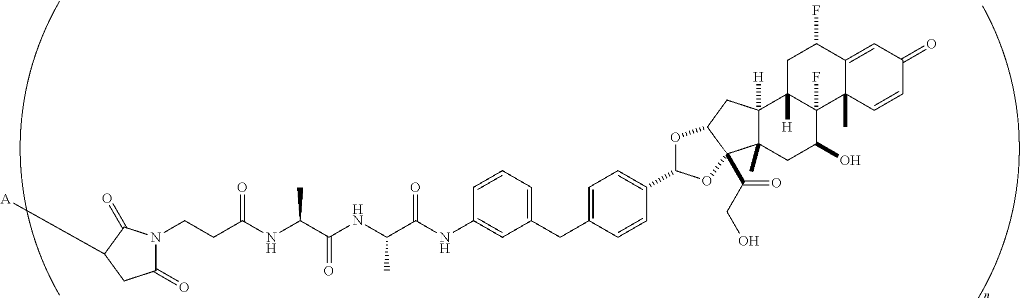 | Example 6 - conjugated |
| 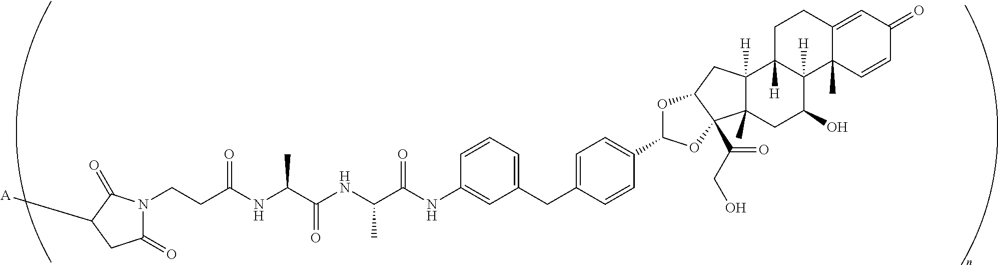 | Example 7 - conjugated |
| 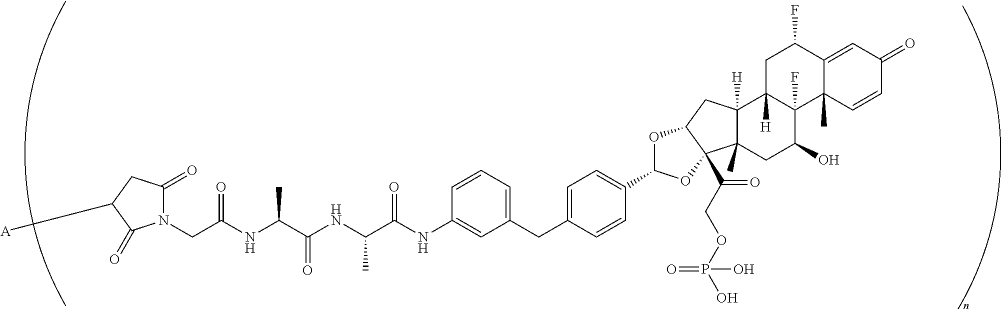 | Example 12 - conjugated |
| 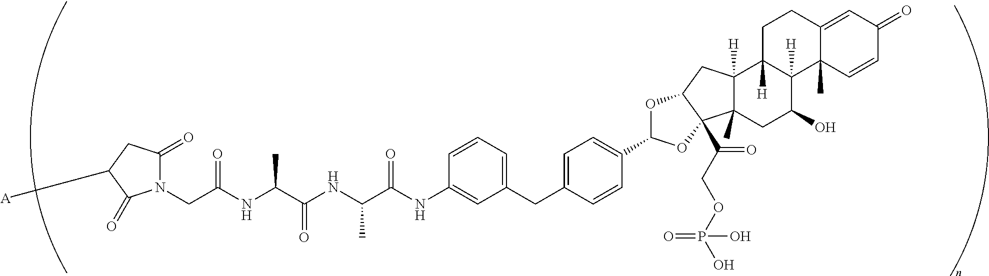 | Example 13 - conjugated |
| 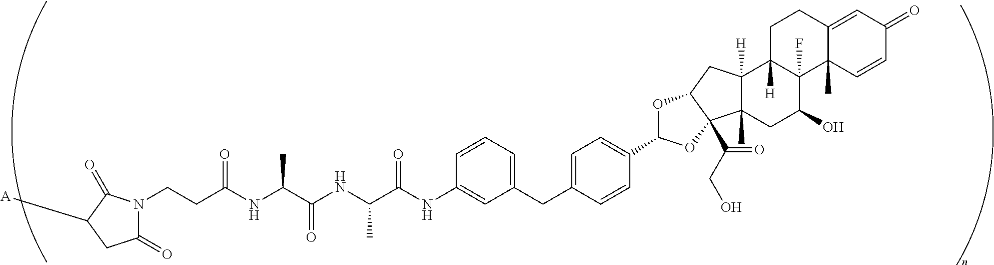 | Example 8 - conjugated |
| 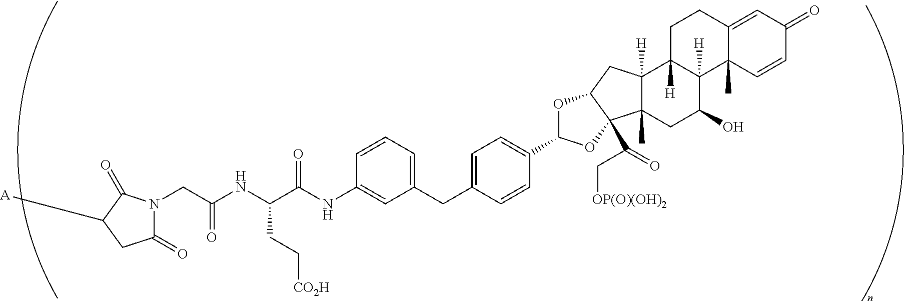 | Example 9 - conjugated |
| 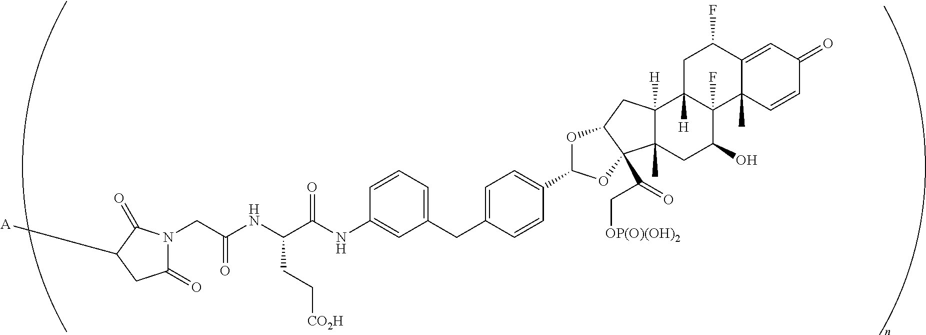 | Example 10 - conjugated |
| 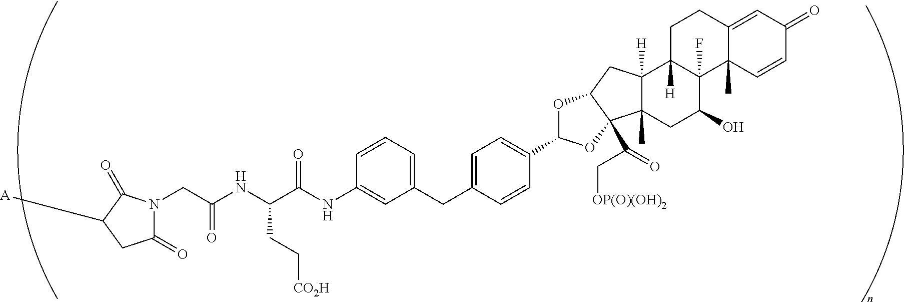 | Example 11 - conjugated |

TABLE 6B
Conjugated and Hydrolyzed ADC

| | |
|---|---|
| 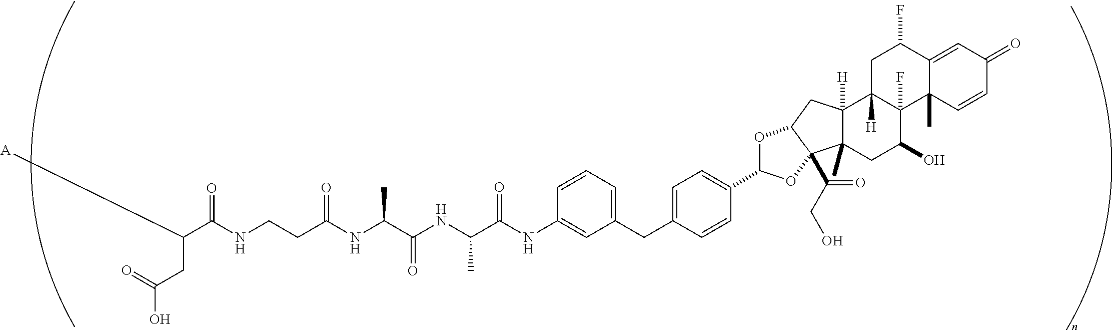 | Example 6 - hydrolyzed |
| 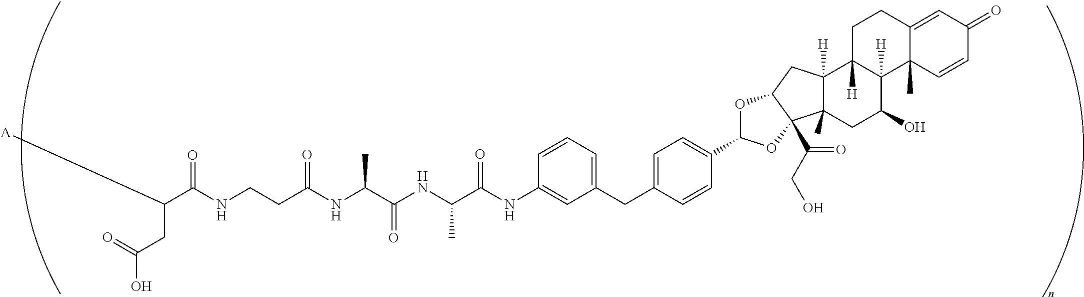 | Example 7 - hydrolyzed |
| 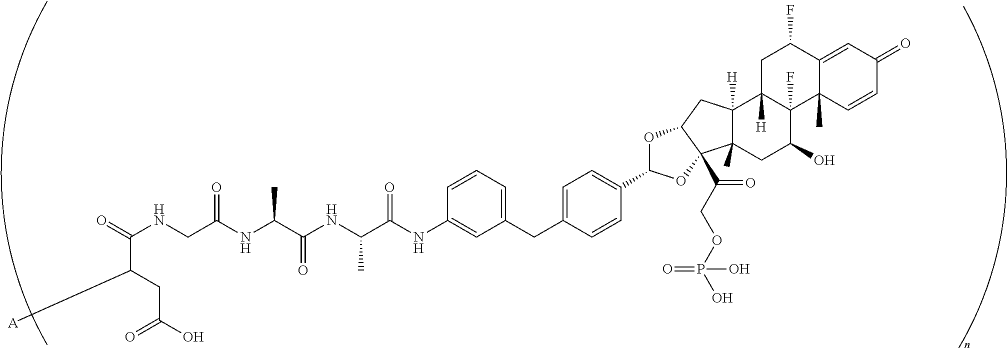 | Example 12 - hydrolyzed |
| 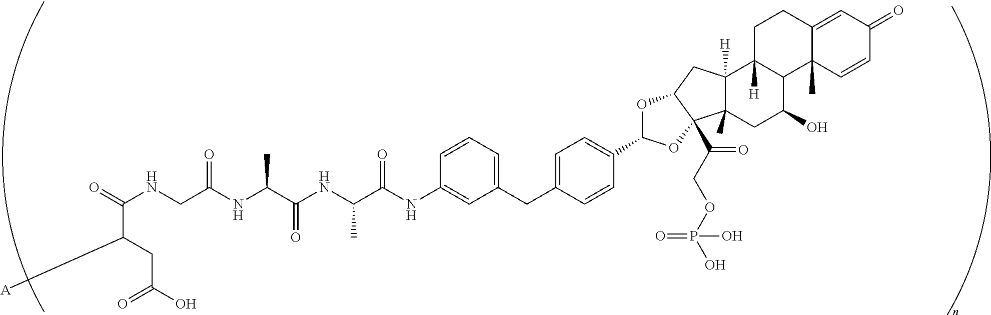 | Example 13 - hydrolyzed |
| 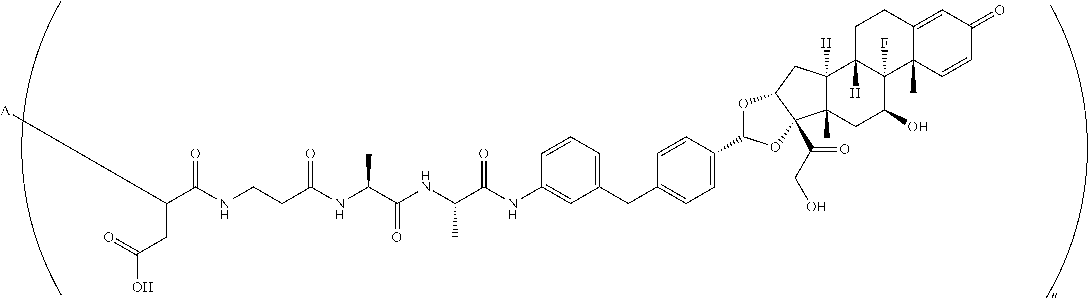 | Example 8 -hydrolyzed |
| 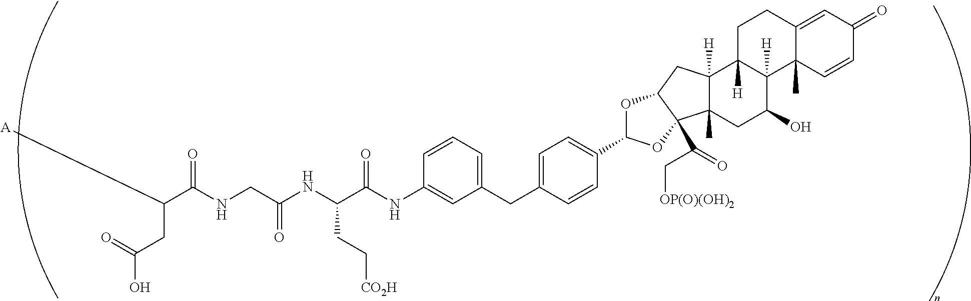 | Example 9 -hydrolyzed |
| 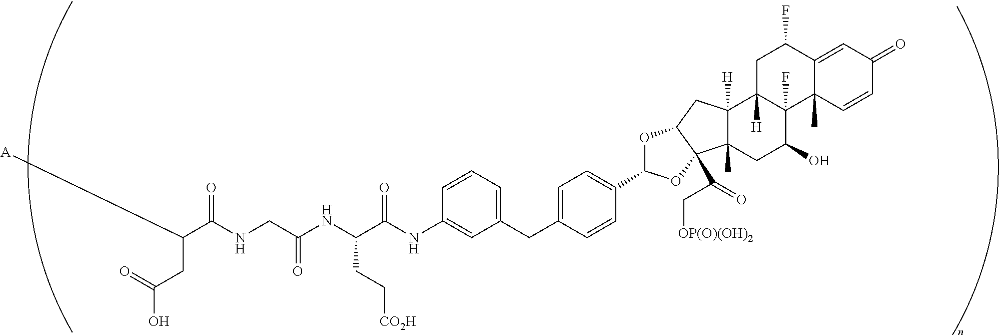 | Example 10 - hydrolyzed |
| 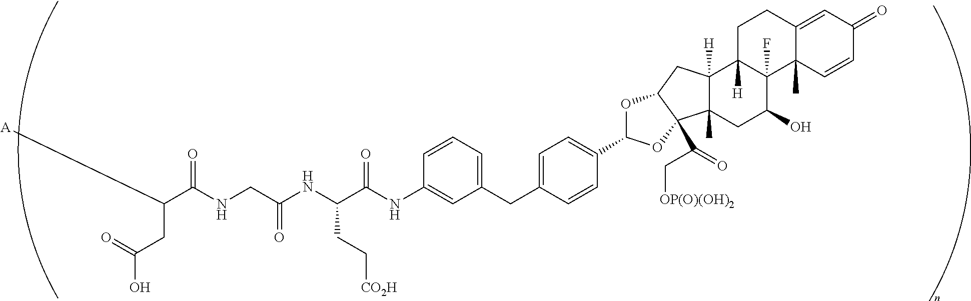 | Example 11 - hydrolyzed |

In certain embodiments of Table 5, the antibody drug conjugate is Example 4-conjugated or Example 28-conjugated. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4. In certain embodiments of Table 5, the antibody drug conjugate is Example 4-conjugated, Example 28-conjugated, or Example 47-conjugated, wherein n is 2 or 4. In certain embodiments of Table 5, the antibody drug conjugate is Example 47-conjugated wherein n is 2. In certain embodiments of Table 5, the antibody drug conjugate is Example 47-conjugated wherein n is 4. In certain embodiments of Table 5, the antibody drug conjugate is Example 28-conjugated wherein n is 2. In certain embodiments of Table 5, the antibody drug conjugate is Example 28-conjugated wherein n is 4.

In certain embodiments of Tables 6A and 6B, the antibody drug conjugate is Example 6-conjugated, Example 6-hydrolyzed, Example 7-conjugated, Example 7-hydrolyzed, Example 12-conjugated, Example 12-hydrolyzed, Example 13-conjugated, or Example 13-hydrolyzed. In certain embodiments, the antibody drug conjugate is Example 6-hydrolyzed, Example 7-hydrolyzed, Example 12-hydrolyzed, or Example 13-hydrolyzed. In certain embodiments, the compound is Example 6-hydrolyzed, Example 7-hydrolyzed, or Example 12-hydrolyzed. In certain embodiments, the antibody drug conjugate is Example 12-hydrolyzed or Example 13-hydrolyzed. In certain embodiments, the anti-CD40 antibody comprises complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12. In certain embodiments, the anti-CD40 antibody comprises a heavy chain variable region set forth as SEQ ID NO: 5 and a light chain variable region set forth as SEQ ID NO: 6. In certain embodiments, the anti-CD40 antibody comprises a heavy chain set forth as SEQ ID NO: 3 and a light chain set forth as SEQ ID NO: 4. In certain embodiments, n is 2. In certain embodiments, n is 4.

III. Methods of Use and Pharmaceutical Compositions

Provided herein are antibody drug conjugates of Formula (I) or (II) that can be used in vitro or in vivo. Accordingly, also provided are compositions, e.g., pharmaceutical compositions for certain in vivo uses, comprising an antibody drug conjugates of Formula (I) or (II) having the desired degree of purity in a physiologically acceptable carrier, excipient or stabilizer (Remington's Pharmaceutical Sciences (1990) Mack Publishing Co., Easton, Pa.). Acceptable carriers, excipients, or stabilizers are nontoxic to recipients at the dosages and concentrations employed.

The compositions (e.g., pharmaceutical compositions) to be used for in vivo administration can be sterile, which can be accomplished by filtration through, e.g., sterile filtration membranes. The compositions (e.g., pharmaceutical compositions) to be used for in vivo administration can comprise a preservative.

The antibody drug conjugates can be formulated in dosage forms and administered (e.g., via intravenous administration or infusion) in accordance with knowledge in the art.

Antibody drug conjugates and/or pharmaceutical compositions comprising antibody drug conjugates described herein can be useful in lysing a cell expressing CD40 (in vitro or in vivo) and/or for the treatment of diseases or disorders characterized by increased CD40. In some embodiments, the antibody drug conjugates and/or compositions are useful in inhibiting cytokine release (in vitro or in vivo) and/or for the treatment of autoimmune or inflammatory diseases.

In certain embodiments, provided is a method of treating a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS), in a subject in need thereof, comprising administering an effective amount of the antibody drug conjugate or pharmaceutical composition, as described herein, to the subject. In certain embodiments, the condition is inflammatory bowel disease (IBD). In certain embodiments, the IBD is ulcerative colitis (UC) or Crohn's disease. In certain embodiments, the condition is systemic lupus erythematosus (SLE). In certain embodiments, the condition is multiple sclerosis. In certain embodiments, the condition is rheumatoid arthritis. In certain embodiments, the condition is Sjogren's syndrome. In certain embodiments, the condition is Hidradenitis suppurativa (HS).

In another embodiment, provided is the antibody drug conjugate or the pharmaceutical composition, as described herein, for use in the treatment of a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS).

In another embodiment, provided is the antibody drug conjugate or the pharmaceutical composition, as described herein, for preparation of a medicament for treating a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS).

Some embodiments comprise methods of delivering a glucocorticoid receptor agonist to a CD40-expressing cell. Such methods can include a step of contacting a CD40-expressing cell with an antibody drug conjugate as described herein. Some embodiments comprise an in vitro method of delivering a glucocorticoid receptor agonist to a CD40-expressing cell.

Also provided are methods of determining anti-inflammatory activity of an antibody drug conjugate. Such methods can include a step of contacting a CD40-expressing cell with an antibody drug conjugate as described herein. Some embodiments comprise contacting a CD40-expressing cell with an antibody drug conjugate as described herein and determining reduced release of pro-inflammatory cytokines from the cell as compared to a control cell. Some embodiments comprise an in vitro method of determining anti-inflammatory activity of an antibody drug conjugate.

Some embodiments comprise screening methods (e.g. in vitro methods) that include contacting, directly or indirectly, cells (e.g., CD40-expressing cells) with an antibody drug conjugate and determining if the antibody drug conjugate modulates an activity or function of the cells, as reflected for example by changes in cell morphology or viability, expression of a marker, differentiation or de-differentiation, cell respiration, mitochondrial activity, membrane integrity, maturation, proliferation, viability, apoptosis or cell death. One example of a direct interaction is physical interaction, while an indirect interaction includes, for example, the action of a composition upon an intermediary molecule that, in turn, acts upon the referenced entity (e.g., cell or cell culture).

Thus, in certain embodiments, provided is a method of delivering a glucocorticoid receptor agonist to a CD40-expressing cell, comprising the step of contacting the cell with the antibody drug conjugate or a pharmaceutical composition as described herein.

In certain further embodiments, provided is a method of determining anti-inflammatory activity of an antibody drug conjugate comprising contacting a CD40-expressing cell with the antibody drug conjugate as described herein; and determining a reduced release of pro-inflammatory cytokines from the cell as compared to a control cell.

IV. Articles of Manufacture

The disclosure also includes pharmaceutical packs and kits comprising one or more containers, wherein a container can comprise one or more doses of an antibody drug conjugate or composition as described herein. In certain embodiments, the pack or kit contains a unit dosage, meaning a predetermined amount of a composition or antibody drug conjugate, with or without one or more additional agents.

In some embodiments, the kits are provided in one or more liquid solutions, which can be a non-aqueous or aqueous solution. In some embodiments, the solution is a sterile solution. The composition in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution.

The kit can comprise one or multiple containers and a label or package insert in, on or associated with the container(s), indicating that the enclosed composition is used for treating the disease condition of choice. Suitable containers include, for example, bottles, vials, syringes, etc. The containers can be formed from a variety of materials such as glass or plastic. The container(s) can comprise a sterile access port, for example, the container may be an intravenous solution bag or a vial having a stopper that can be pierced by a hypodermic injection needle.

In some embodiments the kit can contain a means by which to administer the antibody drug conjugate and any optional components to a subject in need thereof, e.g., one or more needles or syringes (pre-filled or empty), an eye dropper, pipette, or other such like apparatus, from which the composition may be injected or introduced into the subject or applied to a diseased area of the body. The kits of the disclosure will also typically include a means for containing the vials, or such like, and other components in close confinement for commercial sale, such as, e.g., blow-molded plastic containers into which the desired vials and other apparatus are placed and retained.

Thus, in certain embodiments, provided is a kit comprising:

(a) a container comprising the antibody drug conjugate or a pharmaceutical composition as described herein; and

(b) a label or package insert on or associated with the one or more containers, wherein the label or package insert indicates that the antibody drug conjugate or pharmaceutical composition is used for treating a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS).

### EXAMPLES

It is understood that the examples and embodiments described herein are for illustrative purposes only and that various modifications or changes in light thereof will be suggested to persons skilled in the art and are to be included within the spirit and purview of this disclosure.

### ANALYTICAL METHODS

1. Small Molecule Analytical Procedures

Unless otherwise stated, all ¹H and ¹³C NMR (Nuclear magnetic resonance) data were collected on a Varian Mercury Plus 400 MHz or a Bruker AVIII 300 MHz instrument; chemical shifts are quoted in parts per million (ppm). High performance liquid chromatography (HPLC) and LCMS analytical data are either detailed within the experimental or referenced to the conditions listed in Table 7.

TABLE 7
List of LCMS and HPLC Methods

| Method | Conditions |
|---|---|
| a | The gradient was 1-90% B in 3.4 min, 90-100% B in 0.45 min, 100-1% B in 0.01 min, and then |
| | held at 1% B for 0.65 min (0.8 mL/min flow rate). Mobile phase A was 0.0375% Trifluoro |
| | acetic acid in H₂O, mobile phase B was 0.018% trifluoroacetic acid in acetonitrile. The column |
| | used for the chromatography was a 2.0 × 50 mm Phenomenex Luna-C18 column (5 μm |
| | particles). Detection methods are Diode array (DAD) and Evaporative light scattering detector |
| | (ELSD) as well as positive electrospray ionization. |
| b | A gradient of 5-100% acetonitrile (A) and 0.1% Trifluoro acetic acid in water (B) was used, at a |
| | flow rate of 1.5 mL/min (0-0.05 min 5% A, 0.05-1.2 min 5-100% A, 1.2-1.4 min 100% A, 1.4- |
| | 1.5 min 100-5% A. 0.25 min post-run delay). |
| c | The gradient was 5-90% B in 3.4 min, 90-100% B in 0.45 min, 100-5% B in 0.01 min, and then |
| | held at 5% B for 0.65 min (0.8 mL/min flow rate). Mobile phase A was 10 mM NH₄HCO₃, |
| | mobile phase B was HPLC grade acetonitrile. The column used for the chromatography is a |
| | 2.1 × 50 mm Xbridge Shield RPC18 column (5 μm particles). Detection methods are diode array |
| | (DAD) and evaporative light scattering (ELSD) detection as well as positive electrospray |
| | ionization(MS) |
| AA1 | Instrument: Shimadzu LC-8A preparative HPLC. Column: Phenomenex Luna C18 |
| | 200*40 mm*10 um. Mobile phase A: H₂O (0.09% trifluoroacetic acid) and B: acetonitrile. |
| | Gradient: B from 40% to 60% in 20 min. Flow rate: 60 mL/min. Wavelength: 220 & 254 nm. |
| AA2 | Instrument: Gilson 281 semi-preparative HPLC system. Mobile phase A: trifluoroacetic acid/ |
| | H₂O = 0.075% v/v; B: acetonitrile. Column: Nano-micro Kromasil C18 100*30 mm 5 um. |
| | Flow rate: 25 mL/min. Monitor wavelength: 220 & 254 nm. |
| AA3 | Instrument: Gilson 281 semi-preparative HPLC system. Mobile phase A: trifluoroacetic acid/ |
| | H₂O = 0.075% v/v; B: acetonitrile. Column: Phenomenex Synergi C18 100*30 mm*4 um |
| | Flow rate: 25 mL/min. Monitor wavelength: 220 & 254 nm. |
| AA4 | The gradient was 1-90% B in 3.4 min, 90-100% B in 0.45 min, 100-1% B in 0.01 min, and then |
| | held at 1% B for 0.65 min (0.8 mL/min flow rate). Mobile phase A: 0.0375% trifluoroacetic acid |
| | in water, B: 0.018% trifluoroacetic acid in acetonitrile. The column used for the chromatography |
| | was a 2.0 × 50 mm Phenomenex Luna-C18 column (5 μm particles). Detection methods are |
| | DAD and ELSD detection as well as positive electrospray ionization (MS). |
| AA5 | Instrument: Gilson 281 semi-preparative HPLC system. Mobile phase A: trifluoroacetic acid/ |
| | H₂O = 0.075% v/v; B: acetonitrile. Column: Luna C18 100*30 5 um. Flow rate: 25 mL/min. |
| | Monitor wavelength: 220 & 254 nm. Gradient B 25-100% over 10 min. |
| AA6 | Instrument: Shimadzu LC-8A preparative HPLC. Column: Phenomenex Luna C18 200*40 |
| | mm* 10 um. Mobile phase A: H₂O (0.09% trifluoroacetic acid); B: acetonitrile. Gradient: B from |
| | 15% to 45% in 20 min. Flow rate: 60 mL/min. Wavelength: 220 & 254 nm. |
| AA7 | Instrument: Shimadzu LC-8A preparative HPLC. Column: Phenomenex Luna C18 200*40 |
| | mm* 10 um. Mobile phase A: H₂O and B: acetonitrile. Gradient: B from 50% to 100% in 30 min. |
| | Flow rate: 60 mL/min. Wavelength: 220 & 254 nm. |
| AA8 | Column: Phenomenex Luna C18 200*40 mm*10 um. Mobile phase A: H₂O (0.09% |
| | trifluoroacetic acid) and B: acetonitrile. Gradient: B from 35% to 55% in 20 min. Flow rate: 60 |
| | mL/min. Wavelength: 220 & 254 nm. |
| AA9 | Instrument: Gilson 281 semi-preparative HPLC system. Mobile phase A: trifluoroacetic acid/ |
| | H₂O = 0.075% v/v; B: acetonitrile. Column: Luna C18 100*30 5 um. Flow rate: 15 mL/min. |
| | Monitor wavelength: 220 & 254 nm. Gradient B 15-100% over 10 min. |
| AA10 | Instrument: Shimadzu LC-8A preparative HPLC. Column: Phenomenex Luna C18 |
| | 200*40 mm*10 um. Mobile phase A H₂O (0.09% trifluoroacetic acid) and B: acetonitrile. |
| | Gradient: B from 20% to 40% in 20 min. Flow rate: 60 mL/min. Wavelength: 220 & 254 nm. |
| AA11 | Instrument: Gilson 281 semi-preparative HPLC system. Mobile phase A: trifluoroacetic acid/ |
| | H₂O = 0.075% v/v; B: acetonitrile. Column: Luna C18 100*30 5 um. Flow rate: 15 mL/min. |
| | Monitor wavelength: 220 & 254 nm. Gradient B 50-100% over 10 min. |
| AA12 | Instrument: Gilson 281 semi-preparative HPLC system. Mobile phase A: trifluoroacetic acid/ |
| | H₂O = 0.075% v/v; B: acetonitrile. Column: Luna C18 100*30 5 um. Flow rate: 25 mL/min. |
| | Monitor wavelength: 220 & 254 nm. Gradient B 10-100% over 10 min. |
| AA13 | The gradient was 5-95% B in 0.7 min, 95-95% B in 0.45 min, 95-5% B in 0.01 min, and then |
| | held at 0% B for 0.44 min (1.5 mL/min flow rate). Mobile phase A: 0.0375% trifluoroacetic acid |
| | in water, mobile phase B: 0.018% trifluoroacetic acid in acetonitrile. The column used for the |
| | chromatography is a Chromolith Flash RP-18e 25-2 mm column. Detection methods are DAD |
| | and ELSD detection as well as positive electrospray ionization (MS). |
| AA14 | Instrument: Shimadzu LC-8A preparative HPLC. Column: Phenomenex Luna C18 200*40 |
| | mm* 10 um. Mobile phase A: H₂O (0.05% trifluoroacetic acid) and B: acetonitrile. Gradient: B |
| | from 30% to 100% in 30 min. Flow rate: 60 mL/min. Wavelength: 220 & 254 nm. |
| AA15 | The gradient was 10-80% B in 4 min, held at 80% B for 0.9 min, 80-10% B in 0.01 min, and |
| | then held at 10% B for 1 min (0.8 mL/min flow rate). Mobile phase A was 0.0375% |
| | trifluoroacetic acid in water, mobile phase B was 0.018% trifluoroacetic acid in acetonitrile. The |
| | column used for the chromatography was a 2.0 × 50 mm phenomenex Luna-C18 column (5 μm |
| | particles). Detection method is DAD. |
| AA16 | The gradient was 15-100% B in 8.00 min and hold at 100% B for 2 mins, 100-15% B in 0.01 min, |
| | and then held at 15% for 5.00 min, the flow rate was 0.80 ml/min. Mobile phase A was 10 mM |
| | ammonium bicarbonate, mobile phase B was HPLC grade acetonitrile. The column used for |
| | chromatography was a 2.1*50 mm Xbridge Shield RPC18 column (5 um particles). Detection |
| | methods are DAD and ELSD detection as well as positive electrospray ionization. |
| AA17 | Instrument: Shimadzu LC-8A preparative HPLC. Column: Phenomenex Luna C18 200*40 |
| | mm* 10 um. Mobile phase: A for H₂O (0.09% trifluoroacetic acid) and B for acetonitrile. |
| | Gradient: B from 30% to 40% in 20 min. Flow rate: 60 mL/min. Wavelength: 220&254 nm |
| AA18 | The gradient was 5-95% B in 1.0 min, 95-100% B in 0.80 min, 100-5% B in 0.01 min, and then |
| | held at 5% B for 0.39 min (1.0 mL/min flow rate). Mobile phase A was 0.0375% TFA in water, |
| | mobile phase B was 0.018% TFA in MeCN. The column used for the chromatography was a |
| | ZORBAX Eclipse XDB-C18 2.1*30 mm, 3.5 um. Detection methods are diode array (DAD) and |
| | positive electrospray ionization(MS). |
| AA19 | The gradient was 10-90% B in 1.15 min, held at 90% B for 0.50 min, 90-10% B in 0.01 min, and |
| | then held at 10% B for 0.34 min. Mobile phase A was 10 mM NH₄HCO₃ in water, mobile phase |
| | B was MeCN. The column used for the chromatography was a Xbridge Shield RP18 |
| | 2.1*50 mm, 5 um. Dtection methods are diode array (DAD) and positive electrospray |
| | ionization(MS).). The system flow rate was 0.8 mL/min (0.00-1.51 min) 1.2 mL/min (1.52- |
| | 2.00 min). |
| AA20 | Instrument: Gilson 281 semi-preparative HPLC system. Mobile phase: A: NH₄OH/H₂O = 0.040% |
| | v/v; B: MeCN. Column: YMC-Actus Triart C18 100*30 mm*5 um. Flow rate: 25 mL/min. |
| | Monitor wavelength: 220 & 254 nm. Gradient B 10-30% over 12 min. |

2. ADC Analytical Procedures

ADCs were profiled by either anionic exchange chromatography (AEC) or Hydrophobic Interaction Chromatography (HIC) to determine the degree of conjugation and purity of ADC.

### Anionic Exchange Chromatography (AEC).

Approximately 20 ug of ADC was loaded onto an Ultimate 3000 Dual LC system (Thermo Scientific) equipped with a 4×250 mm Propac™ WAX-10 column (Tosoh Bioscience, cat. 054999). Column was equilibrated with 100% buffer A and eluted using a linear gradient from 100% buffer A to 100% buffer B over 18 min at 1.0 mL/min, where buffer A is 20 mM MES, pH 6.7 and buffer B is 20 mM MES, 500 sodium chloride, pH 6.7.

### Hydrophobic Interaction Chromatography (HIC).

Approximately 20 ug of the ADC was loaded onto an Ultimate 3000 Dual LC system (Thermo Scientific) equipped with a 4.6×35 mm butyl-NPR column (Tosoh Bioscience, cat. 14947). Column was equilibrated in 100% buffer A and eluted using a linear gradient from 100% buffer A to 100% buffer B over 12 min at 0.8 mL/min, where buffer A is 25 mM sodium phosphate, 1.5 M ammonium sulfate, pH 7.0 and buffer B is 25 mM sodium phosphate, 25% isopropanol, pH 7.0.

Size exclusion chromatography (SEC).

Size distributions of the ADCs were profiled by size exclusion SEC using an Ultimate 3000 Dual LC system (Thermo Scientific) equipped with a 7.8×300 mm TSK-gel 3000SW_{XL} column (Tosoh Bioscience, cat. 08541). Approximately 20 ug of ADC was loaded onto the column and eluted over 17 min using an isocratic gradient of 100 mM sodium sulfate, 100 mM sodium phosphate, pH 6.8 at a flow rate of 1.0 mL/min.

Aggregation studies can also be conducted using SEC, and the percentage of the aggregate can be measured by integrating the area of the aggregates peak as determined by gel filtration standard (Bio-rad, 151-1901).

### Mass Spectroscopy (MS).

Reduced samples (10 μL) were injected to an Agilent 6550 QT of LC/MS system through a temperature controlled (5° C.) CTC autosampler. Sample elution was achieved on a Waters C-4, 3.5 μm, 300 Å, 2.1×50 mm i.d. HPLC column. The mobile phases were: A: 0.1% formic acid in water, and B: 0.1% formic acid in acetonitrile; the flow rate was 0.45 mL/min, and the column compartment was maintained at 40° C. The HPLC gradient is as follows:

TABLE 8
Gradient for LCMS

| Time (min) | % A | % B |
|---|---|---|
| 0 | 95 | 5 |
| 0.6 | 95 | 5 |
| 1.1 | 10 | 90 |
| 2.2 | 10 | 90 |
| 2.4 | 95 | 5 |
| 3.5 | 95 | 5 |

### Synthesis of Precursor Molecules

### Precursor Example 1: Synthesis of (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

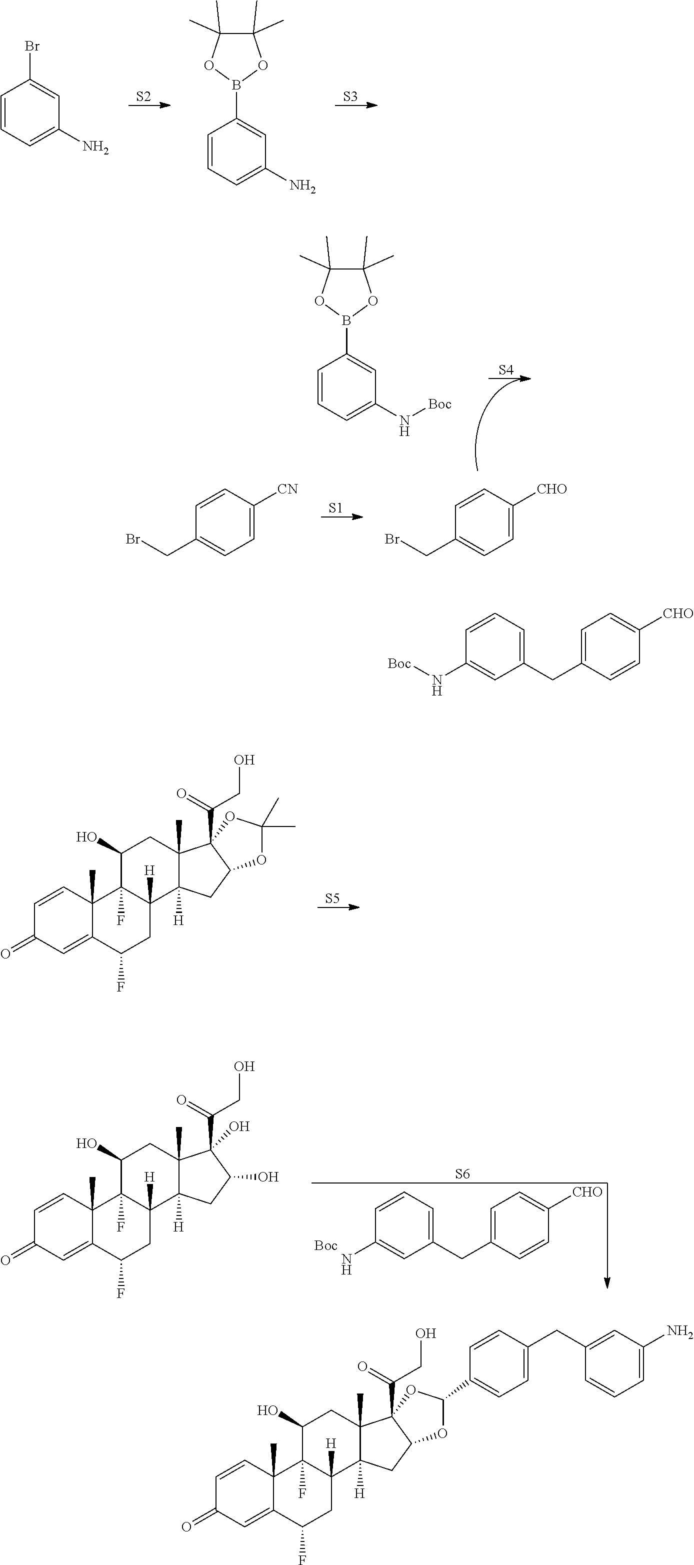

Step 1: Synthesis of 4-(bromomethyl)benzaldehyde. Diisobutylaluminum hydride (153 mL, 153 mmol, 1 M in toluene) was added drop-wise to a 0° C. solution of 4-(bromomethyl)benzonitrile (20 g, 102 mmol) in toluene (400 mL) over 1 hour. Two additional reactions were set up as described above. All three reaction mixtures were combined. To the mixture was added 10% aqueous HCl (1.5 L). The mixture was extracted with dichloromethane (3×500 mL). The organic layer was dried over Na₂SO₄, filtered and concentrated under reduced pressure. The residue was purified by column chromatography on silica gel (eluted with Petroleum ether/Ethyl acetate=10/1) to obtain the title compound (50 g, yield 82%). ¹H NMR (400 MHz, CDCl₃) δ 10.02 (s, 1H), 7.91-7.82 (m, 2H), 7.56 (d, J=7.9 Hz, 2H), 4.55-4.45 (m, 2H).

Step 2: Synthesis of 3-(4,4,5,5-tetramethyl-1,3,2-dioxaborolan-2-yl)aniline. To a solution of 3-bromoaniline (40 g, 233 mmol) in 1,4-dioxane (480 mL) was added 4,4,4′,4′,5,5,5′,5′-tetramethyl-2,2′-bi(1,3,2-dioxaborolane) (94 g, 372 mmol), potassium acetate (45.6 g, 465 mmol), 2-dicyclohexylphosphino-2′,4′,6′-tri-i-propyl-1,1′-biphenyl (8.07 g, 13.95 mmol), tris(dibenzylideneacetone)dipalladium(0) (8.52 g, 9.30 mmol). Then the resulting mixture was heated at 80° C. for 4 hours under nitrogen. An additional reaction was set up as described above. The two reaction mixtures were combined and concentrated and the residue was purified by column chromatography on silica gel (eluted with Petroleum ether: Ethyl acetate=10:1) to obtain the title compound (60 g, yield 55.4%). ¹H NMR (400 MHz, CDCl₃) δ 7.23-7.13 (m, 3H), 6.80 (d, J=7.5 Hz, 1H), 3.82-3.38 (m, 2H), 1.34 (s, 12H).

Step 3: Synthesis of tert-butyl (3-(4,4,5,5-tetramethyl-1,3,2-dioxaborolan-2-yl)phenyl) carbamate. The product from Precursor Example 1, Step 2 (30 g, 137 mmol) and di-tert-butyl dicarbonate (38.9 g, 178 mmol) were mixed in toluene (600 mL) at 100° C. for 24 hours. Another reaction was set up as described above. The two reaction mixtures were combined and the brown mixture was evaporated, dissolved in ethyl acetate (1.5 L), washed with 0.1 N HCl (3×2 L) and brine (3 L), dried over Na₂SO₄, filtered and concentrated under reduced pressure to give the title compound (50 g, yield 57%). ¹H NMR (400 MHz, CDCl₃) δ 7.63 (br m, 2H), 7.48 (d, J=7.1 Hz, 1H), 7.37-7.28 (m, 1H), 1.52 (s, 9H), 1.34 (s, 12H). Boc=tert-butoxycarbonyl.

Step 4: Synthesis of tert-butyl (3-(4-formylbenzyl)phenyl)carbamate. A mixture of the product from Precursor Example 1, Step 1 (24.94 g, 125 mmol), 1,1′-bis(diphenylphosphino) ferrocenedichloro palladium(II) dichloromethane complex (13.75 g, 18.80 mmol), the product from Precursor Example 1, Step 3 (20 g, 62.7 mmol) and potassium carbonate (43.3 g, 313 mmol) in tetrahydrofuran (400 mL) was heated to 80° C. for 12 hours. Another additional reaction was set up as described above. The two reaction mixtures were combined and diluted with water (500 mL). The aqueous mixture was extracted with ethyl acetate (3×500 mL). The organic layers were combined and dried over Na₂SO₄, filtered and concentrated under reduced pressure. The residue was purified by column chromatography on silica gel (eluted with petroleum ether:ethyl acetate=10:1) to obtain the title compound (15 g, yield 38.4%). ¹H NMR (400 MHz, CDCl₃) δ 9.95 (s, 1H), 7.78 (d, J=7.9 Hz, 2H), 7.33 (d, J=7.9 Hz, 2H), 7.27-7.13 (m, 3H), 6.82 (d, J=7.1 Hz, 1H), 6.47 (br. s., 1H), 4.00 (s, 2H), 1.48 (s, 9H).

Step 5: Synthesis of (6S,8S,9R,10S,11S,13S,14S,16R,17S)-6,9-difluoro-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one. (2S,6aS,6bR,7S,8aS,8bS,11aR,12aS,12bS)-2,6b-Difluoro-7-hydroxy-8b-(2-hydroxyacetyl)-6a,8a,10,10-tetramethyl-1,2,6a,6b,7,8,8a,8b,11a,12,12a,12b-dodecahydro-4H-naphtho[2′,1′:4,5]indeno[1,2-d][1,3]dioxol-4-one (20 g, 44.2 mmol) was suspended in 40% aqueous HBF₄ (440 mL) and the mixture was stirred at 25° C. for 48 hours. After the reaction was complete, 2 L of H₂O was added and the solid was collected by filtration. This solid was washed with H₂O (1 L) and then methanol (200 mL) to give the title compound (11 g, yield 60.3%). ¹H NMR (400 MHz, dimethylsulfoxide-d6) δ 7.25 (d, J=10.1 Hz, 1H), 6.28 (d, J=10.1 Hz, 1H), 6.10 (s, 1H), 5.73-5.50 (m, 1H), 5.39 (br. s., 1H), 4.85-4.60 (m, 2H), 4.50 (d, J=19.4 Hz, 1H), 4.20-4.04 (m, 2H), 2.46-2.06 (m, 6H), 1.87-1.75 (m, 1H), 1.56-1.30 (m, 6H), 0.83 (s, 3H), dimethylsulfoxide=dimethylsulfoxide.

Step 6: Synthesis of (2S,6aS,6bR,7S,8aS,8bS,10S,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one. A suspension of the product from Precursor Example 1, Step 5 (4.4 g, 10.67 mmol) and MgSO₄ (6.42 g, 53.3 mmol) in acetonitrile (100 mL) was stirred at 20° C. for 1 hour. A solution of the product from Precursor Example 1, Step 4 (3.65 g, 11.74 mmol) in acetonitrile (100 mL) was added in one portion. Trifluoromethanesulfonic acid (9.01 mL, 53.3 mmol) was added dropwise while maintaining an internal temperature below room temperature using an ice bath. After the addition, the mixture was stirred at 20° C. for 2 hours. Three additional reactions were set up as described above. All four reaction mixtures were combined and concentrated and the residue was purified by Prep HPLC to give the title compound (4.5 g, yield 14.2%). LCMS (Method a, Table 7) Rₜ=2.65 min; MS m/z=606.2 (M+H)⁺. ¹H NMR (400 MHz, dimethylsulfoxide-d6) δ 7.44-7.17 (m, 5H), 6.89 (t, J=7.7 Hz, 1H), 6.44-6.25 (m, 4H), 6.13 (br. s., 1H), 5.79-5.52 (m, 2H), 5.44 (s, 1H), 5.17-4.89 (m, 3H), 4.51 (d, J=19.4 Hz, 1H), 4.25-4.05 (m, 2H), 3.73 (s, 2H), 3.17 (br. s., 1H), 2.75-2.55 (m, 1H), 2.36-1.97 (m, 3H), 1.76-1.64 (m, 3H), 1.59-1.39 (m, 4H), 0.94-0.78 (m, 3H). Prep HPLC Method: Instrument: Gilson 281 semi-preparative HPLC system; Mobile phase: A: Formic Acid/H₂O=0.01% v/v; B: Acetonitrile; Column: Luna C18 150*25 5 micron; Flow rate: 25 mL/min; Monitor wavelength: 220 and 254 nm.

TABLE 9
Mobile phase conditions for elutant B

| Time (min) | 0.0 | 10.5 | 10.6 | 10.7 | 13.7 | 13.8 | 15.0 |
|---|---|---|---|---|---|---|---|
| B % | 15 | 35 | 35 | 100 | 100 | 10 | 10 |

### Precursor Example 2: Synthesis of (6aR,6bS,7S,8aS,8bS,10R,11aR,12aS,12bS)-10-(4-(3-aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

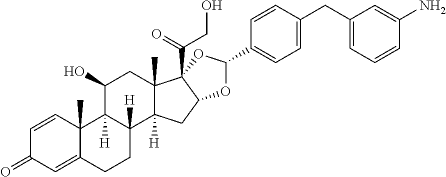

Precursor Example 2 product was synthesized in a similar procedure to Precursor Example 1 using (6aR,6bS,7S,8aS,8bS,11aR,12aS,12bS)-7-hydroxy-8b-(2-hydroxyacetyl)-6a,8a,10,10-tetramethyl-1,2,6a,6b,7,8,8a,8b,11a,12,12a,12b-dodecahydro-4H-naphtho[2′,1′:4,5]indeno[1,2-d][1,3]dioxol-4-one.

¹H NMR (400 MHz, dimethylsulfoxide-d₆) δ 7.36 (d, J=7.9 Hz, 2H), 7.31 (d, J=10.1 Hz, 1H), 7.20 (d, J=7.9 Hz, 2H), 6.89 (t, J=7.9 Hz, 1H), 6.39-6.28 (m, 3H), 6.16 (dd, J=1.5, 9.9 Hz, 1H), 5.93 (s, 1H), 5.39 (s, 1H), 5.08 (t, J=5.7 Hz, 1H), 4.98-4.87 (m, 3H), 4.78 (d, J=3.1 Hz, 1H), 4.49 (dd, J=6.2, 19.4 Hz, 1H), 4.29 (br. s., 1H), 4.17 (dd, J=5.5, 19.6 Hz, 1H), 3.74 (s, 2H), 2.61-2.53 (m, 1H), 2.36-2.26 (m, 1H), 2.11 (d, J=11.0 Hz, 1H), 2.07 (s, 1H), 2.02 (d, J=12.8 Hz, 1H), 1.83-1.54 (m, 5H), 1.39 (s, 3H), 1.16-0.96 (m, 2H), 0.85 (s, 3H). LCMS (Method a, Table 7) Rₜ=2.365 min; m/z=570.2 (M+H)⁺.

### Precursor Example 3: Synthesis of (6aS,6bR,7S,8aS,8bS,10R,11aR,12aS,12bS)-10-(4-(3-aminobenzyl)phenyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

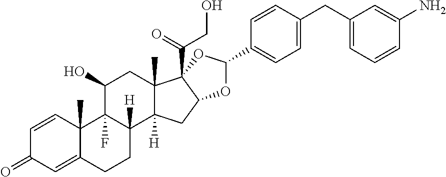

Precursor Example 3 product was synthesized in a similar procedure to Precursor Example 1 using (6aS,6bR,7S,8aS,8bS,11aR,12aS,12bS)-6b-fluoro-7-hydroxy-8b-(2-hydroxyacetyl)-6a,8a,10,10-tetramethyl-1,2,6a,6b,7,8,8a,8b,11a,12,12a,12b-dodecahydro-4H-naphtho[2′,1′:4,5]indeno[1,2-d][1,3]dioxol-4-one.

¹H NMR (400 MHz, dimethylsulfoxide-d₆) δ 7.37-7.26 (m, 3H), 7.21 (d, J=7.9 Hz, 2H), 6.89 (t, J=7.7 Hz, 1H), 6.43-6.30 (m, 3H), 6.23 (d, J=10.1 Hz, 1H), 6.04 (s, 1H), 5.75 (s, 1H), 5.44 (s, 2H), 5.09 (t, J=5.7 Hz, 1H), 4.93 (br. s., 3H), 4.50 (dd, J=6.2, 19.4 Hz, 1H), 4.28-4.09 (m, 2H), 3.74 (s, 2H), 2.73-2.54 (m, 2H), 2.35 (d, J=13.2 Hz, 1H), 2.25-2.12 (m, 1H), 2.05 (d, J=15.0 Hz, 1H), 1.92-1.77 (m, 1H), 1.74-1.58 (m, 3H), 1.50 (s, 3H), 1.45-1.30 (m, 1H), 0.87 (s, 3H). LCMS (Method a, Table 7) Rₜ=2.68 min; m/z=588.1 (M+H)⁺.

### Precursor Example 4: Synthesis of (S)-4-(2-(2-bromoacetamido)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

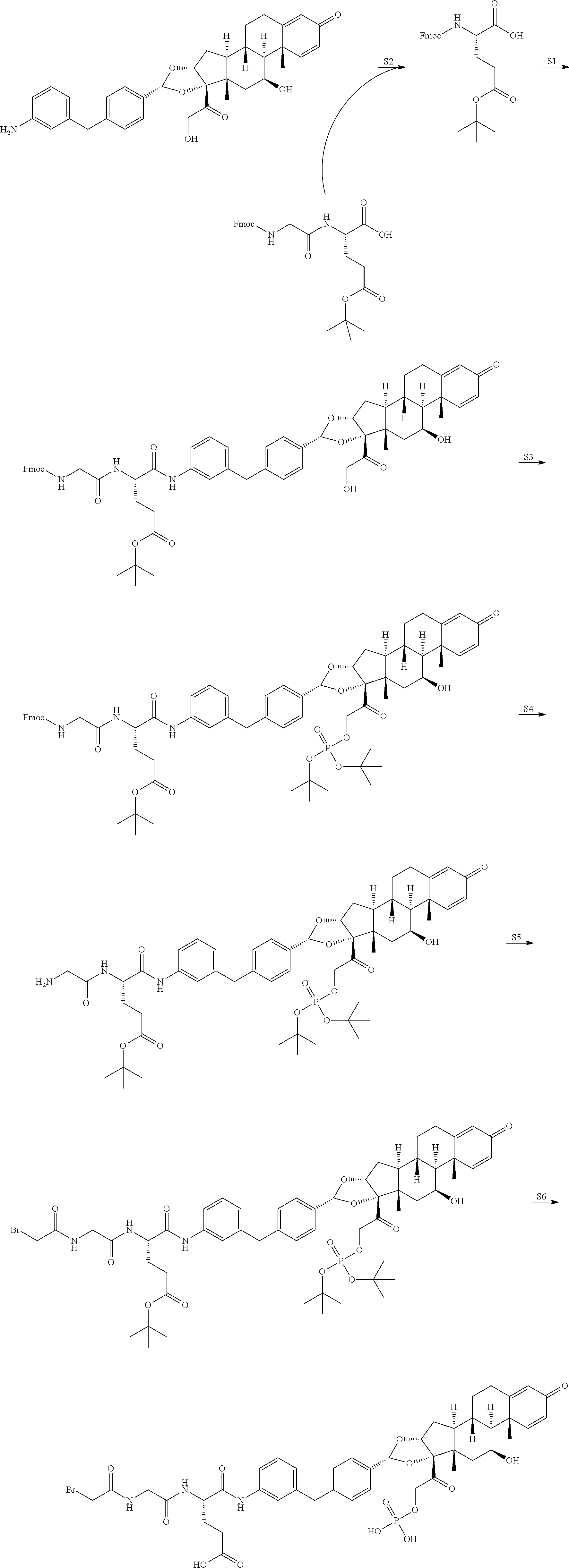

Step 1: Synthesis of (S)-2-(2-((((9H-fluoren-9-yl)methoxy)carbonyl)amino)acetamido)-5-(tert-butoxy)-5-oxopentanoic acid. A mixture of 2-chlorotrityl chloride resin (30 g, 92 mmol), triethylamine (46.4 g, 458 mmol) and (S)-2-((((9H-fluoren-9-yl)methoxy)carbonyl)amino)-5-(tert-butoxy)-5-oxopentanoic acid (25.5 g, 60 mmol) in dry dichloromethane (200 mL) was bubbled with N₂ at 20° C. for 8 hours. The mixture was filtered and the resin was washed with dichloromethane (2×200 mL), methanol (MeOH) (2×200 mL), and dimethyl formamide (2×200 mL). The resin was added a solution of piperidine:dimethyl formamide (1:4, 400 mL) and the mixture was bubbled with N₂ for 8 minutes and then filtered. This operation was repeated five times to give complete removal of the 9-fluorenylmethyloxycarbonyl (Fmoc) protecting group. The resin was washed with dimethyl formamide (5×500 mL) to afford resin bound (S)-2-amino-5-(tert-butoxy)-5-oxopentanoic acid. A mixture of 2-((((9H-fluoren-9-yl)methoxy)carbonyl)amino)acetic acid (13.38 g, 45.0 mmol), N,N-diisopropylethylamine (7.86 mL, 45 mmol), hydroxybenzotriazole (6.89 g, 45 mmol), 2-(6-chloro-1H-benzo[d][1,2,3]triazol-1-yl)-1,1,3,3-tetramethylisouronium hexafluorophosphate (V) (18.62 g, 45.0 mmol) in dimethyl formamide (200 mL) was stirred at 20° C. for 30 min. To the mixture was added the resin bound (S)-2-amino-5-(tert-butoxy)-5-oxopentanoic acid and the resulting mixture was bubbled with N₂ at 25° C. for 1.5 hours. The mixture was filtered and the resin was washed with dimethyl formamide (4×500 mL), and dichloromethane (2×500 mL). To the mixture was added 1% trifluoro acetic acid/dichloromethane (5×500 mL) and bubbled with N₂ for 5 min. The mixture was filtered and the filtrate was added to saturated solution of NaHCO₃ (200 mL) directly. The combined mixture was separated, and the organic phase was washed with saturated citric acid water solution (4×400 mL) and brine (2×300 mL). The final organic solution was dried over Na₂SO₄ (20 g), filtered, concentrated under reduced pressure to afford the title compound (10 g, yield 20%). NMR: (CDCl₃, 400 MHz) δ 7.75 (d, J=7.5 Hz, 2H), 7.59 (br d, J=7.5 Hz, 2H), 7.41-7.36 (m, 2H), 7.30 (t, J=7.0 Hz, 2H), 5.82 (br s, 1H), 4.57 (br d, J=4.8 Hz, 1H), 4.38 (br d, J=7.5 Hz, 2H), 4.27-4.15 (m, 1H), 4.06-3.83 (m, 2H), 2.50-2.29 (m, 2H), 2.26-2.13 (m, 1H), 2.06-2.02 (m, 1H), 1.43 (s, 9H).

Step 2: Synthesis of tert-butyl (S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of the product from Example 4, step 1 (424 mg, 0.878 mmol) in dimethyl formamide (3.5 mL) was added Example 2 (500 mg, 0.878 mmol) and triethylamine (0.3 mL, 2.63 mmol) at 25° C. The solution was cooled to 0° C. and then 2,4,6-tripropyl-1,3,5,2,4,6-trioxatriphosphinane 2,4,6-trioxide (1.12 g, 1.755 mmol) was added. The reaction mixture was stirred for 12 hours at 25° C. LCMS showed the reaction was complete. Fourteen additional reactions were set up as described above. All fifteen reaction mixtures were combined. The mixture was purified by reverse phase column to afford the title compound (5 g, yield 38.4%) as a yellow solid. Reverse phase column method: Instrument: Shimadzu LC-8A preparative HPLC; Column: Phenomenex Luna C18 200*40 mm*10 μm; Mobile phase: A for H₂O (0.05% Trifluoro acetic acid) and B for Acetonitrile; Gradient: B from 30% to 100% in 30 min; Flow rate: 60 mL/min; Wavelength: 220 & 254 nm. LCMS (Method a, Table 7) Rₜ=1.34 min; m/z 1016.6 (M+H-18)⁺.

Step 3: Synthesis of tert-butyl (S)-4-(2-((((9H-fluoren-9-yl)methoxy)carbonyl)amino)acetamido)-5-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of the product from Example 4, step 2 (400 mg, 0.387 mmol) in dimethyl formamide (2.5 mL) was added 1H-tetrazole (271 mg, 3.87 mmol) and di-tert-butyl diethylphosphoramidite (1.16 g, 4.64 mmol). The reaction was stirred at room temperature for 2.5 hours then cooled to 0° C. Hydrogen peroxide (241 mg, 2.127 mmol) was added to the resulting mixture allowed to warm to room temperature and stirred for 1 hour after which time LCMS showed the reaction was complete. Eleven additional reactions were set up as described above. All twelve reaction mixtures were combined. The mixture was purified by reverse phase column to afford the title compound (4.4 g, yield 64.2%). Reverse phase column method: Instrument: Shimadzu LC-8A preparative HPLC; Column: Phenomenex Luna C18 200*40 mm*10 μm; Mobile phase: A for H2O and B for acetonitrile; Gradient: B from 50% to 100% in 30 min; Flow rate: 60 mL/min; Wavelength: 220 & 254 nm. LCMS (Method a, Table 7) Rₜ=1.41 min; m/z 1226.7 (M+H)⁺.

Step 4: Synthesis of tert-butyl (S)-4-(2-((((9H-fluoren-9-yl)methoxy)carbonyl)amino)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of the product from Example 4, Step 3 (1.1 g, 0.897 mmol) in acetonitrile (6 mL) was added piperidine (0.75 mL, 7.58 mmol) at 25° C. The reaction was stirred at room temperature for 20 min after which time LCMS showed the reaction was complete. Three additional reactions were set up as described above. All four reaction mixtures were combined. The mixture was concentrated to afford a residue, which was treated with petroleum ether (10 mL) under stirring for 2 hours. The resulting solid was collected by filtration, and dried under reduced pressure to afford the title compound (3.8 g, yield 90%). LCMS (Method a, Table 7) Rₜ=1.16 min; m/z 1004.6 (M+H)⁺.

Step 5: Synthesis of tert-butyl (S)-4-(2-(2-bromoacetamido)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of 2-bromoacetic acid (97 mg, 0.697 mmol) in dimethyl formamide (2.5 mL) was added 2-Ethoxy-1-ethoxycarbonyl-1,2-dihydroquinoline (EEDQ) (172 mg, 0.697 mmol) at room temperature. The mixture was stirred at room temperature for 1 hour. The product from Example 4, Step 4 (350 mg, 0.349 mmol) was added and the resulting stirred for 2.5 hours after which time LCMS showed the reaction was complete. Seven additional reactions were set up as described above. All eight reaction mixtures were combined. The reaction was diluted with dichloromethane (100 mL), washed with aqueous HBr (1 M, 2×80 mL), aqueous NaHCO₃ (60 mL), brine (60 mL). The organic layer was dried over Na₂SO₄, filtered and concentrated under reduced pressure to afford the title compound (2 g, yield 63.7%). LCMS (Method a, Table 7) Rₜ=1.30 min; m/z 1126.4 (M+H)⁺.

Step 6: Synthesis of (S)-4-(2-(2-bromoacetamido)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid. To a solution of the product from Example 4, Step 5 (2 g, 1.778 mmol) in dichloromethane (16 mL) was added trifluoro acetic acid (8 mL, 104 mmol) and the resulting mixture was stirred at room temperature for 40 min after which time LCMS showed the reaction was complete. The solvent was removed under reduced pressure. The resulting residue was purified by Prep HPLC. The mobile phase was lyophilized directly to afford the title compound (640 mg, yield 35.3%). Prep HPLC method: Instrument: Shimadzu LC-8A preparative HPLC; Column: Phenomenex Luna C18 200*40 mm*10 um; Mobile phase: A for H₂O (0.09% Trifluoro acetic acid) and B for Acetonitrile; Gradient: B from 30% to 40% in 20 min; Flow rate: 60 mL/min; Wavelength: 220 & 254 nm.

¹H NMR: (dimethylsulfoxide-d6, 400 MHz) δ 9.88 (s, 1H), 8.52 (s, 1H), 8.24 (br d, J=8.4 Hz, 1H), 7.46 (br d, J=7.9 Hz, 1H), 7.42 (s, 1H), 7.36 (br d, J=7.9 Hz, 2H), 7.30 (br d, J=9.7 Hz, 1H), 7.23-7.17 (m, 3H), 6.90 (br d, J=6.8 Hz, 1H), 6.16 (br d, J=10.4 Hz, 1H), 5.93 (s, 1H), 5.47 (s, 1H), 4.96-4.85 (m, 3H), 4.58 (br dd, J=7.9, 18.7 Hz, 1H), 4.38 (br d, J=5.3 Hz, 1H), 4.29 (br s, 1H), 3.93 (s, 2H), 3.89 (s, 2H), 3.80 (br s, 2H), 2.30-2.22 (m, 2H), 2.16-1.91 (m, 4H), 1.85-1.62 (m, 6H), 1.39 (s, 3H), 1.00 (br s, 2H), 0.87 (s, 3H). LCMS (Method a, Table 7) Rₜ=2.86 min; m/z 956.0, 958.0 (M+H)⁺.

### Precursor Example 5: Synthesis of 2-((2S,6aS,6bR,7S,8aS,8bS,10R,11aR,12aS,12bS)-10-(4-(3-((S)-6-amino-2-(2-(2-bromoacetamido)acetamido)hexanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

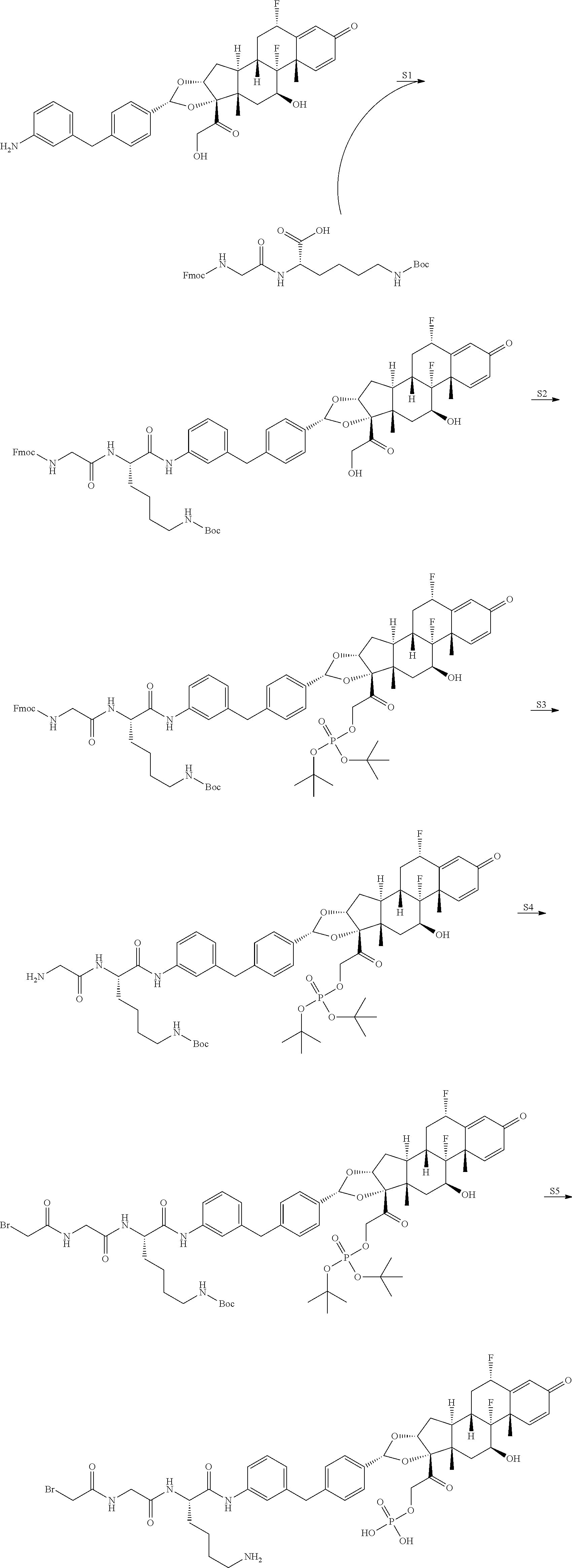

Step 1: Synthesis of tert-butyl ((S)-5-(2-((((9H-fluoren-9-yl)methoxy)carbonyl)amino)acetamido)-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of N²-((((9H-fluoren-9-yl)methoxy)carbonyl)glycyl)-N6-(tert-butoxycarbonyl)-L-lysine (5.58 g, 8.26 mmol) in dimethyl formamide (60 mL) at 0° C. was added 2,4,6-tripropyl-1,3,5,2,4,6-trioxatriphosphinane 2,4,6-trioxide (10.51 g, 16.51 mmol) and triethylamine (3.45 mL, 24.77 mmol). The resulting mixture was stirred at room temperature for 1 hour and then the product from Precursor Example 1, Step 6 (5 g, 8.26 mmol) was added. The resulting mixture was stirred for 5 hours at room temperature after which time LCMS showed the reaction was complete. Six additional reactions were set up as described above. All seven reaction mixtures were combined. The reaction was purified by reverse phase column to afford the title compound (24 g, yield 24.62%). Reverse phase column method: Instrument: Shimadzu LC-8A prep HPLC; Column: Phenomenex Luna C18 200*40 mm*10 μm; Mobile phase: A for H₂O (0.05% Trifluoro acetic acid) and B for acetonitrile; Gradient: B from 30% to 100% in 30 min; Flow rate: 60 mL/min; Wavelength: 220 & 254 nm. LCMS (Method a, Table 7) Rₜ=1.29 min; m/z 1095.6 (M+H-18)⁺. Fmoc=Fluorenylmethyloxycarbonyl; Boc=tertbutoxycarbonyl.

Step 2: Synthesis of tert-butyl ((S)-5-(2-((((9H-fluoren-9-yl)methoxy)carbonyl)amino)acetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of the product from Example 5, Step 1 (3 g, 2.69 mmol) in dimethyl formamide (30 mL) was added 1H tetrazole (1.888 g, 26.9 mmol) and di-tert-butyl diethylphosphoramidite (8.06 g, 32.3 mmol) and the reaction was stirred at room temperature for 3.5 hours. Hydrogen peroxide (224 mg, 1.97 mmol) was added to the reaction and stirred for 0.5 hours after which time LCMS showed the reaction was complete. Six additional reactions were set up as described above. All seven reaction mixtures were combined. The reaction was purified by reverse phase column to afford the title compound (10 g, purity: 78%, yield 37.1%). Reverse phase column method: Instrument: Shimadzu LC-8A prep HPLC; Column: Phenomenex Luna C18 200*40 mm*10 um; Mobile phase: A for H₂O and B for acetonitrile; Gradient: B from 50% to 100% in 30 min; Flow rate: 60 mL/min; Wavelength: 220 & 254 nm. LCMS (Method a, Table 7) Rₜ=1.42 min; m/z 1305.7 (M+H)⁺.

Step 3: Synthesis of tert-butyl ((S)-5-(2-aminoacetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2,′,1′:4,5]indeno[1,2-d][1,3]dioxol-10-yl)benzyl)phenyl)amino)-6-oxohexyl)carbamate. To a solution of the product from Example 5, Step 2 (2.5 g, 1.969 mmol) in acetonitrile (10 mL) was added piperidine (2 mL, 1.969 mmol) and the reaction stirred at room temperature for 1 hour after which time LCMS showed the reaction was complete. Three additional reactions were set up as described above. All four reaction mixtures were combined. The reaction was concentrated to afford a crude product, which was stirred in petroleum ether (30 mL) for 2 hours. The resulting solid was collected by filtration, and dried under reduced pressure to afford the title compound (7 g, purity: 83%, yield 70.4%) as a yellow solid. LCMS (Method a, Table 7) Rₜ=1.17 min; m/z 1083.5 (M+H)⁺.

Step 4: Synthesis of tert-butyl ((S)-5-(2-(2-bromoacetamido)acetamido)-6-((3-(4-42S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of 2-bromoacetic acid (0.929 g, 6.68 mmol) in dimethyl formamide (35 mL) was added 2-ethoxy-1-ethoxycarbonyl-1,2-dihydroquinoline (1.653 g, 6.68 mmol) and the resulting mixture stirred at room temperature for 1 hour. The product from Example 5, Step 3 (3.5 g, 3.34 mmol) was added and the resulting mixture stirred at room temperature for 2 hours. LCMS showed the reaction was completed. The reaction was diluted with dichloromethane (100 mL), washed with aqueous HBr (1 M, 2×80 mL), aqueous NaHCO₃ (60 mL) and brine (60 mL). The organic layer was dried over Na₂SO₄, filtered and concentrated under reduced pressure to afford the title compound (2 g, yield 51.2%). LCMS (Method a, Table 7) Rₜ=1.32 min; m/z 1205.5 (M+H)⁺.

Step 5: Synthesis of 2-((2S,6aS,6bR,7S,8aS,8bS,10R,11aR,12aS,12bS)-10-(4-(3-((S)-6-amino-2-(2-(2-bromoacetamido)acetamido)hexanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate. To a solution of the product from Example 5, Step 4 (2 g, 1.661 mmol) in dichloromethane (10 mL) was added Trifluoro acetic acid (5 mL, 64.9 mmol) and the reaction stirred at rt for 40 min after which time LCMS showed the reaction was complete. The solvent was removed under reduced pressure and the crude product purified by Prep HPLC. The mobile phase was lyophilized directly to afford the title compound (550 mg, purity: 96.9%, yield 32.3%). Prep-HPLC method: Instrument: Shimadzu LC-8A prep HPLC; Column: Phenomenex Luna C18 200*40 mm*10 μm; Mobile phase: A for H₂O (0.09% Trifluoro acetic acid) and B for Acetonitrile; Gradient: B from 30% to 40% in 20 min; Flow rate: 60 mL/min; Wavelength: 220 & 254 nm. LCMS (Method a, Table 7) Rₜ=2.31 min. ¹H NMR: (dimethylsulfoxide-d6, 400 MHz) δ ppm 0.90 (s, 3H) 1.19-1.41 (m, 2H) 1.43-1.62 (m, 7H) 1.64-1.77 (m, 3H) 1.84 (br d, J=14.55 Hz, 1H) 1.95-2.07 (m, 1H) 2.18-2.36 (m, 3H) 2.65-2.78 (m, 3H) 3.71-3.86 (m, 3H) 3.89 (s, 2H) 3.93 (s, 2H) 4.20 (br d, J=9.48 Hz, 1H) 4.33-4.41 (m, 1H) 4.59 (br dd, J=18.41, 8.05 Hz, 1H) 4.81 (br dd, J=18.52, 8.60 Hz, 1H) 4.94 (d, J=4.63 Hz, 1H) 5.50 (s, 1H) 5.54-5.76 (m, 1H) 6.13 (s, 1H) 6.29 (dd, J=10.14, 1.32 Hz, 1H) 6.95 (d, J=7.72 Hz, 1H) 7.15-7.28 (m, 4H) 7.30-7.41 (m, 3H) 7.51 (br d, J=7.94 Hz, 1H) 7.72 (br s, 3H) 8.21 (br d, J=7.72 Hz, 1H) 8.54 (t, J=5.62 Hz, 1H) 9.93 (br d, J=2.65 Hz, 1H).

### Precursor Example 6: Synthesis of (S)—N-(3-(4-((2S,6aS,6bR,7S,8aS,8bS,10R,11aR,12aS,12bS)-2,6b-Difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)-2-((S)-2-(3-(2,5-dioxo-2,5-dihydro-1H-pyrrol-1-yl)propanamido)propanamido)propanamide

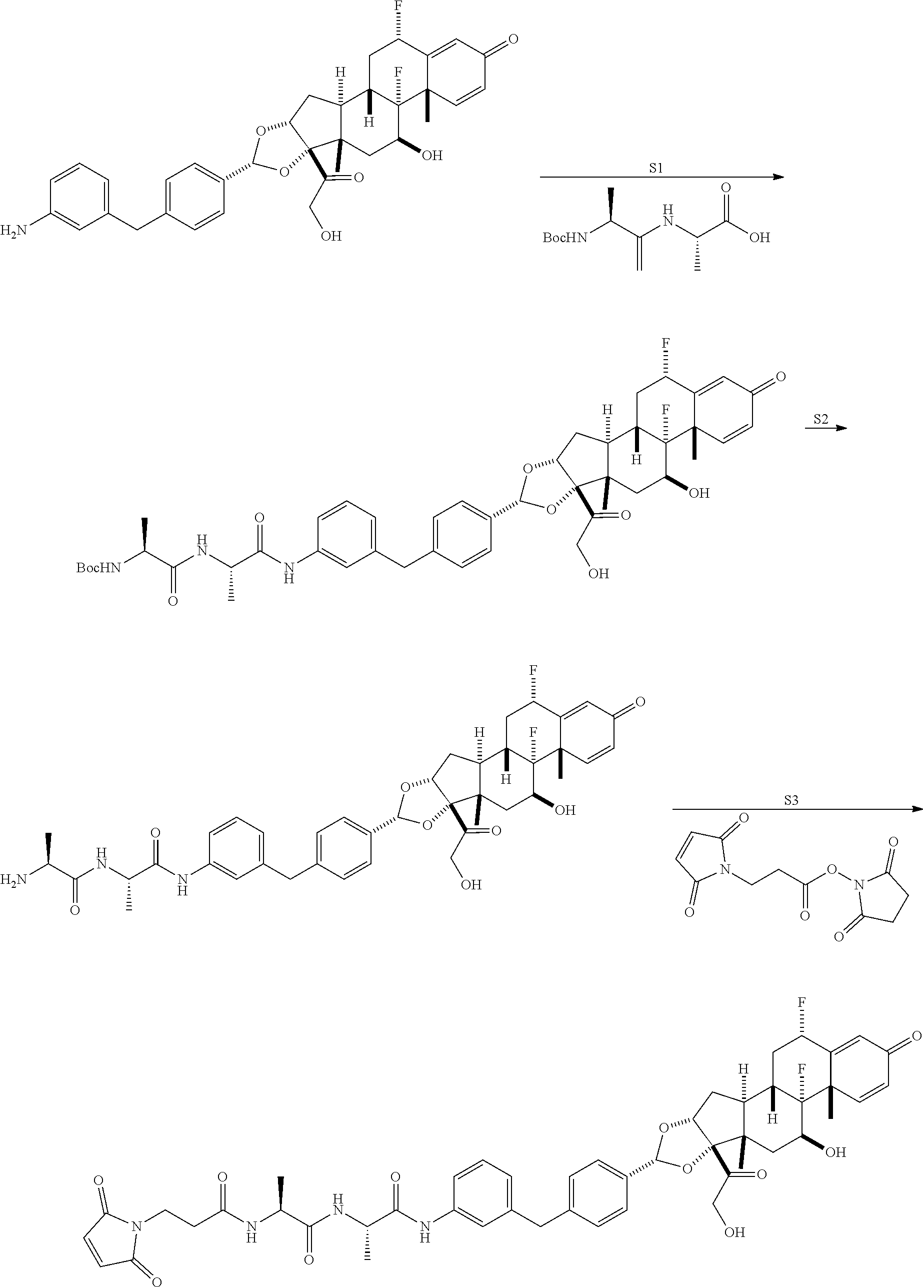

Step 1: Synthesis of tert-butyl ((S)-1-(((S)-1-((3-(4-((2S,6aS,6bR,7S, 8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)carbamate. 1-[Bis(dimethylamino)methylene]-1H-1,2,3-triazolo[4,5-b]pyridinium 3-oxid hexafluorophosphate (HATU) (610 mg, 1.605 mmol) and 2,6-lutidine (0.3 mL, 2.58 mmol) were added to a room temperature a mixture of the product from Precursor Example 1, Step 6 (648.1 mg, 1.070 mmol), and (S)-2-((S)-2-((tert-butoxycarbonyl)amino)propanamido)propanoic acid (334 mg, 1.284 mmol) in THF (11.5 mL). After 9 hours the reaction was diluted with ethyl acetate (16 mL), then washed with a 1N aqueous solution of HCl (3×4 mL), followed by a saturated aqueous solution of brine (4 mL). Purification by chromatography (silica, 40 g) eluting with a gradient of 0-10% methanol/dichloromethane gave the title compound (773.7 mg, 0.912 mmol, 85% yield). LCMS (Method b, Table 7) Rₜ=0.92 min, m/z=848.53 [M+H⁺].

Step 2: Synthesis of (S)-2-amino-N—((S)-1-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide. Trifluoro acetic acid (1.97 mL, 25.6 mmol) was added drop-wise to a room temperature solution of the product from Example 6, Step 1 (0.7683 g, 0.906 mmol) in dichloromethane (6.0 mL). After 50 min solvent was removed under reduced pressure to give a brown syrup. The residue was dissolved in 1:1 dimethyl sulfoxide: methanol (12 mL) and purified by reverse phase HPLC on a Phenomenex C18(2) 10 micron column (250×50 mm column). A gradient of Acetonitrile (A) and 0.1% trifluoroacetic acid in water (B) was used, at a flow rate of 90 mL/min (0-5.0 min 15% A, 5.0-20 min linear gradient 15-75% A, hold 2 min, 22.0-22.5 min linear gradient 75-95% A, hold 4 min). Combined fractions were concentrated under reduced pressure to dryness and the residue was dried overnight in the vacuum oven at 50° C. to give the title compound (230 mg, 0.308 mmol, 34% yield). LC-MS (Method b, Table 7) major acetal isomer Rₜ=0.73 min, m/z=748.78 [M+H⁺]. ¹H NMR (400 MHz, dimethylsulfoxide-d₆) δ 10.01 (s, 1H), 8.62 (d, J=7.2 Hz, 1H), 8.04 (d, J=5.4 Hz, 3H), 7.46-7.31 (m, 4H), 7.31-7.13 (m, 4H), 6.91 (d, J=7.6 Hz, 1H), 6.27 (dd, J=10.2, 1.9 Hz, 1H), 6.11 (s, 1H), 5.76-5.47 (m, 2H), 5.43 (s, 1H), 4.93 (d, J=4.6 Hz, 1H), 4.49 (d, J=19.5 Hz, 1H), 4.42 (q, J=7.1 Hz, 1H), 4.23-4.13 (m, 2H), 2.72-2.54 (m, 1H), 2.33-2.16 (m, 2H), 2.02 (dt, J=13.6, 3.6 Hz, 1H), 1.69 (h, J=5.9, 5.1 Hz, 3H), 1.48 (s, 4H), 1.33 (d, J=7.0 Hz, 3H), 1.30 (d, J=7.1 Hz, 3H), 0.85 (s, 3H).

Step 3: Synthesis of (S)—N-(3-(4-((2S,6aS,6bR,7S,8aS,8bS,10R,11aR,12aS,12bS)-2,6b-Difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)-2-((S)-2-(3-(2,5-dioxo-2,5-dihydro-1H-pyrrol-1-yl)propanamido)propanamido)propanamide. Diisopropylethylamine (0.1 mL, 0.573 mmol) was added to a room temperature solution of the product from Example 6, Step 2 (0.220 g, 0.294 mmol) and N-succinimidyl 3-maleimidopropionate (0.086 g, 0.324 mmol) in dimethyl formamide (2.8 mL). After 30 min the pH of the reaction mixture was adjusted to 4-5 by drop-wise addition of a 7% solution of trifluoroacetic acid in water (1.0 mL). The crude mixture was purified by reverse phase HPLC on a Phenomenex C18(2) 10 micron column (250×50 mm column). A gradient of acetonitrile (A) and 0.1% trifluoroacetic acid in water (B) was used, at a flow rate of 90 mL/min (0-5.0 min 15% A, 5.0-20 min linear gradient 15-85% A, hold 2 min). Combined fractions were concentrated under reduced pressure to remove volatile solvents, and the resulting solution was frozen and lyophilized to give the title compound (175.2 mg, 0.195 mmol, 66% yield). LCMS (Method b, Table 7) Rₜ=0.82 min, m/z=899.87 [M+H⁺]. ¹H NMR (400 MHz, dimethylsulfoxide-d₆) δ 9.70 (s, 1H), 8.14 (d, J=7.0 Hz, 1H), 8.01 (d, J=7.2 Hz, 1H), 7.47-7.35 (m, 2H), 7.32 (d, J=8.1 Hz, 2H), 7.26-7.10 (m, 4H), 6.95 (s, 1H), 6.87 (dt, J=7.6, 1.3 Hz, 1H), 6.26 (dd, J=10.2, 1.9 Hz, 1H), 6.09 (d, J=2.0 Hz, 1H), 5.72-5.51 (m, 1H), 5.48 (s, 1H), 5.41 (s, 1H), 4.91 (d, J=4.9 Hz, 1H), 4.47 (d, J=19.4 Hz, 1H), 4.30 (p, J=7.1 Hz, 1H), 4.25-4.11 (m, 3H), 3.85 (s, 2H), 3.57 (t, J=7.3 Hz, 2H), 2.71-2.48 (m, 1H), 2.36 (dd, J=8.0, 6.7 Hz, 2H), 2.23 (ddt, J=25.1, 12.2, 6.6 Hz, 2H), 2.01 (dt, J=13.7, 3.7 Hz, 1H), 1.75-1.57 (m, 3H), 1.48 (p, J=11.9 Hz, 1H), 1.46 (s, 3H), 1.24 (d, J=7.2 Hz, 3H), 1.13 (d, J=7.2 Hz, 3H), 0.83 (s, 3H).

### Precursor Example 7: Synthesis of 3-(2,5-dioxo-2,5-dihydro-1H-pyrrol-1-yl)-N—((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)propanamide

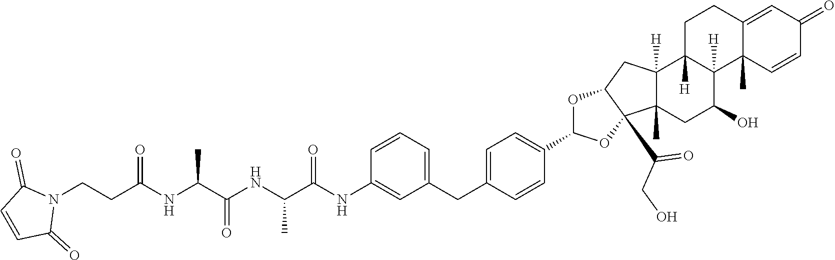

Precursor Example 7 product was synthesized in a similar procedure to Example 6 using (6aR,6bS,7S,8aS,8bS,10R,11aR,12aS,12bS)-10-(4-(3-aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one. LCMS (Method b, Table 7) Rₜ=0.85 min, m/z=863.4 [M+H].

¹H NMR (501 MHz, dimethylsulfoxide-d₆) δ 9.71 (s, 1H), 8.17 (d, J=7.0 Hz, 1H), 8.03 (d, J=7.3 Hz, 1H), 7.43 (dd, J=7.8, 1.1 Hz, 2H), 7.38-7.32 (m, 2H), 7.29 (d, J=10.1 Hz, 1H), 7.22-7.15 (m, 3H), 6.96 (s, 2H), 6.88 (dt, J=7.8, 1.3 Hz, 1H), 6.13 (dd, J=10.1, 1.9 Hz, 1H), 5.90 (t, J=1.6 Hz, 1H), 5.37 (s, 1H), 4.90 (d, J=5.4 Hz, 1H), 4.48 (d, J=19.4 Hz, 1H), 4.32 (p, J=7.1 Hz, 1H), 4.27 (q, J=3.3 Hz, 1H), 4.21 (p, J=7.1 Hz, 1H), 4.16 (d, J=19.4 Hz, 1H), 3.87 (s, 2H), 3.59 (t, J=7.3 Hz, 2H), 2.57-2.49 (m, 1H), 2.38 (dd, J=8.0, 6.6 Hz, 2H), 2.32-2.24 (m, 1H), 2.15-2.04 (m, 1H), 2.04-1.95 (m, 1H), 1.80-1.54 (m, 5H), 1.37 (s, 3H), 1.26 (d, J=7.1 Hz, 3H), 1.15 (d, J=7.1 Hz, 3H), 1.02 (ddd, J=21.2, 12.1, 4.2 Hz, 2H), 0.84 (s, 3H).

### Precursor Example 8: Synthesis of 3-(2,5-dioxo-2,5-dihydro-1H-pyrrol-1-yl)-N—((S)-1-(((S)-1-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)propanamide

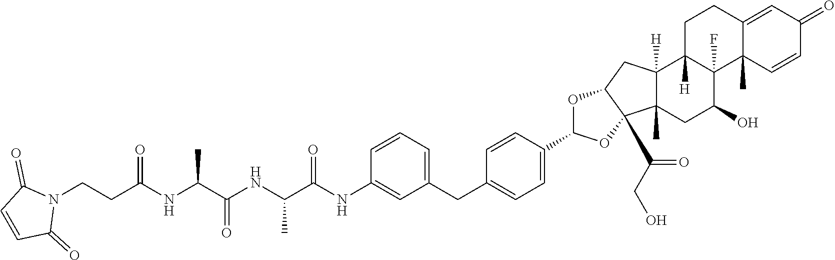

Precursor Example 8 product was synthesized in a similar procedure to Example 6 using (6aS,6bR,7S,8aS,8bS,10R,11aR,12aS,12bS)-10-(4-(3-aminobenzyl)phenyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one. LCMS (Method b, Table 7) Rₜ=0.85 min; m/z=881.46 [M+H]⁺.

¹H NMR (dimethylsulfoxide-d6) δ 0.83 (s, 3H), 1.13 (d, J=7.1 Hz, 3H), 1.24 (d, J=7.1 Hz, 3H), 1.35 (qd, J=13.3, 12.8, 5.1 Hz, 1H), 1.46 (s, 3H), 1.63 (q, J=9.7, 8.5 Hz, 3H), 1.73-1.88 (m, 1H), 2.01 (dt, J=13.7, 3.5 Hz, 1H), 2.14 (td, J=11.8, 7.2 Hz, 1H), 2.26-2.40 (m, 3H), 2.48-2.69 (m, 2H), 3.57 (t, J=7.3 Hz, 2H), 3.85 (s, 2H), 4.17 (ddd, J=17.5, 11.7, 6.2 Hz, 3H), 4.30 (p, J=7.2 Hz, 1H), 4.47 (d, J=19.4 Hz, 1H), 4.83-4.95 (m, 1H), 5.40 (s, 2H), 5.99 (d, J=1.6 Hz, 1H), 6.20 (dd, J=10.1, 1.9 Hz, 1H), 6.87 (d, J=7.5 Hz, 1H), 6.95 (s, 2H), 7.16 (t, J=7.9 Hz, 1H), 7.20 (d, J=8.1 Hz, 2H), 7.25 (d, J=10.1 Hz, 1H), 7.31 (d, J=8.0 Hz, 2H), 7.38 (d, J=1.9 Hz, 1H), 7.43 (dd, J=8.0, 2.0 Hz, 1H), 8.01 (d, J=7.3 Hz, 1H), 8.14 (d, J=7.1 Hz, 1H), 9.70 (s, 1H).

### Precursor Example 9: Synthesis of (S)-4-(2-(2,5-Dioxo-2,5-dihydro-1H-pyrrol-1-yl)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

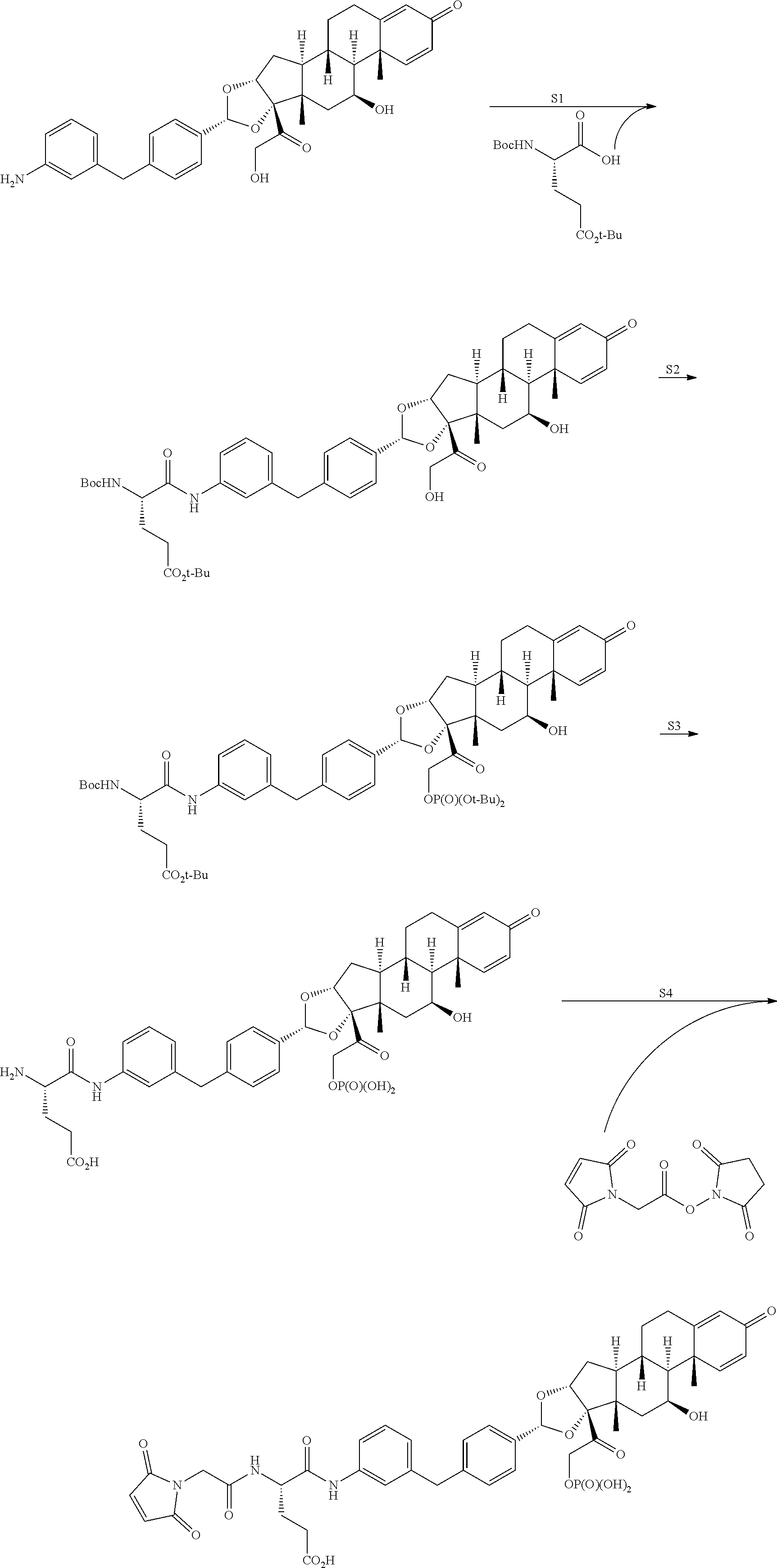

Step 1: Synthesis of tert-Butyl (S)-4-((tert-butoxycarbonyl)amino)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. 1-[Bis(dimethylamino)methylene]-1H-1,2,3-triazolo[4,5-b]pyridinium 3-oxid hexafluorophosphate (HATU) (260 mg, 0.685 mmol) and 2,6-dimethylpyridine (0.184 ml, 1.580 mmol) were added to a room temperature suspension of the product from Precursor Example 2 (300 mg, 0.527 mmol) and (S)-5-(tert-butoxy)-2-((tert-butoxycarbonyl)amino)-5-oxopentanoic acid (168 mg, 0.553 mmol) in dimethyl formamide (6 mL). After 2 hours at room temperature, the reaction was diluted with ethyl acetate (30 mL) and then washed sequentially with a 1 N aqueous solution of HCl (2×15 mL), a saturated aqueous solution of NaHCO₃ (15 mL), and brine (15 mL). The organic layer was dried (Na₂SO₄) and solvent was removed under reduced pressure. Purification by chromatography (silica) eluting with a gradient of 0-10% methanol/dichloromethane provided the title compound (400 mg, 0.47 mmol, 89% yield) as an off-white solid. LCMS (Method b, Table 7) Rt=1.09 min; MS m/z=854.9 [M+H]⁺.

Step 2: Synthesis of tert-Butyl (S)-4-((tert-butoxycarbonyl)amino)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. Di-tert-butyl diethylphosphoramidite (0.42 ml, 1.5 mmol) was added to a room temperature solution of the product from Precursor Example 9, Step 1 (400 mg, 0.468 mmol) and 1H tetrazole (0.35 ml, 2.25 mmol) in dimethyl acetamide (5 mL). The reaction was stirred at room temperature for 2 hours, whereupon a 50% solution of hydrogen peroxide in water (1.5 mL) was added drop-wise. Once LCMS indicated that the oxidation was complete, the reaction was cool to 0° C. and quenched by addition of a 1 M aqueous solution of Na₂S₂O₃ (8 mL). The mixture was extracted with Ethyl acetate (2×30 mL), the combined organic layers washed with brine (15 mL), dried (Na₂SO₄), filtered and solvent was removed under reduced pressure. Purification by preparative reverse phase HPLC gave the title compound (420 mg, 0.40 mmol, 86% yield). LCMS (Method b, Table 7) Rₜ=1.27 min; MS m/z=1047.6 [M+H⁺]. ¹H NMR (400 MHz, dimethylsulfoxide-d₆) δ 9.80 (s, 1H), 7.41 (d, J=8.3 Hz, 1H), 7.37-7.31 (m, 3H), 7.29 (d, J=10.1 Hz, 1H), 7.20 (d, J=8.0 Hz, 2H), 7.17 (t, J=7.9 Hz, 1H), 6.94 (d, J=8.0 Hz, 1H), 6.88 (d, J=7.5 Hz, 1H), 6.11 (dd, J=10.1, 1.9 Hz, 1H), 5.89 (s, 1H), 5.46 (s, 1H), 5.00-4.81 (m, 3H), 4.58 (dd, J=18.0, 9.1 Hz, 1H), 4.26 (s, 1H), 4.00 (d, J=6.7 Hz, 1H), 3.86 (s, 2H), 2.49 (d, J=2.2 Hz, 1H), 2.29 (p, J=2.0 Hz, 1H), 2.27-2.16 (m, 2H), 2.06 (d, J=10.3 Hz, 1H), 1.98 (d, J=11.0 Hz, 1H), 1.91-1.56 (m, 6H), 1.39 (d, J=1.5 Hz, 18H), 1.35 (s, 3H), 1.33 (s, 18H), 1.01 (t, J=13.7 Hz, 2H), 0.85 (s, 3H).

Step 3: Synthesis of (S)-4-Amino-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid. Trifluoroacetic acid (2.0 mL, 0.40 mmol) was added to a room temperature solution of the product from Precursor Example 9, Step 2 (420 mg, 0.401 mmol) in dichloromethane (6 mL). The mixture was stirred at room temperature for 45 minutes, whereupon solvent was removed under reduced pressure. The title compound was carried forward without further purification. LCMS (Method a, Table 7) major acetal isomer Rₜ=0.69 min, MS m/z=779.8 [M+H]⁺; minor acetal isomer Rₜ=0.72 min, MS m/z=779.9 [M+H]⁺.

Step 4: Synthesis of (S)-4-(2-(2,5-Dioxo-2,5-dihydro-1H-pyrrol-1-yl)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid. N,N-Diisopropylethylamine (0.90 mL, 5.1 mmol) and maleimidoacetic acid N-hydroxysuccinimide ester (141 mg, 0.561 mmol) were added sequentially to a room temperature solution of the product from Precursor Example 9, Step 3 (364 mg, 0.467 mmol) in dimethyl formamide (5 mL). LCMS indicated the reaction was complete within 15 minutes, whereupon the reaction was cooled to 0° C. and the pH was adjusted to 1 by addition of 2,2,2-trifluoroacetic acid (0.432 mL, 6.6 mmol). Purification by preparative reverse phase HPLC and lyophilization gave the title compound (146 mg, 0.159 mmol, 34% yield). LCMS (Method b, Table 7) Rₜ=0.79 min; MS m/z=915.9 [M+H]⁺. ¹H NMR (400 MHz, dimethylsulfoxide-d₆) δ 9.92 (s, 1H), 8.45 (d, J=7.8 Hz, 1H), 7.43-7.38 (m, 1H), 7.35 (d, J=8.1 Hz, 3H), 7.28 (d, J=10.1 Hz, 1H), 7.21 (d, J=8.3 Hz, 3H), 7.16 (d, J=7.9 Hz, 1H), 7.05 (s, 2H), 6.89 (d, J=7.6 Hz, 1H), 6.13 (dd, J=10.1, 1.9 Hz, 1H), 5.89 (d, J=1.6 Hz, 1H), 5.44 (s, 1H), 4.93-4.83 (m, 2H), 4.80 (s, 1H), 4.53 (dd, J=18.2, 8.2 Hz, 1H), 4.34 (td, J=8.1, 5.3 Hz, 1H), 4.27 (s, 1H), 4.08 (s, 2H), 3.87 (s, 2H), 2.58-2.48 (m, 1H), 2.34-2.16 (m, 3H), 2.16-2.03 (m, 1H), 2.03-1.84 (m, 2H), 1.84-1.53 (m, 4H), 1.36 (s, 3H), 1.01 (td, J=13.1, 11.3, 4.0 Hz, 2H), 0.84 (s, 3H).

### Precursor Example 10: Synthesis of (S)-4-(2-(2,5-Dioxo-2,5-dihydro-1H-pyrrol-1-yl)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

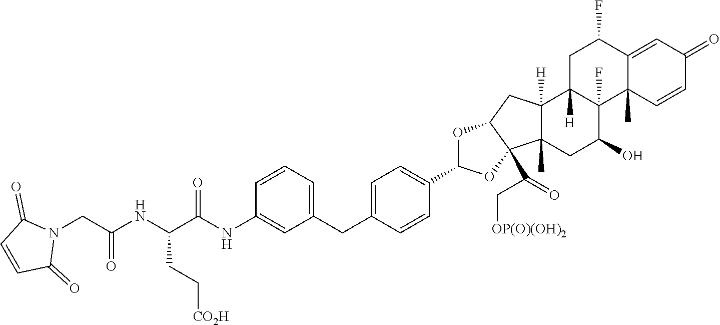

Precursor Example 10 product was synthesized in a similar procedure to Precursor Example 9 using the product from Precursor Example 1, Step 6. LCMS (Method b, Table 7) Rₜ=0.79 min; MW m/z 974.3 [M+Na]⁺.

¹H NMR (400 MHz, dimethylsulfoxide-d6) δ 9.91 (s, 1H), 8.45 (d, J=7.8 Hz, 1H), 7.42 (dd, J=8.0, 1.9 Hz, 1H), 7.37-7.29 (m, 3H), 7.28-7.20 (m, 3H), 7.17 (t, J=7.9 Hz, 1H), 7.04 (s, 1H), 6.89 (d, J=7.6 Hz, 1H), 6.26 (dd, J=10.2, 1.8 Hz, 1H), 6.09 (s, 1H), 5.67 (dd, J=11.2, 6.7 Hz, 1H), 5.60-5.48 (m, 2H), 4.94-4.85 (m, 2H), 4.56 (dd, J=18.2, 8.4 Hz, 1H), 4.34 (td, J=8.2, 5.4 Hz, 1H), 4.23-4.13 (m, 1H), 4.08 (s, 2H), 3.86 (s, 2H), 2.69-2.52 (m, 1H), 2.32-2.12 (m, 4H), 2.03 (dt, J=13.7, 3.7 Hz, 1H), 1.96-1.85 (m, 1H), 1.83-1.60 (m, 4H), 1.55-1.41 (m, 4H), 0.85 (s, 3H).

### Precursor Example 11: Synthesis of (S)-4-(2-(2,5-dioxo-2,5-dihydro-1H-pyrrol-1-yl)acetamido)-5-((3-(4-((6aS,6bR,7S,8aS,8bS,10R,11aR,12aS,12bS)-6b-fluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

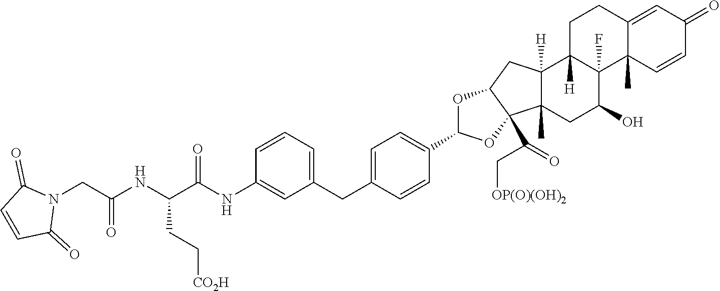

Precursor Example 11 product was synthesized in a similar procedure to Precursor Example 9 using the product from Precursor Example 3. LCMS (Method b, Table 7) Rₜ=0.80 min; MW m/z 934 [M+H]⁺.

¹H NMR (400 MHz, dimethylsulfoxide-d6) δ 0.89 (s, 3H), 1.39 (dd, J=12.7, 5.1 Hz, 1H), 1.50 (s, 3H), 1.67 (q, J=6.2 Hz, 3H), 1.76-2.02 (m, 3H), 2.07 (d, J=13.1 Hz, 1H), 2.22 (dtd, J=16.7, 11.1, 10.4, 4.6 Hz, 3H), 2.31-2.43 (m, 1H), 2.57-2.75 (m, 1H), 3.90 (s, 2H), 4.11 (s, 2H), 4.20 (d, J=8.9 Hz, 1H), 4.38 (td, J=8.2, 5.5 Hz, 1H), 4.58 (dd, J=18.2, 8.3 Hz, 1H), 4.84-5.00 (m, 2H), 5.51 (d, J=8.5 Hz, 2H), 6.04 (d, J=1.7 Hz, 1H), 6.24 (dd, J=10.2, 1.9 Hz, 1H), 6.88-6.99 (m, 1H), 7.09 (s, 2H), 7.15-7.32 (m, 4H), 7.32-7.42 (m, 3H), 7.42-7.53 (m, 1H), 8.48 (d, J=7.9 Hz, 1H), 9.95 (s, 1H).

### Precursor Example 12. 2-((2S,6aS.,6bR,7S,8aS,8bS,10R,11aR,12aS,12bS)-10-(4-(3-((S)-2-((S)-2-(2-(2,5-dioxo-2,5-dihydro-1H-pyrrol-1-yl)acetamido)propanamido)propanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

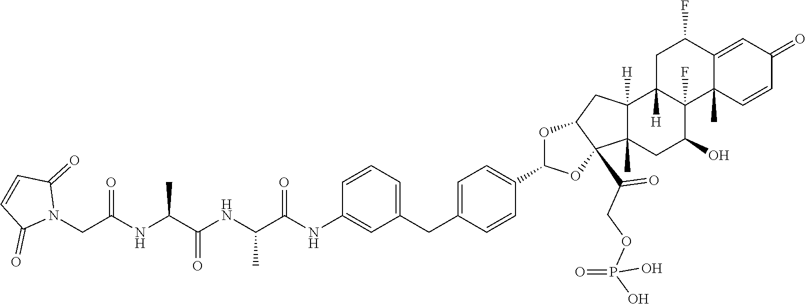

Precursor Example 12 product was synthesized in a similar procedure to Precursor Example 9 using the amino product of Precursor Example 1, dipeptide from Precursor Example 6 (Step 1), and maleimide reagent from Precursor Example 9 (Step 4). LCMS (Method b, Table 7) Rₜ=0.82 min; m/z=965.2 [M+H]⁺.

¹H NMR (400 MHz, dimethylsulfoxide-d6) δ 9.74 (s, 1H), 8.36 (d, J=7.3 Hz, 1H), 8.09 (d, J=7.2 Hz, 1H), 7.40 (dd, J=8.0, 2.0 Hz, 1H), 7.35 (d, J=1.8 Hz, 1H), 7.32 (d, J=7.9 Hz, 2H), 7.26-7.19 (m, 3H), 7.15 (t, J=7.8 Hz, 1H), 7.04 (s, 2H), 6.87 (d, J=7.5 Hz, 1H), 6.26 (dd, J=10.1, 1.9 Hz, 1H), 6.09 (s, 1H), 5.74-5.51 (m, 2H), 5.50 (s, 1H), 4.96-4.83 (m, 2H), 4.56 (dd, J=18.2, 8.4 Hz, 1H), 4.29 (dp, J=14.3, 7.1 Hz, 2H), 4.18 (d, J=9.3 Hz, 1H), 4.11-3.98 (m, 2H), 3.85 (s, 2H), 2.62 (dtd, J=33.1, 12.3, 4.2 Hz, 1H), 2.34-2.14 (m, 2H), 2.03 (dt, J=13.3, 3.7 Hz, 1H), 1.67 (dd, J=13.2, 5.1 Hz, 3H), 1.46 (s, 4H), 1.24 (d, J=7.1 Hz, 3H), 1.17 (d, J=7.0 Hz, 3H), 0.85 (s, 3H).

### Precursor Example 13. 2-((6aR,6bS,7S,8aS,8bS,10R,11aR,12aS,12bS)-10-(4-(3-((S)-2-((S)-2-(2-(2,5-dioxo-2,5-dihydro-1H-pyrrol-1-yl)acetamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

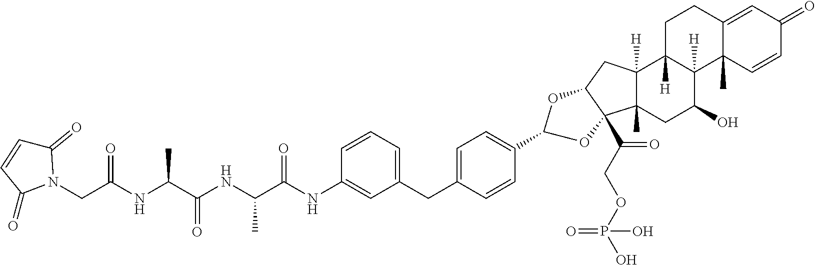

Precursor Example 13 product was synthesized in a similar procedure to Precursor Example 9 using the amino product of Precursor Example 2, dipeptide from Precursor Example 6 (Step 1), and maleimide reagent from Precursor Example 9 (Step 4). LCMS (Method c, Table 7) Rₜ=0.82 min; m/z=929.4 [M+H]⁺.

¹H NMR (400 MHz, dimethylsulfoxide-d₆) δ=9.79 (s, 1H), 8.42 (br d, J=7.5 Hz, 1H), 8.15 (br d, J=7.0 Hz, 1H), 7.49-7.28 (m, 5H), 7.25-7.12 (m, 3H), 7.07 (s, 2H), 6.90 (br d, J=7.5 Hz, 1H), 6.16 (br d, J=10.1 Hz, 1H), 5.92 (s, 1H), 5.48 (s, 1H), 4.96-4.85 (m, 2H), 4.56 (dd, J=8.3, 18.4 Hz, 1H), 4.37-4.23 (m, 3H), 4.08 (br d, J=2.6 Hz, 2H), 3.89 (s, 2H), 2.19-1.95 (m, 3H), 1.84-1.60 (m, 6H), 1.38 (s, 3H), 1.27 (br d, J=7.0 Hz, 3H), 1.20 (br d, J=6.6 Hz, 3H), 1.02 (br d, J=11.4 Hz, 2H), 0.87 (s, 3H), 0.00-0.00 (m, 1H).

### Precursor Example 14A. (S)-2-((2-(2-bromoacetamido)ethyl)amino)-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide

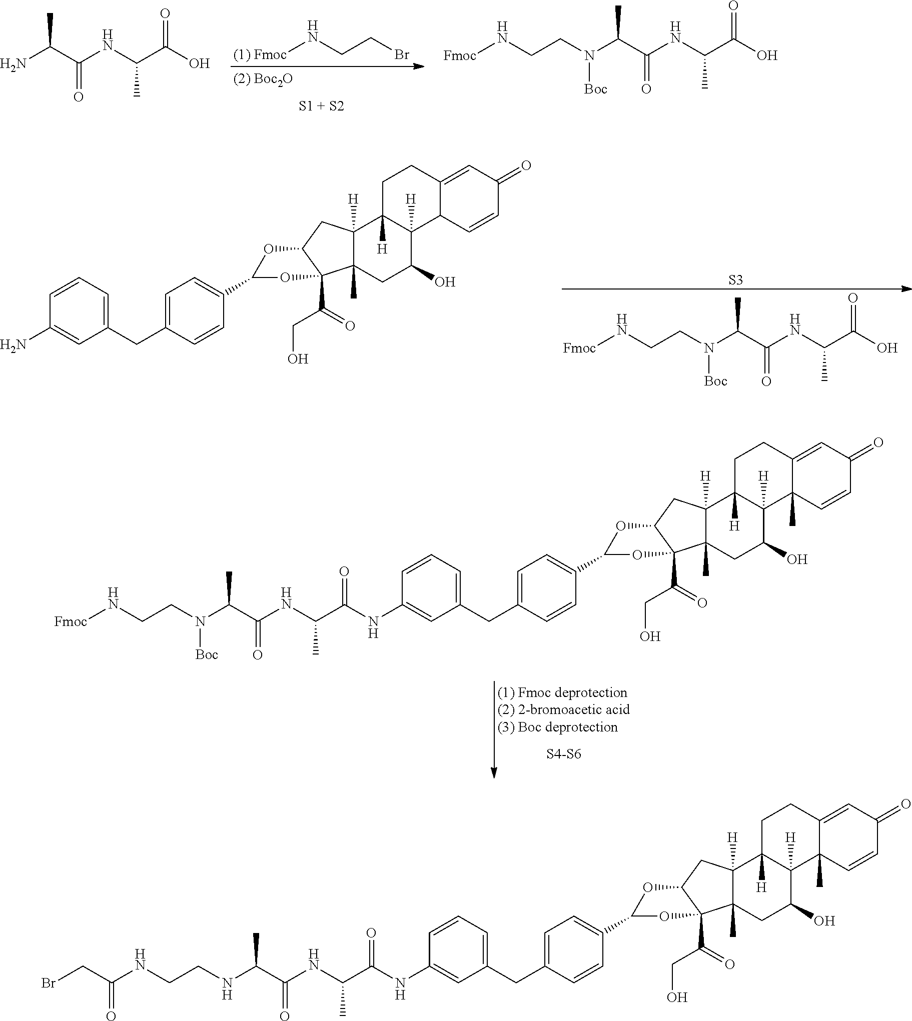

Precursor Example 14A product may be synthesized from coupling of N-(2-(4(9H-fluoren-9-yl)methoxy)carbonyl)amino)ethyl)-N-(tert-butoxycarbonyl)-L-alanyl-L-alanine (the product of steps S1 and S2) to the amino product of Example 2, followed by steps S4-S6: (1) Fmoc deprotection, (2) coupling with 2-bromoacetic acid, and (3) Boc deprotection. Fmoc=Fluorenylmethyloxycarbonyl; Boc=tertbutoxycarbonyl.

### Additional Precursor Examples

Example 14B-48 Bromo Acetamide Products, listed in Table 10, may be synthesized following the procedures described herein.

TABLE 10
Additional Bromo Acetamide Precursors

| | | Synthetic |
|---|---|---|
| Ex | Bromo Acetamide Product | Protocol |
| 14B | 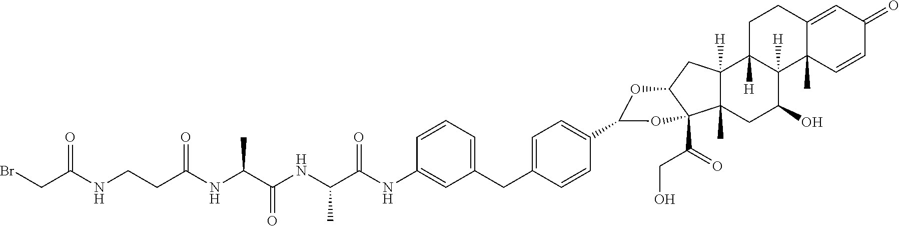 | May be made from Example 2 using a route similar to Example 6 Steps 1 and 2 and Example 4 Step 5. A synthetic protocol and characterization for this Precursor is also provided following this Table. |
| 15 | 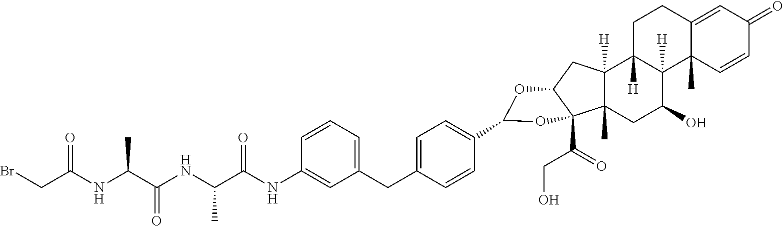 | May be made from Example 2 using a route similar to Example 6 Steps 1 and 2 and Example 4 Step 5. A synthetic protocol and characterization for this Precursor is also provided following this Table. |
| 16 | 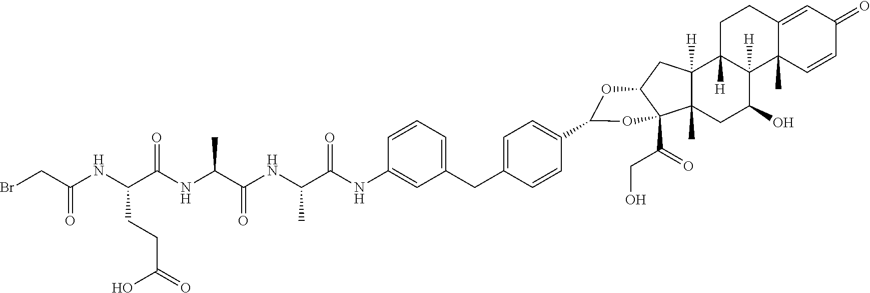 | May be made from Example 2 using a route similar to Example 4. |
| 17 | 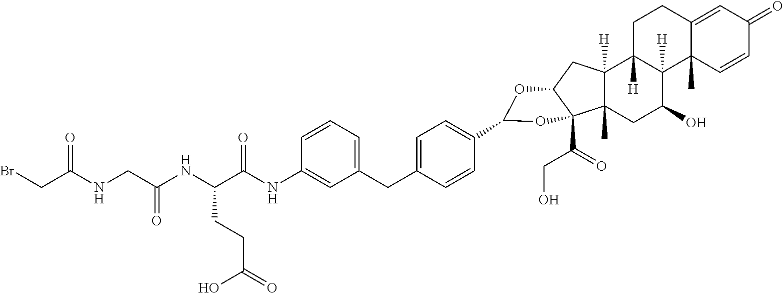 | May be made from Example 2 using a route similar to Example 4. |
| 18A | 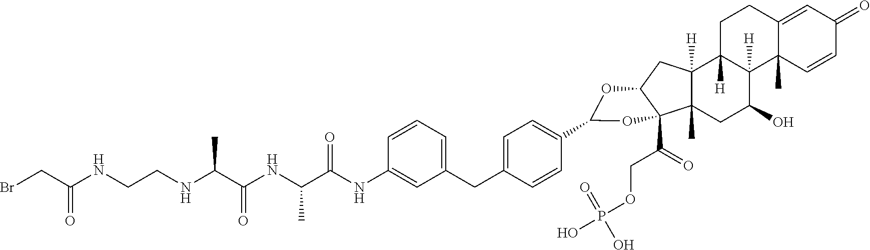 | May be made from Example 2 using a route similar to Example 14A. |
| 18B | 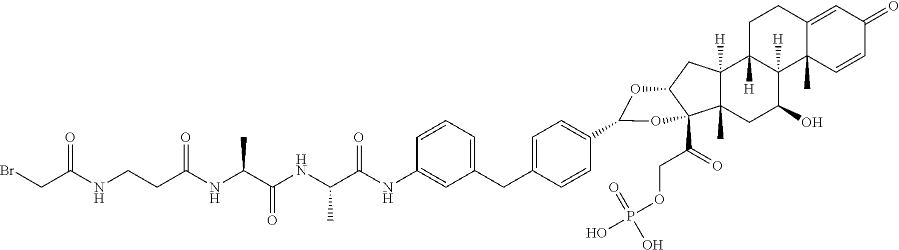 | May be made from Example 2 using a route similar to Example 6 Steps 1 and 2 and Example 4. |
| 19 | 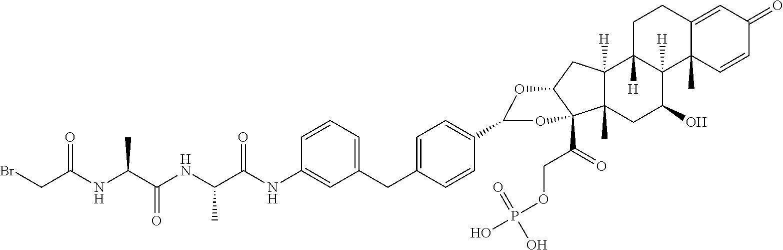 | May be made from Example 2 using a route similar to Example 6 Steps 1 and 2 and Example 4. |
| 20 | 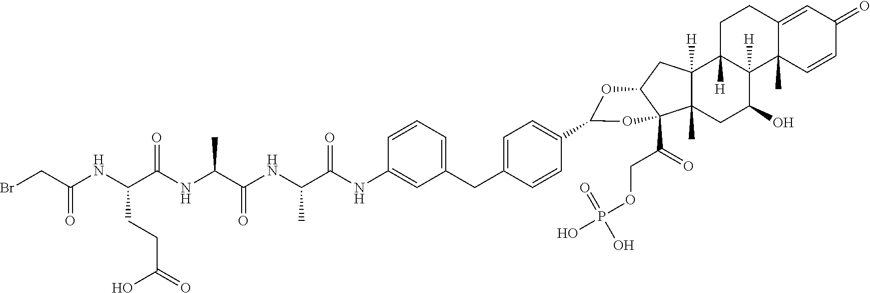 | May be made from Example 2 using a route similar to Example 4. |
| 21A | 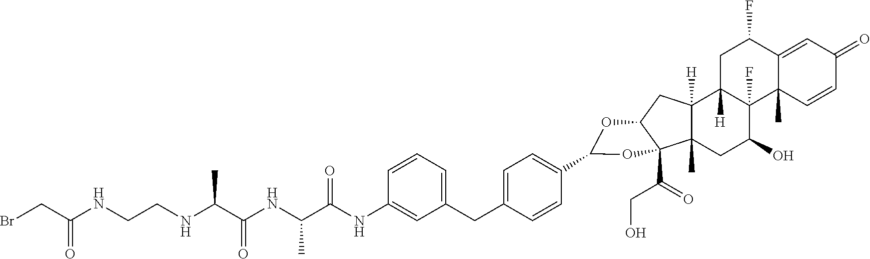 | May be made from Example 1 using a route similar to Example 14A. |
| 21B | 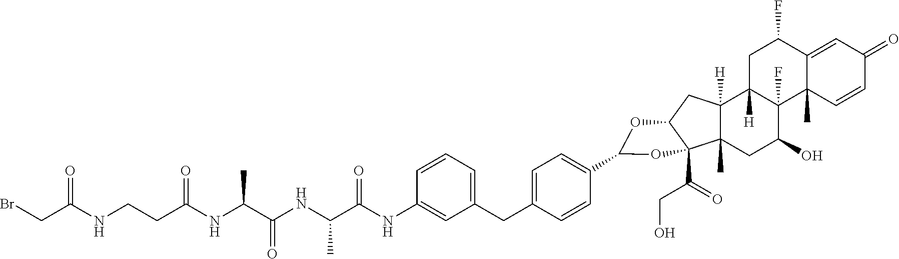 | May be made from Example 1 using a route similar to Example 6 Steps 1 and 2 and Example 4 Step 5. A synthetic protocol and characterization for this Precursor is also provided following this Table. |
| 22 | 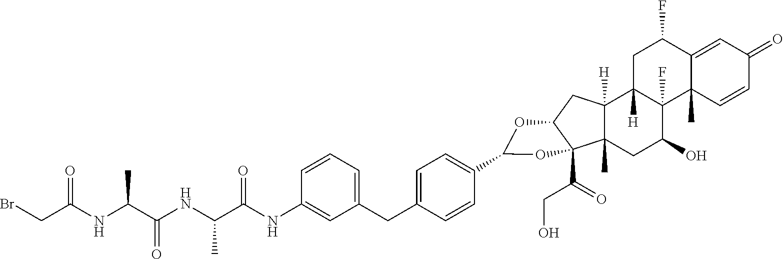 | May be made from Example 1 using a route similar to Example 6 Steps 1 and 2 and Example 4 Step 5. A synthetic protocol and characterization for this Precursor is also provided following this Table. |
| 23 | 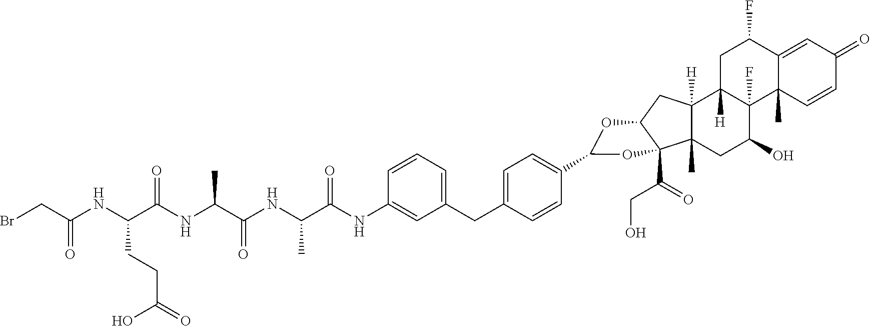 | May be made from Example 1 using a route similar to Example 4. A synthetic protocol and characterization for this Precursor is also provided following this Table. |
| 24 | 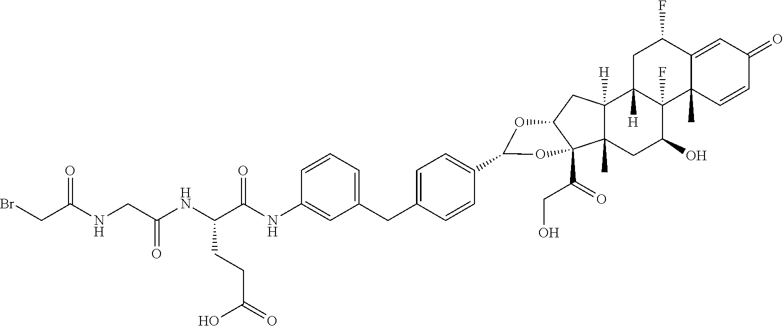 | May be made from Example 1 using a route similar to Example 4. A synthetic protocol and characterization for this Precursor is also provided following this Table. |
| 25A | 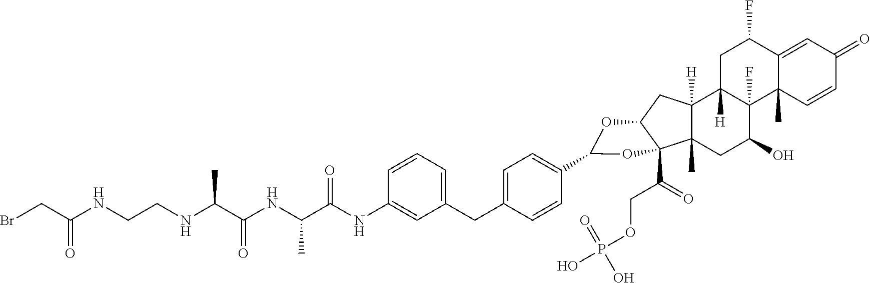 | May be made from Example 1 using a route similar to Example 14A. |
| 25B | 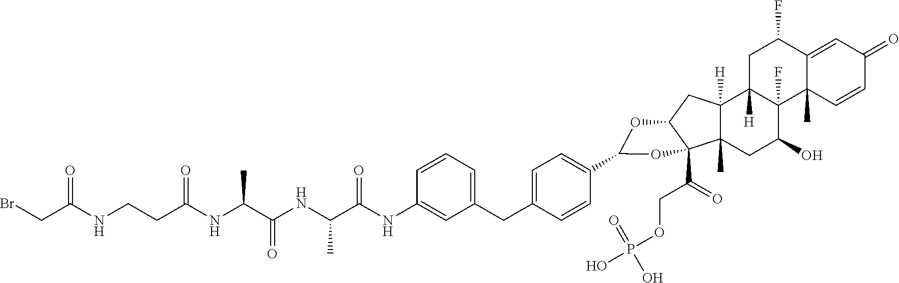 | May be made from Example 1 using a route similar to Example 6 Steps 1 and 2 and Example 4. A synthetic protocol and characterization for this Precursor is also provided following this Table. |
| 26 | 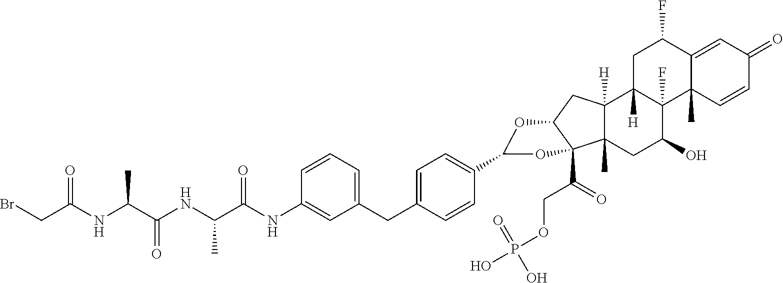 | May be made from Example 1 using a route similar to Example 6 Steps 1 and 2 and Example 4. A synthetic protocol and characterization for this Precursor is also provided following this Table. |
| 27 | 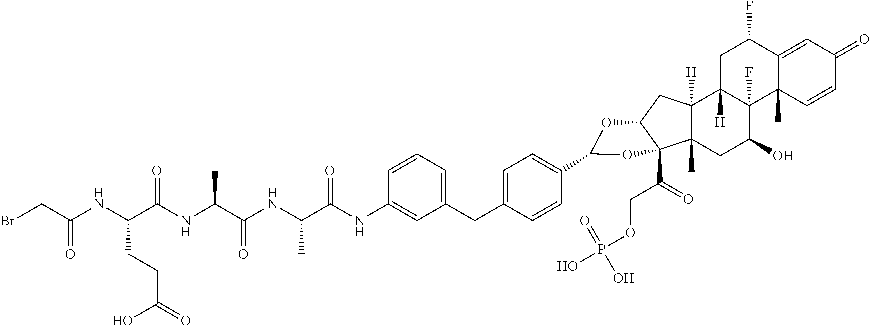 | May be made from Example 1 using a route similar to Example 4. A synthetic protocol and characterization for this Precursor is also provided following this Table. |
| 28 | 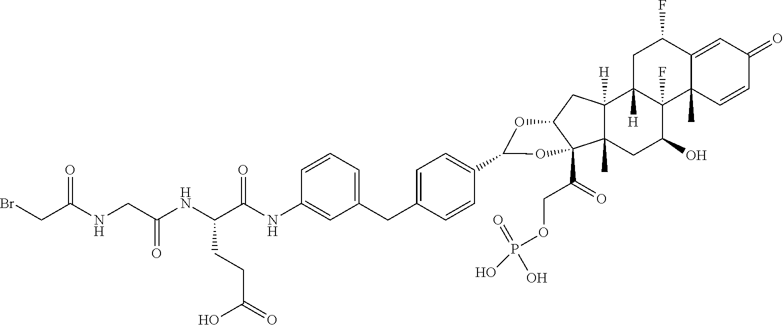 | May be made from Example 1 using a route similar to Example 4. A synthetic protocol and characterization for this Precursor is also provided following this Table. |
| 29A | 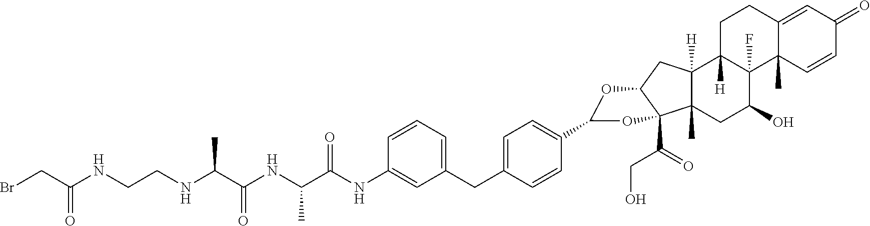 | May be made from Example 3 using a route similar to Example 14A. |
| 29B | 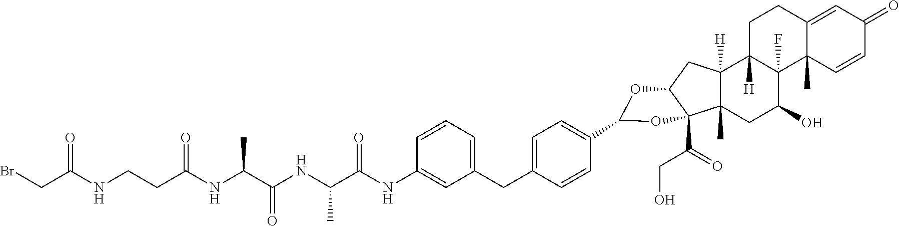 | May be made from Example 3 using a route similar to Example 6 Steps 1 and 2 and Example 4 Step 5. |
| 30 | 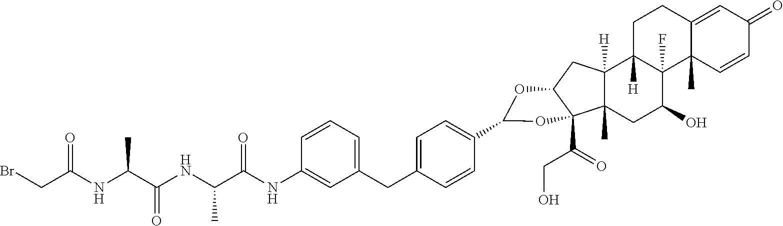 | May be made from Example 3 using a route similar to Example 6 Steps 1 and 2 and Example 4 Step 5. |
| 31 | 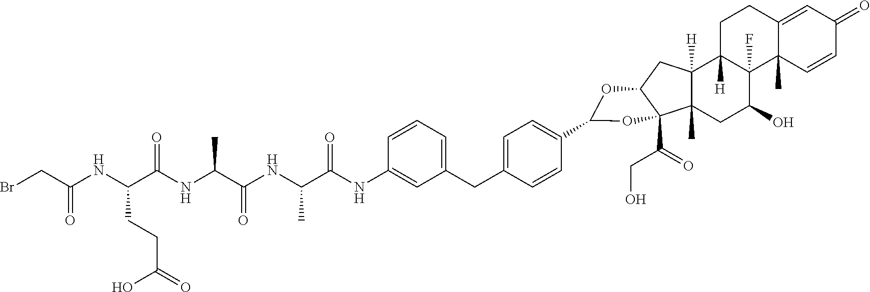 | May be made from Example 3 using a route similar to Example 4. |
| 32 | 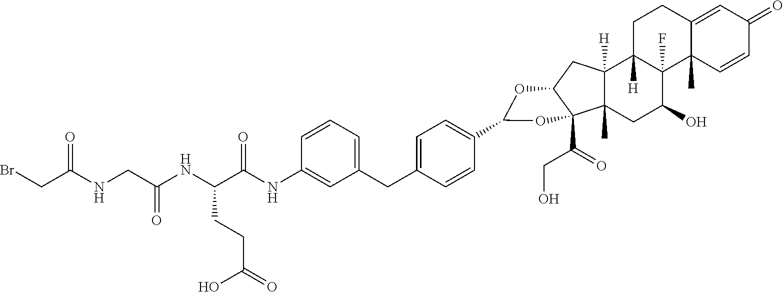 | May be made from Example 3 using a route similar to Example 4. |
| 33A | 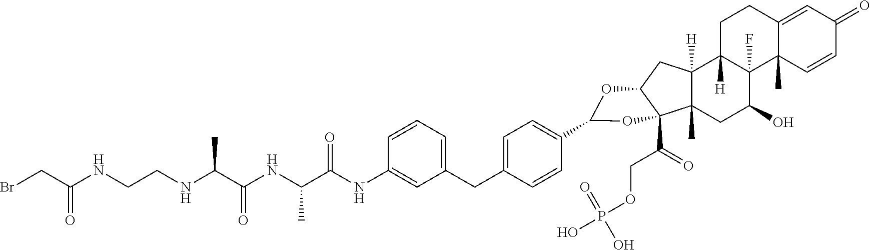 | May be made from Example 3 using a route similar to Example 14A. |
| 33B | 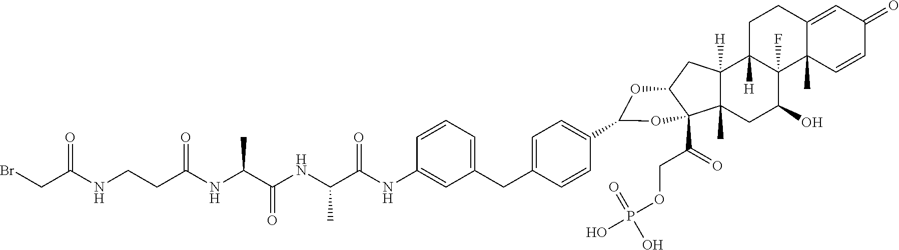 | May be made from Example 3 using a route similar to Example 6 Steps 1 and 2 and Example 4. |
| 34 | 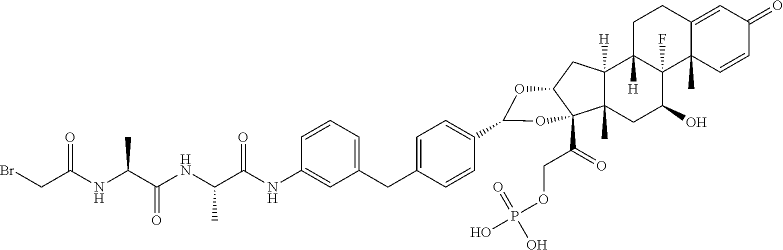 | May be made from Example 3 using a route similar to Example 6 Steps 1 and 2 and Example 4. |
| 35 | 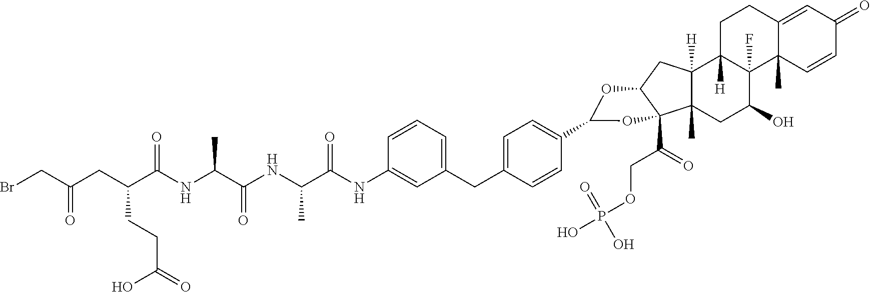 | May be made from Example 3 using a route similar to Example 4. |
| 36 | 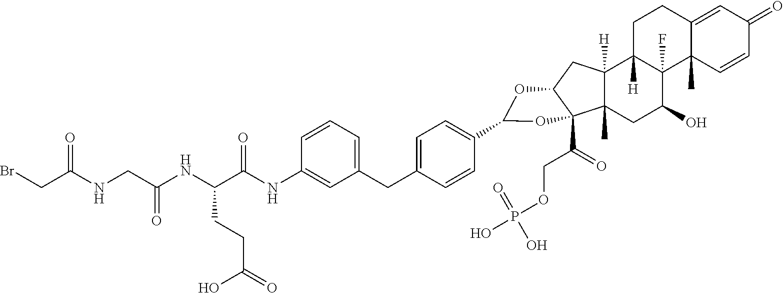 | May be made from Example 3 using a route similar to Example 4. A synthetic protocol and characterization for this Precursor is also provided following this Table. |
| 37 | 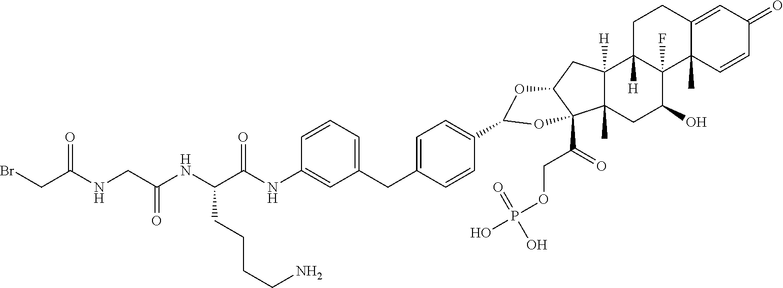 | May be made from Example 3 using a route similar to Example 5. A synthetic protocol and characterization for this Precursor is also provided following this Table. |
| 38 | 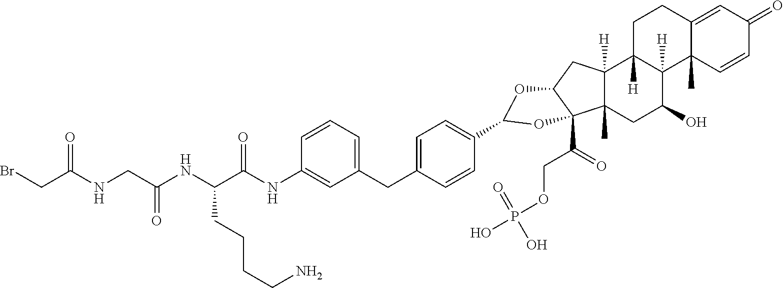 | May be made from Example 2 using a route similar to Example 5. |
| 39 | 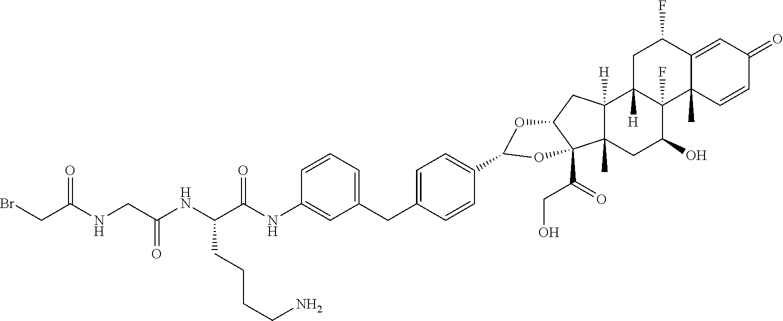 | May be made from Example 2 using a route similar to Example 5. A synthetic protocol and characterizattion for this Precursor is also provided following this Table. |
| 40 | 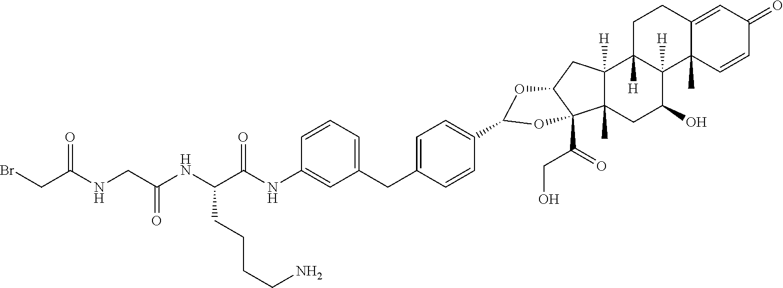 | May be made from Example 2 using a route similar to Example 5. |
| 41 | 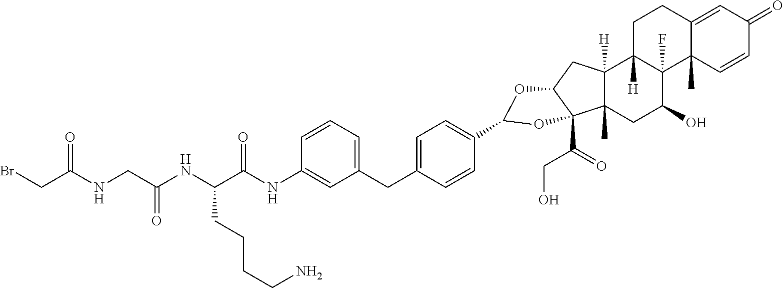 | May be made from Example 3 using a route similar to Example 5. |
| 42 | 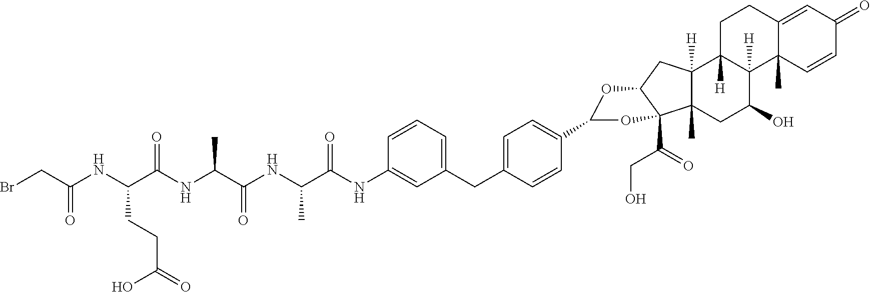 | A synthetic protocol and characterization for this Precursor is also provided following this Table. |
| 43 | 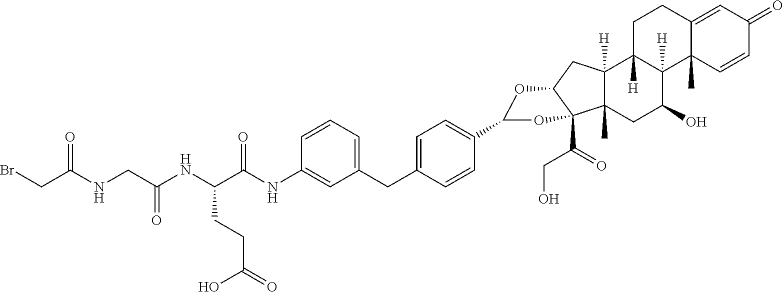 | A synthetic protocol and characterization for this Precursor is also provided following this Table. |
| 44 | 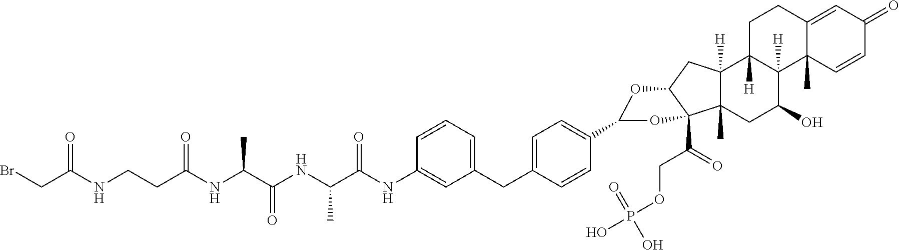 | A synthetic protocol and characterization for this Precursor is also provided following this Table. |
| 45 | 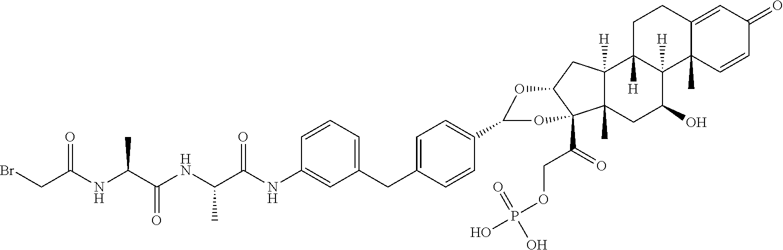 | A synthetic protocol and characterization for this Precursor is also provided following this Table. |
| 46 | 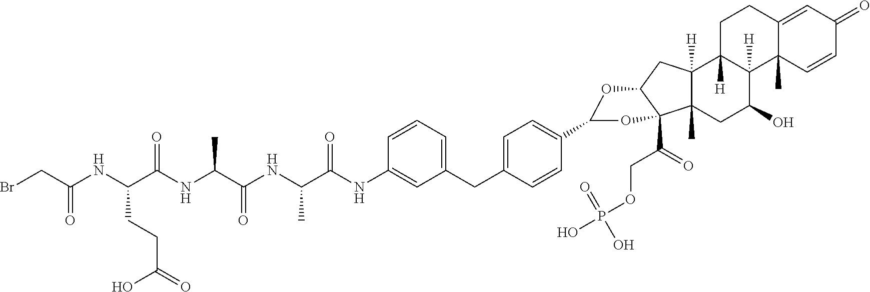 | A synthetic protocol and characterization for this Precursor is also provided following this Table. |
| 47 | 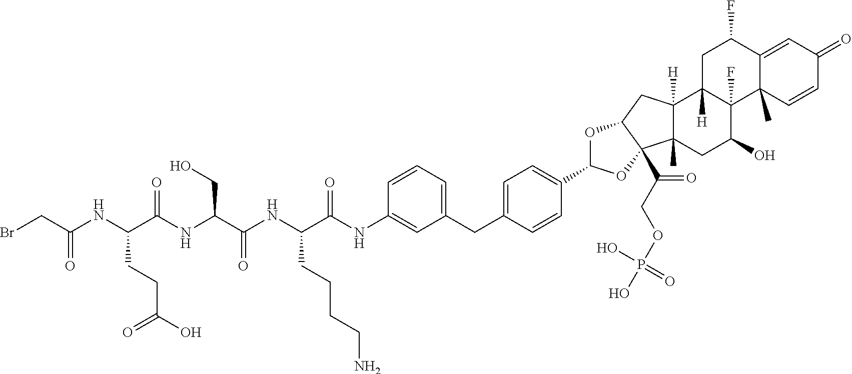 | A synthetic protocol and characterization for this Precursor is also provided following this Table. |
| 48 | 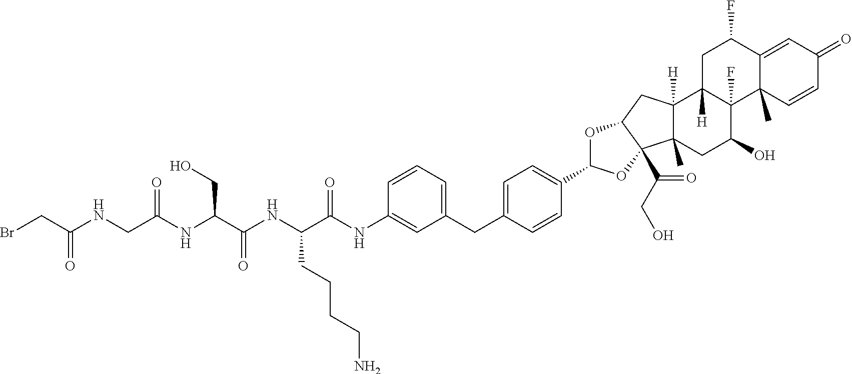 | A synthetic protocol and characterization for this Precursor is also provided following this Table. |

### Precursor Example 14B. 3-(2-Bromoacetamido)-N—((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)propanamide

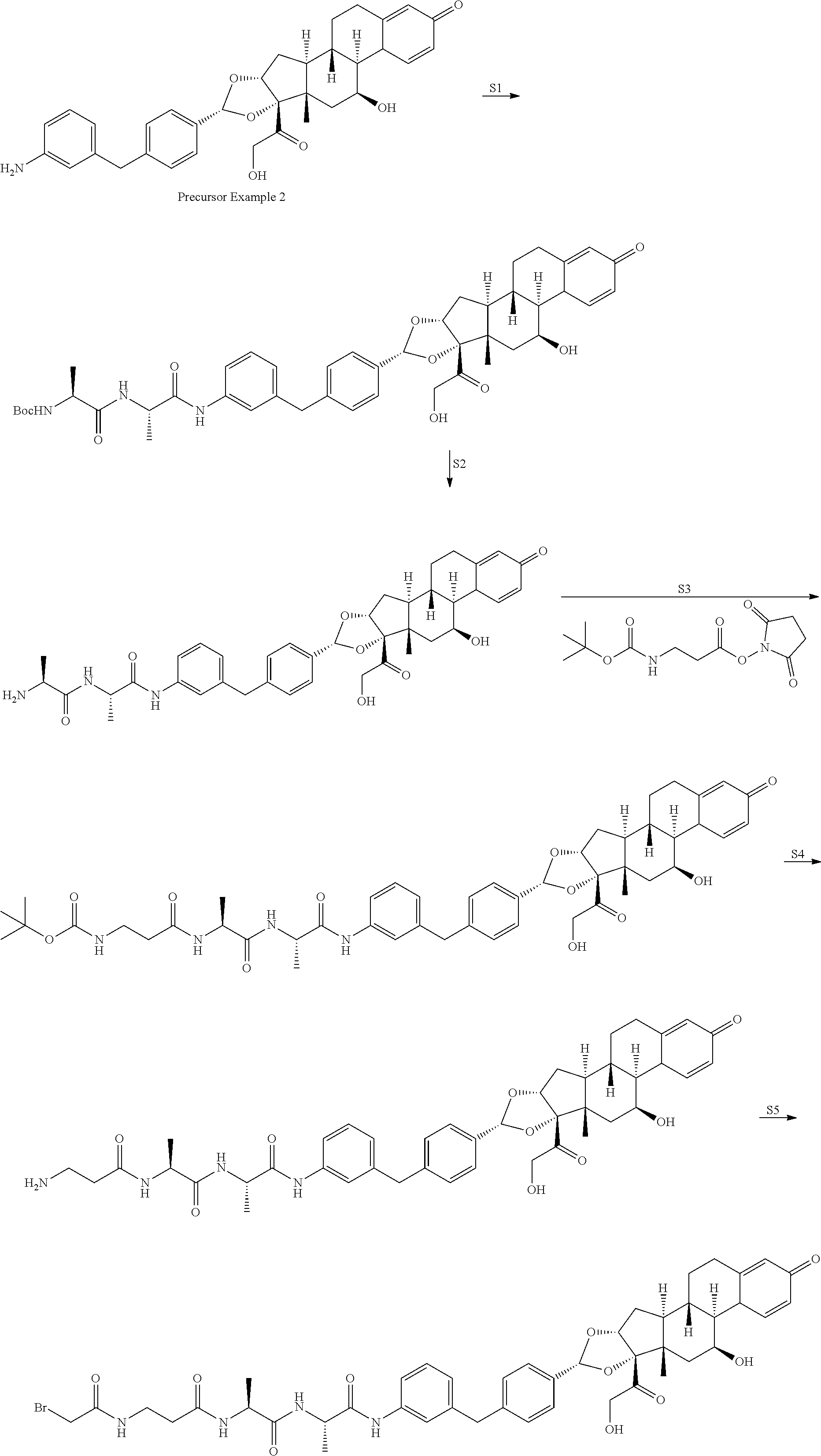

Step 1: Synthesis of tert-butyl ((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)carbamate. To a solution of (tert-butoxycarbonyl)-L-alanyl-L-alanine (11.9 g, 45.6 mmol, 1.30 eq) in tetrahydrofuran (140 mL) was added N-ethoxycarbonyl-2-ethoxy-1,2-dihydroquinoline (11.3 g, 45.6 mmol, 1.30 eq), the reaction mixture was stirred at 15° C. for 0.5 hours. Then the product of Precursor Example 2 ((6aR,6bS,7S,8aS,8bS,10R,11aR,12aS,12bS)-10-(4-(3-aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (20.0 g, 35.1 mmol)) was added, and the mixture was stirred at 15° C. for 2 hour. The reaction mixture was concentrated under reduced pressure to give a residue and purified by column chromatography on silica gel (SiO₂, Petroleum ether/Ethyl acetate=3/1 to 0/1) to afford the title compound (25.0 g, 88% yield). ¹H NMR (400 MHz dimethylsulfoxide-d6) δ 9.85 (s, 1H), 7.96 (d, J=7.2 Hz, 1H), 7.37-7.41 (m, 4H), 7.31 (d, J=10.0 Hz, 1H), 7.21-7.23 (m, 3H), 6.94 (dd, J=23.6, 7.2 Hz, 2H), 6.16 (d, J=10.0 Hz, 1H), 5.93 (s, 1H), 5.41 (s, 1H), 5.08 (t, J=5.6 Hz, 1H), 4.92 (d, J=5.2 Hz, 1H), 4.78 (d, J=3.2 Hz, 1H), 4.50 (dd, J=19.6, 6.4 Hz, 1H), 4.35-4.38 (m, 1H), 4.29 (s, 1H), 4.18 (dd, J=19.6, 5.6 Hz, 1H), 3.98-4.16 (m, 1H), 3.89 (s, 2H), 2.54-2.58 (m, 1H), 2.31 (d, J=10.8 Hz, 1H), 2.03-2.10 (m, 2H), 1.67-1.77 (m, 6H), 1.39 (s, 3H), 1.37 (s, 9H), 1.16-1.19 (m, 3H), 1.01-1.03 (m, 2H), 0.86 (s, 3H).

Step 2: Synthesis of (S)-2-amino-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide hydrochloride. A solution of tert-butyl ((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)carbamate (15.0 g, 18.5 mmol, 1.0 eq) in HCl/methyl tert-butyl ethe (90 mL, 4 M) was stirred at 20° C. for 0.5 hours. The reaction mixture was filtered and the filter cake was dried. The residue was purified by prep-HPLC to afford the title compound (10.2 g, 74% yield). ¹H NMR (400 MHz dimethylsulfoxide-d6) δ 10.0 (s, 1H), 8.68 (d, J=7.2 Hz, 1H), 8.13 (br d, J=3.6 Hz, 3H), 7.43-7.45 (m, 2H), 7.39 (d, J=7.6 Hz, 2H), 7.32 (d, J=10.4 Hz, 1H), 7.21-7.24 (m, 3H), 6.93 (br d, J=7.6 Hz, 1H), 6.16 (dd, J=10.0, 1.6 Hz, 1H), 5.93 (s, 1H), 5.41 (s, 1H), 4.92 (d, J=4.8 Hz, 1H), 4.82 (br s, 1H), 4.41-4.52 (m, 2H), 4.29 (br s, 1H), 4.18 (d, J=19.6 Hz, 1H), 3.87-3.89 (m, 3H), 2.54-2.58 (m, 1H), 2.29-2.33 (m, 1H), 2.01-2.03 (m, 2H), 1.67-1.77 (m, 5H), 1.40 (s, 3H), 1.34 (dd, J=14.4, 6.8 Hz, 6H), 1.02-1.03 (m, 2H), 0.86 (s, 3H).

Step 3: Synthesis of tert-butyl (3-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-3-oxopropyl)carbamate. To a solution of 2,5-dioxopyrrolidin-1-yl 3-((tert-butoxycarbonyl)amino)propanoate (205 mg, 0.716 mmol) in dimethylformamide (3 mL) was added (S)-2-amino-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide (333 mg, 0.477 mmol) and N, N-diisopropylethylamine (0.167 mL, 0.954 mmol) at 25° C. The reaction was stirred at 25° C. for 2 hours. Two additional vials were set up as described above. All three reaction mixtures were combined and purified by Prep-HPLC (Method AA1) to afford the title compound (300 mg, 24% yield). LCMS (Method AA13) Rt=1.152 min, m/z 883.5 (M+H)⁺.

Step 4: Synthesis of 3-amino-N—((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)propanamide. To a solution of tert-butyl (3-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-3-oxopropyl)carbamate (100 mg, 0.113 mmol) in dichloromethane (1 mL) was added trifluoroacetic acid (0.33 mL, 4.28 mmol) at 25° C. The reaction was stirred at 25° C. for 1 hour. One additional vial was set up as described above. Both reaction mixtures were combined and purified by Prep-HPLC (Method AA2) to afford the title compound (50 mg, 28% yield). LCMS (Method AA13) Rt=0.987 min, m/z 783.4 (M+H)⁺.

Step 5: Synthesis of 3-(2-bromoacetamido)-N—((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)propanamide. To a solution of 2-bromoacetic acid (35.5 mg, 0.255 mmol) in dimethylformamide (1 mL) was added 2-ethoxy-1-ethoxycarbonyl-1, 2-dihydroquinoline (63.2 mg, 0.255 mmol) at 25° C. The reaction was stirred at 25° C. for 30 minutes then 3-amino-N—((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)propanamide (100 mg, 0.128 mmol) was added at 25° C. The reaction was stirred at 25° C. for 2 hours. One additional vial was set up as described above. Both reaction mixtures were combined and purified by Prep-HPLC (Method AA3) to afford the title compound (80 mg, 35% yield). LCMS (Method AA4) Rt: 2.993 min. m/z 905.3 (M+H)⁺. ¹H NMR (methanol-d4, 400 MHz) δ 7.53-7.48 (m, 1H), 7.46-7.40 (m, 2H), 7.34 (dd, J=1.5, 8.2 Hz, 2H), 7.23-7.14 (m, 3H), 6.92 (t, J=7.5 Hz, 1H), 6.24 (d, J=9.5 Hz, 1H), 6.01 (s, 1H), 5.42 (d, J=1.3 Hz, 1H), 5.04 (d, J=4.9 Hz, 1H), 4.62 (d, J=19.4 Hz, 1H), 4.48-4.21 (m, 4H), 3.93 (d, J=2.2 Hz, 2H), 3.76 (s, 1H), 3.65 (s, 1H), 3.45 (quind, J=6.4, 12.8 Hz, 2H), 2.65 (dt, J=5.6, 13.1 Hz, 1H), 2.52-2.34 (m, 3H), 2.25 (dq, J=3.9, 10.8 Hz, 1H), 2.13 (br dd, J=5.7, 12.6 Hz, 1H), 1.95 (br d, J=13.7 Hz, 1H), 1.89-1.64 (m, 5H), 1.48 (s, 3H), 1.43 (dd, J=2.2, 7.3 Hz, 3H), 1.36 (dd, J=7.2, 10.7 Hz, 3H), 1.20-1.07 (m, 1H), 1.03 (ddd, J=3.6, 7.5, 11.1 Hz, 1H), 0.98 (s, 3H).

### Precursor Example 15: (S)-2-(2-Bromoacetamido)-N—((S)-14(3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide

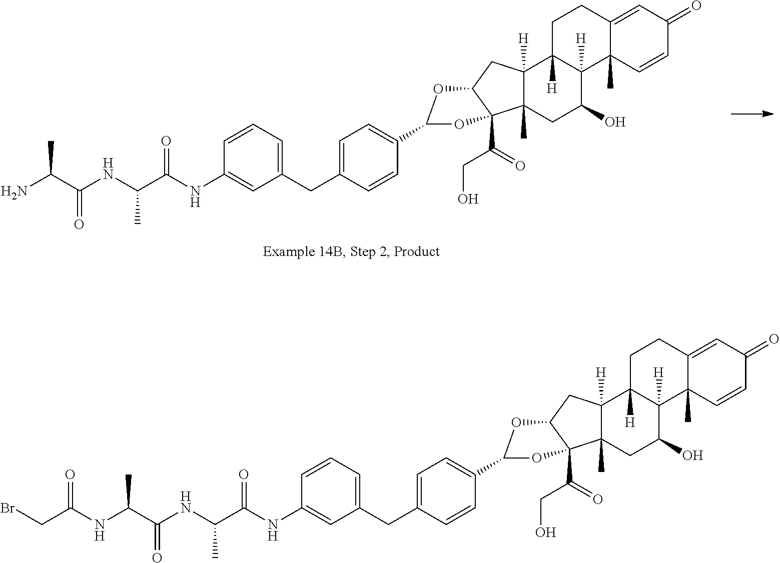

Step 1: Synthesis of (S)-2-(2-bromoacetamido)-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide. To a solution of 2-bromoacetic acid (109 mg, 0.787 mmol) in dimethylformamide (2 mL) was added 2-ethoxy-1-ethoxycarbonyl-1, 2-dihydroquinoline (195 mg, 0.787 mmol) at 25° C. The reaction was stirred at 25° C. for 30 minutes whereupon the product of Precursor Example 14B, Step 2 ((S)-2-amino-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide)(280 mg, 0.393 mmol) was added to the mixture. The reaction was stirred at 25° C. for 2 hours and then purified by Prep-HPLC (Method AA5) to afford the title compound (85 mg, 26% yield). LCMS (Method AA4) Rt=3.10 min, m/z 834.3 (M+H)⁺. ¹H NMR (methanol-d4, 400 MHz) δ 0.98 (s, 3H), 1.02 (br s, 1H), 1.12 (br d, J=10.5 Hz, 1H), 1.40 (br dd, J=7.0, 10.5 Hz, 6H), 1.48 (s, 3H), 1.89-1.66 (m, 4H), 1.98-1.89 (m, 1H), 2.12 (br d, J=12.7 Hz, 1H), 2.24 (br d, J=10.5 Hz, 1H), 2.37 (br d, J=11.0 Hz, 1H), 2.70-2.58 (m, 1H), 3.93-3.80 (m, 4H), 4.46-4.24 (m, 4H), 4.61 (d, J=19.3 Hz, 1H), 5.04 (d, J=4.8 Hz, 1H), 5.43 (s, 1H), 6.01 (s, 1H), 6.24 (br d, J=8.8 Hz, 1H), 6.90 (br d, J=7.0 Hz, 1H), 7.23-7.12 (m, 3H), 7.47-7.30 (m, 5H).

### Precursor Example 21B: (S)-24(2-(2-Bromoacetamido)ethyl)amino)-N—((S)-14(3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide

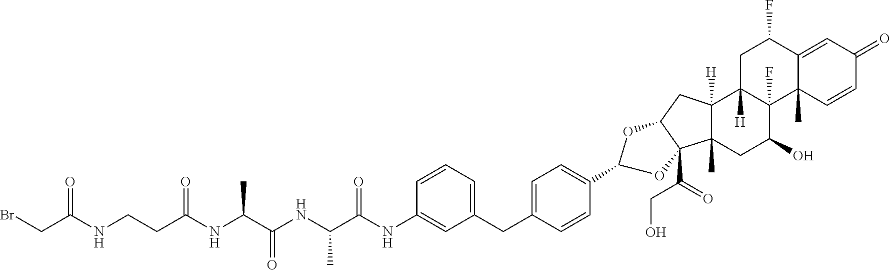

Prepared using similar route to Precursor Example 14B using (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one.

LCMS (Method AA4) Rt=2.942 min, m/z 940.3 (M+H)⁺. ¹H NMR (dimethylsulfoxide-d6) 6=9.81-9.68 (m, 1H), 8.31-8.17 (m, 2H), 8.15-8.04 (m, 1H), 7.56-7.39 (m, 2H), 7.35 (d, J=7.9 Hz, 2H), 7.30-7.16 (m, 4H), 6.91 (d, J=7.5 Hz, 1H), 6.30 (dd, J=2.0, 10.3 Hz, 1H), 6.13 (s, 1H), 5.74-5.49 (m, 2H), 5.45 (s, 1H), 5.46-5.43 (m, 1H), 4.94 (d, J=4.8 Hz, 1H), 4.51 (d, J=19.7 Hz, 1H), 4.40-4.15 (m, 4H), 3.88 (s, 2H), 3.82 (d, J=3.9 Hz, 2H), 3.32-3.22 (m, 2H), 2.72-2.57 (m, 1H), 2.36-2.19 (m, 4H), 2.09-2.00 (m, 1H), 1.78-1.62 (m, 3H), 1.57-1.45 (m, 4H), 1.27 (dd, J=2.4, 7.2 Hz, 3H), 1.20 (d, J=7.0 Hz, 3H), 0.86 (s, 3H).

### Precursor Example 22: (S)-2-(2-Bromoacetamido)-N—((S)-14(3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)propanamide

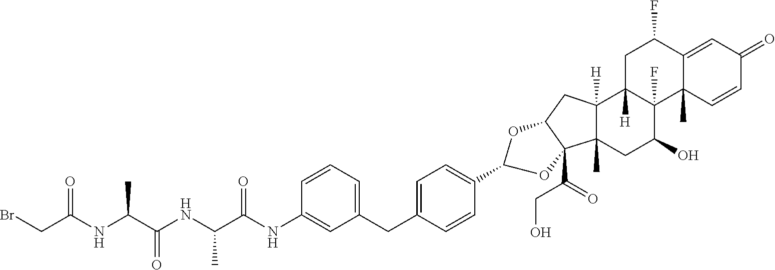

Prepared using similar route to Precursor Example 15 using (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one.

LCMS (Method AA4) Rt=3.089 min, m/z 869.3 (M+H)⁺. ¹H NMR (methanol-d4) δ 7.29-7.57 (m, 5H), 7.15-7.26 (m, 3H), 6.93 (br d, J=7.02 Hz, 1H), 6.30-6.38 (m, 2H), 5.47-5.68 (m, 1H), 5.43-5.45 (m, 1H), 5.04 (br d, J=3.95 Hz, 1H), 4.62 (br d, J=19.29 Hz, 1H), 4.25-4.46 (m, 4H), 3.93 (br s, 2H), 3.77-3.90 (m, 2H), 2.60-2.78 (m, 1H), 2.21-2.49 (m, 3H), 1.63-1.85 (m, 4H), 1.58 (s, 3H), 1.35-1.46 (m, 6H), 0.98 (s, 3H).

### Precursor Example 42: (S)-4-(2-Bromoacetamido)-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoic acid

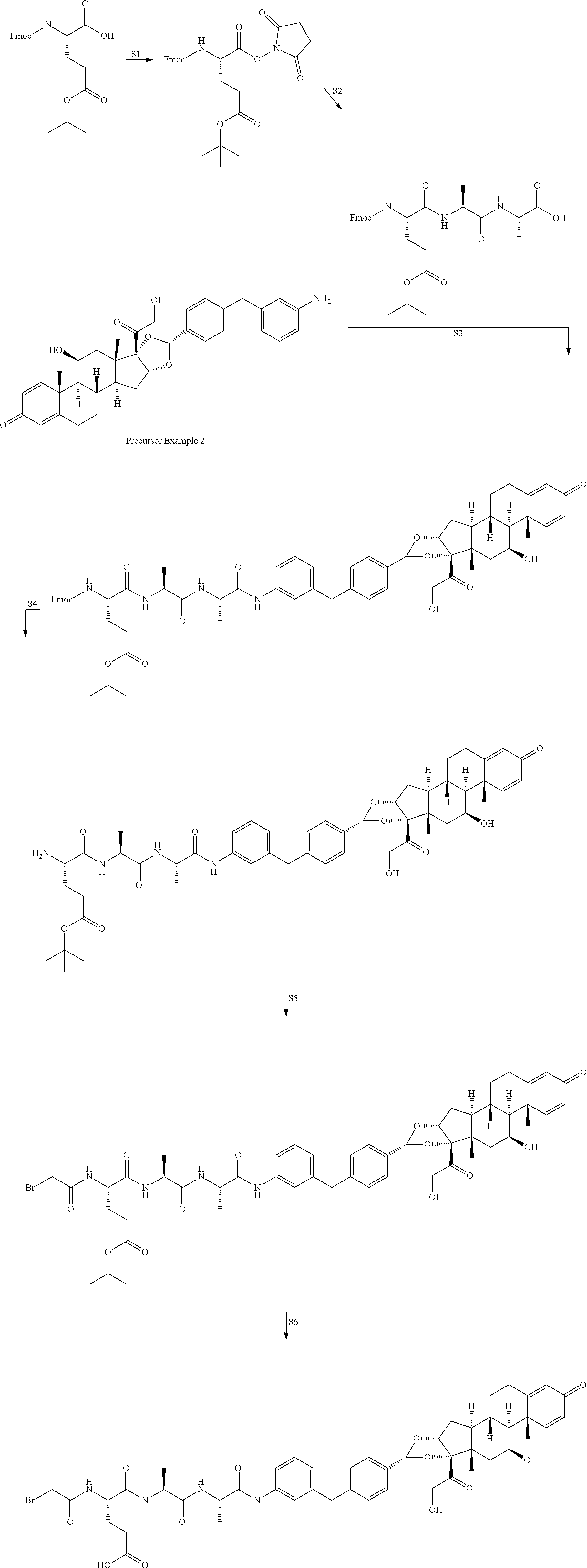

Step 1: Synthesis of 5-(tert-butyl) 1-(2,5-dioxopyrrolidin-1-yl) (((9H-fluoren-9-yl)methoxy)carbonyl)-L-glutamate. To a solution of (S)-2-((((9H-fluoren-9-yl)methoxy)carbonyl)amino)-5-(tert-butoxy)-5-oxopentanoic acid (50 g, 118 mmol) and 1-hydroxypyrrolidine-2,5-dione (13.52 g, 118 mmol) in dichloromethane (600 mL) was added N,N′-methanediylidenedicyclohexanamine (DCC) (24.25 mg, 118 mmol) at 0° C. and the mixture stirred at 25° C. for 4 hours. The mixture was filtered through a sintered glass funnel, washed with dichloromethane (100 mL). The solvent was removed under vacuum to afford the title compound (60 g, 96% yield). LCMS (Method AA13) Rt=1.663 min, m/z 545.0 (M+Na)⁺. ¹H NMR (CDCl₃, 400 MHz) δ 1.40-1.54 (m, 9H) 2.15 (dq, J=14.53, 7.36 Hz, 1H) 2.25-2.38 (m, 1H) 2.39-2.53 (m, 2H) 2.82 (s, 4H) 4.17-4.27 (m, 1H) 4.30-4.49 (m, 2H) 4.72-4.83 (m, 1H) 5.71 (br d, J=8.16 Hz, 1H) 7.24-7.34 (m, 2H) 7.36-7.44 (m, 2H) 7.55-7.63 (m, 2H) 7.76 (d, J=7.50 Hz, 2H). Fmoc=Fluorenylmethyloxycarbonyl

Step 2: Synthesis of ((S)-2-((((9H-fluoren-9-yl)methoxy)carbonyl)amino)-5-(tert-butoxy)-5-oxopentanoyl)-L-alanyl-L-alanine. To a solution of (S)-2-((S)-2-aminopropanamido)propanoic acid (6.13 g, 38.3 mmol) in 1,2-dimethoxyethane (200 mL) and water (133 mL) was added NaHCO₃ (12.86 g, 153 mmol) and 5-(tert-butyl) 1-(2,5-dioxopyrrolidin-1-yl) (((9H-fluoren-9-yl)methoxy)carbonyl)-L-glutamate (20 g, 38.3 mmol). The mixture was stirred at 25° C. for 4 hours. The mixture was concentrated under reduced pressure to remove the solvent. Saturated NaHCO₃ solution (250 mL) and ethyl acetate (250 mL) were added and the layers separated. Aqueous HCl (1M, 250 mL) was added to the aqueous layer and extracted with ethyl acetate (300 mL). The organic layer was dried (Na₂SO₄), filtered and solvent was removed under vacuum to afford the title compound (15 g, 69% yield). LCMS (Method AA13) Rt=1.170 min, m/z 568.4 (M+H)⁺. ¹H NMR (dimethylsulfoxide-d6, 400 MHz) δ 1.15 (t, J=7.06 Hz, 1H) 1.21 (br d, J=7.06 Hz, 3H) 1.26 (br d, J=7.28 Hz, 3H) 1.37 (s, 9H) 1.65-1.78 (m, 1H) 1.81-1.93 (m, 1H) 1.96 (s, 1H) 2.23 (br t, J=7.83 Hz, 2H) 4.01 (quin, J=7.11 Hz, 2H) 4.15-4.23 (m, 2H) 4.23-4.32 (m, 2H) 7.26-7.34 (m, 2H) 7.36-7.43 (m, 2H) 7.53 (br d, J=8.16 Hz, 1H) 7.71 (br t, J=6.73 Hz, 2H) 7.86 (d, J=7.50 Hz, 2H) 7.99 (br d, J=7.28 Hz, 1H) 8.14 (br d, J=7.28 Hz, 1H) 12.53 (br s, 1H).

Step 3: Synthesis of tert-butyl (S)-4-((((9H-fluoren-9-yl)methoxy)carbonyl)amino)-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate. To a solution of ((S)-2-((((9H-fluoren-9-yl)methoxy)carbonyl)amino)-5-(tert-butoxy)-5-oxopentanoyl)-L-alanyl-L-alanine (1.495 g, 2.63 mmol) in dimethylformamide (5 mL) was added 2,4,6-tripropyl-1,3,5,2,4,6-trioxatriphosphinane 2,4,6-trioxide (2.234 g, 3.51 mmol) and triethylamine (0.533 g, 5.27 mmol) at 0° C. The mixture was stirred at 25° C. for 30 minutes. Precursor Example 2 ((6aR,6bS,7S,8aS,8bS,10R,11aR,12aS,12bS)-10-(4-(3-Aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one) (1 g, 1.755 mmol) was added to the mixture at 25° C. and the reaction stirred for 12 hours. The mixture was added to ice-cold water (50 mL) and the precipitate collected by filtration to afford the title compound (1.68 g, 86% yield). LCMS (Method AA13) Rt=1.344 min, m/z 1119.5 (M+H)⁺.

Step 4: Synthesis of tert-butyl (S)-4-amino-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate. To a solution of tert-butyl (S)-4-((((9H-fluoren-9-yl)methoxy)carbonyl)amino)-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate (1.68 g, 1.501 mmol) in acetonitrile (3 mL) was added piperidine (0.6 mL, 1.501 mmol) at 0° C. The mixture was stirred at 0° C. for 10 minutes. Trifluoroacetic acid (0.5 mL) was added and the mixture purified by Prep-HPLC (Method AA6) to afford the title compound (1.03 g, 76% yield). LCMS (Method AA13) Rt=1.080 min, m/z 897.5 (M+H)⁺.

Step 5: Synthesis of tert-butyl (S)-4-(2-bromoacetamido)-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate. To a solution of tert-butyl (S)-4-amino-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate (160 mg, 0.178 mmol) in dimethylformamide (3 mL) was added 2-bromoacetic acid (37.2 mg, 0.268 mmol) and ethyl 2-ethoxyquinoline-1(2H)-carboxylate (52.9 mg, 0.214 mmol). The mixture was stirred at 25° C. for 2 hours. The reaction was diluted with ethyl acetate (50 mL), washed with aqueous HBr (1 M, 2×40 mL), saturated aqueous NaHCO₃ (30 mL) and brine (30 mL). The organic layer was dried (Na₂SO₄), filtered and concentrated to afford the title compound (140 mg, 77% yield). LCMS (Method AA13) Rt=1.232 min, m/z 1019.4 (M+H)⁺.

Step 6: Synthesis of (S)-4-(2-bromoacetamido)-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoic acid. To a solution of tert-butyl (S)-4-(2-bromoacetamido)-5-(((S)-1-(((S)-1-((3-(4-((6aR,6bS,7S,8aS,8bS,10R,11aR,12aS,12bS)-7-hydroxy-8b -(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate (140 mg, 0.138 mmol) in dichloromethane (3 mL) was added trifluoroacetic acid (1 mL). The mixture was stirred at 20° C. for 1 hour. The solvent was removed under vacuum and the crude product was purified by Prep-HPLC (Method AA5) to afford the title compound (36 mg, 26% yield). LCMS (Method AA4) Rt=2.975 min, m/z 961.9 (M+H)⁺. ¹H NMR (dimethylsulfoxide-d6, 400 MHz). δ 0.84 (s, 3H) 0.95-1.13 (m, 2H) 1.18-1.28 (m, 6H) 1.38 (s, 3H) 1.56-1.82 (m, 6H) 1.83-1.95 (m, 1H) 2.00 (br d, J=12.13 Hz, 1H) 2.06-2.16 (m, 1H) 2.18-2.34 (m, 4H) 2.52-2.72 (m, 1H) 3.82-3.94 (m, 4H) 4.09-4.39 (m, 5H) 4.48 (d, J=19.40 Hz, 1H) 4.78 (br s, 1H) 4.90 (d, J=5.07 Hz, 1H) 5.38 (s, 1H) 5.92 (s, 1H) 6.15 (dd, J=10.03, 1.65 Hz, 1H) 6.89 (d, J=7.72 Hz, 1H) 7.15-7.24 (m, 3H) 7.30 (d, J=9.92 Hz, 1H) 7.37 (d, J=7.94 Hz, 2H) 7.39-7.49 (m, 2H) 8.00-8.31 (m, 2H) 8.47 (dd, J=7.50, 4.19 Hz, 1H) 9.62-9.90 (m, 1H).

### Precursor Example 23: (S)-4-(2-Bromoacetamido)-5-(((S)-1-(((S)-1-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoic acid

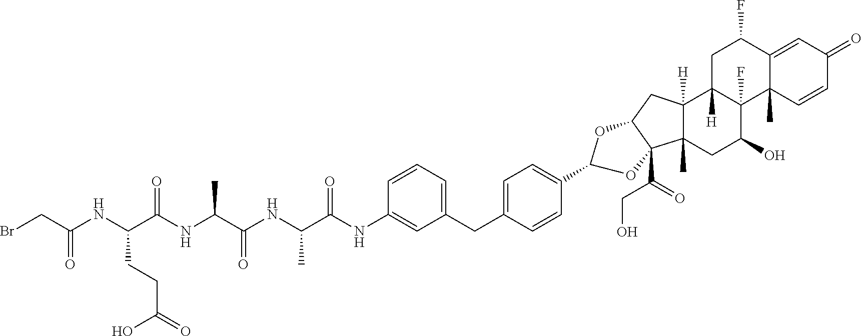

Prepared using similar route to Precusor Example 42 using (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one.

LCMS (Method AA4) Rt=2.668 min, m/z 999.3 (M+H)⁺. ¹H NMR (dimethylsulfoxide-d6) δ=12.12 (br s, 1H), 9.89-9.72 (m, 1H), 8.50-8.45 (m, 1H), 8.40-8.24 (m, 1H), 8.18 (br d, J=7.1 Hz, 1H), 8.07 (d, J=7.2 Hz, 1H), 7.49-7.40 (m, 2H), 7.36 (d, J=7.8 Hz, 2H), 7.29-7.17 (m, 4H), 6.91 (br d, J=7.3 Hz, 1H), 6.30 (d, J=10.3 Hz, 1H), 6.13 (s, 1H), 5.75-5.56 (m, 1H), 5.53 (br d, J=3.1 Hz, 1H), 5.45 (s, 1H), 4.95 (d, J=4.8 Hz, 1H), 4.51 (d, J=19.6 Hz, 1H), 4.40-4.09 (m, 6H), 3.96-3.85 (m, 4H), 2.27-2.20 (m, 3H), 2.09-2.01 (m, 2H), 1.90 (br d, J=7.5 Hz, 2H), 1.78-1.65 (m, 4H), 1.63-1.63 (m, 1H), 1.50 (s, 4H), 1.30-1.19 (m, 6H), 0.86 (s, 3H).

### Precursor Example 43: (S)-4-(2-(2-Bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

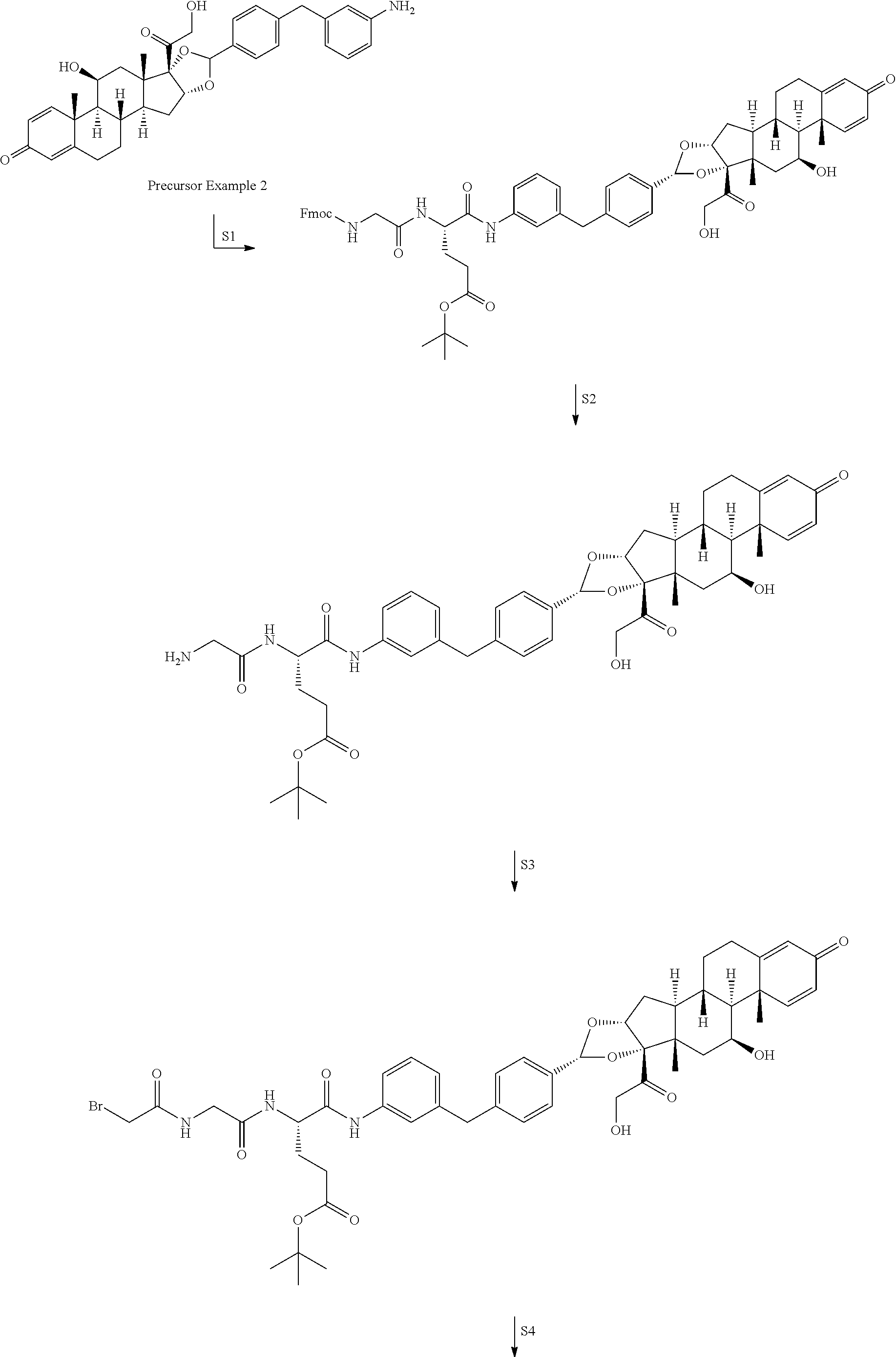

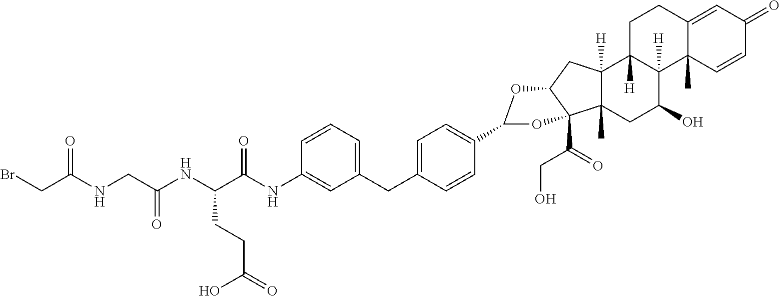

Step 1: Synthesis of tert-butyl (S)-4-(2-((((9H-fluoren-9-yl)methoxy)carbonyl)amino)acetamido)-5-((3-(4-((6aR,6b S,7S,8aS,8bS,10R,11aR,12aS,12bS)-7-hydroxy-8b -(2-hydroxyacetyl)-6a,8a-dimethyl-4-oxo -2,4,6a,6b,7,8,8a,8b,11a,12,12a,12b-dodecahydro-1H-naphtho[2,′,1′:4,5]indeno[1,2-d][1,3]dioxol-10-yl)benzyl)phenyl)amino)-5-oxopentanoate. To a solution of (S)-2-(2-((((9H-fluoren-9-yl)methoxy)carbonyl)amino)acetamido)-5-(tert-butoxy)-5-oxopentanoic acid (508 mg, 1.053 mmol) in dimethylformamide (5 mL) was added 2,4,6-tripropyl-1,3,5,2,4,6-trioxatriphosphinane 2,4,6-trioxide (1117 mg, 1.775 mmol) and triethylamine (0.367 mL, 2.63 mmol) at 0° C. The reaction was stirred at 25° C. for 30 minutes. Precursor Example 2 ((6aR,6bS,7S,8aS,8bS,10R,11aR,12aS,12bS)-10-(4-(3-Aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one) (500 mg, 0.878 mmol) was added at 25° C. and the reaction stirred for 2 hours at 25° C. Six additional vials were set up as described above. All seven reaction mixtures were combined and purified by Prep-HPLC (Method AA7) to afford the title compound (2 g, 31% yield). LCMS (Method AA13) Rt=1.370 min, m/z 1016.5 (M+H-18)⁺. Fmoc=Fluorenylmethyloxycarbonyl.

Step 2: Synthesis of tert-butyl (S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of tert-butyl (S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (350 mg, 0.338 mmol) in acetonitrile (4 mL) was added piperidine (1 mL, 5.05 mmol) at 25° C. The reaction was stirred at 25° C. for 15 minutes then trifluoroacetic acid was added to pH=5. One additional vial was set up as described above. Both reaction mixtures were combined and purified by Prep-HPLC (Method AA8) and the mobile phase lyophilized directly to afford the title compound (200 mg, 13% yield). LCMS (Method AA13) Rt=1.063 min, m/z 812.4 (M+H)⁺.

Step 3: Synthesis of tert-butyl (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of 2-bromoacetic acid (68.5 mg, 0.493 mmol) in dimethylformamide (2 mL) was added 2-ethoxy-1-ethoxycarbonyl-1,2-dihydroquinoline (122 mg, 0.493 mmol) at 25° C. The mixture was stirred at 25° C. for 30 minutes and then tert-butyl (S)-4-(2-aminoacetamido)-5-((3-(4-((6aR,6b S,7S,8aS,8bS,10R,11aR,12aS,12bS)-7-hydroxy-8b -(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (200 mg, 0.246 mmol) was added. The reaction was stirred at 25° C. for 1.5 hours. The reaction was diluted with ethyl acetate (100 mL), washed with aqueous HBr (1 M, 2×150 mL), aqueous NaHCO₃ (200 mL), and brine (200 mL). The organic layer was (Na₂SO₄), filtered and concentrated to afford the title compound (200 mg, 87% yield). LCMS (Method AA13) Rt=1.201 min, m/z 934.3 (M+H)⁺.

Step 4: Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid. To a solution of tert-butyl (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (200 mg, 0.214 mmol) in dichloromethane (2 mL) was added trifluoroacetic acid (0.7 mL, 9.09 mmol) at 25° C. The reaction was stirred at 25° C. for 1 hour. The solvent was removed under reduced pressure and the resulting residue purified by Prep-HPLP (Method AA2). The mobile phase was lyophilized to afford the title compound (44 mg, 23% yield). LCMS (Method AA4) Rt=2.976 min, m/z 876.1 (M+H)⁺. 1H NMR (dimethylsulfoxide-d6, 400 MHz). δ 9.88 (br s, 1H), 8.52 (br s, 1H), 8.24 (br d, J=7.3 Hz, 1H), 7.51-7.14 (m, 9H), 6.92 (br d, J=7.1 Hz, 1H), 6.16 (br d, J=9.9 Hz, 1H), 5.93 (br s, 1H), 5.39 (s, 1H), 4.91 (br d, J=4.2 Hz, 1H), 4.77 (br s, 1H), 4.49 (br d, J=19.6 Hz, 1H), 4.38 (br d, J=5.7 Hz, 1H), 4.29 (br s, 1H), 4.17 (br d, J=19.4 Hz, 1H), 3.91 (br d, J=16.8 Hz, 3H), 3.79 (br s, 2H), 2.37-2.18 (m, 4H), 2.15-1.92 (m, 4H), 1.87-1.65 (m, 6H), 1.39 (br s, 3H), 1.13-0.96 (m, 2H), 0.86 (br s, 3H).

### Precursor Example 24: (S)-4-(2-(2-bromoacetamido)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

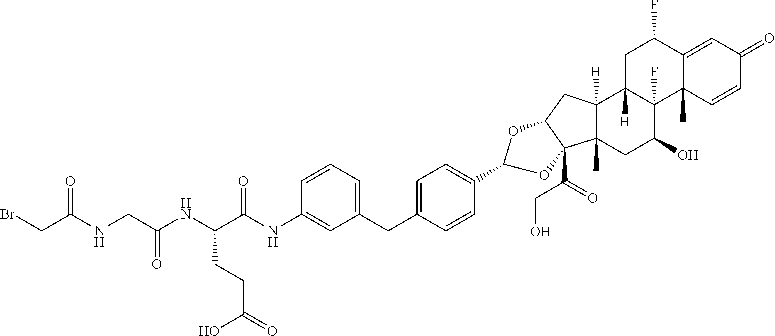

Prepared using similar route to Precursor Example 43 using (2S,6aS,6bR,7S,8aS,8bS,10R,11aR,12aS,12bS)-10-(4-(3-aminobenzyl)phenyl)-2,6b-difluoro-7-hydroxy-8b -(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one.

LCMS (Method AA4) Rt=2.948 min, m/z 914.2 (M+H)⁺. ¹H NMR (dimethylsulfoxide-d6, 400 MHz) δ=9.89 (s, 1H), 8.53 (t, J=5.6 Hz, 1H), 8.25 (d, J=7.9 Hz, 1H), 7.47 (br d, J=8.4 Hz, 1H), 7.41 (s, 1H), 7.36 (d, J=8.2 Hz, 2H), 7.29-7.23 (m, 3H), 7.20 (t, J=7.8 Hz, 1H), 7.02-6.86 (m, 1H), 6.30 (dd, J=1.9, 10.3 Hz, 1H), 6.13 (s, 1H), 5.76-5.60 (m, 1H), 5.63-5.56 (m, 1H), 5.54-5.47 (m, 1H), 5.45 (s, 1H), 4.94 (d, J=5.1 Hz, 1H), 4.51 (d, J=19.6 Hz, 1H), 4.42-4.35 (m, 1H), 4.25-4.11 (m, 2H), 3.93 (s, 2H), 3.89 (s, 2H), 3.85-3.74 (m, 3H), 2.63-2.52 (m, 2H), 2.42 (br d, J=1.8 Hz, 1H), 2.30-2.16 (m, 4H), 2.07-1.90 (m, 2H), 1.87-1.61 (m, 4H), 1.55 (br s, 1H), 1.49 (s, 3H), 0.86 (s, 3H).

### Precursor Example 44: 2-((6aR,6bS,7S,8aS,8bS,10R,11aR,12aS,12bS)-10-(4-(3-((S)-2-((S)-2-(3-(2-Bromoacetamido)propanamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

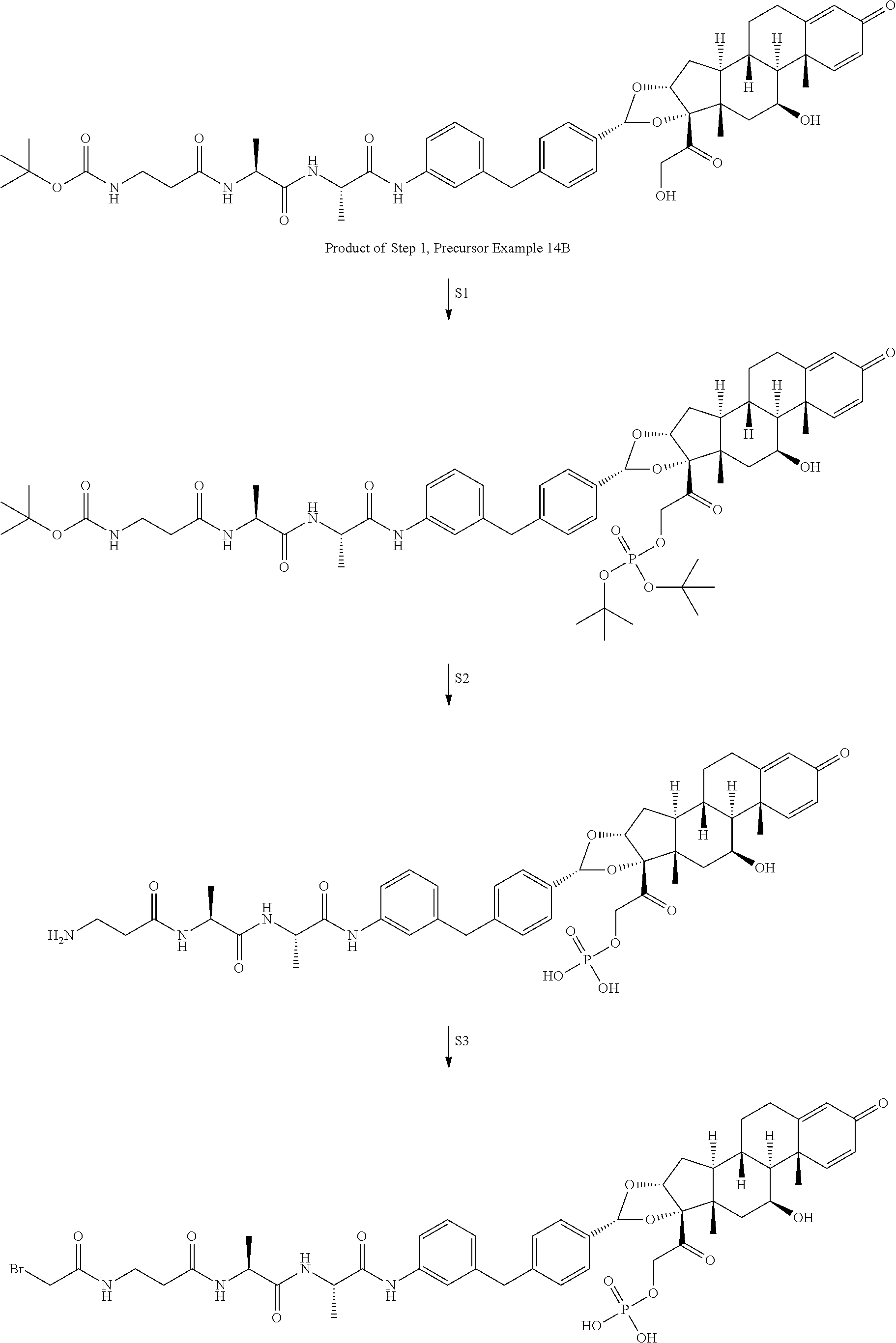

Step 1: Synthesis of tert-butyl (3-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-3-oxopropyl)carbamate. To a solution of the product of Precursor Example 14B, Step 1 (tert-butyl (3-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-3-oxopropyl)carbamate) (500 mg, 0.566 mmol) in dichloromethane (5 mL) was added 1H-tetrazole (397 mg, 5.66 mmol) and di-tert-butyl diethylphosphoramidite (1.694 g, 6.79 mmol) at 25° C. The reaction was stirred at 25° C. for 1 hour and then hydrogen peroxide (353 mg, 3.11 mmol) was added. The reaction was stirred for 2 hours. One additional vial was set up as described above. Both reaction mixtures were combined and purified by Prep-HPLC (Method AA7) to afford the title compound (1 g, 82% yield). LCMS (Method AA13) Rt=1.300 min, m/z 1075.8 (M+H)⁺.

Step 2: Synthesis of 2-((6aR,6bS,7S,8aS,8bS,10R,11aR,12aS,12bS)-10-(4-(3-((S)-2-((S)-2-(3-aminopropanamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate. To a solution of tert-butyl (3-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-3-oxopropyl)carbamate (600 mg, 0.558 mmol) in dichloromethane (6 mL) was added trifluoroacetic acid (2 mL, 26.0 mmol) at 25° C. and the reaction stirred at 25° C. for 1 hour. The solvent was removed under reduced pressure and the resulting residue was purified by Prep-HPLC (Method AA6). The mobile phase was lyophilized to afford the title compound (350 mg, 73% yield). LCMS (Method AA13) Rt=0.986 min, m/z 863.3 (M+H)⁺.

Step 3: Synthesis of 2-((6aR,6bS,7S,8aS,8bS,10R,11aR,12aS,12bS)-10-(4-(3-((S)-2-((S)-2-(3-(2-bromoacetamido)propanamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate. To a solution of 2-bromoacetic acid (16.1 mg, 0.116 mmol) in dimethylformamide (1 mL) was added 2-((6aR,6bS,7S,8aS,8bS,10R,11aR,12aS,12bS)-10-(4-(3-((S)-2-((S)-2-(3-aminopropanamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate (100 mg, 0.116 mmol), 2-bromo-1-ethylpyridin-1-ium tetrafluoroborate (34.9 mg, 0.127 mmol) and N-ethyl-N-isopropylpropan-2-amine (30.0 mg, 0.232 mmol) at 25° C. and the mixture stirred at 25° C. for 2 hours. The resulting residue was purified by Prep-HPLC (Method AA2). The mobile phase was lyophilized afford the title compound (65 mg, 30% yield). LCMS (Method AA4) Rt=2.932 min, m/z 985.2 (M+H)⁺. ¹H NMR (dimethylsulfoxide-d6, 400 MHz). δ 9.79-9.63 (m, 1H), 8.31-8.24 (m, 1H), 8.21-8.04 (m, 2H), 7.51-7.41 (m, 2H), 7.36 (d, J=7.9 Hz, 2H), 7.29 (d, J=10.1 Hz, 1H), 7.24-7.12 (m, 3H), 6.89 (br d, J=7.7 Hz, 1H), 6.14 (dd, J=1.5, 10.1 Hz, 1H), 5.91 (s, 1H), 5.46 (s, 1H), 4.95-4.78 (m, 3H), 4.54 (br dd, J=8.0, 18.2 Hz, 1H), 4.38-4.12 (m, 4H), 3.87 (s, 2H), 3.80 (d, J=3.3 Hz, 2H), 3.25 (br d, J=6.8 Hz, 2H), 2.60-2.50 (m, 2H), 2.13-1.96 (m, 2H), 1.84-1.58 (m, 6H), 1.37 (s, 3H), 1.25 (dd, J=2.1, 7.2 Hz, 3H), 1.19 (s, 3H), 1.06-0.98 (m, 2H), 0.85 (s, 3H).

### Example 25B: 2-((2S,6aS,6bR,7S,8aS,8bS,10R,11aR,12aS,12bS)-10-(4-(3-((S)-2-((S)-2-(3-(2-bromoacetamido)propanamido)propanamido)propanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

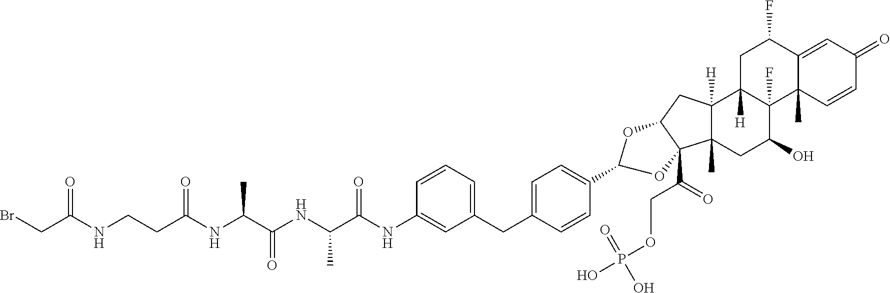

Prepared using similar route to Precursor Example 44 using 2-((2S,6aS,6bR,7S,8aS,8bS,10R,11aR,12aS,12bS)-10-(4-(3-aminobenzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate.

LCMS (Method AA4) Rt=2.958 min, m/z 1021.3 (M+H)⁺. ¹H (dimethylsulfoxide-d6, 400 MHz) δ=9.83-9.70 (m, 1H), 8.34-8.16 (m, 2H), 8.15-8.06 (m, 1H), 7.56-7.40 (m, 2H), 7.36 (d, J=7.9 Hz, 2H), 7.30-7.16 (m, 4H), 6.91 (br d, J=7.3 Hz, 1H), 6.30 (dd, J=1.5, 10.1 Hz, 1H), 6.12 (s, 1H), 5.75-5.56 (m, 2H), 5.53 (s, 1H), 4.99-4.87 (m, 2H), 4.59 (dd, J=8.4, 18.1 Hz, 1H), 4.43-4.15 (m, 4H), 3.89 (s, 2H), 3.82 (d, J=3.5 Hz, 2H), 3.33-3.21 (m, 2H), 2.73-2.60 (m, 1H), 2.39-2.14 (m, 5H), 2.11-2.00 (m, 1H), 1.78-1.63 (m, 3H), 1.57-1.45 (m, 4H), 1.27 (dd, J=2.3, 6.9 Hz, 3H), 1.19 (d, J=7.1 Hz, 3H), 0.88 (s, 3H).

### Precursor Example 45: 2-((6aR,6bS,7S,8aS,8bS,10R,11aR,12aS,12bS)-10-(4-(3-((S)-2-((S)-2-(2-Bromoacetamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

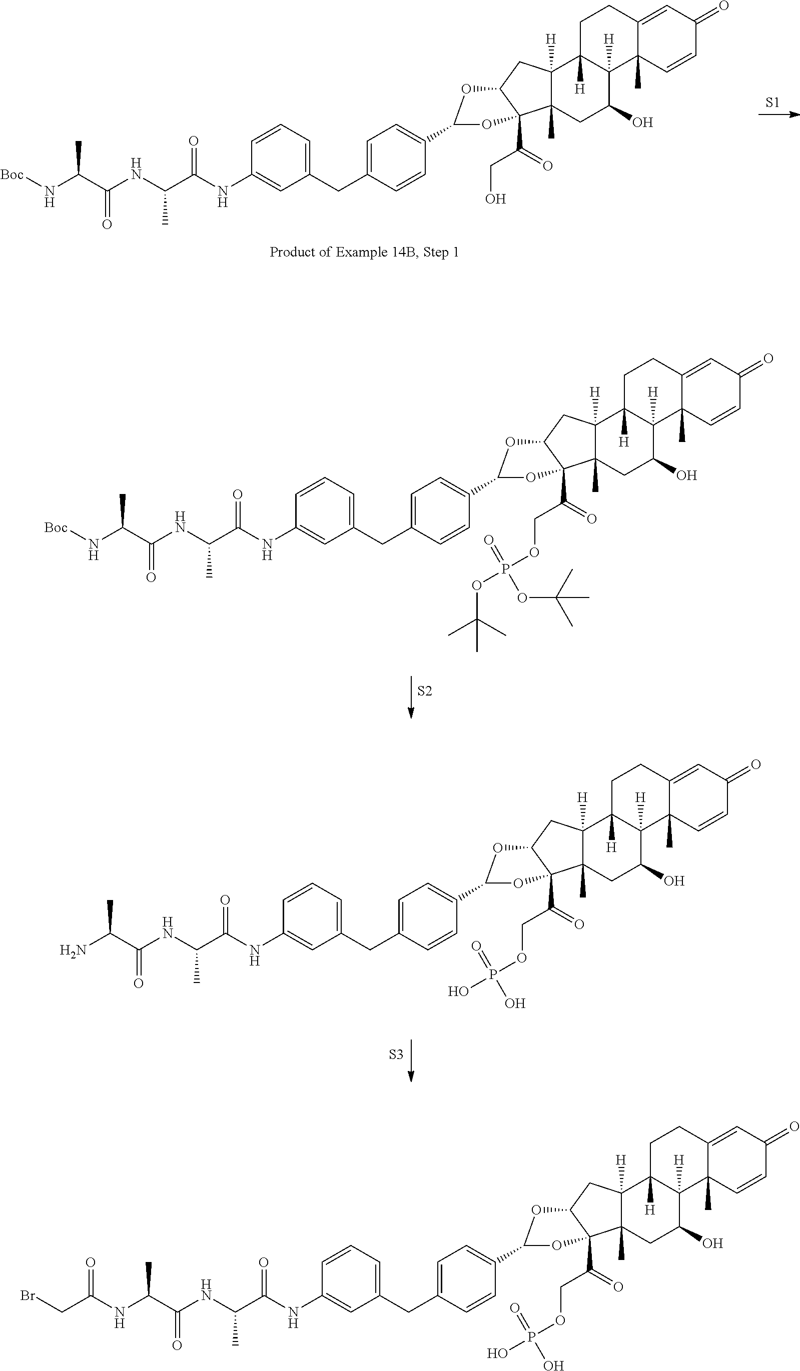

Step 1: Synthesis of tert-butyl ((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)carbamate. To a solution of the product of Example 14B, Step 1 (tert-butyl ((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)carbamate) (400 mg, 0.493 mmol) in dimethylformamide (4 mL) was added 1H tetrazole (345 mg, 4.93 mmol) and di-tert-butyl diethylphosphoramidite (1474 mg, 5.91 mmol) at 25° C. The mixture was stirred at 25° C. for 2 hours and then hydrogen peroxide (307 mg, 2.71 mmol) was added to the mixture at 0° C. The reaction was stirred at 25° C. for 2 hours. Four additional vials were set up as described above. All five reaction mixtures were combined, poured onto ice water (1 L) and filtered to afford the title compound (1.6 g, 65% yield). LCMS (Method AA13) Rt=1.344 min, m/z 1004.5 (M+H)⁺. Boc=tert-butoxycarbonyl.

Step 2: Synthesis of 2-((6aR,6bS,7S,8aS,8bS,10R,11aR,12aS,12bS)-10-(4-(3-((S)-2-((S)-2-aminopropanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate. To a solution of tert-butyl ((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)carbamate (1.5 g, 1.494 mmol) in dichloromethane (10 mL) was added trifluoroacetic acid (3 mL, 38.9 mmol) at 25° C. The reaction was stirred at 25° C. for 2 hours. The mixture was purified by Prep-HPLC (Method AA10) to afford the title compound (400 mg, 34% yield). LCMS (Method AA13) Rt 1.602 min, m/z 792.4 (M+H)⁺.

Step 3: Synthesis of 2-((6aR,6bS,7S,8aS,8bS,10R,11aR,12aS,12bS)-10-(4-(3-((S)-2-((S)-2-(2-bromoacetamido)propanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate. To a solution of 2-bromoacetic acid (63.2 mg, 0.455 mmol) in dimethylformamide (1.5 mL) was added 2-bromo-1-ethylpyridin-1-ium tetrafluoroborate (125 mg, 0.455 mmol), N,N-diisopropylethylamine (0.159 mL, 0.909 mmol) and 2-((6aR,6bS,7S,8aS,8bS,10R,11aR,12aS,12bS)-10-(4-(3-((S)-2-((S)-2-aminopropanamido)propanamido)benzyl)phenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b -yl)-2-oxoethyl dihydrogen phosphate (240 mg, 0.303 mmol) at 25° C. and the mixture stirred at 25° C. for 2 hours. The resulting mixture was purified by Pre-HPLC (Method AA9) to afford the title compound (100 mg, yield 36.0% yield). LCMS (Method AA4) Rt=2.890 min, m/z 914.2 (M+H)⁺. ¹H NMR (dimethylsulfoxide-d6, 400 MHz) δ 0.84-0.90 (m, 3H), 0.98-1.11 (m, 2H), 1.18-1.23 (m, 3H), 1.28 (d, J=7.09 Hz, 3H), 1.39 (s, 3H), 1.60-1.86 (m, 5H), 1.96-2.17 (m, 2H), 2.32 (br d, J=1.71 Hz, 1H), 2.52-2.59 (m, 2H), 3.86-3.94 (m, 4H), 4.26-4.40 (m, 3H), 4.56 (dd, J=18.22, 8.07 Hz, 1H), 4.82-4.96 (m, 3H), 5.48 (s, 1H), 5.93 (s, 1H), 6.16 (dd, J=10.15, 1.71 Hz, 1H), 6.91 (br d, J=7.58 Hz, 1H), 7.17-7.26 (m, 3H), 7.31 (d, J=10.03 Hz, 1H), 7.35-7.52 (m, 4H), 8.19-8.42 (m, 1H), 8.45-8.57 (m, 1H), 9.71-9.88 (m, 1H).

### Precursor Example 26: Synthesis of 2-((2S,6aS,6bR,7S,8aS,8bS,10R,11aR,12aS,12bS)-10-(4-(3-((S)-2-((S)-2-(2-bromoacetamido)propanamido)propanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

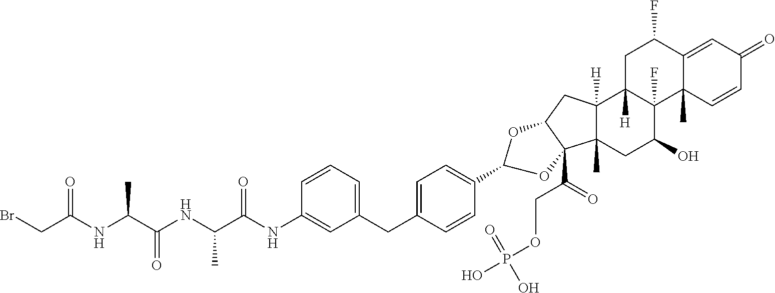

Prepared using similar route to Precursor Example 45 using 2-((2S,6aS,6bR,7S,8aS,8bS,10R,11aR,12aS,12bS)-10-(4-(3-aminobenzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate.

LCMS (Method AA4) Rt=2.924 min, m/z 950.3 (M+H)⁺. ¹H NMR (methanol-d4, 400 MHz) δ=7.44-7.38 (m, 1H), 7.36-7.31 (m, 4H), 7.23-7.17 (m, 3H), 6.93 (br d, J=7.6 Hz, 1H), 6.35-6.33 (m, 2H), 5.61-5.47 (m, 2H), 5.03 (s, 1H), 5.01-4.97 (m, 1H), 4.80-4.76 (m, 1H), 4.43-4.30 (m, 3H), 3.94 (s, 2H), 3.88-3.81 (m, 2H), 2.76-2.66 (m, 1H), 2.42-2.38 (m, 3H), 1.81-1.79 (m, 3H), 1.78-1.75 (m, 1H), 1.58 (s, 3H), 1.42-1.37 (m, 6H), 1.01 (s, 3H).

### Precursor Example 46: (S)-4-(2-Bromoacetamido)-5-(((S)-1-(((S)-1-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoic acid

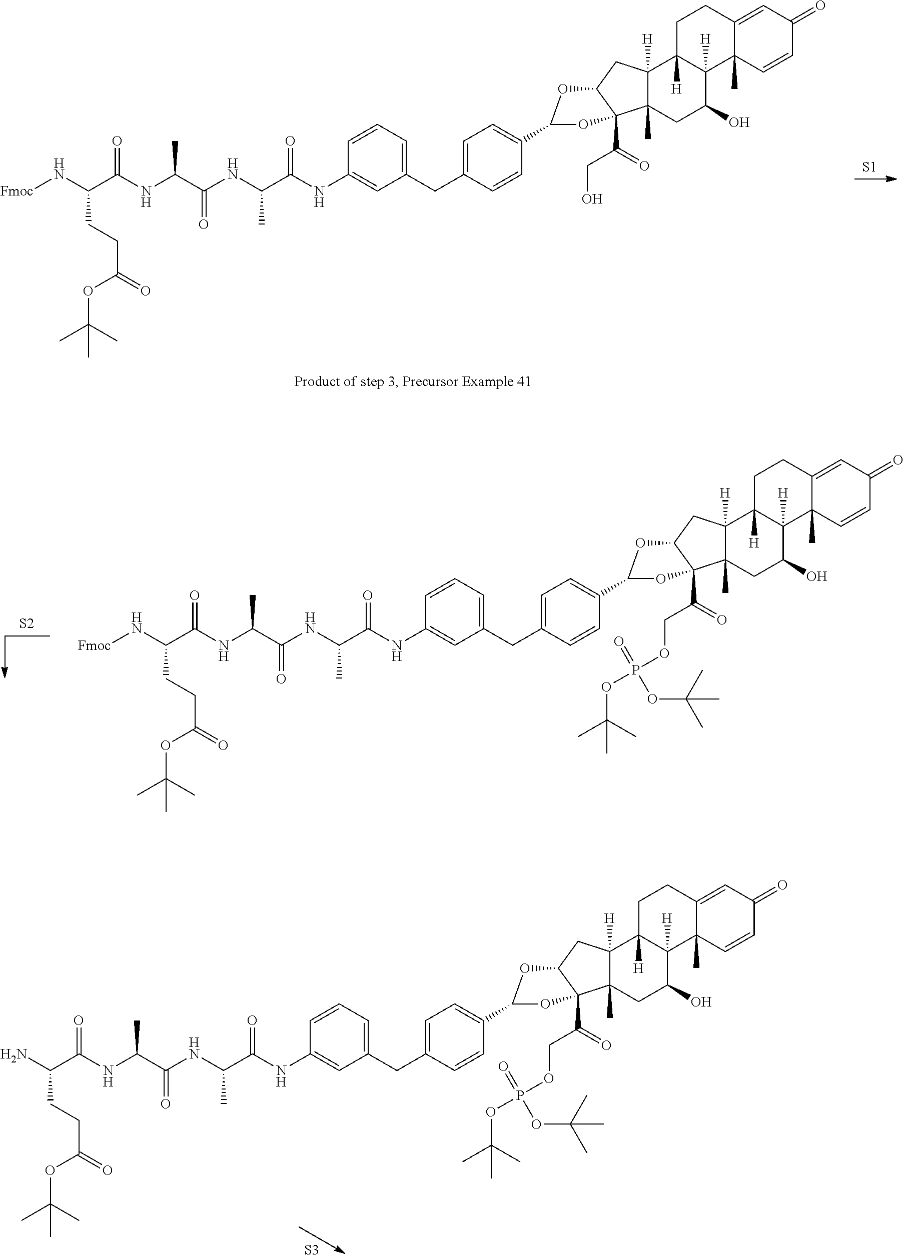

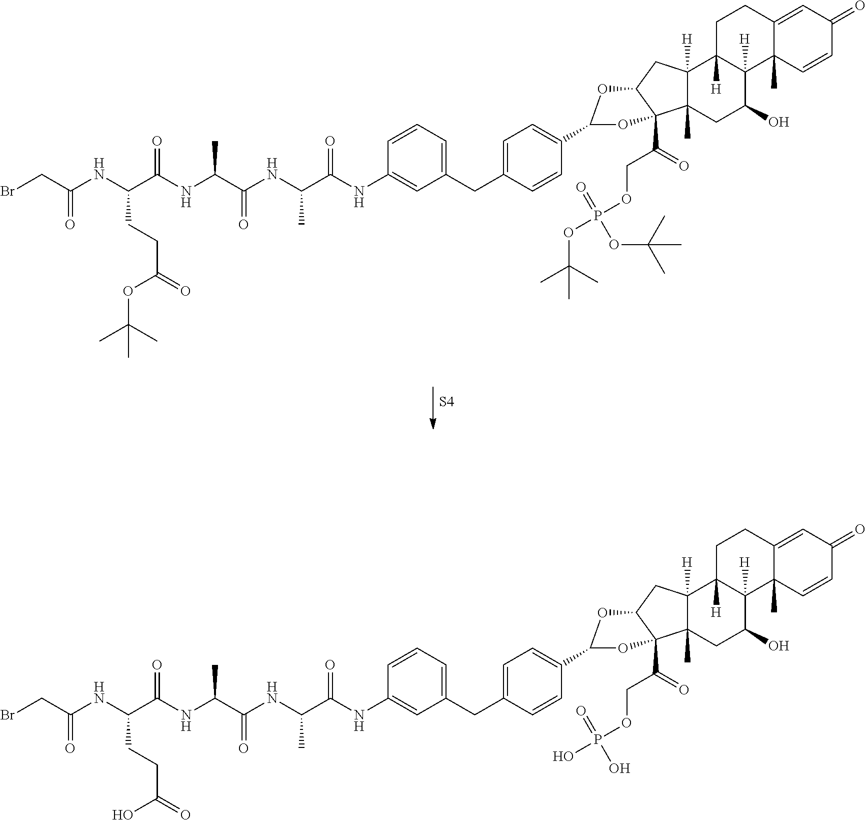

Step 1: Synthesis of tert-butyl (S)-4-((((9H-fluoren-9-yl)methoxy)carbonyl)amino)-5-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate. To a solution of the product of Precursor Example 42, Step 3 (tert-butyl (S)-4-((((9H-fluoren-9-yl)methoxy)carbonyl)amino)-5-(((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate) (500 mg, 0.447 mmol) in dimethylformamide (2 mL) was added 1H-tetrazole (313 mg, 4.47 mmol) and di-tert-butyl diethylphosphoramidite (1.337 g, 5.36 mmol) at 20° C. The reaction was stirred at 20° C. for 1 hour then hydrogen peroxide (279 mg, 2.457 mmol) was added and the reaction stirred for an additional 1 hour. The reaction was purified by Prep-HPLC (Method AA6) to afford the title compound (450 mg, 77% yield). LCMS (Method AA13) Rt=1.524 min, m/z 1311.6 (M+H)⁺.

Step 2. Synthesis of tert-butyl (S)-4-amino-5-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate. To a solution of tert-butyl (S)-4-((((9H-fluoren-9-yl)methoxy)carbonyl)amino)-5-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate (450 mg, 0.343 mmol) in acetonitrile (2 mL) was added piperidine (0.4 mL) at 0° C. The reaction was stirred at 0° C. for 20 minutes and then concentrated to afford a crude product, which was stirred in petroleum ether (20 mL) for 1 hour. The solid was collected by filtration, and dried under reduced pressure to afford the title compound (250 mg, 67% yield). LCMS (Method AA13) Rt=1.244 min, m/z 1089.5 (M+H)⁺.

Step 3: Synthesis of tert-butyl (S)-4-(2-bromoacetamido)-5-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate. To a solution of tert-butyl (S)-4-amino-5-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate (250 mg, 0.23 mmol) in dimethylformamide (3 mL) was added 2-bromoacetic acid (47.8 mg, 0.344 mmol) and ethyl 2-ethoxyquinoline-1(2H)-carboxylate (68.1 mg, 0.275 mmol). The mixture was stirred at 25° C. for 2 hours then purified by Prep-HPLC (Method AA11) to afford the title compound (120 mg, 43% yield). LCMS (Method AA13) Rt=1.351 min, m/z 1211.4 (M+H)⁺.

Step 4: Synthesis of (S)-4-(2-bromoacetamido)-5-(((S)-1-(((S)-1-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoic acid. To a solution of tert-butyl (S)-4-(2-bromoacetamido)-5-(((S)-1-(((S)-1-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoate (120 mg, 0.099 mmol) in dichloromethane (3 mL) was added trifluoroacetic acid (1 mL) and the mixture stirred at 20° C. for 2 hours. The solvent was removed under reduced pressure and the crude product was purified by Prep-HPLC (Method AA12) to afford the title compound (32 mg, 30% yield). LCMS (Method AA4) Rt=2.909 min, m/z=1041.9 (M+H)⁺. ¹H NMR (dimethylsulfoxide-d6, 400 MHz) δ 9.88-9.67 (m, 2H), 8.47 (dd, J=4.3, 7.6 Hz, 2H), 8.26 (d, J=7.1 Hz, 1H), 8.21-8.12 (m, 1H), 8.28-8.03 (m, 1H), 8.06 (d, J=7.1 Hz, 1H), 7.49-7.39 (m, 5H), 7.36 (d, J=8.2 Hz, 5H), 7.30 (d, J=10.1 Hz, 2H), 7.22 (br d, J=8.2 Hz, 1H), 7.24-7.15 (m, 1H), 7.19-7.14 (m, 1H), 6.88 (d, J=7.7 Hz, 2H), 6.15 (d, J=11.7 Hz, 2H), 5.91 (s, 2H), 5.46 (s, 2H), 4.95-4.80 (m, 7H), 4.55 (dd, J=8.0, 18.0 Hz, 2H), 4.38-4.31 (m, 1H), 4.31-4.19 (m, 3H), 3.93-3.85 (m, 4H), 2.66 (s, 2H), 2.31 (br s, 1H), 2.24 (br t, J=8.2 Hz, 2H), 2.33-2.18 (m, 1H), 2.17-1.94 (m, 5H), 1.87 (br s, 2H), 1.83-1.59 (m, 14H), 1.37 (s, 7H), 1.28-1.17 (m, 15H), 1.00 (br d, J=11.2 Hz, 5H), 0.86 (s, 7H).

### Precursor Example 27: (S)-4-(2-Bromoacetamido)-5-(((S)-1-(((S)-1-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxopropan-2-yl)amino)-1-oxopropan-2-yl)amino)-5-oxopentanoic acid

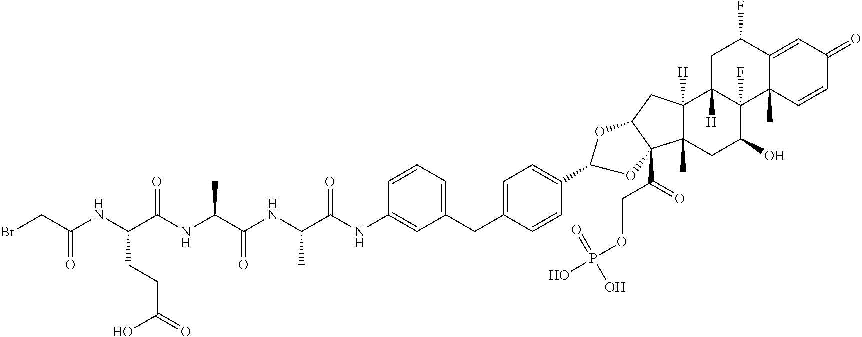

Prepared using similar route to Precursor Example 46 using (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one.

LCMS (Method AA4) Rt=2.905 min, m/z 1079.1 (M+H)⁺. ¹H NMR (methanol-d4, 400 MHz) δ=7.48-7.44 (m, 1H), 7.40-7.30 (m, 4H), 7.25-7.16 (m, 3H), 6.93 (br d, J=7.6 Hz, 1H), 6.37-6.30 (m, 2H), 5.64-5.52 (m, 2H), 5.04 (br d, J=4.4 Hz, 1H), 5.01-4.95 (m, 1H), 4.81-4.72 (m, 1H), 4.43-4.25 (m, 4H), 4.13-3.99 (m, 1H), 3.97-3.91 (m, 2H), 3.91-3.77 (m, 2H), 2.79-2.61 (m, 1H), 2.45-2.33 (m, 4H), 2.27 (br d, J=13.7 Hz, 1H), 2.16-2.03 (m, 1H), 2.00-1.88 (m, 1H), 1.83-1.74 (m, 3H), 1.68-1.54 (m, 4H), 1.46-1.36 (m, 6H), 1.01 (s, 3H).

### Precursor Example 39: 2-((2S,6aS,6bR,7S,8aS,8bS,10R,11aR,12aS,12bS)-10-(4-(3-((S)-6-Amino-2-(2-(2-bromoacetamido)acetamido)hexanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

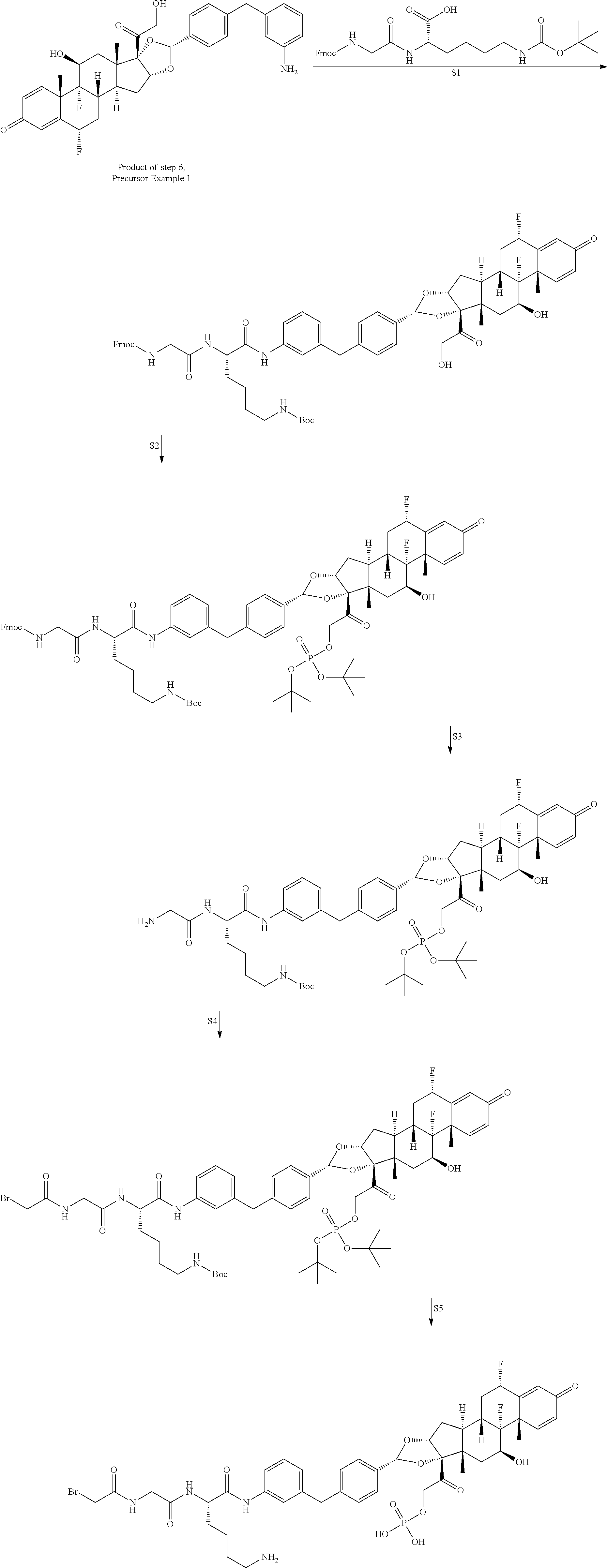

Step 1: Synthesis of tert-butyl ((S)-5-(2-((((9H-fluoren-9-yl)methoxy)carbonyl)amino)acetamido)-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of N²-((((9H-fluoren-9-yl)methoxy)carbonyl)glycyl)-N⁶-(tert-butoxycarbonyl)-L-lysine (5.58 g, 8.26 mmol) in dimethylformamide (60 mL) was added 2,4,6-tripropyl-1,3,5,2,4,6-trioxatriphosphinane 2,4,6-trioxide (10.51 g, 16.51 mmol) and triethylamine (3.45 mL, 24.77 mmol) at 0° C. The reaction was stirred at 25° C. for 1 hour then the product of Precursor Example 1, Step 6 ((2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one) (5 g, 8.26 mmol) was added to the reaction at 25° C. The reaction was stirred for 5 hours at 25° C. Six additional vials were set up as described above. All seven reactions were combined and purified by Prep-HPLC (Method AA14) to afford the title compound (24 g, 25% yield). LCMS (Method AA13) Rt=1.295 min, m/z 1095.6 (M+H-18)⁺.

Step 2: Synthesis of tert-butyl ((S)-5-(2-((((9H-fluoren-9-yl)methoxy)carbonyl)amino)acetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of tert-butyl ((S)-5-(2-(4(9H-fluoren-9-yl)methoxy)carbonyl)amino)acetamido)-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (3 g, 2.69 mmol) in Dimethyl formamide (30 mL) was added 1H-tetrazole (1.888 g, 26.9 mmol) and di-tert-butyl diethylphosphoramidite (8.06 g, 32.3 mmol) at 25° C. The reaction was stirred at 25° C. for 3.5 hours then hydrogen peroxide (224 mg, 1.976 mmol) was added and the mixture stirred for 30 minutes. Six additional vials were set up as described above. All seven reactions were combined and purified by Prep-HPLC (Method AA7) to afford the title compound (10 g, 37% yield). LCMS (Method AA13) Rt=1.421 min, m/z 1305.7 (M+H)⁺.

Step 3: Synthesis of tert-butyl ((S)-5-(2-aminoacetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of tert-butyl ((S)-5-(2-((((9H-fluoren-9-yl)methoxy)carbonyl)amino)acetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (2.5 g, 1.969 mmol) in Acetonitrile (10 mL) was added piperidine (2 mL, 1.969 mmol) at 25° C. The reaction was stirred at 25° C. for 1 hour. Three additional vials were set up as described above. All four reactions were combined and concentrated to afford a residue that was stirred in petroleum ether (30 mL) for 2 hours. The solid was collected by filtration, and dried under reduced pressure to give the title compound (7 g, 70% yield). LCMS for reaction (ESI+): m/z 1083.5 (M+H)⁺, Rt: 1.175 min.

Step 4: Synthesis of tert-butyl ((S)-5-(2-(2-bromoacetamido)acetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. To a solution of 2-bromoacetic acid (0.929 g, 6.68 mmol) in dimethyl formamide (35 mL) was added 2-ethoxy-1-ethoxycarbonyl-1,2-dihydroquinoline (1.653 g, 6.68 mmol) at 25° C. The mixture was stirred at 25° C. for 1 hour. Then tert-butyl ((S)-5-(2-aminoacetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (3.5 g, 3.34 mmol) was added to the reaction. The reaction was stirred at 25° C. for 2 hours. LCMS showed the reaction was completed. The reaction was diluted with dichloromethane (100 mL), washed with aqueous HBr (1 M, 2×80 mL), aqueous NaHCO₃ (60 mL), and brine (60 mL). The organic layer was dried (Na₂SO₄) and concentrated to afford the title compound (2 g, 51% yield) which was used to next step directly. LCMS (Method AA13) Rt=1.318 min, m/z 1205.5 (M+H)⁺.

Step 5: Synthesis of 2-((2S,6aS,6bR,7S,8aS,8bS,10R,11aR,12aS,12bS)-10-(4-(3-((S)-6-amino-2-(2-(2-bromoacetamido)acetamido)hexanamido)benzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate. To a solution of tert-butyl ((S)-5-(2-(2-bromoacetamido)acetamido)-6-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (2 g, 1.661 mmol) in dichloromethane (10 mL) was added trifluoroacetic acid (5 mL, 64.9 mmol) at 25° C. The reaction was stirred at 25° C. for 40 minutes then evaporated to dryness to afford a residue that was purified by Prep-HPLC (Method AA17) to afford the title compound (550 mg, 32% yield). LCMS (Method AA13) Rt=2.313 min, m/z 993.1 (M+H)⁺. ¹H NMR (dimethylsulfoxide-d6, 400 MHz) δ ppm 0.90 (s, 3H) 1.19-1.41 (m, 2H) 1.43-1.62 (m, 7H) 1.64-1.77 (m, 3H) 1.84 (br d, J=14.55 Hz, 1H) 1.95-2.07 (m, 1H) 2.18-2.36 (m, 3H) 2.65-2.78 (m, 3H) 3.71-3.86 (m, 3H) 3.89 (s, 2H) 3.93 (s, 2H) 4.20 (br d, J=9.48 Hz, 1H) 4.33-4.41 (m, 1H) 4.59 (br dd, J=18.41, 8.05 Hz, 1H) 4.81 (br dd, J=18.52, 8.60 Hz, 1H) 4.94 (d, J=4.63 Hz, 1H) 5.50 (s, 1H) 5.54-5.76 (m, 1H) 6.13 (s, 1H) 6.29 (dd, J=10.14, 1.32 Hz, 1H) 6.95 (d, J=7.72 Hz, 1H) 7.15-7.28 (m, 4H) 7.30-7.41 (m, 3H) 7.51 (br d, J=7.94 Hz, 1H) 7.72 (br s, 3H) 8.21 (br d, J=7.72 Hz, 1H) 8.54 (t, J=5.62 Hz, 1H) 9.93 (br d, J=2.65 Hz, 1H).

### Precursor Example 28: (S)-4-(2-(2-Bromoacetamido)acetamido)-5-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

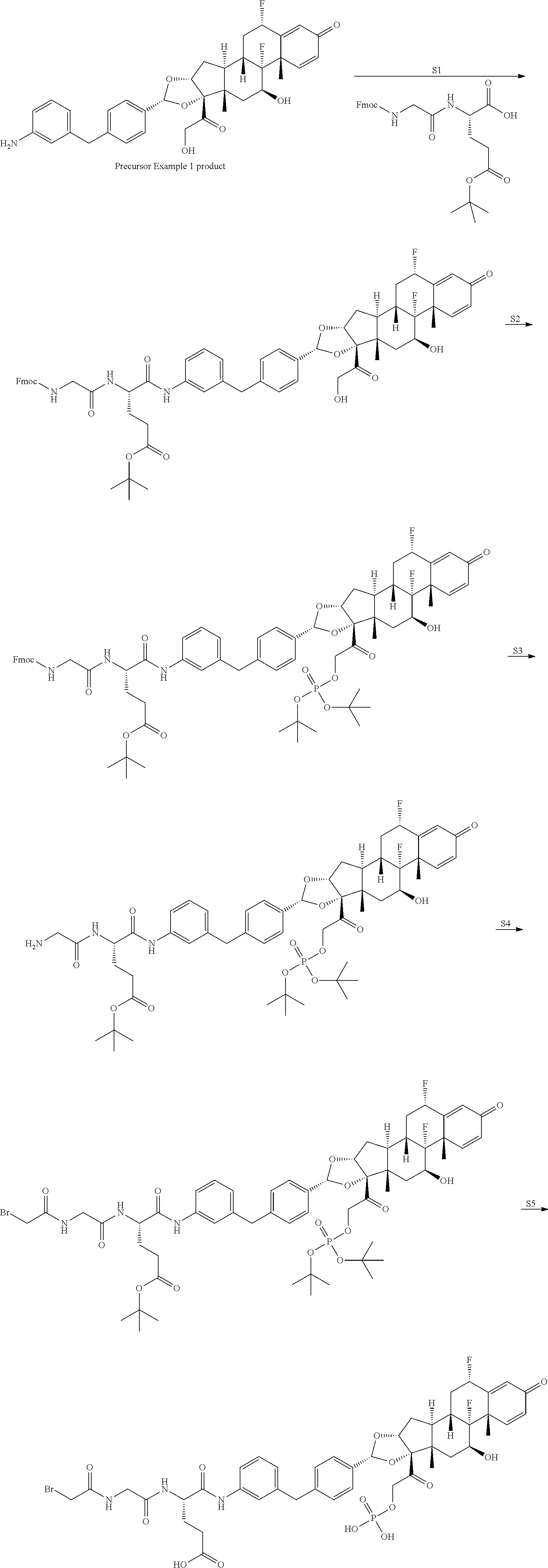

Step 1: Synthesis of tert-butyl (S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of the product of Precursor Example 1 ((2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-6a,6b,7,8,8a,8b,11a,12,12a,12b-decahydro-1H-naphtho[2′,1′:4,5]indeno[1,2-d][1,3]dioxol-4(2H)-one) (500 mg, 0.826 mmol) in dimethylformamide (10 mL) was added (S)-2-(2-((((9H-fluoren-9-yl)methoxy)carbonyl)amino)acetamido)-5-(tert-butoxy)-5-oxopentanoic acid (500 mg, 1.036 mmol), 2,4,6-tripropyl-1,3,5,2,4,6-trioxatriphosphinane 2,4,6-trioxide (1800 mg, 2.83 mmol) and triethylamine (0.689 ml, 4.94 mmol) at 0° C. The reaction mixture was stirred for 30 minutes at 20° C., and then additional product of Precursor Example 1 (500 mg, 0.826 mmol) was added. The reaction was stirred for 12 hr at 25° C. 16 identical reactions were performed and the reactions combined. The mixture was added to water (3 L) and extracted with ethyl acetate (3×500 mL). The layers were separated and the organic layer dried over (Na₂SO₄), filtered and concentrated under reduced pressure to afford the title compound (12 g, 11.21 mmol, 48.0% yield) as yellow solid. TLC (ethyl acetate) Rf 0.48.

Step 2: Synthesis of tert-butyl (S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of (5)-tert-butyl 4-(2-((((9H-fluoren-9-yl)methoxy)carbonyl)amino)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (1 g, 0.934 mmol) in dimethylformamide (5 mL) was added 1H tetrazole (0.655 g, 9.34 mmol) and di-tert-butyl diethylphosphoramidite (1.864 g, 7.48 mmol) at 25° C. The reaction was stirred at 25° C. for 2.5 hours then hydrogen peroxide (0.583 g, 5.14 mmol) was added at 0° C. The mixture was stirred at 25° C. for 1 hour. 19 identical reactions were performed and combined. The mixture was added to water and the solid collected by filtration and purified by prep-HPLC to afford the title compound (10 g, 85% yield). LCMS (Method AA18) Rt=1.434 min, m/z 1262.5 (M+H)⁺.

Step 3: Synthesis of tert-butyl (S)-4-(2-aminoacetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To (S)-tert-butyl 4-(2-((((9H-fluoren-9-yl)methoxy)carbonyl)amino)acetamido)-5-((3-(4-42S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (10 g, 7.92 mmol) in acetonitrile (20 mL) was added piperidine (4 mL, 7.92 mmol) at 25° C. The mixture stirred for 20 minutes, concentrated and washed with petroleum ether (2×300 mL) and dried under reduced pressure to afford the title compound (5 g, 61% yield). LCMS (Method AA19) Rt=1.604 min, m/z 1040.7 (M+H)⁺.

Step 4: Synthesis of tert-butyl (S)-4-(2-(2-bromoacetamido)acetamido)-5-((3-(4-42S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate. To a solution of 2-bromoacetic acid (0.134 g, 0.961 mmol) in dimethylformamide (4 mL) was added N-ethoxycarbonyl-2-ethoxy-1,2-dihydroquinoline (0.238 g, 0.961 mmol) at 25° C. The mixture was stirred at 25° C. for 1 hour then (5)-tert-butyl 4-(2-aminoacetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (0.5 g, 0.481 mmol) was added and the mixture stirred at 25° C. for 2.5 hours. 9 identical reactions were performed and combined. The mixture was concentrated and washed with 1M HBr in water (200 mL), aqueous of NaHCO₃ (200 mL), brine (200 mL), dried (Na₂SO₄), filtered and concentrated to afford the title compound (5 g, 90% yield). LCMS (Method AA18) Rt=1.32 min, m/z 1162 (M+H)⁺.

Step 5: Synthesis of (S)-4-(2-(2-bromoacetamido)acetamido)-5-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid. To (S)-tert-butyl 4-(2-(2-bromoacetamido)acetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (3 g, 2.58 mmol) in dichloromethane (20 mL) was added trifluoroacetic acid (10 mL) at 25° C. and the mixture stirred for 2 hours. The reaction mixture was concentrated and purified by prep-HPLC (Method AA20) to afford the title compound (1.09 g, 42% yield). LCMS (Method AA4) Rt=2.919 min, m/z 994.2 (M+H)⁺. ¹H NMR (methanol-d4, 400 MHz) δ=7.45 (br d, J=7.9 Hz, 1H), 7.40-7.29 (m, 4H), 7.25-7.16 (m, 3H), 6.95 (br d, J=7.5 Hz, 1H), 6.38-6.30 (m, 2H), 5.66-5.57 (m, 1H), 5.55-5.44 (m, 1H), 5.08-4.94 (m, 2H), 4.81-4.72 (m, 1H), 4.49 (br dd, J=5.0, 9.0 Hz, 1H), 4.32 (br d, J=8.6 Hz, 1H), 3.96-3.90 (m, 5H), 2.79-2.60 (m, 1H), 2.48-2.32 (m, 4H), 2.28 (br d, J=13.3 Hz, 1H), 2.17 (dt, J=7.6, 13.4 Hz, 1H), 2.04-1.92 (m, 1H), 1.85-1.72 (m, 3H), 1.70-1.53 (m, 4H), 1.01 (s, 3H).

### Precursor Example 36: (S)-4-(2-(2-Bromoacetamido)acetamido)-5-((3-(4-((6aS,6bR,7S,8aS,8bS,10R,11aR,12aS,12bS)-6b-fluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid

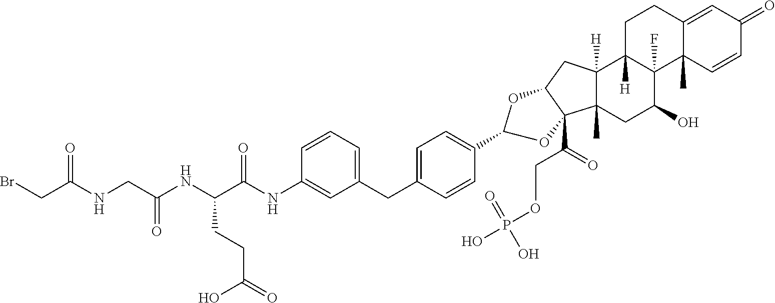

Prepared using similar route to Precursor Example 4 using 2-((6aS,6bR,7S,8aS,8bS,10R,11aR,12aS,12bS)-10-(4-(3-aminobenzyl)phenyl)-6b-fluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate.

LCMS (Method AA15) Rt=1.690 min, m/z 976.1 (M+H)⁺. ¹H NMR (methanol-d4, 400 MHz) δ=7.47 (br d, J=8.6 Hz, 1H), 7.44-7.36 (m, 4H), 7.28-7.19 (m, 3H), 6.97 (d, J=7.6 Hz, 1H), 6.33 (dd, J=1.7, 10.1 Hz, 1H), 6.14 (s, 1H), 5.53 (s, 1H), 5.07-4.96 (m, 2H), 4.83-4.73 (m, 1H), 4.50 (dd, J=4.8, 9.0 Hz, 1H), 4.34 (br d, J=8.7 Hz, 1H), 4.00-3.91 (m, 6H), 2.83-2.71 (m, 1H), 2.68-2.54 (m, 1H), 2.45 (br t, J=7.6 Hz, 3H), 2.40-2.26 (m, 2H), 2.25-2.13 (m, 1H), 2.06-1.91 (m, 2H), 1.84-1.72 (m, 3H), 1.65-1.50 (m, 4H), 1.04 (s, 3H).

### Precursor Example 37: 2-((6aS,6bR,7S,8aS,8bS,10R,11aR,12aS,12bS)-10-(4-(3-((S)-6-Amino-2-(2-(2-bromoacetamido)acetamido)hexanamido)benzyl)phenyl)-6b-fluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate

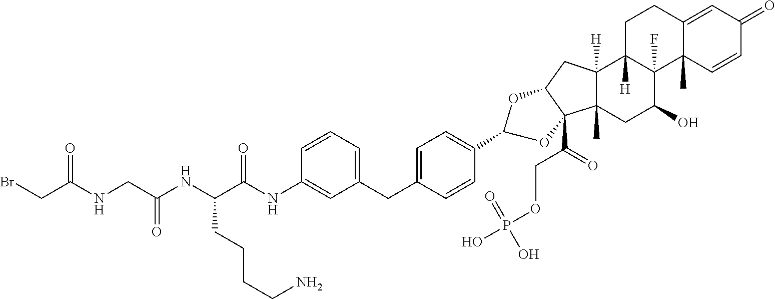

Prepared using similar route to Precursor Example 5 using 2-((6aS,6bR,7S,8aS,8bS,10R,11aR,12aS,12bS)-10-(4-(3-aminobenzyl)phenyl)-6b-fluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate.

LCMS (Method AA16) Rt=2.437 min, m/z 975.2 (M+H)⁺. ¹H NMR (dimethylsulfoxide-d6, 400 MHz) δ=9.94 (br s, 1H), 8.54 (br t, J=5.4 Hz, 1H), 8.21 (br d, J=7.9 Hz, 1H), 7.75 (br s, 3H), 7.51 (br d, J=7.3 Hz, 1H), 7.39-7.30 (m, 3H), 7.30-7.17 (m, 4H), 6.95 (br d, J=7.5 Hz, 1H), 6.22 (br d, J=10.1 Hz, 1H), 6.03 (s, 1H), 5.49 (s, 1H), 4.92 (br s, 1H), 4.79 (br dd, J=8.3, 18.2 Hz, 1H), 4.58 (br dd, J=8.0, 18.4 Hz, 1H), 4.42-4.32 (m, 1H), 4.19 (br d, J=9.0 Hz, 1H), 3.94 (s, 2H), 3.89 (br s, 2H), 3.86-3.71 (m, 3H), 2.74 (br s, 2H), 2.33 (br s, 1H), 2.23-1.96 (m, 2H), 1.83 (br d, J=10.6 Hz, 2H), 1.76-1.46 (m, 10H), 1.45-1.22 (m, 3H), 0.90 (s, 3H).

### Precursor Example 47: (S)-5-(((S)-1-(((S)-6-Amino-1-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxohexan-2-yl)amino)-3-hydroxy-1-oxopropan-2-yl)amino)-4-(2-bromoacetamido)-5-oxopentanoic acid

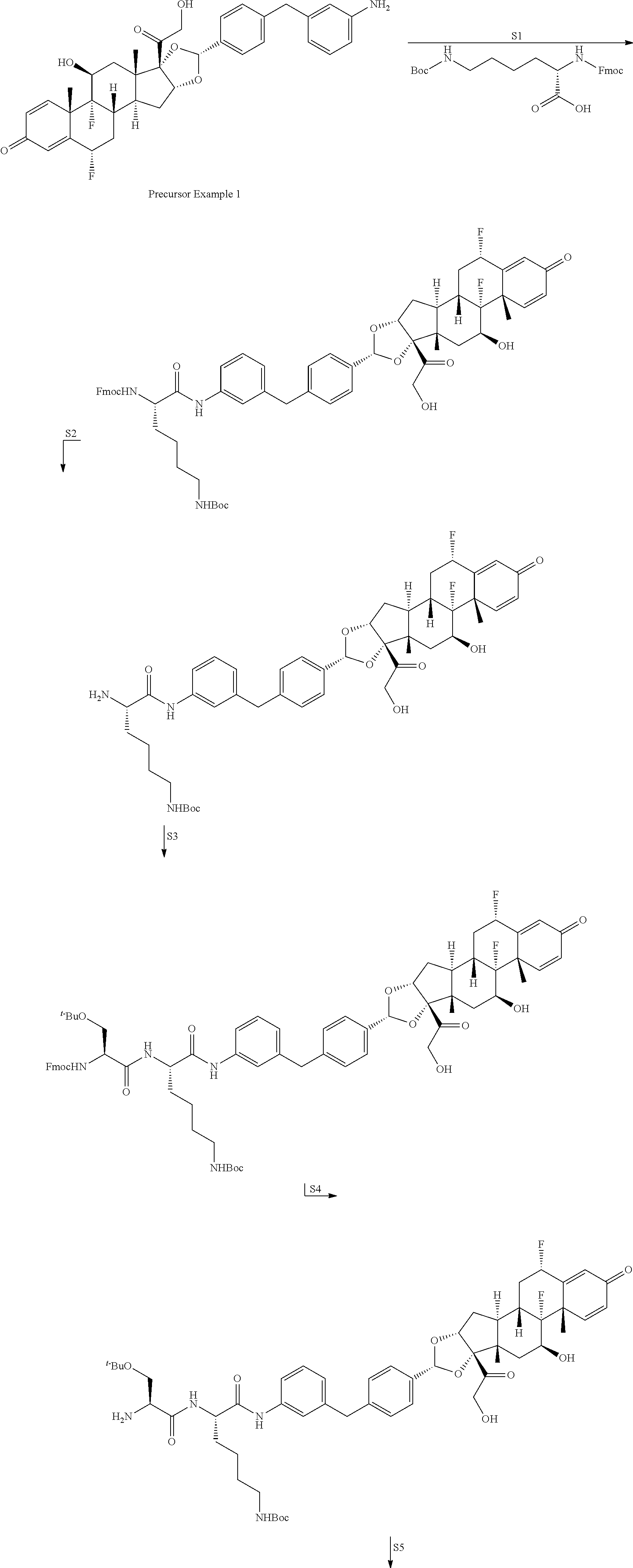

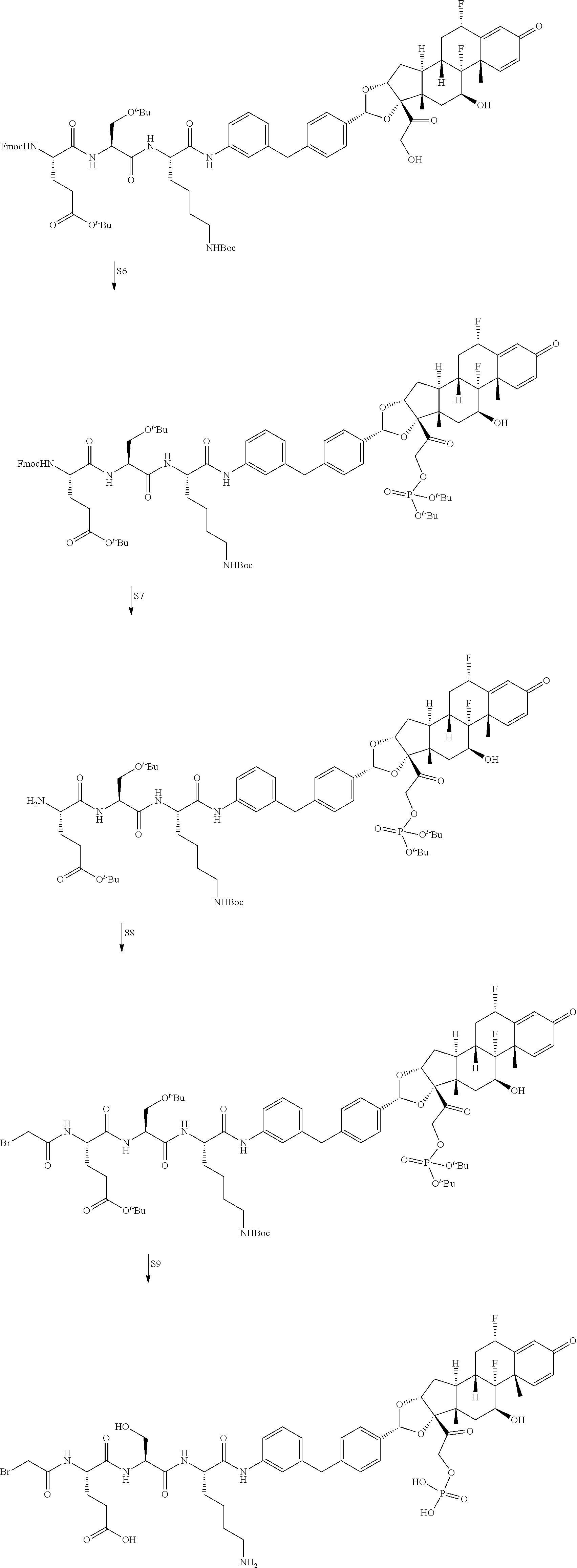

Step 1: Synthesis of (9H-fluoren-9-yl)methyl tert-butyl ((S)-6-((3-(4-42S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexane-1,5-diyl)dicarbamate. A mixture of (S)-2-((((9H-fluoren-9-yl)methoxy)carbonyl)amino)-6-((tert-butoxycarbonyl)amino)hexanoic acid (0.390 g, 0.832 mmol), the product of Precursor Example 1 ((2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-6a,6b,7,8,8a,8b,11a,12,12a,12b-decahydro-1H-naphtho[2′,1′:4,5]indeno[1,2-d][1,3]dioxol-4(2H)-one) (0.504 g, 0.832 mmol), 1-[bis(dimethylamino)methylene]-1H-1,2,3-triazolo[4,5-b]pyridinium 3-oxid hexafluorophosphate (HATU) (0.380 g, 0.999 mmol) and 2,6-dimethylpyridine (0.291 mL, 2.496 mmol) in dimethylformamide (4 mL) was stirred at ambient temperature for 30 hours. The reaction mixture was diluted with ethyl acetate (100 mL), washed with a 1N aqueous solution of HCl (50 mL), a saturated aqueous solution NaHCO₃ (50 mL) and saturated brine solution (50 mL). The organic phase was dried (Na₂SO₄), filtered and solvent was removed under reduced pressure. The resulting residue was purified by chromatography (silica) eluting with a gradient of 0-70% ethyl acetate/heptanes to afford the title compound (0.780 g, 89% yield). LCMS (Method AA17): Rt=1.10 min, m/z 1056.9 (M+H)⁺. ¹H NMR (dimethylsulfoxide-d₆, 400 MHz) δ 9.87 (s, 1H), 7.85 (d, J=7.5 Hz, 2H), 7.69 (dd, J=7.6, 4.8 Hz, 2H), 7.52 (d, J=7.9 Hz, 1H), 7.44 (d, J=8.2 Hz, 1H), 7.41-7.24 (m, 7H), 7.24-7.12 (m, 4H), 6.88 (d, J=7.6 Hz, 1H), 6.71 (s, 1H), 6.24 (dd, J=10.1, 1.9 Hz, 1H), 6.09 (s, 1H), 5.61 (ddd, J=48.9, 11.0, 6.6 Hz, 1H), 5.48 (d, J=3.6 Hz, 1H), 5.41 (s, 1H), 5.06 (t, J=5.9 Hz, 1H), 4.91 (d, J=4.8 Hz, 1H), 4.47 (dd, J=19.4, 6.3 Hz, 1H), 4.27-4.11 (m, 4H), 4.07-3.95 (m, 1H), 3.85 (s, 2H), 2.89-2.83 (m, 2H), 2.66-2.51 (m, 1H), 2.28-2.23 (m, 2H), 2.20 (dt, J=12.2, 6.3 Hz, 1H), 2.01 (d, J=13.9 Hz, 1H), 1.74-1.62 (m, 2H), 1.66-1.49 (m, 4H), 1.46 (s, 3H), 1.31 (s, 9H), 1.22 (d, J=9.3 Hz, 1H), 0.83 (s, 3H).

Step 2: Synthesis of tert-butyl ((S)-5-amino-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. Diethylamine (0.540 g, 7.39 mmol) was added to a degassed solution of (9H-fluoren-9-yl)methyl tert-butyl ((S)-6-((3-(4-42S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexane-1,5-diyl)dicarbamate (0.780 g, 0.739 mmol) in tetrahydrofuran (10 mL). The reaction mixture was stirred at ambient temperature for 2 hours, whereupon the solvent was removed under reduced pressure. The resulting residue was treated with toluene (3×50 mL), which was removed under reduced pressure to drive off as much diethylamine as possible. The crude title compound was used immediately without further purification. LCMS (Method AA17): Rt=0.86 min, m/z 834.0 (M+H)⁺.

Step 3: Synthesis of tert-butyl ((S)-5-((S)-2-((((9H-fluoren-9-yl)methoxy)carbonyl)amino)-3-(tert-butoxy)propanamido)-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. A mixture of (S)-2-((((9H-fluoren-9-yl)methoxy)carbonyl)amino)-3-(tert-butoxy)propanoic acid (0.283 g, 0.739 mmol), tert-butyl ((S)-5-amino-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (0.616 g, 0.739 mmol), 1-[bis(dimethylamino)methylene]-1H-1,2,3-triazolo[4,5-b]pyridinium 3-oxid hexafluorophosphate) (HATU) (0.337 g, 0.887 mmol) and 2,6-dimethylpyridine (0.258 mL, 2.217 mmol) in dimethylformamide (4 mL) was stirred at 0° C. for 0.5 hours. The reaction mixture was diluted with ethyl acetate (100 mL), washed with a 1N aqueous solution of HCl (50 mL), a saturated aqueous solution of NaHCO₃ (50 mL) and saturated brine solution (50 mL). The organic phase was dried (Na₂SO₄), filtered and solvent was removed under reduced pressure. The resulting residue was purified by chromatography (silica) eluting with a gradient of 0-70% ethyl acetate/heptanes to afford the title compound (0.500 g, 56% yield). LCMS (Method AA17): Rt=1.18 min, m/z 1199.2 (M+H)⁺. ¹H NMR (dimethylsulfoxide-d₆, 500 MHz) δ 9.84 (s, 1H), 7.95 (d, J=8.0 Hz, 1H), 7.86 (d, J=7.5 Hz, 2H), 7.70 (t, J=7.1 Hz, 2H), 7.46-7.26 (m, 7H), 7.26-7.13 (m, 5H), 6.89 (d, J=7.6 Hz, 1H), 6.67 (s, 1H), 6.27 (dd, J=10.2, 1.9 Hz, 1H), 6.11 (s, 1H), 5.62 (dt, J=48.6, 9.1 Hz, 1H), 5.50 (s, 1H), 5.41 (s, 1H), 5.08 (t, J=5.9 Hz, 1H), 4.92 (d, J=4.9 Hz, 1H), 4.48 (dd, J=19.4, 5.8 Hz, 1H), 4.36 (d, J=6.9 Hz, 1H), 4.33-4.07 (m, 5H), 3.84 (s, 2H), 3.44 (d, J=6.1 Hz, 2H), 2.85 (q, J=6.5 Hz, 2H), 2.68-2.53 (m, 1H), 2.32-2.16 (m, 2H), 2.02 (d, J=13.7 Hz, 1H), 1.67 (d, J=13.5 Hz, 3H), 1.60-1.48 (m, 1H), 1.47 (s, 3H), 1.31 (s, 10H), 1.25-1.16 (m, 1H), 1.05 (s, 9H), 0.84 (s, 3H).

Step 4: Synthesis of tert-butyl ((S)-5-((S)-2-amino-3-(tert-butoxy)propanamido)-6-((3-(4-42S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate. Diethylamine (0.305 g, 4.17 mmol) was added to a degassed solution of tert-butyl ((S)-5-((S)-2-((((9H-fluoren-9-yl)methoxy)carbonyl)amino)-3-(tert-butoxy)propanamido)-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (0.500 g, 0.417 mmol) in tetrahydrofuran (10 mL). The reaction mixture was stirred at ambient temperature for 2 hours, whereupon the solvent was removed under reduced pressure. The resulting residue was treated with toluene (3×50 mL), which was removed under reduced pressure to drive off as much diethylamine as possible. The crude title compound was used immediately without further purification. LCMS (Method AA17): Rt=0.92 min, m/z 976.9 (M+H)⁺.

Step 5: Synthesis of (10S,13S,16S)-tert-butyl 16-((((9H-fluoren-9-yl)methoxy)carbonyl)amino)-13-(tert-butoxymethyl)-10-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate. A mixture of (S)-2-((((9H-fluoren-9-yl)methoxy)carbonyl)amino)-5-(tert-butoxy)-5-oxopentanoic acid (0.177 g, 0.417 mmol), tert-butyl ((S)-5-((S)-2-amino-3-(tert-butoxy)propanamido)-6-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-6-oxohexyl)carbamate (0.407 g, 0.417 mmol), (1-[bis(dimethylamino)methylene]-1H-1,2,3-triazolo[4,5-b]pyridinium 3-oxid hexafluorophosphate) (HATU) (0.190 g, 0.500 mmol) and 2,6-dimethylpyridine (0.146 ml, 1.251 mmol) in dimethylformamide (4 mL) was stirred at 0° C. for 0.5 hours. The reaction mixture was diluted with ethyl acetate (100 mL), washed with a 1M aqueous solution of HCl (50 mL), a saturated aqueous solution of NaHCO₃ (50 mL) and saturated brine solution (50 mL). The organic phase was dried (Na₂SO₄), filtered and solvent was removed under reduced pressure. The resulting residue was purified by chromatography (silica) eluting with a gradient of 0-70% ethyl acetate/heptanes to afford the title compound (0.455 g, 79% yield). LCMS (Method AA17): Rt=1.03 min, m/z not observed. ¹H NMR (dimethylsulfoxide-d6, 500 MHz) δ 9.77 (s, 1H), 7.91 (s, 1H), 7.85 (d, J=7.6 Hz, 3H), 7.77 (d, J=7.7 Hz, 1H), 7.68 (t, J=6.7 Hz, 2H), 7.58 (d, J=8.0 Hz, 1H), 7.44-7.34 (m, 4H), 7.34-7.22 (m, 4H), 7.16 (dd, J=22.5, 8.0 Hz, 3H), 6.87 (d, J=7.6 Hz, 1H), 6.65 (s, 1H), 6.25 (dd, J=10.1, 1.9 Hz, 1H), 6.09 (s, 1H), 5.70-5.50 (m, 1H), 5.48 (d, J=3.8 Hz, 1H), 5.40 (s, 1H), 5.06 (t, J=6.0 Hz, 1H), 4.90 (d, J=4.7 Hz, 1H), 4.47 (dd, J=19.4, 6.2 Hz, 1H), 4.29 (dt, J=19.5, 6.5 Hz, 3H), 4.23-4.10 (m, 3H), 4.03 (d, J=6.3 Hz, 1H), 3.84 (s, 2H), 3.53 (s, OH), 3.52-3.36 (m, 1H), 2.84-2.78 (m, 2H), 2.67-2.50 (m, 1H), 2.22 (t, J=8.4 Hz, 3H), 2.01 (d, J=13.6 Hz, 1H), 1.88 (s, 1H), 1.80-1.58 (m, 6H), 1.56-1.48 (m, 1H), 1.46 (s, 3H), 1.34 (s, 9H), 1.29 (s, 9H), 1.20 (s, 1H), 1.00 (s, 9H), 0.93 (s, 1H), 0.82 (s, 3H).

Step 6: Synthesis of (10S,13S,16S)-tert-butyl 16-((((9H-fluoren-9-yl)methoxy)carbonyl)amino)-13-(tert-butoxymethyl)-10-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate. A mixture of (10S,13S,16S)-tert-butyl 16-((((9H-fluoren-9-yl)methoxy)carbonyl)amino)-13-(tert-butoxymethyl)-10-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate (0.452 g, 0.326 mmol) and 1H-tetrazole (0.105 g, 1.502 mmol) in dimethylformamide (1.5 mL) was treated with di-tert-butyl diethylphosphoramidite (0.291 mL, 1.045 mmol). The reaction mixture was stirred at ambient temperature for 4 h, whereupon an aqueous solution of H₂O₂ (50 wt %, 0.3 mL) was added and stirring continued for 1 h. Purification by prep-HPLC eluting with a gradient of acetonitrile and 10 mM aqueous ammonium acetate afforded the title compound (0.455 g, 79% yield). LCMS (Method AA17): Rt=1.35 min, m/z 1575.8 (M+H)⁺. ¹H NMR (dimethylsulfoxide-d6, 400 MHz) δ 9.76 (s, 1H), 7.85 (d, J=7.7 Hz, 3H), 7.77 (d, J=7.7 Hz, 1H), 7.67 (t, J=6.7 Hz, 2H), 7.58 (d, J=8.0 Hz, 1H), 7.44-7.18 (m, 10H), 7.14 (t, J=7.8 Hz, 1H), 6.88 (d, J=7.5 Hz, 1H), 6.65 (s, 1H), 6.25 (dd, J=10.1, 1.9 Hz, 1H), 6.09 (s, 1H), 5.70-5.48 (m, 3H), 4.98-4.87 (m, 2H), 4.61 (dd, J=17.9, 9.3 Hz, 1H), 4.29 (dt, J=19.4, 6.8 Hz, 2H), 4.19 (d, J=7.0 Hz, 2H), 4.03 (d, J=7.0 Hz, 1H), 3.84 (s, 2H), 3.54-3.32 (m, 1H), 2.82 (d, J=6.5 Hz, 2H), 2.74-2.50 (m, 1H), 2.22 (t, J=7.9 Hz, 3H), 2.03 (d, J=13.1 Hz, 1H), 1.94-1.80 (m, 1H), 1.76-1.60 (m, 5H), 1.55-1.41 (m, 2H), 1.45 (s, 3H), 1.39 (s, 9H), 1.38 (s, 9H), 1.34 (s, 9H), 1.29 (s, 9H), 1.25-1.12 (m, 1H), 0.99 (s, 9H), 0.91 (s, 1H), 0.85 (s, 3H).

Step 7: Synthesis of (10S,13S,16S)-tert-butyl 16-amino-13-(tert-butoxymethyl)-10-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate. Diethylamine (0.100 g, 1.364 mmol) was added to a degassed solution of (10S,13S,16S)-tert-butyl 16-((((9H-fluoren-9-yl)methoxy)carbonyl)amino)-13-(tert-butoxymethyl)-10-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate (0.215 g, 0.136 mmol) in THF (8 mL). The reaction mixture was stirred at ambient temperature for 2 hours, whereupon the solvent was removed under reduced pressure. The resulting residue was treated with toluene (3×50 mL), which was removed under reduced pressure to drive off as much diethylamine as possible. The crude title compound was used immediately without further purification. LCMS (Method AA17): Rt=1.11 min, m/z 1354.8 (M+H)⁺.

Step 8: Synthesis of (10S,13S,16S)-tert-butyl 16-(2-bromoacetamido)-13-(tert-butoxymethyl)-10-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate. A mixture of bromoacetic acid (0.0347 g, 0.250 mmol) and ethyl 2-ethoxyquinoline-1(2H)-carboxylate (0.07736 g, 0.298 mmol) in dimethylformamide (0.2 mL) was stirred at ambient temperature for 30 minutes, whereupon a solution of (10S,13S,16S)-tert-butyl 16-amino-13-(tert-butoxymethyl)-10-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate (0.130 g, 0.096 mmol) in dimethylformamide (0.3 mL) was added to the solution of bromoacetic acid. The reaction was stirred at ambient temperature for 20 minutes. Purification by preparative HPLC eluting with a gradient of acetonitrile and 0.1% (v/v) trifluoroacetic acid in water afforded the title compound (0.103 g, 73% yield). LCMS (Method AA17): Rt=1.20 min, m/z 1474.4, 1476.5 (M+H)⁺. ¹H NMR (dimethylsulfoxide-d6, 400 MHz) δ 9.77 (s, 1H), 8.46 (d, J=7.8 Hz, 1H), 7.94 (d, J=7.8 Hz, 1H), 7.85 (d, J=8.0 Hz, 1H), 7.41 (d, J=8.3 Hz, 1H), 7.35 (s, 1H), 7.31 (d, J=8.0 Hz, 2H), 7.22 (dd, J=14.0, 9.0 Hz, 3H), 7.15 (t, J=7.9 Hz, 1H), 6.88 (d, J=7.5 Hz, 1H), 6.66 (s, 1H), 6.26 (dd, J=10.2, 1.9 Hz, 1H), 6.09 (s, 1H), 5.58 (d, J=51.4 Hz, 3H), 4.98-4.87 (m, 2H), 4.61 (dd, J=18.0, 9.2 Hz, 1H), 4.33 (s, 1H), 4.17 (s, 1H), 3.93-3.86 (m, 2H), 3.85 (s, 2H), 3.63 (s, 10H), 2.87-2.79 (m, 2H), 2.28-2.16 (m, 4H), 2.04 (d, J=13.2 Hz, 1H), 1.87 (s, 1H), 1.69 (s, 3H), 1.63 (d, J=13.6 Hz, 2H), 1.46 (s, 3H), 1.39 (s, 9H), 1.38 (s, 9H), 1.33 (s, 9H), 1.30 (s, 9H), 1.19 (d, J=9.7 Hz, 1H), 1.02 (s, 9H), 0.85 (s, 3H).

Step 9: Synthesis of (S)-5-(((S)-1-(((S)-6-amino-1-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-1-oxohexan-2-yl)amino)-3-hydroxy-1-oxopropan-2-yl)amino)-4-(2-bromoacetamido)-5-oxopentanoic acid. Trifluoroacetic acid (2 mL, 0.068 mmol) was added to a 0° C. solution of (10S,13S,16S)-tert-butyl 16-(2-bromoacetamido)-13-(tert-butoxymethyl)-10-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)carbamoyl)-2,2-dimethyl-4,12,15-trioxo-3-oxa-5,11,14-triazanonadecan-19-oate (0.100 g, 0.068 mmol) in dichloromethane (2 mL). The reaction was stirred at 0° C. for 10 minutes, whereupon the ice bath was removed the ice-bath and stirring was continued at ambient temperature for an additional 90 minutes. Purification by preparative HPLC eluting with a gradient of acetonitrile and 0.1% (v/v) trifluoroacetic acid in water afforded the title compound (0.056 g, 72% yield). LCMS (Method AA17) Major acetal isomer: Rt=0.75 min, m/z 1149.7, 1151.8 (M+H)⁺; minor acetal isomer Rt=0.78 min, m/z 1149.7, 1151.7 (M+H⁺). ¹H NMR (dimethylsulfoxide-d6, 500 MHz) δ 9.70 (s, 1H), 8.61 (d, J=7.7 Hz, 1H), 8.13 (d, J=7.2 Hz, 1H), 8.06 (d, J=7.7 Hz, 1H), 7.81 (s, 3H), 7.51 (d, J=8.3 Hz, 1H), 7.35 (d, J=7.9 Hz, 2H), 7.23 (t, J=8.7 Hz, 3H), 7.18 (t, J=7.8 Hz, 1H), 7.13 (s, 1H), 6.97 (d, J=7.6 Hz, 1H), 6.27 (dd, J=10.1, 1.9 Hz, 1H), 6.11 (s, 1H), 5.77-5.53 (m, 1H), 5.43 (s, 1H), 4.92 (d, J=4.7 Hz, 1H), 4.70 (dd, J=19.0, 8.1 Hz, 1H), 4.53 (dd, J=19.0, 7.9 Hz, 1H), 4.27 (q, J=6.6 Hz, 3H), 4.19 (d, J=9.6 Hz, 1H), 3.96-3.86 (m, 4H), 3.58 (qd, J=10.9, 5.9 Hz, 2H), 2.77-2.52 (m, 3H), 2.68-2.54 (m, OH), 2.49 (s, 2H), 2.23 (q, J=8.3 Hz, 3H), 2.00 (d, J=13.2 Hz, 1H), 1.93-1.85 (m, 2H), 1.77-1.69 (m, 1H), 1.72-1.65 (m, 3H), 1.57 (s, 1H), 1.49 (s, 4H), 1.54-1.44 (m, 2H), 1.30 (s, 2H), 0.88 (s, 3H).

### Precursor Example 48: (S)-6-Amino-2-((S)-2-(2-(2-bromoacetamido)acetamido)-3-hydroxypropanamido)-N-(3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)hexanamide

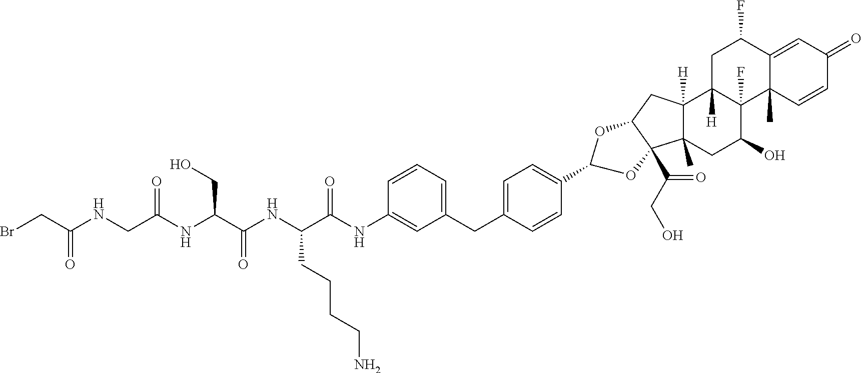

Prepared using similar route to Precursor Example 47 using 2-((2S,6aS,6bR,7S,8aS,8bS,10R,11aR,12aS,12bS)-10-(4-(3-aminobenzyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate.

LCMS (Method AA17) Rt=0.79 min, m/z 998.7, 1000.9 (M+H)⁺. ¹H NMR (dimethylsulfoxide-d6, 500 MHz) δ 9.66 (s, 1H), 8.55 (t, J=5.7 Hz, 1H), 8.16 (dd, J=12.8, 7.7 Hz, 2H), 7.62 (s, 3H), 7.48-7.40 (m, 2H), 7.40-7.32 (m, 2H), 7.31-7.23 (m, 3H), 7.23-7.17 (m, 2H), 6.92 (d, J=7.6 Hz, 1H), 6.54 (s, 1H), 6.30 (dd, J=10.2, 1.9 Hz, 1H), 6.13 (s, 1H), 5.66 (dt, J=48.7, 10.2 Hz, 1H), 5.54 (dd, J=4.5, 1.7 Hz, 1H), 5.45 (s, 1H), 5.21 (s, 1H), 5.13 (s, 1H), 4.95 (d, J=4.8 Hz, 1H), 4.51 (d, J=19.4 Hz, 1H), 4.36 (td, J=12.8, 7.2 Hz, 2H), 4.20 (d, J=19.2 Hz, 1H), 3.92 (d, J=1.7 Hz, 2H), 3.89 (d, J=2.4 Hz, 2H), 3.82 (qd, J=16.7, 5.7 Hz, 2H), 3.65 (t, J=8.2 Hz, 1H), 3.58 (dd, J=10.5, 5.9 Hz, 1H), 2.78 (q, J=6.7 Hz, 2H), 2.73-2.58 (m, 1H), 2.35-2.28 (m, 1H), 2.24 (td, J=12.3, 6.8 Hz, 1H), 2.04 (d, J=13.3 Hz, 1H), 1.85-1.50 (m, 4H), 1.50 (s, 3H), 1.36 (dq, J=16.0, 8.3 Hz, 2H), 0.87 (s, 3H).

### ADC Example 1. Conjugation with Maleimide-Derived Products (General Method)

1. Cysteine Conjugation Protocol with Maleimide

An approximate 10 mg/mL solution of the antibody was prepared in phosphate buffer saline (PBS), pH 7.4 as well as a 10 mM TCEP (tris(2-carboxyethyl)phosphine) solution in PBS (Pierce Bond-Breaker, cat. 77720). Anti-CD40 antibody (Table 3) was then partially reduced by adding approximately two molar equivalents (eq) of 10 mM TCEP, briefly mixing, and incubating for 60 min at 37° C. Dimethyl sulfoxide (DMSO) was then added to the partially reduced antibodies in sufficient quantity to 15% total DMSO. For the conjugations, 8 molar equivalents (eq) of the maleimide products of Examples 6 to 13 (10 mM in PBS) were then added and incubated for 30 min at room temperature with the partially reduced antibodies. Excess maleimide product and DMSO were then removed using NAP-5 desalting columns (GE Healthcare, cat. 17-0853-02) previously equilibrated with phosphate buffer saline buffer, pH 7.4. Desalted samples were then analyzed by size exclusion chromatography (SEC), Hydrophobic Interaction Chromatography (HIC), and reduced mass spectrometry.

2. Thiosuccinimide Hydrolysis

Hydrolysis of the thiosuccinimide ring of ADC was accomplished by incubating the ADC at an elevated pH. Briefly, a 0.7 M arginine, pH 9.0 solution was prepared and added to each ADC in phosphate buffer saline (PBS) buffer to bring the total arginine concentration to 50 mM (pH 8.9). The material was then incubated at 25° C. for 72 hours. Hydrolysis of the succinimide ring was then confirmed by reduced mass spectrometry, after which, hydrolysis was quenched with the addition of a 0.1 M acetic acid solution to 12.5 mM total acetic acid (pH 7.1).

Table 11 provides ADC conjugates synthesized following this General Method (aggregation data provided for an exemplary number of the ADC conjugates). Table 12 provides ADC conjugates which may be synthesized following this General Method. Variable (A) corresponds to the anti-CD40 antibody; n=1, 2, 3, 4, 5, 6, 7, 8, 9, or 10. Human anti-CD40 antibody corresponds to Ab102 (Table 3). Mouse anti-CD40 antibody corresponds to Antibody 138 described in US 20160347850, incorporated herein by reference. P=Precursor Example. Antibody 138 has similar characteristics to Ab102, e.g., antibody 138 is an antagonist antibody with no substantial agonist activity like Ab102. Thus, antibody 138 is representative of Ab102 activity in mouse models.

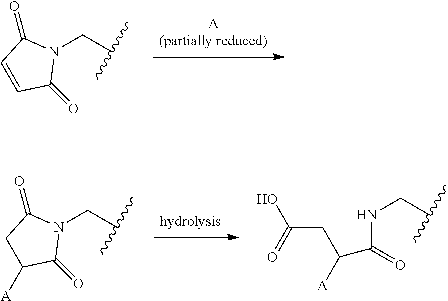

TABLE 11
Maleimide-Derived ADCs Synthesized

| P | ADC Product | n |
|---|---|---|
| 6 | 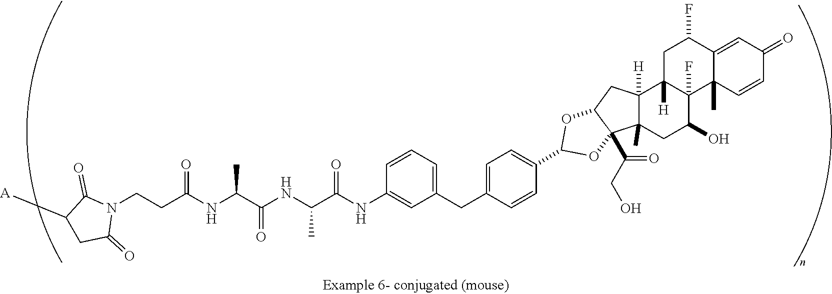 | 4 |
| 6 | 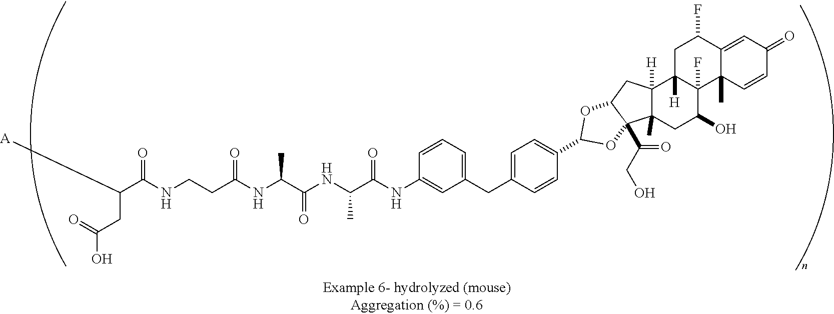 | 4 |
| 7 | 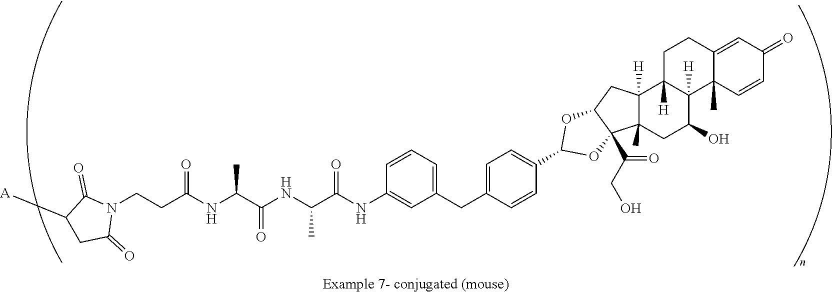 | 4 |
| 7 | 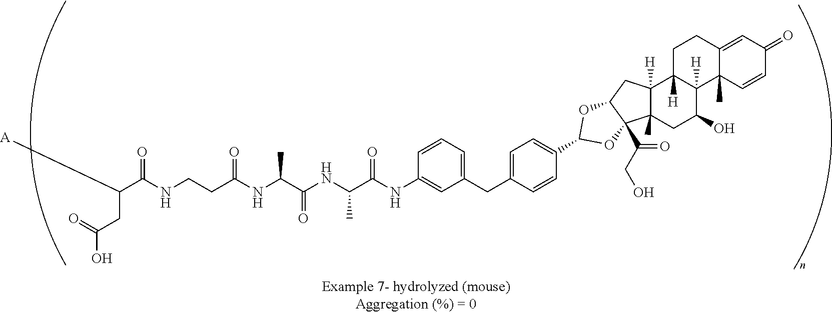 | 4 |
| 12 | 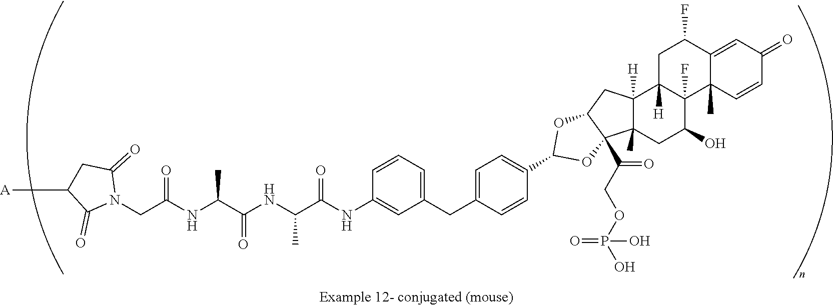 | 2 |
| 12 | 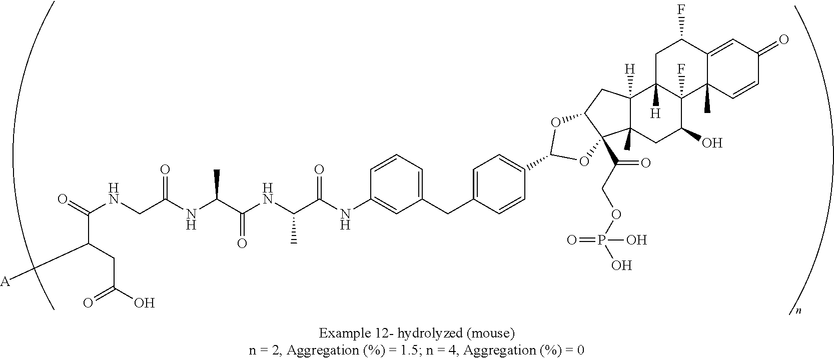 | 2 4 |
| 12 | 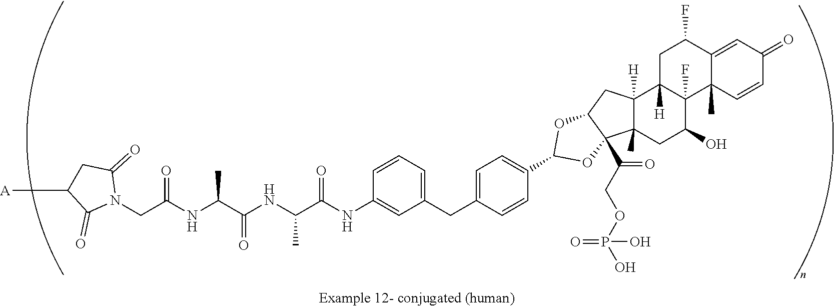 | 4 |
| 12 | 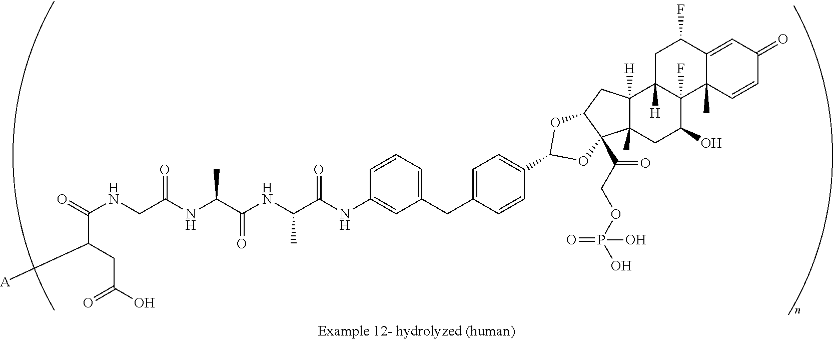 | 4 |
| 13 | 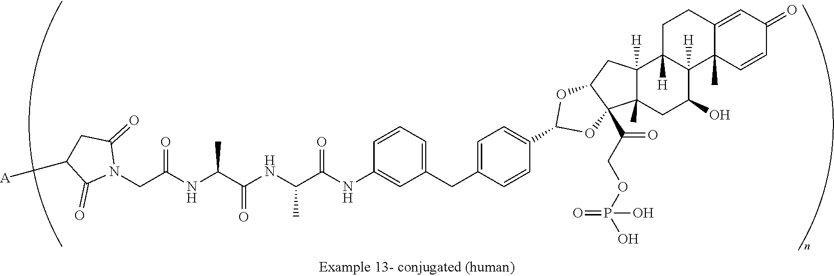 | 4 |
| 13 | 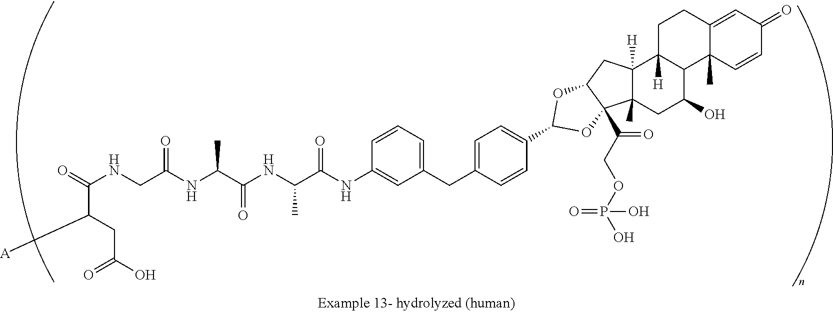 | 4 |

TABLE 13
Additional Maleimide-Derived ADCs

| P | ADC Product |
|---|---|
| 8 | 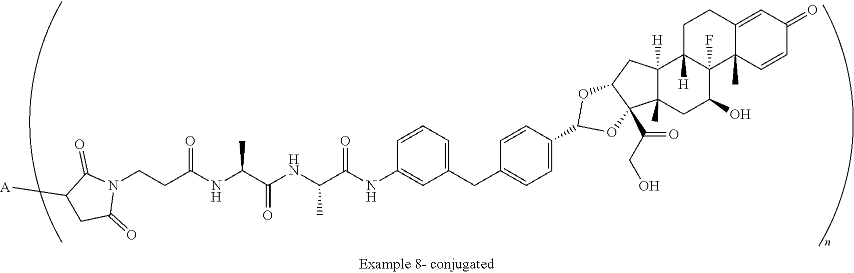 |
| 8 | 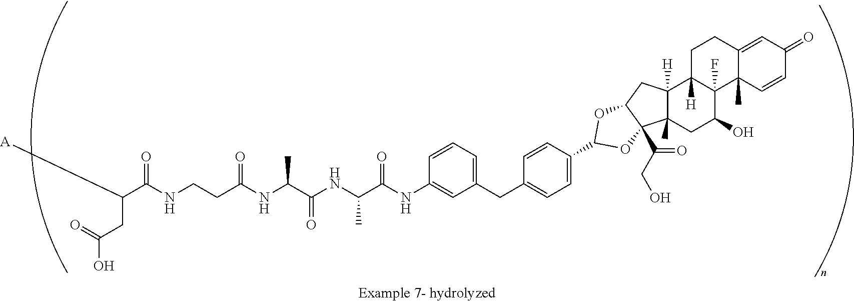 |
| 9 | 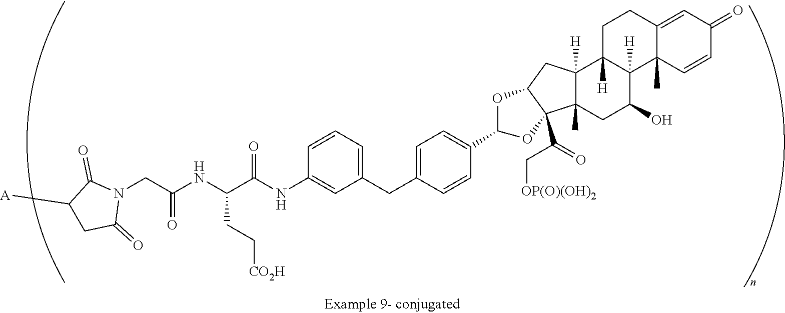 |
| 9 | 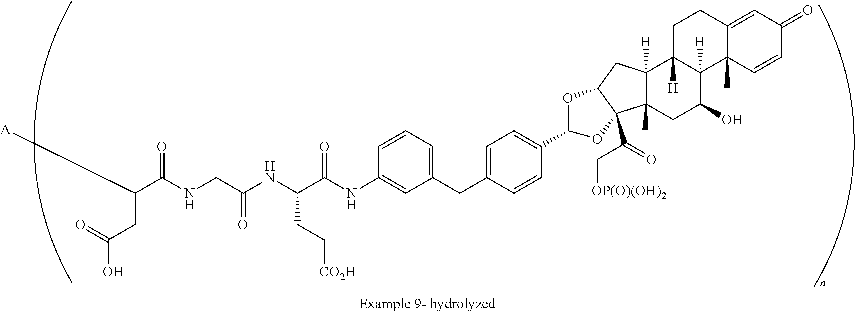 |
| 10 | 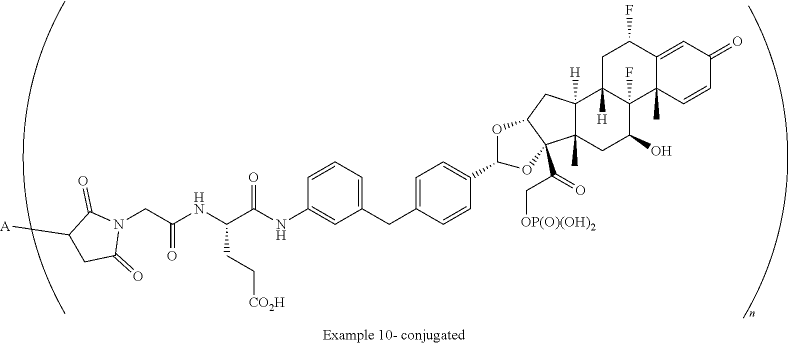 |
| 10 | 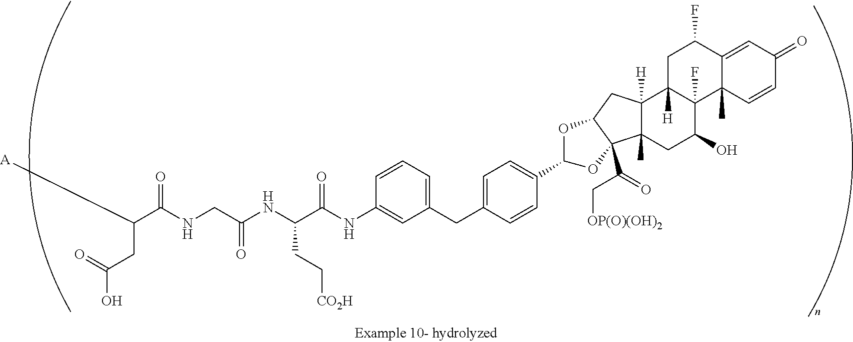 |
| 11 | 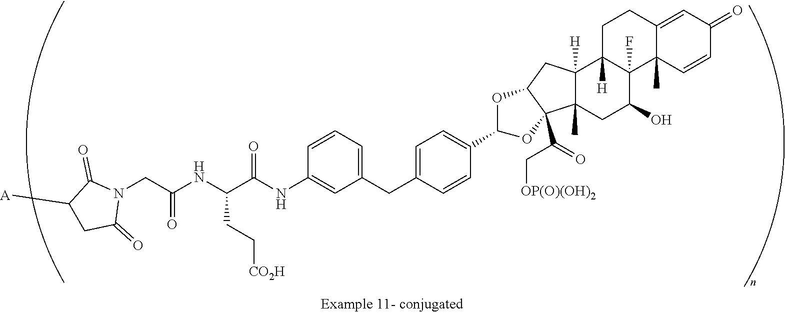 |
| 11 | 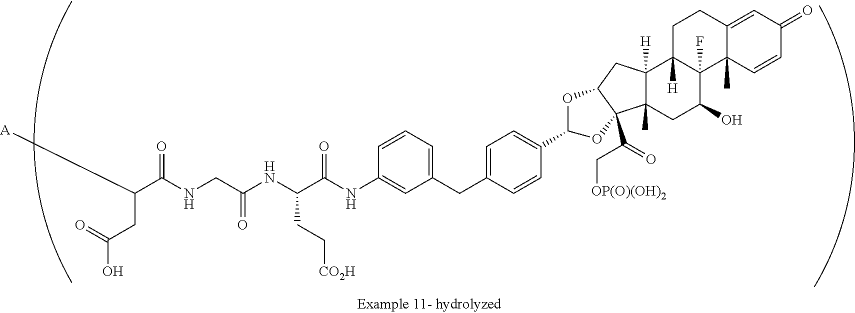 |

### ADC Example 2. Conjugation with Precursor Bromo Acetamide Products (General Method)

1. General Procedure

An approximate 5-20 mg/mL solution of the desired antibody was prepared in phosphate buffer saline (PBS), pH 6-7.4. A reducing agent of choice, such as TCEP (tris(2-carboxyethyl)phosphine), was diluted or dissolved in solvents like H₂O, dimethyl sulfoxide (DMSO), dimethyl acetamide (DMA) or dimethyl formamide (DMF) to give a solution with concentration range between 1 to 25 mM. anti-CD40 antibody was then partially reduced by adding about 2-3.5 eq of reducing agent, briefly mixing, and incubating overnight at 0-4° C. Tris buffer, pH 8-8.5 (20-50 mM) was then added, followed by the bromo acetamide product of Examples 4-5 in dimethyl sulfoxide (DMSO) or dimethyl acetamide (DMA) (less than 15% total) and the mixture was incubated for 2-3 hours at room temperature. Excess bromo acetamide product and organic solvent were then removed by purification. Purified ADC samples were then analyzed by Size exclusion chromatography (SEC), Hydrophobic Interaction Chromatography (HIC) and reduced mass spectrometry.

2. Preparation of Human ADC of Precursor Example 4

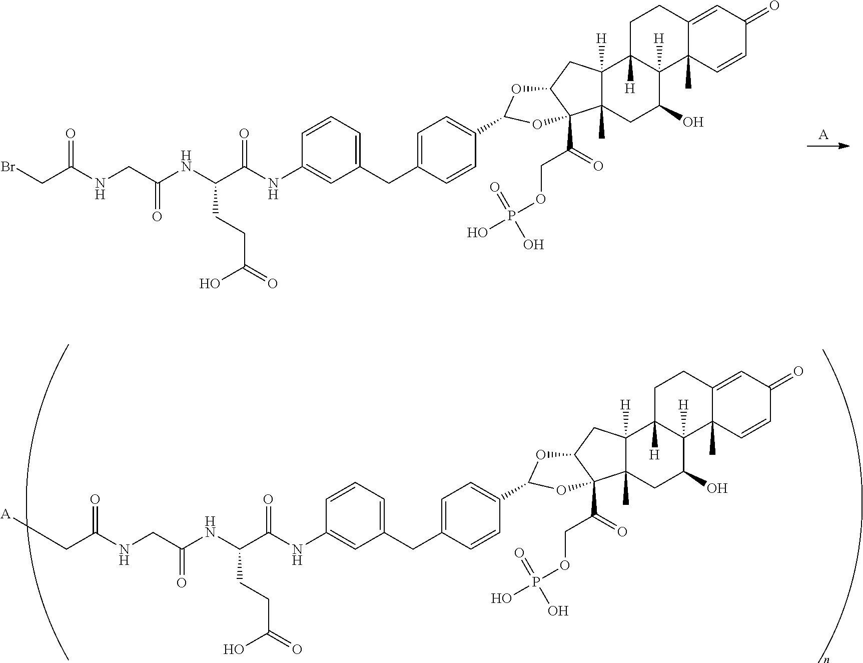

100 mg of human anti-CD40 antibody (Ab102, Table 3) at concentration of 20 mg/mL was reduced with diphenylphosphinoacetic acid (2.9-3.0 equivalents (eq)) at 0° C. overnight. Partially-reduced CD40 Ab was then conjugated to Example 4 bromo acetamide product (10 equivalents (eq)) in dimethyl sulfoxide (DMSO) for 3 hours at room temperature. The conjugation mixture was first buffer exchanged into 20 mM Tris Buffer, 50 mM NaCl, pH 7.8 using multiple NAP 25 desalting columns. The desalted ADC solution was purified by AEC to afford the DAR2 (n=2) and DAR4 (n=4) components of the ADC (number of drug linked molecules depends upon the number of interchain disulfide bonds reduced).

AEC Conditions

The AEC conditions used were: The column was Propac™ WAX-10, 4×250 mm (Thermo Fisher Scientific, cat. 054999) and the column temperature was 37° C. Wavelength was 280 nm, run time was 18 minutes, injection amount was 20 ug, and flow rate was 1.0 mL/minute. Mobile Phase A: 20 mM MES, pH 6.7, Mobile Phase B: 20 mM MES, 500 mM NaCl, pH 6.7. The DAR2 ADC had a retention time of 7.70 min with 0% aggregation, and the DAR4 a retention time of 10.88 min with 0% aggregation.

TABLE 13A
Gradient Profile

| Time (min) | Mobile Phase A (%) | Mobile Phase B (%) |
|---|---|---|
| 0 | 100 | 0 |
| 16 | 0 | 100 |
| 18 | 0 | 100 |
| 18.1 | 0 | 10 |

Mass Spectroscopy

ADC samples were fully reduced before MS analysis. The mass spectrometry conditions used are as follows: HPLC column=Water BEH300 C4, 2.1×50 mm, 3.5 micron particle size; Mobile phase A: 0.1% formic acid in water; Mobile phase B: 0.1% formic acid in acetonitrile; Flow rate: 450 μL/min; Gradient: 0-0.6 min, 5% B, 0.6 to 1.1 min 5-90% B, 1.1 to 2.2 min 90% B, 2.2 to 2.4 min, 90-5% B, 2.4 to 3.5 min 5% B; Column temperature: 40° C.; MS ionization source: ESI

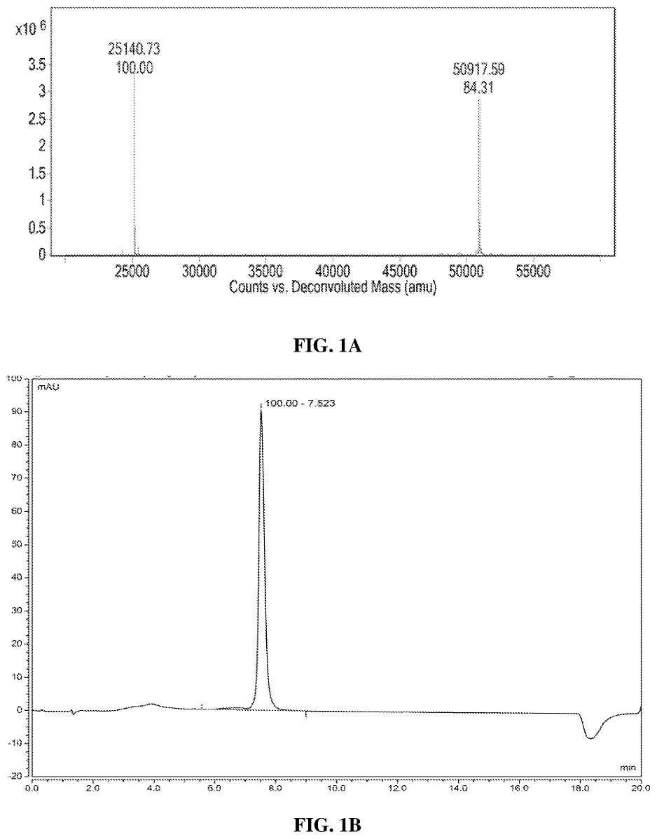

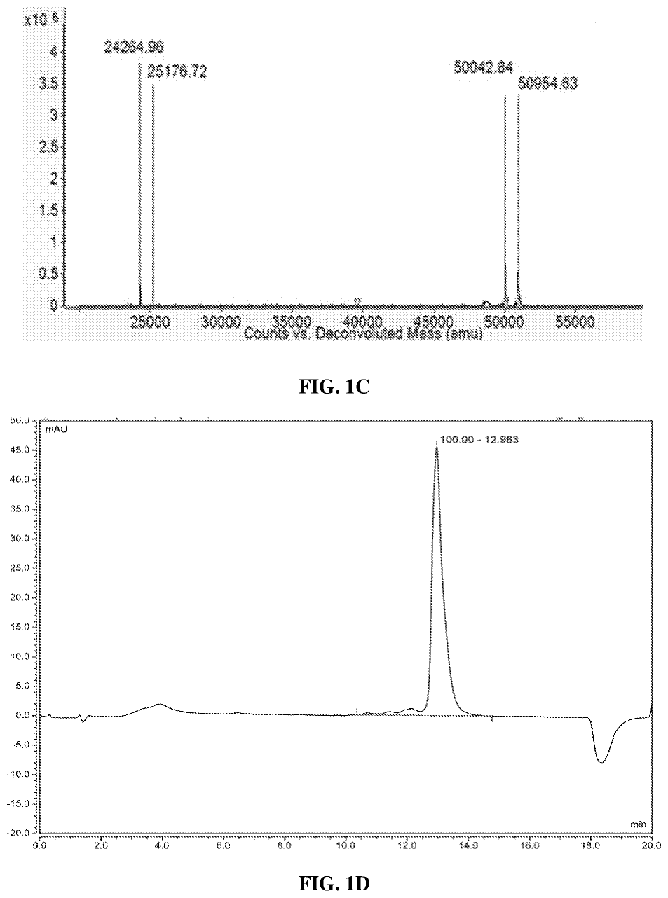

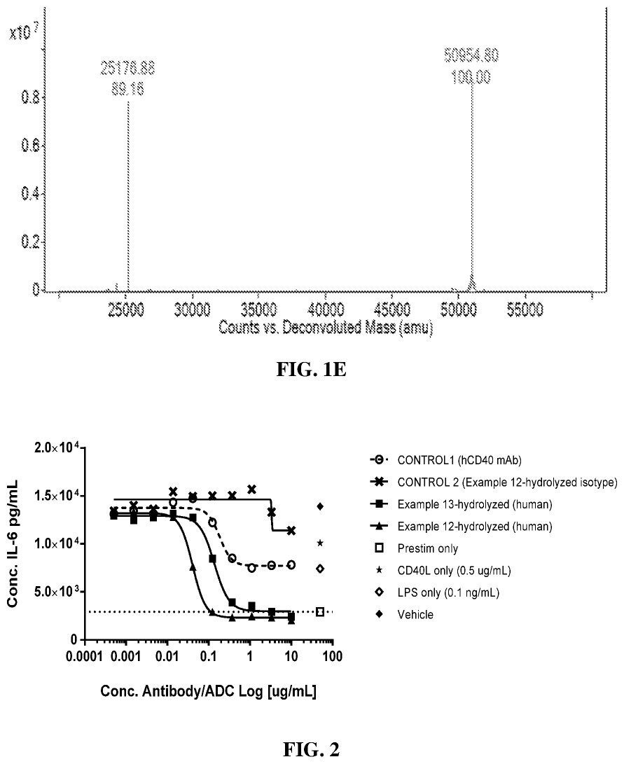

Deconvoluted mass spectroscopy data of Example 4-conjugated (human) is shown in FIG. 1A (n=4). The 25140.73 peak corresponds to the light chain (SEQ ID NO: 2) with one drug linker molecule conjugated. The 50917.59 peak corresponds to the heavy chain (SEQ ID NO: 1) with one drug linker molecule conjugated.

3. Preparation of Mouse and Human ADC of Precursor Example 28

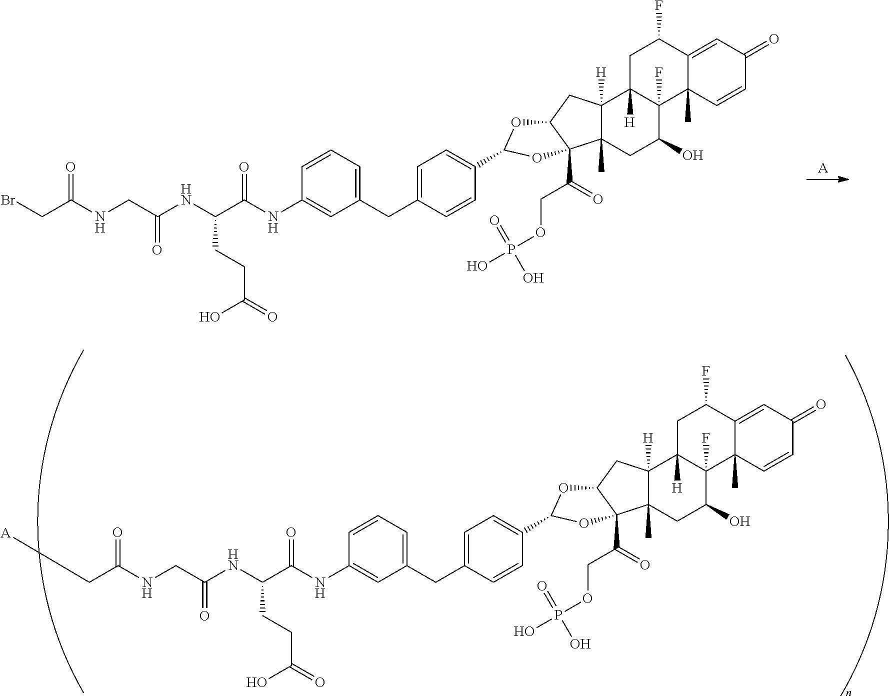

### Mouse ADC

The Example 28 conjugated (mouse) ADC was synthesized following the Example 4 ADC, using mouse anti-CD40 antibody (Antibody 138). DAR2 ADC had a retention time of 7.17 minutes with 0% aggregation, and the DAR4 a retention time of 10.50 minutes with 0% aggregation.

### Human ADC

410 mg of human anti-CD40 antibody (Ab102, Table 3) at concentration of 20 mg/mL was reduced with diphenylphosphinoacetic acid (2.7 eq) at ˜4° C. overnight. 2% (v/v) of 2 M Tris buffer (pH 8.5) was added to the partially-reduced antibody, followed by addition of 10 eq of Precursor Example 28 product in dimethylsulfoxide. After conjugation at room temperature for 3 hours, the mixture was buffer exchanged into 20 mM Tris, 50 mM NaCl, pH 8.0 using NAP25 desalting columns and was further purified by AEC to afford the DAR2 (n=2) and DAR4 (n=4) components of the ADC (number of drug linked molecules depends upon the number of interchain disulfide bonds reduced). Instrument: AKTA pure; Column: 4× Hitrap Q HP 5 mL; Mobile phase: A for 20 mM Tris Buffer, pH 8.0; B for 20 mM Tris Buffer, 500 mM NaCl, pH 8.0; Gradient: B from 10% to 40% over 45 CV; Flow rate: 5 mL/min; Wavelength: 280 & 260 nm.

### AEC Conditions

ProPac™ SAX-10, 4×250 mm, 10 μm (Thermo Fisher Scientific, Cat #054997) at room temperature; wavelength=280 nm; run time=20 minutes; injection amount=˜20 μg; flow rate=1.0 mL/minute; Mobile Phase A: 20 mM Tris, pH 8.0; Mobile Phase B: 20 mM Tris, 1 M NaCl, pH 8.0.

TABLE 13B
Gradient Profile

| Time (min) | Mobile Phase A (%) | Mobile Phase B (%) |
|---|---|---|
| 0 | 100 | 0 |
| 16.0 | 50 | 50 |
| 16.1 | 0 | 100 |
| 18.0 | 0 | 100 |
| 18.1 | 100 | 0 |
| 20.0 | 100 | 0 |

AEC data of Example 28-conjugated (human) is shown in FIG. 1B (n=2) and FIG. 1D (n=4), with a retention time of about 7.5 minutes with 0% aggregation, and about 13 minutes with 0% aggregation, respectively.

Mass Spectroscopy

ADC samples were fully reduced before MS analysis. The mass spectrometry conditions used are as follows: HPLC column=Water BEH300 C4, 2.1×50 mm, 3.5 micron particle size; Mobile phase A: 0.1% formic acid in water; Mobile phase B: 0.1% formic acid in acetonitrile; Flow rate: 450 μL/min; Gradient: 0-0.6 min, 5% B, 0.6 to 1.1 min 5-90% B, 1.1 to 2.2 min 90% B, 2.2 to 2.4 min, 90-5% B, 2.4 to 3.5 min 5% B; Column temperature: 40° C.; MS ionization source: ESI.

Deconvoluted mass spectroscopy data of Example 28-conjugated (human) is shown in FIG. 1C (n=2) and FIG. 1E (n=4).

For FIG. 1C (n=2), the 25176.72 peak corresponds to the light chain (SEQ ID NO: 2) with one drug linker molecule conjugated. The 50954.63 peak corresponds to the heavy chain (SEQ ID NO: 1) with one drug linker molecule conjugated.

For FIG. 1E (n=4), the 25176.88 peak corresponds to the light chain (SEQ ID NO: 2) with one drug linker molecule conjugated. The 50954.80 peak corresponds to the heavy chain (SEQ ID NO: 1) with one drug linker molecule conjugated.

4. Preparation of ADCs Following General Procedure and Example 4 and 28 ADCs

Table 14A provides ADC conjugates synthesized following this General Method, tested from fractions of a population of ADC conjugates (DAR value provided; population comprising a mixture of ADCs, e.g., mixture of n=2, 4, and/or 6; aggregation data also provided). Table 14B provides ADC conjugates which may be synthesized from the Bromo Acetamide Products of Precursor Examples 14A and various Precursors listed in Table 10, following the above General Method. Variable (A) corresponds to the anti-CD40 antibody (human or mouse); n=1, 2, 3, 4, 5, 6, 7, 8, 9, or 10. Human anti-CD40 antibody corresponds to Ab102 (Table 3). Mouse anti-CD40 antibody corresponds to Antibody 138 described in US 20160347850, incorporated herein by reference. P=Precursor Example. Antibody 138 has similar characteristics to Ab102, e.g., antibody 138 is an antagonist antibody with no substantial agonist activity like Ab102. Thus, antibody 138 is representative of Ab102 activity in mouse models.

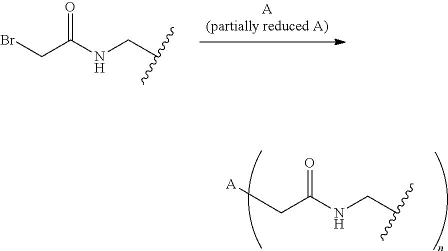

TABLE 14A

| Bromoacetamide ADCs Synthesized | | DAR |
|---|---|---|
| 14B | 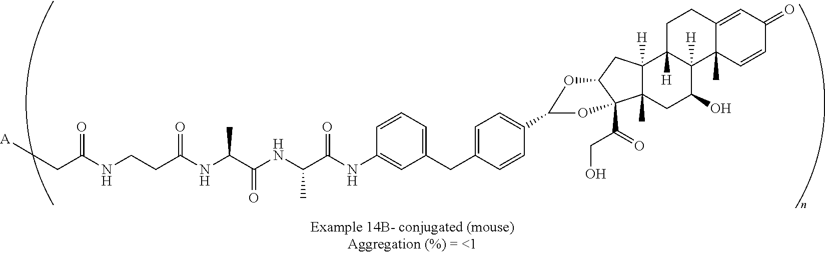 | 3.35 |
| 15 | 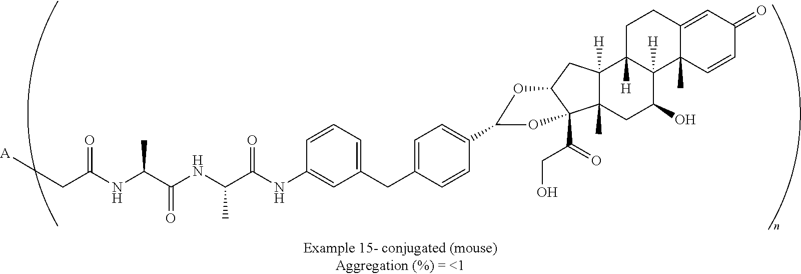 | 3.72 |
| 16 | 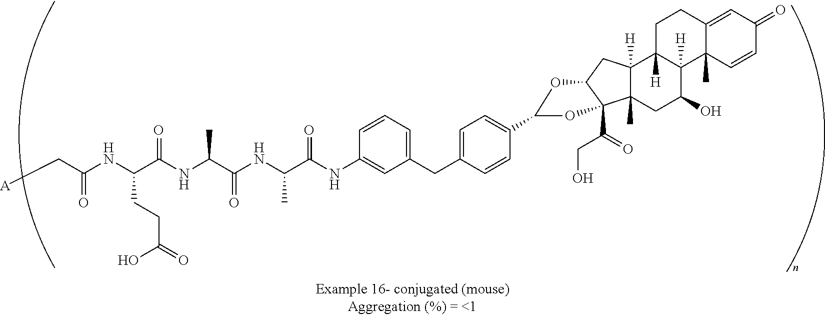 | 3.39 |
| 17 | 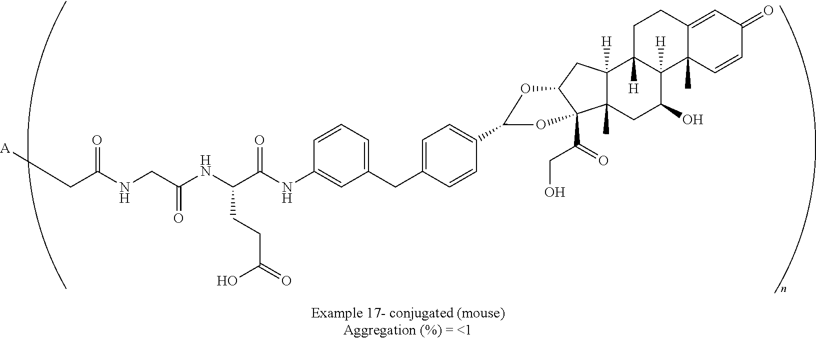 | 3.19 |
| 18B | 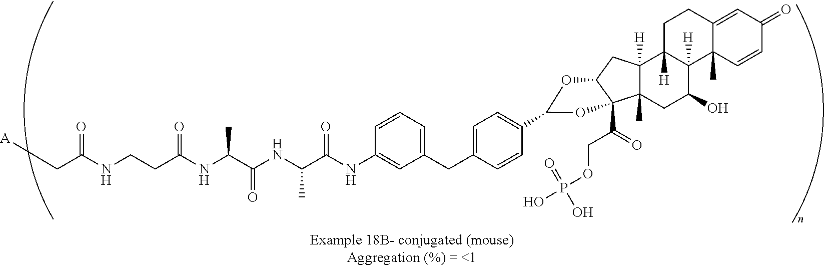 | 3.65 |
| 19 | 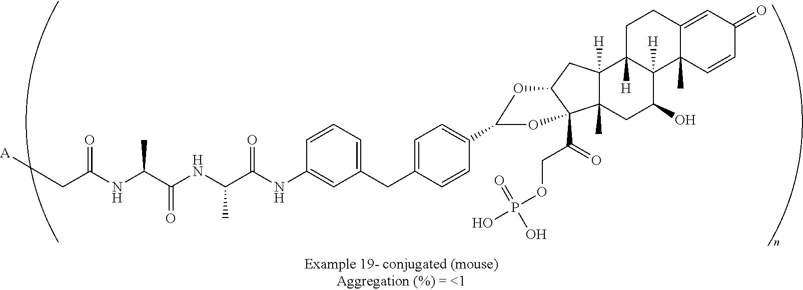 | 3.44 |
| 20 | 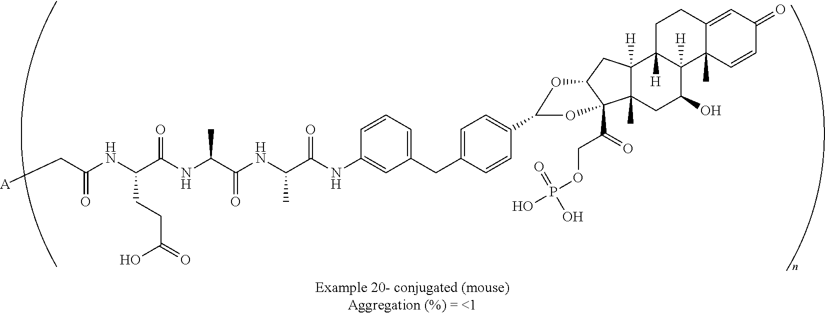 | 3.23 |
| 21B | 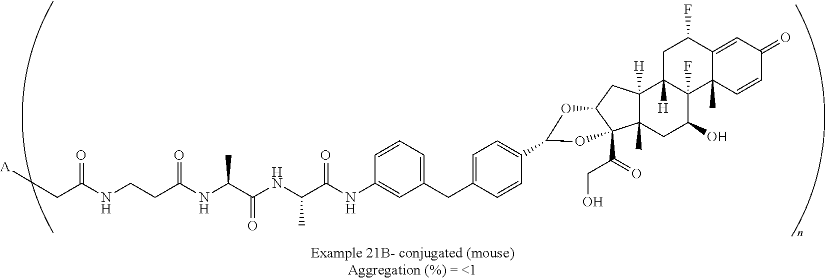 | 3.59 |
| 22 | 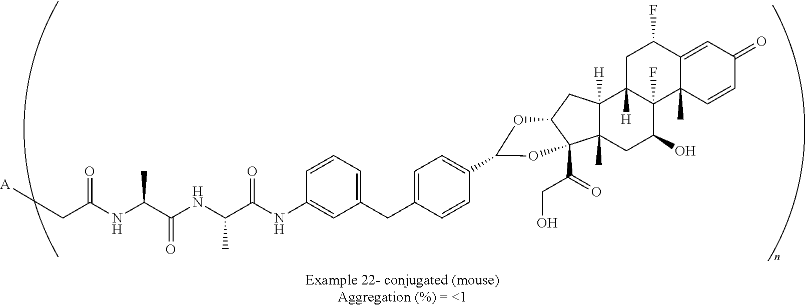 | 3.50 |
| 23 | 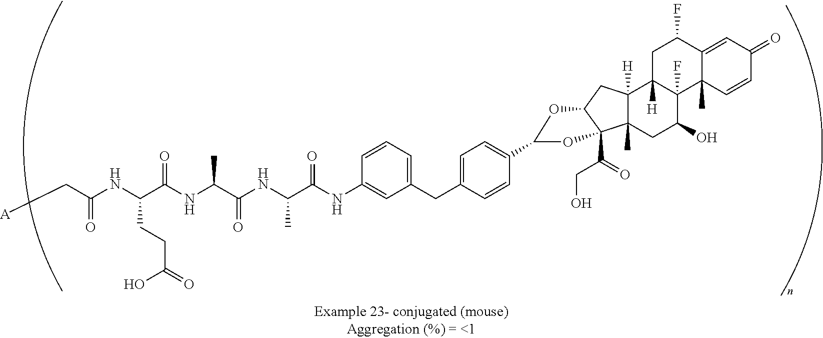 | 3.56 |
| 24 | 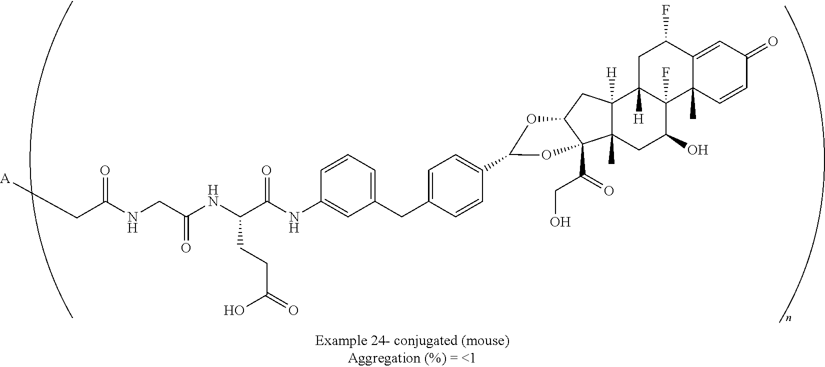 | 3.59 |
| 25B | 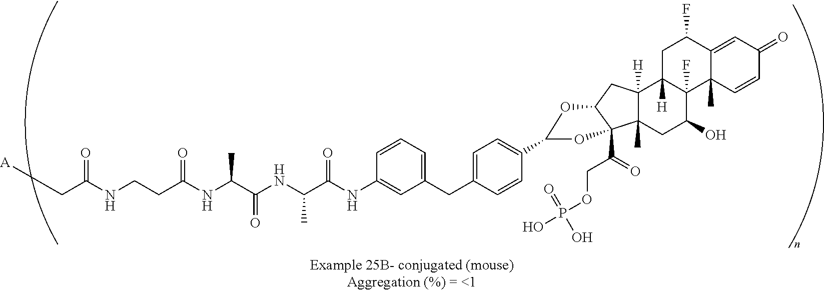 | 3.31 |
| 27 | 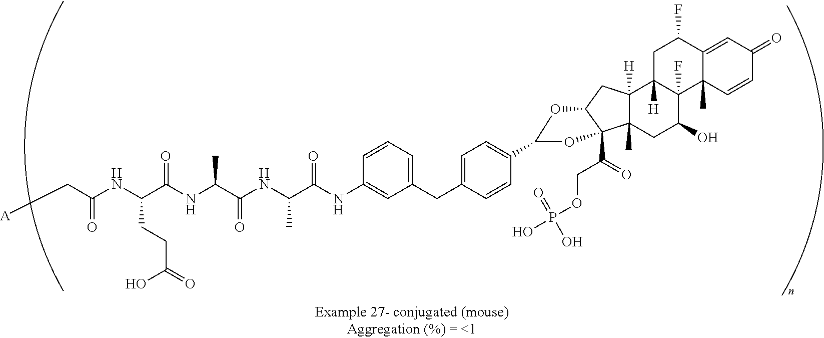 | 3.10 |
| 36 | 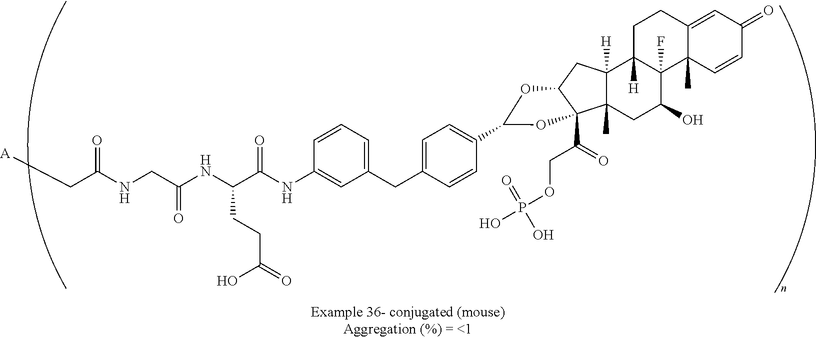 | 3.62 |
| 37 | 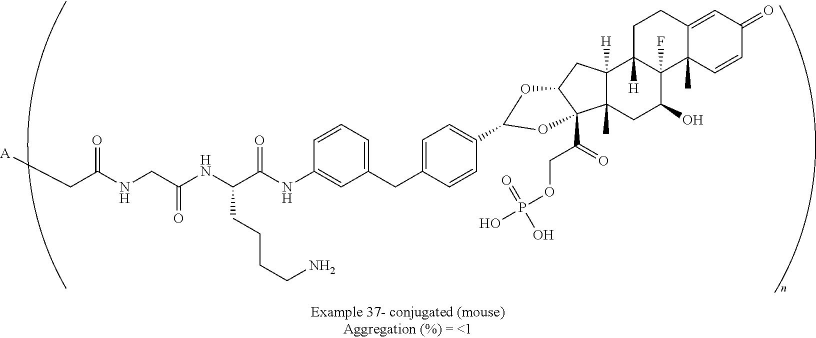 | 3.44 |
| 38 | 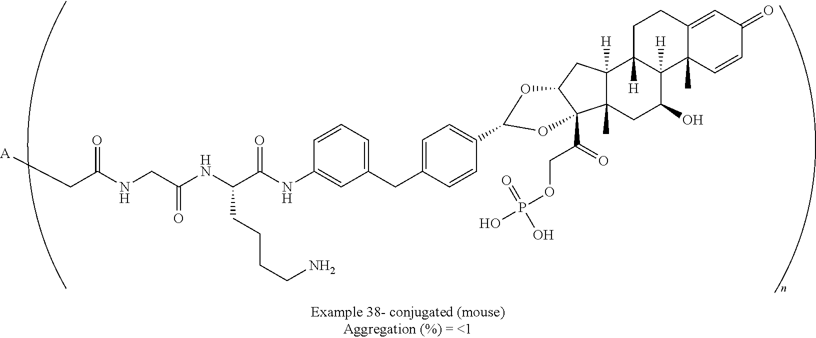 | 3.45 |
| 47 | 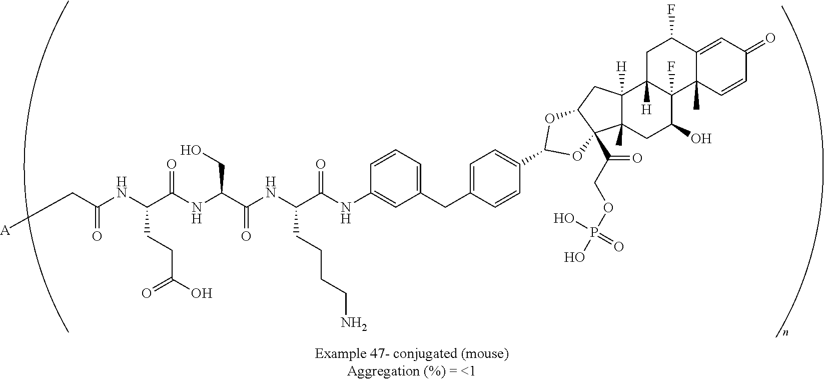 | 3.47 |
| 48 | 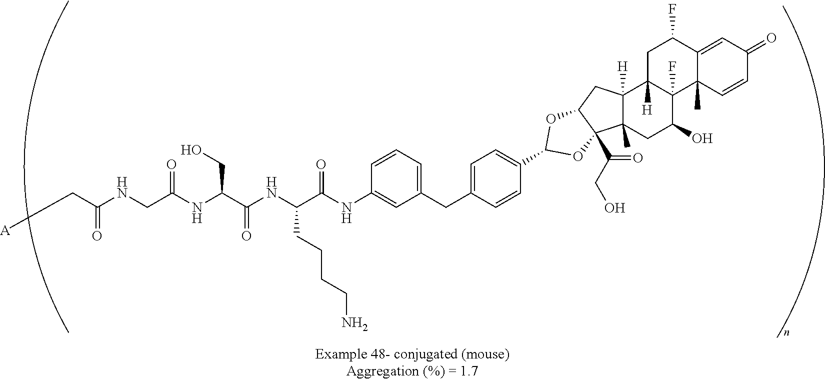 | 2.12 |

TABLE 14B
Additional Bromoacetamide ADCs

| 14A | 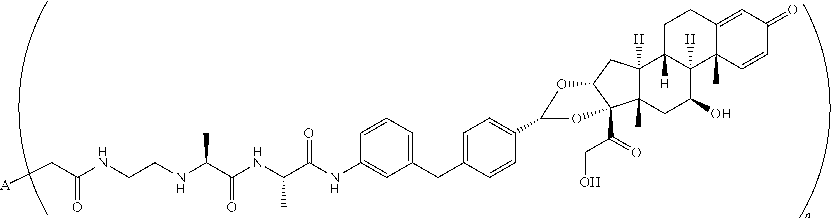 |
|---|---|
| 18A | 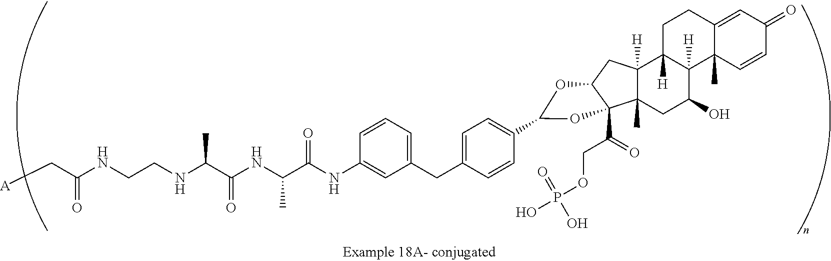 |
| 21A | 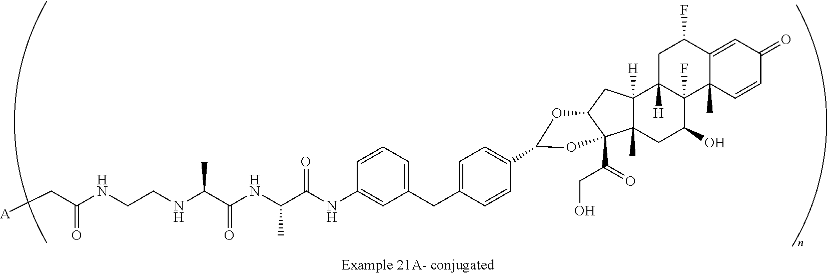 |
| 25A | 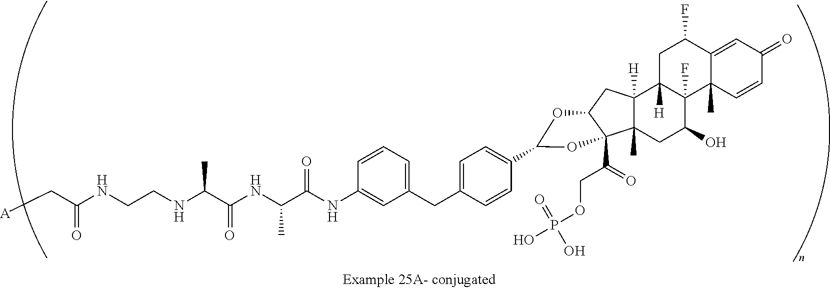 |
| 26 | 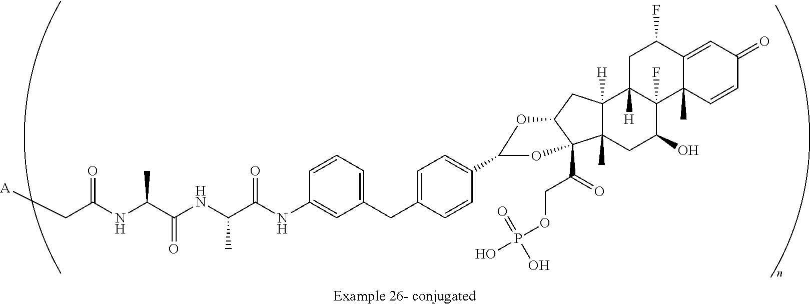 |
| 29A | 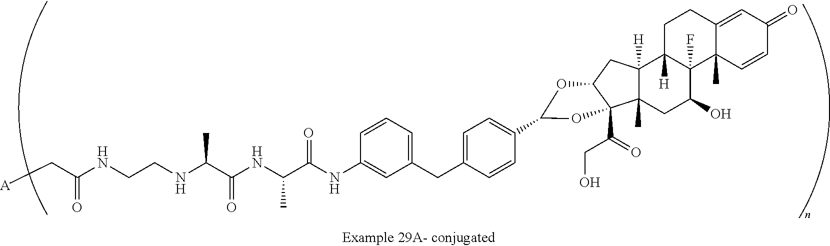 |
| 29B | 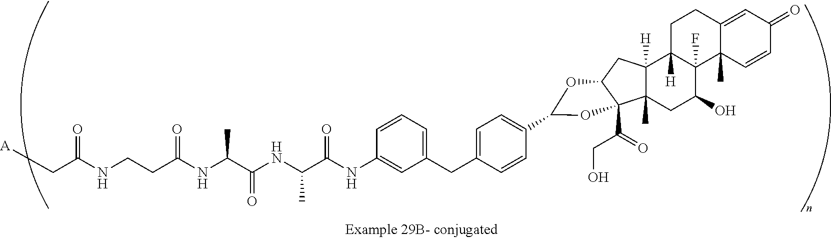 |
| 30 | 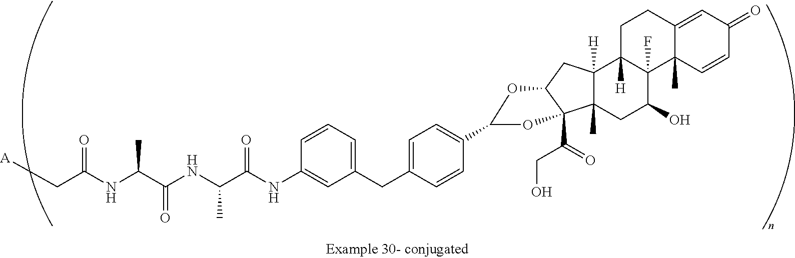 |
| 31 | 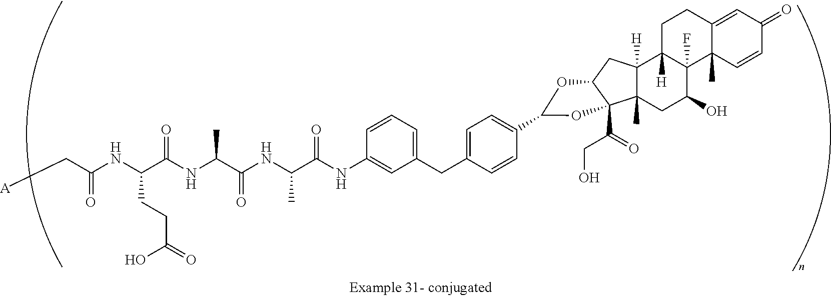 |
| 32 | 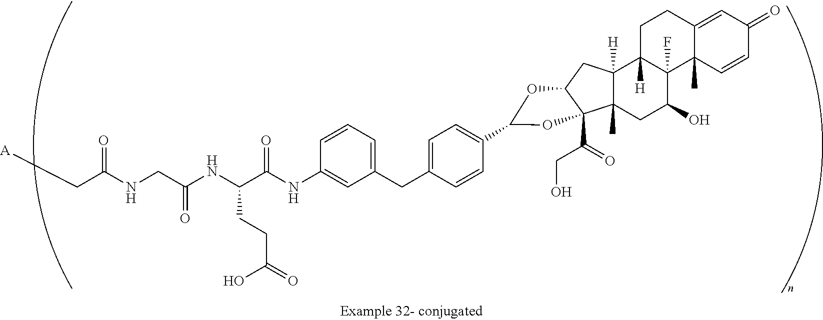 |
| 33A | 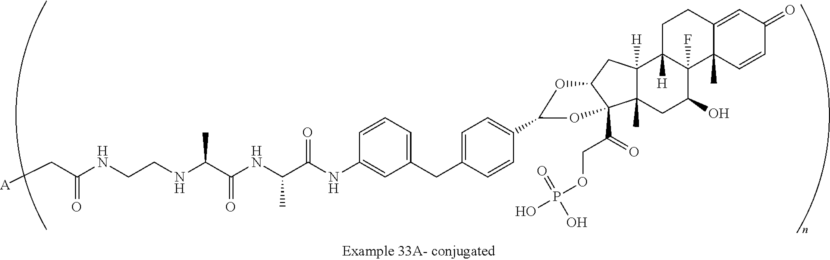 |
| 33B | 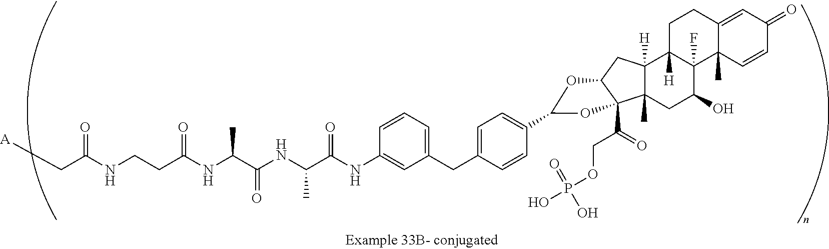 |
| 34 | 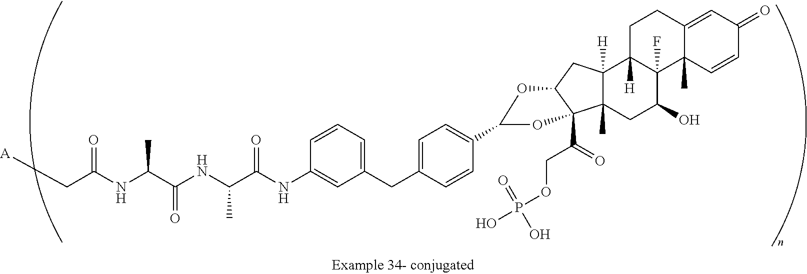 |
| 35 | 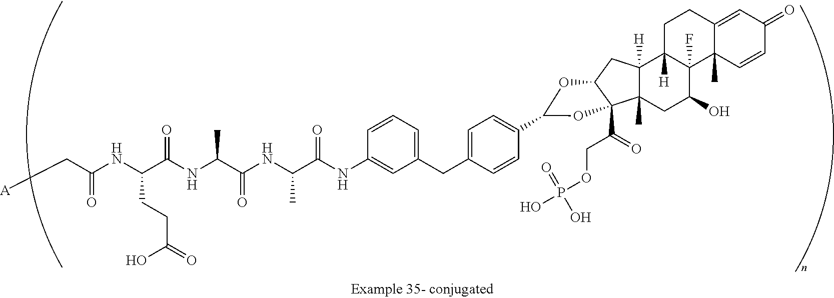 |
| 39 | 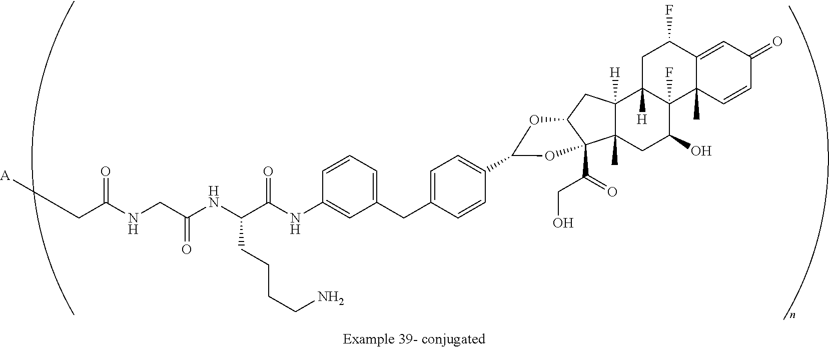 |
| 40 | 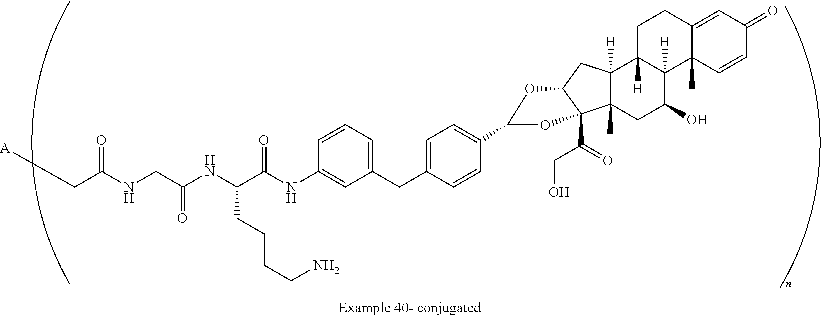 |
| 41 | 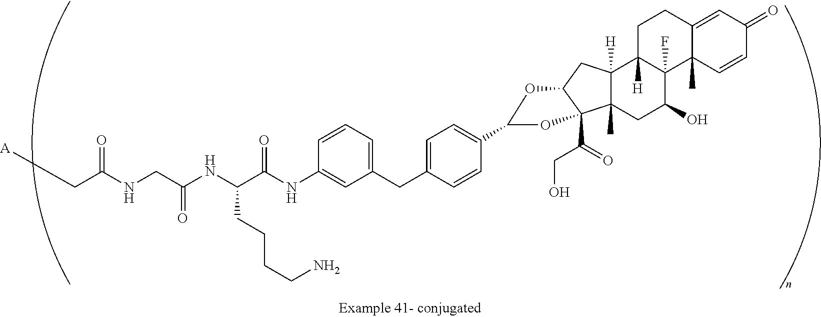 |
| 42 | 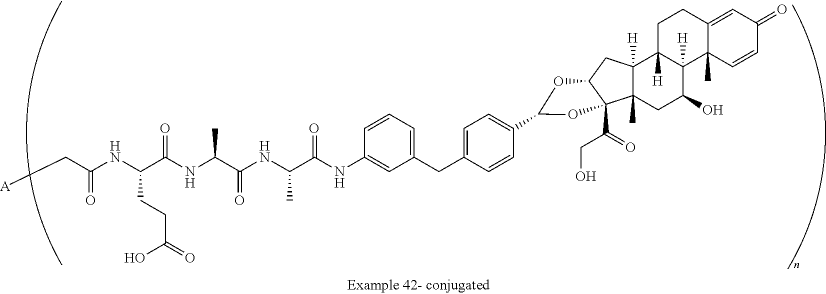 |
| 43 | 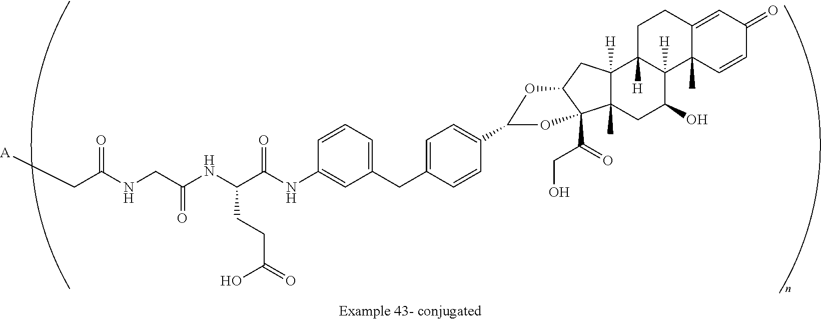 |
| 44 | 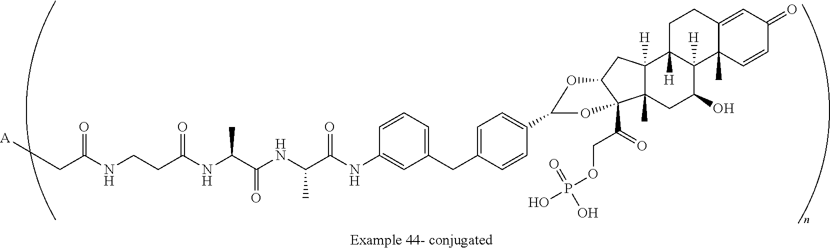 |
| 45 | 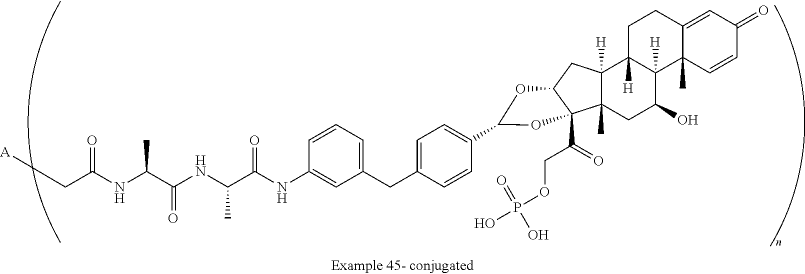 |
| 46 | 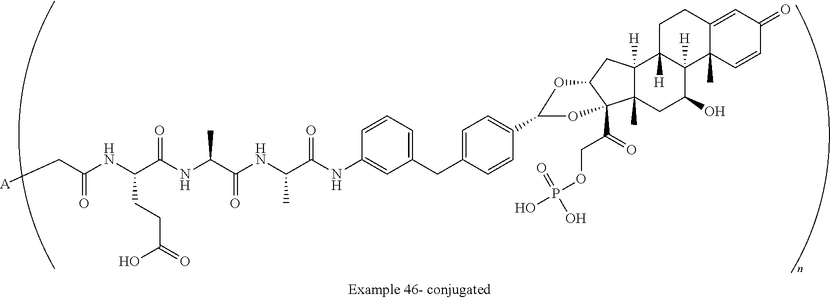 |

### BIOLOGICAL ASSAYS

TABLE 15
Abbreviations

| | | | |
|---|---|---|---|
| MEM | Minimal Essential Media | CS | Charcoal Stripped Fetal |
| | | FBS | Bovine Serum |
| NEAA | Non-essential amino | FBS | Fetal Bovine Serum |
| | acids | | |
| RPMI | Roswell Park Memorial | DBP | Dibutyl phthalate |
| | Institute | | |
| DTT | Dithiothreitol | HEPES | 4-(2-hydroxyethyl)-1- |
| | | | piperazineethanesulfonic |
| | | | acid |
| GM- | Granulocyte-macrophage | CFA | Complete Freund's |
| CSF | colony-stimulating factor | | Adjuvant |
| P1NP | Procollagen type 1 | ACTH | Adrenocorticotropic |
| | amino-terminal propeptide | | Hormone |
| DBP | Dibutyl phthalate | DTT | Dithiothreitol |

### Example A. Generation of Human and Mouse CD40 GRE Reporter Cell Lines

In order to create a parental cell line, HEK293 cells were seeded onto a 6 well dish (Costar: 3516) with 2 mL of complete growth medium (RPMI, 10% FBS, 1% L-glutamine, 1% Na Pyruvate and 1% MEM NEAA) at 250,000 cells per well for 24 hours at 37° C., 5% CO₂. The next day, 3 μg of pGL4.36[Luc2P/MMTV/Hygro] (Promega: E316) and 3 μl of PLUS reagent (Invitrogen: 10964-021) were diluted into 244 μL, Opti-MEM (Gibco: 31985-070) and incubated at room temperature for 15 minutes. The pGL4.36[luc2P/MMTV/Hygro] vector contains MMTV LTR (Murine Mammary Tumor Virus Long Terminal Repeat) that drives the transcription of the luciferase reporter gene luc2P in response to activation of several nuclear receptors such as glucocorticoid receptor and androgen receptor. After incubation, diluted DNA solution was pre-incubated with 1:1 Lipofectamine LTX solution (Invitrogen: 94756) (13.2 μl+256.8 μl Opti-MEM) and incubated at room temperature for 25 minutes to form DNA-Lipofectamine LTX complexes. After incubation, 500 μl of DNA-Lipofectamine complexes were added directly to the well containing cells. HEK293 cells were transfected for 24 hours at 37° C., 5% CO₂. After incubation, cells were washed with 3 mL of phosphate buffer saline (PBS) and selected with complete growth medium containing 100 μg/mL of hygromycin B (Invitrogen: 10687-010) for two weeks. “HEK293 GRE pGL4.36[Luc2P/MMTV/Hygro]” cells were produced.

In order to create a murine CD40 transfected cell line, HEK293 cells were seeded onto 6 well dish (Costar: 3516) with 2 mL of complete growth medium (RPMI, 10% FBS, 1% L-glutamine, 1% Na Pyruvate and 1% MEM NEAA) at 250,000 cells per well for 24 h at 37°, 5% CO₂. The next day, 3 μL of FuGENE 6 Transfection Reagent (Promega: E2311) were diluted into 96 μL of unsupplemented RPMI medium and incubated at room temperature for 5 minutes. After incubation, 1 μg of NEF39 muCD40 HA ICD4 (PDL/FACET Biopharma) was added to the transfection mixture and incubated at room temperature for 30 minutes. After incubation, diluted DNA solution was added dropwise to the well containing cells at 100 μL per well. HEK293 cells were transfected for 24 h at 37°, 5% CO₂. After incubation, cells were washed with 3 mL of PBS and selected with complete growth medium containing 500 ug/mL G418 (Gibco: 10131-027) for two weeks. The resulting cell line was designated “mCD40_HEK293”.

In order to create a murine CD40 GRE reporter cell line, HEK293 cells stably transfected with mCD40 were seeded onto 6 well dish (Costar: 3516) with 2 mL of complete growth medium (RPMI, 10% FBS, 1% L-glutamine, 1% Na Pyruvate and 1% MEM NEAA) at 250,000 cells per well for 24 h at 37°, 5% CO₂. The next day, 3 pg of pGL4.361Luc2P/MMTV/Hygrol (Promega: E316) and 3 uL of PLUS reagent (Invitrogen: 10964-021) were diluted into 244 μL Opti-MEM (Gibco: 31985-070) and incubated at room temperature for 15 minutes. After incubation, diluted DNA solution was pre-incubated with 1:1 Lipofectamine LTX solution (Invitrogen: 94756) (13.2 μL+256.8 μL Opti-MEM) and incubated at room temperature for 25 minutes to form DNA-Lipofectamine LTX complexes. After incubation, 500 μL of DNA-Lipofectamine complexes were added directly to the well containing cells. HEK293 cells were transfected for 24 h at 37°, 5% CO₂. After incubation, cells were washed with 3 mL of PBS and selected with complete growth medium containing 100 μg/mL of hygromycin B (Invitrogen: 10687-010) and 500 μg/mL G418 (Gibco: 10131-027) for two weeks. The resulting cell line was designated “mCD40_HEK293 GRE pGL4.36[Luc2P/MMTV/Hygro]”

In order to create a human CD40 GRE reporter cell line, HEK293 pGL4.361Luc2P/MMTV/Hygrol cells were seeded onto 6 well dish (Costar: 3516) with 1 mL of complete growth medium (RPMI, 10% FBS, 1% L-glutamine, 1% Na Pyruvate and 1% MEM NEAA) at 250,000 cells per well. Then 3 μg of human CD40 Transcript 1 (Myc-DDK-tagged) DNA (Origene Cat #RC201977) and 3 μL of PLUS reagent (Invitrogen: 10964-021) were diluted into 500 μL Opti-MEM (Gibco: 31985-070). The DNA solution was pre-incubated with 1:1 Lipofectamine LTX solution (Invitrogen: 94756) (11 μL+500 μL Opti-MEM) and incubated at room temperature for 15 minutes to form DNA-Lipofectamine LTX complexes. After incubation, 1,000 μL of DNA-Lipofectamine complexes were added directly to the well containing cells. HEK293 pGL4.361Luc2P/MMTV/Hygrol cells were transfected for 24 h at 37°, 5% CO₂. After incubation, cells were washed with 3 mL of PBS and selected with complete growth medium containing 100 μg/mL of hygromycin B (Invitrogen: 10687-010) and 500 μg/mL G418 (Gibco: 10131-027) for two weeks. The resulting cell line was designated “hCD40 transcript 1_HEK293 GRE pGL4.36 [Luc2P/MMTV/Hygro]”

### Example B. Activity of Anti-CD40 ADCs in GRE Reporter Assays

HEK293 parental GRE (pGL4.36[luc2P/MMTV/Hygro]) cells and HEK293 mCD40 or hCD40 GRE (pGL4.36[luc2P/MMTV/Hygro]) cells were plated onto 96 well tissue culture treated white plates (Costar: 3917) at 20,000 cells per well in 75 μL of assay medium (RPMI, 1% CSFBS, 1% L-glutamine, 1% Na Pyruvate and 1% MEAA) and incubated for 24h at 37° C., 5% CO2. The next day, cells were treated with 25 μL of 4× serial diluted murine or human anti-CD40 antibody drug conjugates in assay medium, steroid compound, or media alone and incubated for 72 hours at 37° C., 5% CO₂. After 72 hours of incubation, cells were treated with 100 μL of Dual-Glo Luciferase Assay System (Promega-E2920) for 10 minutes and analyzed for luminescence using the Microbeta (PerkinElmer). Data were analyzed using a four parameter curve fit to generate EC₅₀ values. Percent (%) maximum activation was normalized to 100 nM dexamethasone, which was considered maximum activation. EC₅₀ values for each both anti-mouse CD40 ADC and anti-human CD40 ADC provided in Tables 16 and 17 respectively.

TABLE 16
In vitro activity of anti-mouse CD40
ADCs in human CD40 GRE reporter assay

| | | % | mCD40 | | HEK293 | |
|---|---|---|---|---|---|---|
| | | mono- | GRE | mCD40 | GRE | HEK293 |
| | | mer | EC₅₀ | GRE (% | EC₅₀ | GRE (% |
| ADC | n | (SEC) | (μg/ml) | max) | (μg/ml) | max) |
| Example 6- | 4 | 99.4 | 0.11 | 84.2 | 5.7 | 100 |
| hydrolyzed | | | | | | |
| (mouse) | | | | | | |
| Example 7- | 4 | 100 | 0.42 | 82.2 | >50 | 52.3 |
| hydrolyzed | | | | | | |
| (mouse) | | | | | | |
| Example 12- | 2 | 100 | 0.15 | 104 | 9.79 | 93 |
| hydrolyzed | | | | | | |
| (mouse) | | | | | | |
| Example 12- | 4 | 100 | 0.14 | 108 | 6.6 | 96 |
| hydrolyzed | | | | | | |
| (mouse) | | | | | | |
| Example 28 | 4 | 99.8 | 0.23 | 118 | 19.15 | 83 |
| (mouse) | | | | | | |

TABLE 17
In vitro activity of anti-human CD40
ADCs in human CD40 GRE reporter assay

| | | | hCD40 | | HEK293 | |
|---|---|---|---|---|---|---|
| | | % | GRE | hCD40 | GRE | HEK293 |
| | | mono- | EC₅₀ | GRE (% | EC₅₀ | GRE (% |
| ADC | n | mer | (μg/ml) | max) | (μg/ml) | max) |
| Example 12- | 4 | 98.2 | 0.19 | 163 | >17 | 117 |
| hydrolyzed | | | | | | |
| (human) | | | | | | |
| Example 13- | 4 | 99.8 | 0.29 | 67 | >50 | 27 |
| hydrolyzed | | | | | | |
| (human) | | | | | | |
| Example 28 | 2 | 100 | 16.5 | 90 | >50 | 82 |
| (human) | | | | | | |
| Example 28 | 4 | 100 | 0.53 | 77 | >50 | 64 |
| (human) | | | | | | |
| Example 4 | 4 | 100 | 6.57 | 88 | >50 | 21 |
| (human) | | | | | | |

SEC = as measured by size exclusion chromatography

### Example C. Activity of Anti-CD40 ADCs in Lipopolysacharride and Soluble CD40 Ligand-Stimulated Human Monocyte-Derived DC Cytokine Release Assay

Primary human peripheral blood mononuclear cells (PBMCs) were purchased from Sanguine Biosciences, washed in 50 mL phosphate buffer saline (PBS) (pH 7.2), re-suspended in 100% FBS with 5% DMSO, aliquoted and cryopreserved in liquid nitrogen until use. The PBMCs were thawed and washed in PBS (pH 7.2) with 0.5% FBS and 2 mM EDTA. Monocytes from PBMCs were enriched by positive selection of CD14+ cells using the Miltenyi Whole Blood CD14 MicroBeads kit (Cat #130-090-879) and the Miltenyi autoMACS Pro Separator according to manufacturer's protocol. Purified monocytes were washed and re-suspended in RPMI supplemented with 10% FBS, L-glutamine (Gibco Cat #25030081), sodium pyruvate (Gibco Cat #11360070), MEM non-essential amino acids solution (Gibco Cat #11140050), Penicillin-Streptomycin (Gibco Cat #15140122), HEPES buffer (Gibco Cat #15630080), 2-mercaptoethanol (Gibco Cat #21985023). The cells were transferred to 6-well plates (Corning Cat #3506) at 1.00E+06 cells per mL and 3 mL per well, and incubated with 100 ng/mL rhGM-CSF (R&D Systems, Cat #215-GM-010/CF) and 100 ng/mL rhIL-4 (R&D Systems, Cat #204-IL-010/CF) at 37° C. and 5% CO₂ for 5 days to induce differentiation of monocytes into dendritic cells (DCs). On Day 5, semi-adherent monocyte-derived DCs (MoDCs) were pooled and the efficiency of their differentiation was confirmed by phenotyping for CD1a-positive CD14-negative cells (Biolegend Cat #300106, Cat #325628) using flow cytometry. MoDCs were washed and re-suspended in supplemented RPMI media and plated into a cell assay plate (Costar Cat #3799) at 1.0E+05 cells per well. The cells were stimulated with lipopolysaccharide (LPS) (Sigma Cat #L4391-1MG) at 0.1 ng/mL for 2 hours to induce up-regulation of cell-surface CD40 expression on MoDCs. Following stimulation, the culture supernatant was washed and cells were incubated with varying concentrations of anti-human CD40 antibody or anti-human CD40 ADCs at 37° C. and 5% CO₂ for 2 hours. Cells were then stimulated with 0.2 ng/mL LPS and 0.5 ug/mL soluble CD40-ligand (CD40L) (Adipogen Cat #AG-40B-0010) for 20 hours. Following incubation, plate was spun for five minutes at 1200 rpm, and 150 uL of supernatant media was directly transferred to an additional 96-well plate and analyzed for IL-6 (MSD, #K151AKB) concentrations. The dose response data were fitted to a sigmoidal curve using nonlinear regression, and the IC₅₀ values calculated with the aid of GraphPad Prism 6 (GraphPad Software, Inc.). The results shown in Table 18 demonstrate that the anti-human CD40 ADC has potent activity in inhibiting the release of pro-inflammatory cytokine IL-6 from activated primary immune cells and the potency difference between Example 13-hydrolized (human) and Example 12-hydrolyzed (human) ADCs, wherein n is 4, corresponds to the potency differences between the two payload compounds. Table 18 also provides similar results for Example 28-conjugated (human) ADC wherein n is 2, and Example 28-conjugated (human) ADC wherein n is 4. A representative example of results shown in FIG. 2 demonstrates that the maximum capacity to inhibit immune cell activation by either of Example 13-hydrolized (human) and Example 12-hydrolyzed (human), wherein n is 4, exceeds inhibition provided by the parental antagonist antibody.

TABLE 18
In vitro activity of anti-human CD40 ADC in LPS and CD40L-
stimulated human MoDC cytokine release assay (N = 3)

| | % | IL-6 | Max | |
|---|---|---|---|---|
| | monomer | release | Inhibition | |
| Control or ADC | (SEC) | IC₅₀ (μg/ml) | (%) | n |
| CONTROL 1 | 100 | 0.18 | 44.5 | NA |
| hCD40 mAb | | | | |
| CONTROL 2* | 97.7 | 3.3 | 18.2 | 4 |
| Example 12 - hydrolyzed | | | | |
| isotype | | | | |
| (Ab = anti-tetanus toxoid, | | | | |
| human isotype)* | | | | |
| Example 13-hydrolyzed | 99.8 | 0.14 | 83.3 | 4 |
| (human) | | | | |
| Example 12-hydrolyzed | 98.2 | 0.04 | 99.5 | 4 |
| (human) | | | | |
| Example 28-conjugated | 100 | 0.03 | 81.8 | 2 |
| (human) | | | | |
| Example 28-conjugated | 100 | 0.05 | 87.6 | 4 |
| (human) | | | | |

*Isotype antibodiesare antibodies that targets tetanus toxoid and is used as a control for effect of administering IgG that does not recognize an antigen present in the xenograft model. See, e.g., US 20170182179. The above described human isotype ADC was derived from the cloned variable domains of a human antibody that recognizes the tetanus toxoid vaccine. This is an antigen not expected to be expressed by human cells in vitro or in vivo.
SEC = as measured by size exclusion chromatography

### Example D. Activity of Anti-Mouse CD40 ADCs in Bone-Marrow Derived DC Activation Assay

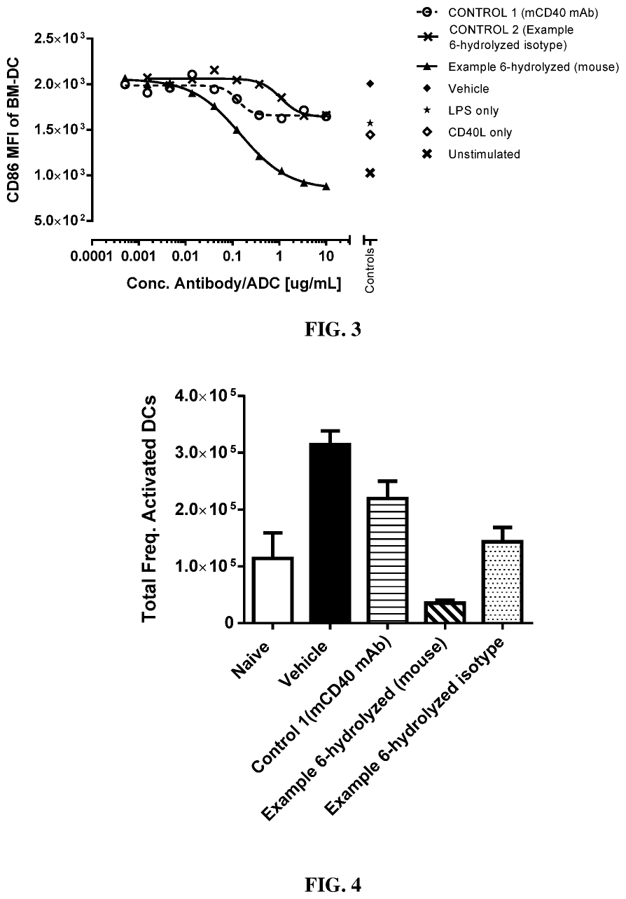

Murine bone marrow (BM) cells were extruded from femurs and tibias of C57BL/6 mice and re-suspended in supplemented RPMI media. The cells were transferred to 6-well plates (Corning Cat #3506) at 1.00E+06 cells per mL and 5 mL per well and incubated with 10 ng/mL murine GM-CSF (R&D Systems Cat #415-ML-010) at 37° C. and 5% CO₂ for 8 days. On days 3 and 5 of culture, ⅔ of the culture media was replaced with fresh GM-CSF containing medium supplemented with 20 ng/mL IL-4 to induce differentiation of BM cells into dendritic cells (DCs). Following incubation, these BM-derived DCs (BMDCs) were washed and re-suspended in supplemented RPMI media and plated into a cell assay plate (Costar Cat #3799). The cells were stimulated with lipopolysaccharide (LPS) (Sigma Cat #L4391-1MG) at 0.1 ng/mL for 2 hours to induce up-regulation of cell-surface CD40 expression on BMDCs. Following stimulation, the culture supernatant was washed and cells were incubated with varying concentrations of anti-mouse CD40 antibody or Example 6-hydrolyzed (mouse) at 37° C. and 5% CO₂ for 2 hours. Cells were then stimulated with 0.1 ng/mL LPS and 0.5 ug/mL soluble CD40-ligand (CD40L) (Enzo Life Sciences, Inc. Cat #ALX-522-120-C010) for 20 hours. In some experiments, LPS treatment was tested at varying concentrations (0.1, 1.0, 10 ng/mL) while soluble CD40L remained at 0.5 μg/mL. Following incubation, plate was spun for five minutes at 1200 rpm, and 150 uL of supernatant media was directly transferred to an additional 96-well plate and analyzed for IL-6 (MSD, Cat #K152TXK) concentrations. To quantify up-regulation of DC activation markers, cultured cells remaining in the assay plate were washed, stained with anti-mouse CD86 antibody (GL-1, Biolegend Cat #105018) and assessed by flow cytometry. The dose response data were fitted to a sigmoidal curve using nonlinear regression, and the IC50 values calculated with the aid of GraphPad Prism 6 (GraphPad Software, Inc.). Additional experiments were conducted with Example 12-hydrolyzed (mouse) and Example 28 (mouse). The results shown in Table 19 demonstrate that the anti-mouse CD40 ADCs exhibit potent activity in suppressing up-regulation of co-stimulatory molecule expression on activated primary immune cells and the potency differences between the ADCs corresponds to the potency differences between the drug-linker payload. The results shown in FIG. 3 demonstrate that the maximum capacity to inhibit immune cell activation by Example 6-hydrolyzed (mouse) exceeds inhibition provided by the parental antagonist antibody.

TABLE 19
In vitro activity of anti-mouse CD40 ADC in LPS and
CD40L-stimulated mouse BMDC activation assay (N = 3)

| | % | CD86 | Max | |
|---|---|---|---|---|
| | monomer | expression | Inhibition | |
| CONTROL OR ADC | (SEC) | IC₅₀ (μg/ml) | (%) | n |
| CONTROL 1 | 100 | 0.13 | 35.6 | NA |
| mCD40 mAb | | | | |
| CONTROL 2* | 100 | 1.06 | 37.5 | 4 |
| Example 6- hydrolyzed | | | | |
| isotype | | | | |
| (Ab = anti-tetanus toxoid, | | | | |
| mouse isotype) | | | | |
| Example 6-hydrolyzed | 100 | 0.15 | 118.4 | 4 |
| (mouse) | | | | |
| Example 12-hydrolyzed | 100 | 0.06 | 94.2 | 4 |
| (mouse) | | | | |
| Example 28 (mouse) | 99.8 | 0.10 | 90.8 | 4 |

*Isotype antibodiesare antibodies that targets tetanus toxoid and is used as a control for effect of administering IgG that does not recognize an antigen present in the xenograft model. See, e.g., US 20170182179. The above-described mouse isotype ADC was derived from the cloned variable domains of a mouse antibody that recognizes the tetanus toxoid vaccine. This is an antigen not expected to be expressed by mouse cells in vitro or in vivo.
SEC = as measured by size exclusion chromatography

### Example E. Activity of Anti-Mouse CD40 ADC in LPS-Induced Acute Inflammation Model In Vivo

C57BL/6 female mice (n=3) were dosed intraperitoneally with 100 μL of phosphate buffer saline (PBS) (pH7.2) containing 1 μg of LPS and either (1) parental antagonist antibody (mCD40 mAb) as Control 1, (2) Example 6-hydrolyzed isotype (Ab=anti-tetanus toxoid, mouse isotype) (n=4), or (3) Example 6-hydrolyzed (mouse) as the ADC (10 mg/kg) (n=4). At 24 hours post injection, the spleens were harvested from treated mice and processed to obtain single-cell suspension from each individual mouse. Cells were stained with the following fluorochrome-labeled antibodies to phenotype specific antigen-presenting cell populations using flow cytometry: anti-mouse CD4 PE (Biolegend Cat #100408), anti-mouse CD8 BUV395 (BD Cat #563786), anti-mouse CD19 PE-Cy7, anti-mouse CD11c PerCp-Cy5.5, anti-mouse CD11b BV510, anti-mouse IAIE Pacific Blue, anti-mouse CD40 APC, anti-mouse CD86 Alexa-488. Cells were stained at 1.0E+07 cells per mL in PBS (pH7.2) containing 1% FBS and FcR-blocking reagent (BD, Cat #553142). The total frequency of activated dendritic cells (DC) per spleen were calculated and plotted with the aid of GraphPad Prism 6 (GraphPad Software, Inc.). The results shown in FIG. 4 demonstrate that the CD40 ADC exhibits greater efficacy in suppressing DC activation in vivo than the parental antagonist antibody or isotype ADC.

### Example F. Activity of Anti-Mouse CD40 ADCs in Delayed Type IV Hypersensitivity Model

Anti-mouse CD40 ADCs were evaluated in an acute delayed type-IV hypersensitivity (DTH) model. A T cell-driven acute inflammatory response of the skin elicited by re-exposure to sensitized protein antigen (BSA). The efficacy of anti-mouse CD40 ADCs was measured by the ability to inhibit paw swelling.

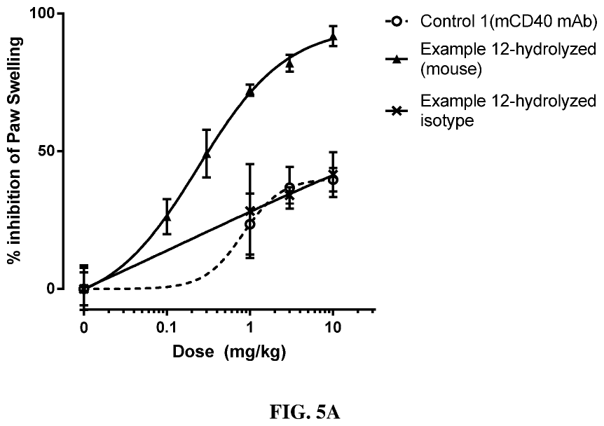

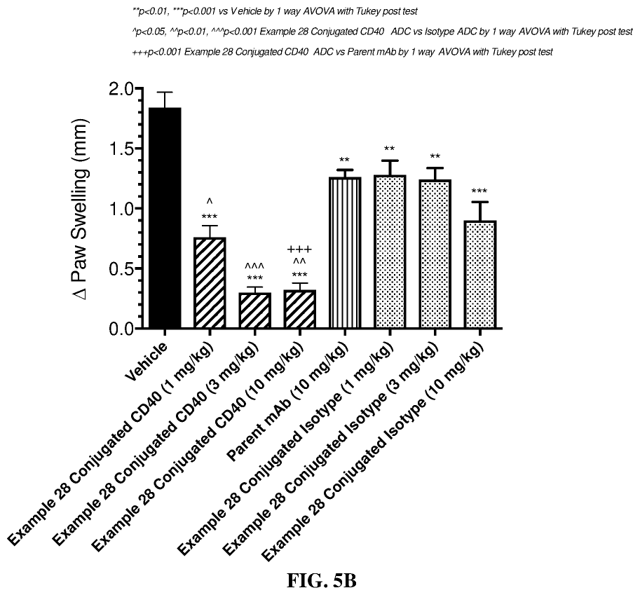

C57BL/6 female mice were dosed intraperitoneally on Day −1 with (1) mCD40 mAb as Control 1; (2) Example 12 hydrolyzed isotype (Ab=anti-tetanus toxoid, mouse isotype) as Control 2 (n=4); or (3) Example 12-hydrolyzed (mouse) as the ADC (n=4). On Day 0, mice were sensitized via immunization using 200 μg of methylated BSA (Sigma-Aldrich, Cat #1009) emulsified in CFA H37Ra (Becton Dickenson, Cat #231131). On Day 7, baseline thickness of both hind paws was measured. Right foot pad was challenged with 100 μg mBSA in phosphate buffer saline (PBS), while the left foot pad was treated with PBS alone. 24 hours post challenge, rear paws were evaluated for paw swelling using Dyer spring calipers (Dyer 310-115) and changes in thickness relative to baseline are plotted in FIG. 5A. After paw swelling measurement, mice were injected with ACTH at 1 mpk IP, and terminally bled at 30 min post-ACTH. Plasma was collected and analyzed for P1NP, corticosterone, free steroid, and large molecule levels. Data in FIG. 5A demonstrates the enhanced efficacy of CD40 ADC to more potently inhibit T-cell mediated inflammation in vivo than parental antagonist antibody or non-targeted ADC alone.

The activity of anti-mouse CD40 ADC consisting of Example 28-conjugated (mouse) to anti-mouse CD40 or isotype (Ab=anti-ovalbumin, mouse isotype) were also evaluated in the DTH assay following the procedure set forth above. FIG. 5B demonstrates enhanced efficacy of CD40 ADC to inhibit T-cell mediated inflammation in vivo than parental antagonist antibody or non-targeted ADC alone.

### Example G. Steroid Biomarkers in DTH Model of Inflammation

1. Plasma P1NP

Quantification of plasma P1NP was conducted on a LC/MS platform based on protein trypsin digestion. Plasma samples were partially precipitated and fully reduced by adding MeCN/0.1M ammonium bicarbonate/DTT mixture. Supernatant was collected and alkylated by adding iodoacetic acid. The alkylated proteins were digested by trypsin and resulting tryptic peptides were analyzed by LC/MS.

Calibration curves were generated by using synthetic tryptic peptide spiked into horse serum (noninterfering surrogate matrix). Stable isotope labeled flanking peptide (3-6 amino acids extension on both termini of the tryptic peptide) was used as internal standard added in the MeCN/DTT protein precipitation mixture to normalize both digestion efficiency and LC/MS injection. Columnex Chromenta BB-C18, 2.1×150 mm, 5 μm column was used for chromatography separation. The mobile phase A was 0.1% formic acid in Milli Q HPLC water and mobile phase B was 0.1% formic acid in MeCN. A linear gradient from 2% of mobile phase B to 65% mobile phase B was applied from 0.6 to 3 min. The total run time was 8 min at a flow rate of 0.45 mL/min. An AB Sciex 4000Qtrap mass spectrometer was used in positive MRM mode to quantify P1NP peptides, at source temperature of 700° C.

2. Released Free Steroid and Endogenous Corticosterone

Calibration curve of steroid was prepared in mouse plasma with final concentrations from 0.03 nM to 0.1 μM at 8 different concentration levels. Corticosterone calibration curve ranging from 0.3 nM to 1 μM final corticosterone concentrations was prepared in 70 mg/mL bovine serum albumin solution in phosphate buffer saline (PBS). A solution of 160 μL MeCN with 0.1% formic acid was added to 40 μL study plasma samples or calibration standards. Supernatants were diluted with distilled water and 30 μL final sample solution was injected for LC/MS analysis

Quantification of released free steroid and corticosterone was conducted on an AB Sciex 5500 triple quadruple mass spectrometer connected to a Shimadzu AC20 HPLC system interfaced with an electrospray ionization source operating in positive mode. A Waters XBridge BEH C18, 2.1×30 mm, 3.5 μm column was used for chromatography separation. The mobile phase A was 0.1% formic acid in Milli Q HPLC water, and mobile phase B was 0.1% formic acid in MeCN. A linear gradient from 2% of mobile phase B to 98% mobile phase B was applied from 0.6 to 1.2 minutes. The total run time was 2.6 min at a flow rate of 0.8 mL/min. The mass spectrometer was operated in positive MRM mode at source temperature of 700° C. The data in Table 20 demonstrates that ADC treatment in the DTH model does not significantly impact the serum levels of steroid biomarkers, P1NP and corticosterone.

TABLE 20
Effect of anti-mouse CD40 ADC activity on steroid biomarkers

| | | P1NP | Corticosterone |
|---|---|---|---|
| Control/ADC | n | (ng/ml ± SD) | (ng/ml ± SD) |
| Vehicle | 4 | 1294 ± 183 | 292.832 ± 40 |
| Example 12-hydrolyzed | 4 | 1096.4 ± 306 | 312.526 ± 46 |
| (mouse), 10 mpk | | | |
| Example 12-hydrolyzed | 4 | 1486.6 ± 313 | 332.304 ± 27 |
| (mouse), 3 mpk | | | |
| Example 12-hydrolyzed | 4 | 1255.4 ± 318 | 303.742 ± 40 |
| (mouse), 1 mpk | | | |
| Example 12-hydrolyzed | 4 | 1736 ± 197 | 271.182 ± 83 |
| (mouse), 0.3 mpk | | | |
| Example 12-hydrolyzed | 4 | 1311.2 ± 418 | 282.856 ± 43 |
| (mouse), 0.1 mpk | | | |

### Example H. Activity of Anti-Mouse CD40 Immunoconjugate in Collagen-Induced Arthritis (CIA)

The ability of Example 6-hydrolyzed (mouse) ADC to impact disease was assessed in the collagen-induced arthritis (CIA) model of arthritis.

In these experiments, male DBA/1J mice were obtained from Jackson Labs (Bar Harbor, Me.). Mice were used at 6 to 12 weeks of age. All animals were maintained at constant temperature and humidity under a 12-hour light/dark cycle and fed with rodent chow (Lab Diet 5010 PharmaServ, Framingham, Mass.) and water ad libitum. AbbVie is AAALAC (Association for Assessment and Accreditation of Laboratory Animal Care) accredited, and all procedures were approved by the Institutional Animal Care and Use Committee (IACUC) and monitored by an attending veterinarian. Body weight and condition were monitored, and animals were euthanized if exhibiting >20% weight loss.

The male DBA/J mice were immunized intradermally (i.d.) at the base of the tail with 100 μL of emulsion containing 100 μg of type II bovine collagen (MD Biosciences) dissolved in 0.1 N acetic acid and 200 μg of heat-inactivated Mycobacterium tuberculosis H37Ra (Complete Freund's Adjuvant, Difco, Laurence, Kans.). Twenty-one days after immunization with collagen, mice were boosted IP with 1 mg of Zymosan A (Sigma, St. Louis, Mo.) in phosphate buffer saline (PBS). Following the boost, mice were monitored 3 to 5 times per week for arthritis. Rear paws were evaluated for paw swelling using Dyer spring calipers (Dyer 310-115).

Mice were enrolled between days 24 and 28 at the first clinical signs of disease and distributed into groups of equivalent arthritic severity. Early therapeutic treatment began at the time of enrollment.

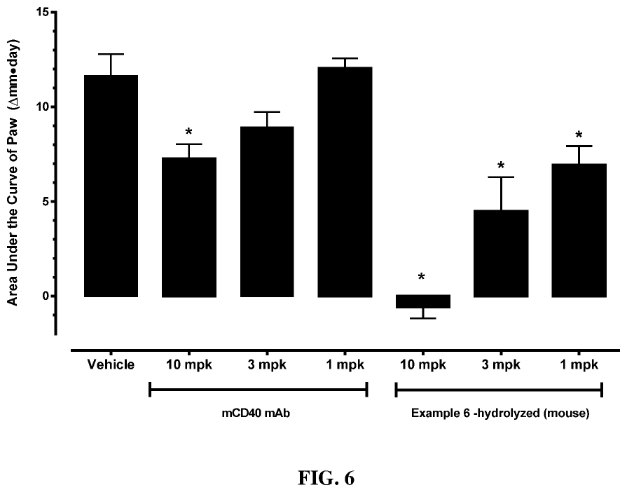

Animals were dosed intraperitoneally with anti-mouse CD40 antagonist antibody at 10 mg/kg or Example 6-hydrolyzed (mouse) ADC (n=4) in 0.9% saline. Blood was collected for antibody exposure by tail nick at 24 and 72 hours after dose. Paws were collected at the terminal timepoint for histopathology. Blood was collected at the terminal timepoint by cardiac puncture for complete blood counts (Sysmex XT-2000iV). Statistical significance was determined by ANOVA. Example 6 hydrolyzed isotype (Ab=anti-tetanus toxoid, mouse isotype) (n=4) and parental anti-mCD40 mAb were used as Controls 1 and 2. The results shown in FIG. 6 demonstrate that a single dose of anti-mouse CD40 steroid ADC can exhibit an extended duration of action through amelioration of paw swelling for ˜6 weeks compared to the Controls 1 and 2.

### INCORPORATION BY REFERENCE

All publications, including patents and published applications, referred to in the Detailed Description and Examples are incorporated by reference herein in their entirety.

### OTHER EMBODIMENTS

The foregoing has been described of certain non-limiting embodiments of the present disclosure. Those of ordinary skill in the art will appreciate that various changes and modifications to this description may be made without departing from the spirit or scope of the present disclosure, as defined in the following claims.

1. An antibody drug conjugate comprising:
(a) an anti-CD40 antibody comprising complementarity determining regions (CDRs) as set forth as SEQ ID NO: 7, SEQ ID NO: 8, SEQ ID NO: 9, SEQ ID NO: 10, SEQ ID NO: 11, and SEQ ID NO: 12; and
(b) a radical of a glucocorticoid receptor agonist of Formula (I): 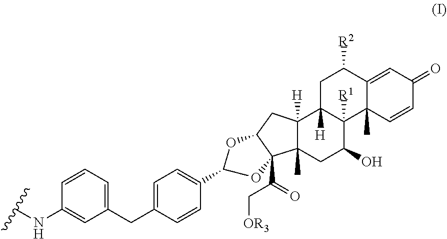
wherein:
R¹ is hydrogen or fluoro;
R² is hydrogen or fluoro; and
R³ is hydrogen or —P(═O)(OH)₂;
further wherein the antibody is conjugated to the glucocorticoid receptor agonist via a linker represented by the following formula: 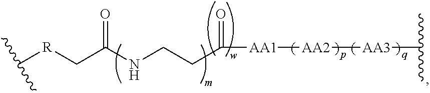
wherein R is a bond, 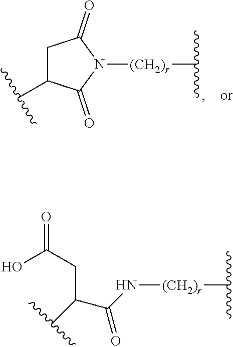
and r is 0 or 1;
AA1, AA2, and AA3 are independently selected from the group consisting of Alanine (Ala), Glycine (Gly), Isoleucine (Ile), Leucine (Leu), Proline (Pro), Valine (Val), Phenylalanine (Phe), Tryptophan (Trp), Tyrosine (Tyr), Aspartic acid (Asp), Glutamic acid (Glu), Arginine (Arg), Histidine (His), Lysine (Lys), Serine (Ser), Threonine (Thr), Cysteine (Cys), Methionine (Met), Asparagine (Asn), and Glutamine (Gln);
m is 0 or 1;
w is 0 or 1;
p is 0 or 1; and
q is 0 or 1.

2. The antibody drug conjugate of claim 1, according to the formula: 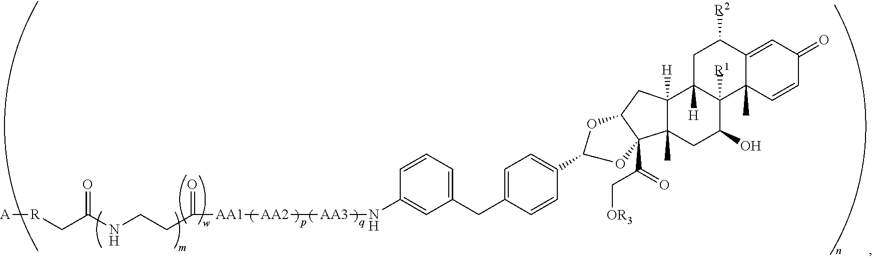
wherein A is the anti-CD40 antibody and n is 1, 2, 3, 4, 5, 6, 7, 8, 9, or 10.

3. The antibody drug conjugate of claim 1, wherein R¹ is hydrogen and R² is hydrogen.

4. The antibody drug conjugate of claim 1, wherein R¹ is fluoro and R² is hydrogen.

5. The antibody drug conjugate of claim 1, wherein R¹ is fluoro and R² is fluoro.

6. The antibody drug conjugate of claim 1, wherein R³ is —P(═O)(OH)₂.

7. The antibody drug conjugate of claim 1, wherein R³ is hydrogen.

8. The antibody drug conjugate of claim 1, wherein -AA1-(AA2)ₚ-(AA3)_{q}- is selected from the group consisting of -Gly-Glu-; -Ala-Ala-; -Glu-Ala-Ala-; -Gly-Lys-; -Glu-; -Glu-Ser-Lys-; and -Gly-Ser-Lys-.

9.-12. (canceled)

13. The antibody drug conjugate of claim 2, wherein:
m is 0 or 1;
p is 1; and
R is a bond

14. The antibody drug conjugate of claim 2, wherein R is a bond, p is 1, m is 0, w is 0, and q is 0.

15. The antibody drug conjugate of claim 2, wherein R is a bond, p is 1, m is 0, w is 0, and q is 1.

16.-17. (canceled)

18. The antibody drug conjugate of claim 1, selected from the group consisting of compounds listed in Table 5, wherein n is 1, 2, 3, 4, 5, 6, 7, 8, 9, or 10.

19. (canceled)

20. The antibody drug conjugate of claim 1, selected from the group consisting of compounds listed in Table 6A, wherein n is 1, 2, 3, 4, 5, 6, 7, 8, 9, or 10.

21. (canceled)

22. The antibody drug conjugate of claim 1, selected from the group consisting of compounds listed in Table 6B, wherein n is 1, 2, 3, 4, 5, 6, 7, 8, 9, or 10.

23.-24. (canceled)

25. The antibody drug conjugate of claim 1, wherein n is 2, 4, 6, or 8.

26. The antibody drug conjugate of claim 25, wherein n is 2.

27. The antibody drug conjugate of claim 25, wherein n is 4.

28.-31. (canceled)

32. The antibody drug conjugate of claim 1, wherein the antibody comprises a heavy chain variable region set forth as SEQ ID NO: 5 and a light chain variable region set forth as SEQ ID NO: 6.

33.-34. (canceled)

35. The antibody drug conjugate of claim 1, wherein the antibody comprises a heavy chain set forth as SEQ ID NO: 3 and a light chain set forth as SEQ ID NO: 4.

36. A pharmaceutical composition comprising the antibody drug conjugate of claim 1 and a pharmaceutically acceptable carrier.

37. A method of treating a condition selected from the group consisting of inflammatory bowel disease (IBD), systemic lupus erythematosus (SLE), multiple sclerosis, rheumatoid arthritis, Sjogren's syndrome, and Hidradenitis suppurativa (HS), in a subject in need thereof, comprising administering an effective amount of the antibody drug conjugate of claim 1 to the subject.


SEQUENCE LISTING (13 sequences)

SEQ ID NO: 1 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 100 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCV

SEQ ID NO: 3 - protein, 446 aa, Artificial Sequence
  Description of Artificial Sequence Synthetic polypeptide
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 4 - protein, 220 aa, Artificial Sequence
  Description of Artificial Sequence Synthetic polypeptide
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 116 aa, Artificial Sequence
  Description of Artificial Sequence Synthetic polypeptide
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSS

SEQ ID NO: 6 - protein, 113 aa, Artificial Sequence
  Description of Artificial Sequence Synthetic polypeptide
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIK

SEQ ID NO: 7 - protein, 10 aa, Artificial Sequence
  Description of Artificial Sequence Synthetic peptide
GFTFSDYGMN

SEQ ID NO: 8 - protein, 17 aa, Artificial Sequence
  Description of Artificial Sequence Synthetic peptide
YISSGRGNIYYADTVKG

SEQ ID NO: 9 - protein, 7 aa, Artificial Sequence
  Description of Artificial Sequence Synthetic peptide
SWGYFDV

SEQ ID NO: 10 - protein, 17 aa, Artificial Sequence
  Description of Artificial Sequence Synthetic peptide
KSSQSLLNRGNQKNYLT

SEQ ID NO: 11 - protein, 7 aa, Artificial Sequence
  Description of Artificial Sequence Synthetic peptide
WASTRES

SEQ ID NO: 12 - protein, 9 aa, Artificial Sequence
  Description of Artificial Sequence Synthetic peptide
QNDYTYPLT

SEQ ID NO: 13 - protein, 6 aa, Artificial Sequence
  Description of Artificial Sequence Synthetic 6xHis tag
HHHHHH